SEQUENCE LISTING (6 sequences)

SEQ ID NO: 1 - DNA, 6318 bp, Homo sapiens
  PPARGC1A melanoma cell line NM_013261
tagtaagacaggtgccttcagttcactctcagtaaggggctggttgcctgcatgagtgtgtgctctgtgtcactgtggattggagttgaaaaagcttgactggcgtcattcaggagctggatggcgtgggacatgtgcaaccaggactctgagtctgtatggagtgacatcgagtgtgctgctctggttggtgaagaccagcctctttgcccagatcttcctgaacttgatctttctgaactagatgtgaacgacttggatacagacagctttctgggtggactcaagtggtgcagtgaccaatcagaaataatatccaatcagtacaacaatgagccttcaaacatatttgagaagatagatgaagagaatgaggcaaacttgctagcagtcctcacagagacactagacagtctccctgtggatgaagacggattgccctcatttgatgcgctgacagatggagacgtgaccactgacaatgaggctagtccttcctccatgcctgacggcacccctccaccccaggaggcagaagagccgtctctacttaagaagctcttactggcaccagccaacactcagctaagttataatgaatgcagtggtctcagtacccagaaccatgcaaatcacaatcacaggatcagaacaaaccctgcaattgttaagactgagaattcatggagcaataaagcgaagagtatttgtcaacagcaaaagccacaaagacgtccctgctcggagcttctcaaatatctgaccacaaacgatgaccctcctcacaccaaacccacagagaacagaaacagcagcagagacaaatgcacctccaaaaagaagtcccacacacagtcgcagtcacaacacttacaagccaaaccaacaactttatctcttcctctgaccccagagtcaccaaatgaccccaagggttccccatttgagaacaagactattgaacgcaccttaagtgtggaactctctggaactgcaggcctaactccacccaccactcctcctcataaagccaaccaagataacccttttagggcttctccaaagctgaagtcctcttgcaagactgtggtgccaccaccatcaaagaagcccaggtacagtgagtcttctggtacacaaggcaataactccaccaagaaagggccggagcaatccgagttgtatgcacaactcagcaagtcctcagtcctcactggtggacacgaggaaaggaagaccaagcggcccagtctgcggctgtttggtgaccatgactattgccagtcaattaattccaaaacagaaatactcattaatatatcacaggagctccaagactctagacaactagaaaataaagatgtctcctctgattggcaggggcagatttgttcttccacagattcagaccagtgctacctgagagagactttggaggcaagcaagcaggtctctccttgcagcacaagaaaacagctccaagaccaggaaatccgagccgagctgaacaagcacttcggtcatcccagtcaagctgtttttgacgacgaagcagacaagaccggtgaactgagggacagtgatttcagtaatgaacaattctccaaactacctatgtttataaattcaggactagccatggatggcctgtttgatgacagcgaagatgaaagtgataaactgagctacccttgggatggcacgcaatcctattcattgttcaatgtgtctccttcttgttcttcttttaactctccatgtagagattctgtgtcaccacccaaatccttattttctcaaagaccccaaaggatgcgctctcgttcaaggtccttttctcgacacaggtcgtgttcccgatcaccatattccaggtcaagatcaaggtctccaggcagtagatcctcttcaagatcctgctattactatgagtcaagccactacagacaccgcacgcaccgaaattctcccttgtatgtgagatcacgttcaagatcgccctacagccgtcggcccaggtatgacagctacgaggaatatcagcacgagaggctgaagagggaagaatatcgcagagagtatgagaagcgagagtctgagagggccaagcaaagggagaggcagaggcagaaggcaattgaagagcgccgtgtgatttatgtcggtaaaatcagacctgacacaacacggacagaactgagggaccgttttgaagtttttggtgaaattgaggagtgcacagtaaatctgcgggatgatggagacagctatggtttcattacctaccgttatacctgtgatgcttttgctgctcttgaaaatggatacactttgcgcaggtcaaacgaaactgactttgagctgtacttttgtggacgcaagcaatttttcaagtctaactatgcagacctagattcaaactcagatgactttgaccctgcttccaccaagagcaagtatgactctctggattttgatagtttactgaaagaagctcagagaagcttgcgcaggtaacatgttccctagctgaggatgacagagggatggcgaatacctcatgggacagcgcgtccttccctaaagactattgcaagtcatacttaggaatttctcctactttacactctctgtacaaaaacaaaacaaaacaacaacaatacaacaagaacaacaacaacaataacaacaatggtttacatgaacacagctgctgaagaggcaagagacagaatgatatccagtaagcacatgtttattcatgggtgtcagctttgcttttcctggagtctcttggtgatggagtgtgcgtgtgtgcatgtatgtgtgtgtgtatgtatgtgtgtggtgtgtgtgcttggtttaggggaagtatgtgtgggtacatgtgaggactgggggcacctgaccagaatgcgcaagggcaaaccatttcaaatggcagcagttccatgaagacacgcttaaaacctagaacttcaaaatgttcgtattctattcaaaaggaaatatatatatatatatatatatatatatatatatatataaattaaaaaggaaagaaaactaacaaccaaccaaccaaccaaccaaccacaaaccaccctaaaatgacagccgctgatgtctgggcatcagcctttgtactctgtttttttaagaaagtgcagaatcaacttgaagcaagctttctctcataacgtaatgattatatgacaatcctgaagaaaccacaggttccatagaactaatatcctgtctctctctctctctctctctctctcttttttttttctttttccttttgccatggaatctgggtgggagaggatactgcgggcaccagaatgctaaagtttcctaacattttgaagtttctgtagttcatccttaatcctgacacccatgtaaatgtccaaaatgttgatcttccactgcaaatttcaaaagccttgtcaatggtcaagcgtgcagcttgttcagcggttctttctgaggagcggacaccgggttacattactaatgagagttgggtagaactctctgagatgtgttcagatagtgtaattgctacattctctgatgtagttaagtatttacagatgttaaatggagtatttttattttatgtatatactatacaacaatgttcttttttgttacagctatgcactgtaaatgcagccttcttttcaaaactgctaaatttttcttaatcaagaatattcaaatgtaattatgaggtgaaacaattattgtacactaacatatttagaagctgaacttactgcttatatatatttgattgtaaaaacaaaaagacagtgtgtgtgtctgttgagtgcaacaagagcaaaatgatgctttccgcacatccatcccttaggtgagcttcaatctaagcatcttgtcaagaaatatcctagtcccctaaaggtattaaccacttctgcgatatttttccacattttcttgtcgcttgtttttctttgaagttttatacactggatttgttaggggaatgaaattttctcatctaaaatttttctagaagatatcatgattttatgtaaagtctctcaatgggtaaccattaagaaatgtttttattttctctatcaacagtagttttgaaactagaagtcaaaaatctttttaaaatgctgttttgttttaatttttgtgattttaatttgatacaaaatgctgaggtaataattatagtatgatttttacaataattaatgtgtgtctgaagactatctttgaagccagtatttctttcccttggcagagtatgacgatggtatttatctgtattttttacagttatgcatcctgtataaatactgatatttcattcctttgtttactaaagagacatatttatcagttgcagatagcctatttattataaattatgagatgatgaaaataataaagccagtggaaattttctacctaggatgcatgacaattgtcaggttggagtgtaagtgcttcatttgggaaattcagcttttgcagaagcagtgtttctacttgcactagcatggcctctgacgtgaccatggtgttgttcttgatgacattgcttctgctaaatttaataaaaacttcagaaaaacctccattttgatcatcaggatttcatctgagtgtggagtccctggaatggaattcagtaacatttggagtgtgtattcaagtttctaaattgagattcgattactgtttggctgacatgacttttctggaagacatgatacacctactactcaattgttcttttcctttctctcgcccaacacgatcttgtaagatggatttcacccccaggccaatgcagctaattttgatagctgcattcatttatcaccagcatattgtgttctgagtgaatccactgtttgtcctgtcggatgcttgcttgattttttggcttcttatttctaagtagatagaaagcaataaaaatactatgaaatgaaagaacttgttcacaggttctgcgttacaacagtaacacatctttaatccgcctaattcttgttgttctgtaggttaaatgcaggtattttaactgtgtgaacgccaaactaaagtttacagtctttctttctgaattttgagtatcttctgttgtagaataataataaaaagactattaagagcaataaattatttttaagaaatcgagatttagtaaatcctattatgtgttcaaggaccacatgtgttctctattttgcctttaaatttttgtgaaccaattttaaatacattctcctttttgccctggattgttgacatgagtggaatacttggtttcttttcttacttatcaaaagacagcactacagatatcatattgaggattaatttatcccccctacccccagcctgacaaatattgttaccatgaagatagttttcctcaatggacttcaaattgcatctagaattagtggagcttttgtatcttctgcagacactgtgggtagcccatcaaaatgtaagctgtgctcctctcatttttatttttatttttttgggagagaatatttcaaatgaacacgtgcaccccatcatcactggaggcaaatttcagcatagatctgtaggatttttagaagaccgtgggccattgccttcatgccgtggtaagtaccacatctacaattttggtaaccgaactggtgctttagtaatgtggatttttttcttttttaaaagagatgtagcagaataattcttccagtgcaacaaaatcaattttttgctaaacgactccgagaacaacagttgggctgtcaacattcaaagcagcagagagggaactttgcactattggggtatgatgtttgggtcagttgataaaaggaaaccttttcatgcctttagatgtgagcttccagtaggtaatgattatgtgtcctttcttgatggctgtaatgagaacttcaatcactgtagtctaagacctgatctatagatgacctagaatagccatgtactataatgtgatgattctaaatttgtacctatgtgacagacattttcaataatgtgaactgctgatttgatggagctactttaagatttgtaggtgaaagtgtaatactgttggttgaactatgctgaagagggaaagtgagcgattagttgagcccttgccgggccttttttccacctgccaattctacatgtattgttgtggttttattcattgtatgaaaattcctgtgattttttttaaatgtgcagtacacatcagcctcactgagctaataaagggaaacgaatgtttcaaatcta

SEQ ID NO: 2 - DNA, 10642 bp, Homo sapiens
  PPARGC1B melanoma cell line NM_133263
ctcctccctcctcccttgctcgctcgctggctccctccccccgggccggctcggcgttgactccgccgcacgctgcagccgcggctggaagatggcggggaacgactgcggcgcgctgctggacgaagagctctcctccttcttcctcaactatctcgctgacacgcagggtggagggtccggggaggagcaactctatgctgactttccagaacttgacctctcccagctggatgccagcgactttgactcggccacctgctttggggagctgcagtggtgcccagagaactcagagactgaacccaaccagtacagccccgatgactccgagctcttccagattgacagtgagaatgaggccctcctggcagagctcaccaagaccctggatgacatccctgaagatgacgtgggtctggctgccttcccagccctggatggtggagacgctctatcatgcacctcagcttcgcctgccccctcatctgcaccccccagccctgccccggagaagccctcggccccagcccctgaggtggacgagctctcactgctgcagaagctcctcctggccacatcctacccaacatcaagctctgacacccagaaggaagggaccgcctggcgccaggcaggcctcagatctaaaagtcaacggccttgtgttaaggcggacagcacccaagacaagaaggctcccatgatgcagtctcagagccgaagttgtacagaactacataagcacctcacctcggcacagtgctgcctgcaggatcggggtctgcagccaccatgcctccagagtccccggctccctgccaaggaggacaaggagccgggtgaggactgcccgagcccccagccagctccagcctctccccgggactccctagctctgggcagggcagaccccggtgccccggtttcccaggaagacatgcaggcgatggtgcaactcatacgctacatgcacacctactgcctcccccagaggaagctgcccccacagacccctgagccactccccaaggcctgcagcaacccctcccagcaggtcagatcccggccctggtcccggcaccactccaaagcctcctgggctgagttctccattctgagggaacttctggctcaagacgtgctctgtgatgtcagcaaaccctaccgtctggccacgcctgtttatgcctccctcacacctcggtcaaggcccaggccccccaaagacagtcaggcctcccctggtcgcccgtcctcggtggaggaggtaaggatcgcagcttcacccaagagcaccgggcccagaccaagcctgcgcccactgcggctggaggtgaaaagggaggtccgccggcctgccagactgcagcagcaggaggaggaagacgaggaagaagaggaggaggaagaggaagaagaaaaagaggaggaggaggagtggggcaggaaaaggccaggccgaggcctgccatggacgaagctggggaggaagctggagagctctgtgtgccccgtgcggcgttctcggagactgaaccctgagctgggcccctggctgacatttgcagatgagccgctggtcccctcggagccccaaggtgctctgccctcactgtgcctggctcccaaggcctacgacgtagagcgggagctgggcagccccacggacgaggacagtggccaagaccagcagctcctacggggaccccagatccctgccctggagagcccctgtgagagtgggtgtggggacatggatgaggaccccagctgcccgcagctccctcccagagactctcccaggtgcctcatgctggccttgtcacaaagcgacccaacttttggcaagaagagctttgagcagaccttgacagtggagctctgtggcacagcaggactcaccccacccaccacaccaccgtacaagcccacagaggaggatcccttcaaaccagacatcaagcatagtctaggcaaagaaatagctctcagcctcccctcccctgagggcctctcactcaaggccaccccaggggctgcccacaagctgccaaagaagcacccagagcgaagtgagctcctgtcccacctgcgacatgccacagcccagccagcctcccaggctggccagaagcgtcccttctcctgttcctttggagaccatgactactgccaggtgctccgaccagaaggcgtcctgcaaaggaaggtgctgaggtcctgggagccgtctggggttcaccttgaggactggccccagcagggtgccccttgggctgaggcacaggcccctggcagggaggaagacagaagctgtgatgctggcgccccacccaaggacagcacgctgctgagagaccatgagatccgtgccagcctcaccaaacactttgggctgctggagaccgccctggaggaggaagacctggcctcctgcaagagccctgagtatgacactgtctttgaagacagcagcagcagcagcggcgagagcagcttcctcccagaggaggaagaggaagaaggggaggaggaggaggaggacgatgaagaagaggactcaggggtcagccccacttgctctgaccactgcccctaccagagcccaccaagcaaggccaaccggcagctctgttcccgcagccgctcaagctctggctcttcaccctgccactcctggtcaccagccactcgaaggaacttcagatgtgagagcagagggccgtgttcagacagaacgccaagcatccggcacgccaggaagcggcgggaaaaggccattggggaaggccgcgtggtgtacattcaaaatctctccagcgacatgagctcccgagagctgaagaggcgctttgaagtgtttggtgagattgaggagtgcgaggtgctgacaagaaataggagaggcgagaagtacggcttcatcacctaccggtgttctgagcacgcggccctctctttgacaaagggcgctgccctgaggaagcgcaacgagccctccttccagctgagctacggagggctccggcacttctgctggcccagatacactgactacgattccaattcagaagaggcccttcctgcgtcagggaaaagcaagtatgaagccatggattttgacagcttactgaaagaggcccagcagagcctgcattgataacagccttaaccctcgaggaatacctcaatacctcagacaaggcccttccaatatgtttacgttttcaaagaaatcaagtatatgaggagagcgagcgagcgtgagagaacacccgtgagagagacttgaaactgctgtcctttaaaaaaaaaaaaaatcaatgtttacattgaacaaagctgcttctgtctgtgagtttccatggtgttgacgttccactgccacattagtgtcctcgcttccaacgggttgtcccgggtgcacctcgaagtgccgggtccgtcacccatcgccccttccttcccgactgacttcctctcgtagacttgcagctgtgttcaccataacatttcttgtctgtagtgtgtgatgatgaaattgttacttgtgaatagaatcaggactataaacttcatttttaattgaaaaaaaaagtatatccttaaaataatgtatttatggctcagatgtactgtgcctgggattattgtattgcttccttgattttttaactatgcactgtcatgaggtgtttgccactgagctgccctgctccccttgccagattgccctggaggtgctgggtggccgctaggctggtctgcaggaaagcgcggcctgccgtttccgggccgtatctgccaagccctgccttgtctcttactgagcaagtttggctcaaattataggagcccccatcttgtgcccagctcatgctccaagtgtgtgtctatccatttgtactcagactcttgagtaccttgtaaggaaggcggggcaagctgcatcattcctgttttccaggggaggctggcagctcctcaagaggcgaaatgactgtgggaggtccggttaccagtgaggaggcagagcggtgacccagaccaggccttctggttcttggtcccgtgcttccgtagtagctggggtaaagacaccgtttcagggactggtagaggtgagttcggctaaattgggcaccgggctagaagcctaagggctcattttaggggttacattaggtgttgattcaccagcatcaggtgaattcaagccctggcatgtgtcttggatgcaccatcagctttgatcctgagtggtcctgcggtttgtctgtgcctgtggacacactgtcagaacttcagtgacacccctggcagcggtacagacaggtggtctgggagcagtcatcttttttgggccagccaccagcccatcctactccctcaggtagtccttcgtctttaccttgtccttgtctgtaaagttgttttggtggctggggcaggggagccaggaggagggagtgaaggttgggaatagataggacaatctcctagctctcctccaattgagaaaacactccaattgggctttgctttaaactttgtgttcttaagtgatgtcaaagccatttccagcttaatgttctgtgggtaccttgggggccattcatgcagggagcatggccaggcagggtatgagtacattgtttctgatttctttcatacatcagggttcctcgggaaatttttgtattttttttttaagtcctgctgctttaaaaatttgaaagtggctcattaaactaaacaggctaatgtaatttgttgcttatgccaagcctagactgttgagaattgacgtttttaaagattatcaaatacctcagtaggtaaaatgagcccatgatcttccactgagtggtgagcatactcccagcccatggacaaggccggaagagacaggctttagtaggggtagggaatttgaactgttgtgtgtcacagcagttgacctctctggactccaatttcctttcctgtgaaatgaactgattagacatgtttcaacattgttagcttctgctgaggcagtgtctagcccaagatggcaaatacatagctcatgtgccactactcccacctccttgaccaatacagacataactaatcaatcacaccactcaggttccctgagcctggatgtgctataagaatcctgaaatcagtgctctggtaagtcattactaattgattagagttcaatctatttgacatcttgggctaatctttggaaggtttccaacaatcacacaaaaccatatgctggctgggtttcatgctggcctatccctgtctgtgatgttccgttccatgagagaaaactcccctaatgctattccatggcgtaacactcccaatactattttgacgcccacgtccccttgcagagggtgcagggggcggtagacgaatgacagacaggaacatatttggggaaggcagggcttaggaagatggaccaaaaagggacttcccacagcacagacctgatcattcggatttcctctttagctattcactgcctagcacatagtaggcacacaataaatgattatggaatgggataaaatttagatctttctgctgcctccactaagttaagtcctgatttacatcaaggagagaactgagataggaaagaacactagattccaagtctggagagttgggggagtccagattctaccaagaatttcctttgtaactttggtaagtcccttttactccctggcaccccggtgtgctgaaaggagttggtccatatatgatctcttagcccctcctatttgcttcttccttgattgctcttggtcaaagggtcagccttgggctggtgatactttagagtaaagaaatggagagttttagcaaaggaccagtctgtccctccctgctttggggtcagctaaagctgtcctttcatgtcagattaacctaggacacttgtagttagcttagacgttggcccttgagcagagacctgagcgtggcattgggacatgacatacctaaagtcagggctaggggacgctgcctgccaagggcatcgagtagtctctacttgctatcccgtacataaaatgctacaagttctaaaatttaccgaccctgcagacaacctctatcccgaaggactcattcggtgctgtgtattatttagggcaactccaaggtctattcagaaaaacgagtgaaccttggtctctttcccaccaaattgaggagtaacccagagggagcagctgccattggcaaccatctcgttgtagctctgtcctagtgtttgctcttgatgatgtttacatgtgatcgccataaagcttgctgtagactgtgtcgatagccgcccgcacagggcaggtcgtactgtccgtttctgtgccgtgctggtgttttccaaaaatgtctgatccaaccactaagtggaattcttccatctccttcctcagtctgtacaaggctgaatcagaatccccattctcgggggctctggttaccgaaggaaaatgcatcaaagagttaaagaatatgagtggatggagtgcagctaaggcccccaccccctgctccgtcacaacttgccccctcaaccaaaaagctgctttgagtcaaaaagcacccataagatacctgcatctgccttgaaatcttgcagcatggagtgtcatatgtactcaggagagaggcagggctttgcgggcaggagaaggaagggaggaatgctctgagctgcaaagacccagtactcaagttctgacgtgggaggagatgcagtgagacgtctcttgttgcctaaagcctgttcctgttggttttcttagagtgatttctcctagacatgtgcagtaggcccactggggctgctgtgcagtggtgagtaaaagggcagggaaggcatggacagcctggtccttctgcatggacagctcagtccatggcccatcccaggtatagagttcagttaatcccatttgagcctgcagcttaagagatggctcatcctaactgtgaagcaaaatcagccccagaggatgtattgatctgactcactgatgtcaaaattgcagtatttttttagcatttgagatttagcagctgccttcagtttggggttacccacatcccagcatcagatatgattaaggaaagaaattggatgtacaacagcaaagaaagtgaatgtcatggtttccctggccaaagaagagggaccctgtcatccttaccaatggggaagaagaaaactagtgcatgtgcaatatgtcaaagttagtcccctagtccctgaggggtttttacacacagatgggctccaggtctgctcgtcaagtttggaggtaccgggtaaatggaggggagctgcagagttggaaacccacatgcatggatgtgtccttggcccagaaccaccatgggatgggggaggccctgagccggctacaagacacccaggaagtaggcaaaggctgactttgcattaaacaataaaagcactttgagaaaaccccaacacttcagcctgggtccgtgtttctacactggaaaatacgagtctcctttggctgtgtgaagtgatcttctagagactgggacagggagtttgggaatggggctgctgtcaggtaggagagagcagagatgcctttggagatgtcagcagcaggagagccagtgctggggccaaccctttgctggccttttgttggaagcccttgaaacagggagccatgggtttagatcttggtacctacctttacagaaagatgaaaacagcccagctgagtgaaatgagtttgtagagtaagtcacttaactgtaagccatctcagaatcagaaaccctaatgtttcttacttgctatgtgaccttgggcccctgtttcctcatctaccaaatgagaatgttgaatatgagcattaaagtccctttcacctctgagaggctcagatccccaaccaggagcattgggaatccatcactcctccttgaaactgattccattctctgacttgacccagctcctgttcagggtgagggttctctgcaagaaccaaccagcagtaggttcaatcccactgtgtcctggctgagttgccttatccaagaagaccagctccccgggacagatctaagccatagtttctagtggggacagtaaggaattaaacccccaacttggctaggtaacgatgtcaaatctcacattaaccttgtctttgtccccactggatagctgttaatccgaatgttgtgaccatttggctgtttctctcttgttctcagacaatactagcaatacactttttttttttttttttaaagaaaaacagcttaggagcttttcacacatttctttcaaatgattgtaaaacatatggggcaacaggaggcattgatcgcgctgcatatgtttagggcagcttttgttttttgtttctttaatggtatagcagcagtgactgagccttcgtgattcctggggacagcttttcagatactctgtttcatcagtatgctttgcacatccggaaggagtacaaaaatccaactgcccaaatttggggcttggaaaataggttttataggtggtcggtccctgggctgtgcaacaactcctcaaagaggggtttatataactagaacccccctgggctgtatttttggtcaaaggagtctccaaggcggcttacaaaagcttcctttttcacttgaccacccttgctcattggttacttgtgaagggaattggtcagtttccacctcagcactttgccttatcaacatgcggtcgccatctagtggccaaaggttgtctccaccagctacccagatggaaggcaaataaatcctttcggccaccctgctgtccatcgtgaactttgggaatgaaatataatggcctgaacgaactgcctttgtgttcagagatcagtgcaacactagggtcagaagactccagaagcagccacttagtagactctcacgcagaactgagaaatgcactagctgtcctgtgggcagaagagacaggagtggaccaggagaggtccaggtgcccgggaagggtttactgtaactgcaatactggcagcccagctgctgaccttgttaagtaaacctttgctgggtggtccgaattctgccctcaaggcaagataagaagttgggtgtaaggattttgtggggggcctggccatgatctttgatatgatccccgaatagccaaatagttttttttgttcaattttttgtttctgtattttgtatttttaaaatcttgtcaaatgtttttgtgttaggaataaaaagtcataaactattcccaactttgtttcttgagggatgttctgattccaatggaaacaggtgggaaatctcaaggggagcgtggacaaggtggtatgtgcagcaggggaatagactgcttggatttccaaatggtttctggggaagatgaccatccagaagtccagcttagtgcagtctgctctggaattcacacccaccccctcgcctccttgtgccatgttgttagcattggcttggagcatctgcttcttccagaggcagctgctaatgttgaaaccaacacgagccctctccccaaccccaggtttctaaagaaggtgtctgtagccagccttaatcaactgggcaaggtggtccctatggtcctttccagcatttccaaatcttggactcaaattattttctcttggtgtgaccacacagcctagagaattctgagcaataggagccagggctttccctgactctgcgacagggtcaaaccaaggaatggctaaacctgtgaggttttgtcatccccgggggtactactgtagggggcattatttattaggaagcttaacaaggtaactacggcctgagtgcgtgagtgtaaggctgtgtttgtggtgggggtgtgtgtgtgtgtatctgtgcacacatacacacgtctgtgcctgtgtgtgtgtgtttgtgtgtgtgtgtgtgtgtggaattacattgatgcatttattgagaaaggtgcaagaatttcacctacacagagggacacatctgctttgttatttataatagaaagctaaattttaattttttaaaggacactgctaatgattgagaatcaagtttttagttttgctattttttttaattggtagaggatttttatatattttttccattttgttgggttgtgtccttatttatataaatactttatccgtaagaggcaaggaggaaaccttctttgcttttacatattgtggttgtcatcgtccctattttatttctggtgtgatttctctgtcttaccttctaaatgagaaaatgttttcttgtatttgtacattgtcagattctatagtttcctagataatttaaccaaattgctctatgtattattattctgtgagtataaagttctattttaatgtctgtaaatacttcagaactggcttcttttctcaaactcccactgtggggttattgtttacatcacagaaactgtagaatctctatgctcatgtactgtaaatagtgaagtgatctgcttataaataaacttaacaaatacactatggagattaaaaacaaaataccacccacaaaaaaaaaaaaaaaa

SEQ ID NO: 3 - DNA, 4815 bp, Homo sapiens
  MITF Isoform NM_198159
gtaaactccccgcgctggggcgggcggccgcgagccggcgagcgggcagagctcggcactgcgccggggcgcacggctcgggggacccaggcccagctaccttccctccgcccccgggctctgttctcactttccagcagtggaaggacgggaagcgggagccatgcagtccgaatcggggatcgtgccggatttcgaagtcggggaggagtttcatgaagagcccaaaacctattacgaactcaaaagtcaaccgctgaagagcagcagttccgccgagcatcctggggcctccaagcctccgataagctcctccagtatgacatcacgcatcttgctacgccagcaactcatgcgtgagcagatgcaggagcaggagcgcagggagcagcagcagaagctgcaggcggcccagttcatgcaacagagagtgcccgtgagtcagacaccagccataaacgtcagtgtgcccaccacccttccctctgccacgcaggtgccgatggaagtccttaaggtgcagacccacctcgaaaaccccaccaagtaccacatacagcaagcccaacggcagcaggtaaagcagtacctttctaccactttagcaaataaacatgccaaccaagtcctgagcttgccatgtccaaaccagcctggcgatcatgtcatgccaccggtgccggggagcagcgcacccaacagccccatggctatgcttacgcttaactccaactgtgaaaaagagggattttataagtttgaagagcaaaacagggcagagagcgagtgcccaggcatgaacacacattcacgagcgtcctgtatgcagatggatgatgtaatcgatgacatcattagcctagaatcaagttataatgaggaaatcttgggcttgatggatcctgctttgcaaatggcaaatacgttgcctgtctcgggaaacttgattgatctttatggaaaccaaggtctgcccccaccaggcctcaccatcagcaactcctgtccagccaaccttcccaacataaaaagggagctcacagagtctgaagcaagagcactggccaaagagaggcagaaaaaggacaatcacaacctgattgaacgaagaagaagatttaacataaatgaccgcattaaagaactaggtactttgattcccaagtcaaatgatccagacatgcgctggaacaagggaaccatcttaaaagcatccgtggactatatccgaaagttgcaacgagaacagcaacgcgcaaaagaacttgaaaaccgacagaagaaactggagcacgccaaccggcatttgttgctcagaatacaggaacttgaaatgcaggctcgagctcatggactttcccttattccatccacgggtctctgctctccagatttggtgaatcggatcatcaagcaagaacccgttcttgagaactgcagccaagacctccttcagcatcatgcagacctaacctgtacaacaactctcgatctcacggatggcaccatcaccttcaacaacaacctcggaactgggactgaggccaaccaagcctatagtgtccccacaaaaatgggatccaaactggaagacatcctgatggacgacaccctttctcccgtcggtgtcactgatccactcctttcctcagtgtcccccggagcttccaaaacaagcagccggaggagcagtatgagcatggaagagacggagcacacttgttagcgaatcctccctgcactgcattcgcacaaactgcttcctttcttgattcgtagatttaataacttacctgaaggggttttcttgataattttcctttaatatgaaattttttttcatgctttatcaatagcccaggatatattttatttttagaattttgtgaaacagacttgtatattctattttacaactacaaatgcctccaaagtattgtacaaataagtgtgcagtatctgtgaactgaattcaccacagactttagctttctgagcaagaggattttgcgtcagagaaatgtctgtccatttttattcaggggaaacttgatttgagatttttatgcctgtgacttccttggaaatcaaatgtaaagtttaattgaaagaatgtaaagcaaccaaaaagaaaaaaaaaaagaaagaaagaggaaaagaaatccatactaacccttttccattttataaatgtattgattcattggtactgccttaaagatacagtacccctctagctttgtttagtctttatactgcaaactatttaaagaaatatgtattctgtaaaagaaaaaaaaaatgcggccttttcatgaggatcgtctggttagaaaacataactgataccaaccgaaactgaagggagttagaccaaggctctgaaatataaagtctaatcttgctctcttttattctgtgctgttacagttttcttcatcaatgagtgtgatccagtttttcataagatattttattttgaaatggaaattaatgtcctctcaaagtaaaatattgaggagcactgaaagtatgttttactttttttttattttatttttgcttttgataagaaaaccgaactgggcatatttctaattggctttactatttttatttttaaattatgttttactgttcatttgatttgtacagattctttattatcattgttcttttcaatatatttgtattaatttgtaagaatatgcatcttaaaatggcaagttttccatatttttacaactcactggtggttttccgcattctttgtacacccatgaaagaaaacttttatgcaaggtcttgcatttaaaagacagctttgcgaatattttgtaaattacagtctcactcagaactgtttttggacacatttaaggtgtagtattaataggttaaaaccaggctttctagaaagaataaacttacatatttatttttaggacatgaaaatagcaatattcttggagattgataaccatagcattaatacgcccattatggtcatttaaattggggtttatttcagcaaacttgttgaatttatttttaagaaagaaatactgtattgggaagttactgttacttgataacaatgttttaacaagaagcaatgttataaagttagtttcagtgcattatctacttgtgtagtcctatgcaataacagtagtgttacatgtatcaagcctagatgttttatacagatgccatatagtgttatgagccaggctgttgaatggaatttctcagtagcagcctacaactgaatagcaagtggcataaagcatatccattcagaatgaagtgccttaaatatagcagtagtcttttttggactagcactgactgaactgtaatgtaggggaaagtttcatgatggtatctatagtcaagacgaacatgtagcatggtgcctatgtagacaatataagagcttccaattttccttcagatatttttaatattaaatatattttagtgacagagtgccaacttctttcatcaggaaaccttattcaggagggtttttaaaaagtgtttaaatgtcaaatgtgaattggtgatgggtgatggagggttcagagaggagtgatcgtcagatgtgtgaatggacggtttaggtgaaaataatcaactgcatagttcccatgcacgctgggcaatgagaatccttggaaacattggtgatgctatcagttttatagctttatttcttaagggggtagggaaaattagttcccattctttcaacccccttaactgtatagctcttttcctagaatagtgacgcaaatctgcatgaacagctaattgtaccatagtgttcattgatacaatcatagcattgtctatttttctcttcatatttatatgggggggagggcgctggatgcaaaagttgaagatcgtgatgctatgatgttagttttccttagctgattttgagggtttttaaaaataaagcaaggttgactaacctacggccacgggaacaggaccatggttaagcaaccatatagaaagctttgttgaaagaaagtatggcatcttgtaccactgccctgactgtcacaactcctaaccttgccattgcctgcctccccctccccttctccttaagagacaatttctgcaggtggcaggtgagcaagcccaggagaatgctgcaatcttgggggtggttttatttatttcttttttgccaaatagagtgtggattcatttcaggggctagctaagccaagaggcagtggtttgggcttgttgtttgtaacaagaaaatgatccacaccactcccccgattcccgggtgcagaattgtaactcggggttgggcctctatatggagtgaccaaaatgccaaaattgtccatctgcctctgagtagggcaatggaaataccaaaccttctgactttgccaaaaagcatacaagcaacctggtcatacataggatgacaaaattctttctggttgtttttaaacaataaagcaataagaacaaatacaatacataggaagttaaaagcacaaaggaatgaacttattaatatttttgaaaaatgcactgggaaaaagttgatgtcaataacagtataaaacagccctatttcttgataaaaaatgacaaatgactgtctcttgcggatgcttggtactgtaatgttaataatagtcacctgctgttggatgcagcaataatttctgtatggtccatagcactgtatattatggatcgatattaatgtatccaatgaaataatcgacttgttcttgatagcctcattaaagcatttggtttttcacatag

SEQ ID NO: 4 - protein, 798 aa, Homo sapiens
  PPARGC1A protein Q9UBK2
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 5 - protein, 984 aa, Homo sapiens [sequence omitted]

SEQ ID NO: 6 - protein, 526 aa, Homo sapiens
  MITF protein NP-937802
MQSESGIVPDFEVGEEFHEEPKTYYELKSQPLKSSSSAEHPGASKPPISSSSMTSRILLRQQLMREQMQEQERREQQQKLQAAQFMQQRVPVSQTPAINVSVPTTLPSATQVPMEVLKVQTHLENPTKYHIQQAQRQQVKQYLSTTLANKHANQVLSLPCPNQPGDHVMPPVPGSSAPNSPMAMLTLNSNCEKEGFYKFEEQNRAESECPGMNTHSRASCMQMDDVIDDIISLESSYNEEILGLMDPALQMANTLPVSGNLIDLYGNQGLPPPGLTISNSCPANLPNIKRELTACIFPTESEARALAKERQKKDNHNLIERRRRFNINDRIKELGTLIPKSNDPDMRWNKGTILKASVDYIRKLQREQQRAKELENRQKKLEHANRHLLLRIQELEMQARAHGLSLIPSTGLCSPDLVNRIIKQEPVLENCSQDLLQHHADLTCTTTLDLTDGTITFNNNLGTGTEANQAYSVPTKMGSKLEDILMDDTLSPVGVTDPLLSSVSPGASKTSSRRSSMSMEETEHTC

(19) United States
(12) Patent Application Publication
(10) Pub. No.: US 2018/0164317 A1
(43) Pub. Date: Jun. 14, 2018

(54) METHOD FOR STRATIFICATION OF MELANOMA PATIENTS BY DETERMINATION OF OXYGEN CONSUMPTION, PPARGC1A, PPARGC1B AND MITF LEVELS

(71) Applicant: Bayer Pharma Aktiengesellschaft, Berlin (DE)

(72) Inventors: Bernard HAENDLER, Berlin (DE); Kathy Ann GELATO, Berlin (DE); Laura SCHÖCKEL, Berlin (DE); Melanie HEROULT, Düsseldorf (DE)

(73) Assignee: Bayer Pharma Aktiengesellschaft, Berlin (DE)

(21) Appl. No.: 15/577,625

(22) PCT Filed: May 25, 2016
(86) PCT No.: PCT/EP2016/061818

§ 371 Date: Nov. 28, 2017

(30) Foreign Application Priority Data
May 28, 2015 (EP) 15169617.6

(51) Int. Cl. G01N 33/574; A61K 31/551; A61K 31/497; A61K 31/381; A61K 31/17; A61K 31/215; A61P 35/00

(52) U.S. Cl. CPC G01N 33/5743; A61K 31/551; A61K 31/497; A61P 35/00; A61K 31/17; A61K 31/215; A61K 31/381

(57) ABSTRACT

The present invention refers to a method and kit for stratification of melanoma patients by determining the OCR and levels of PPARGC1A, PPARGC1B and MITF RNA, derived cDNA, or corresponding protein. Especially, the invention is related to stratification kits to determine whether a patient with melanoma will respond to treatment with a BET inhibitor. In a further aspect, the invention is related to the use of a BET inhibitor for the treatment of melanoma in a patient by stratifying a sample of body fluid or tumor tissue in vitro and determining whether a patient suffering from melanoma will respond to treatment with a BET inhibitor.

The present invention refers to a method and kit for stratification of melanoma patients by determining the oxygen consumption in the tumor and levels of PPARGC1A, PPARGC1B and MITF RNA or protein. Especially, the invention is related to stratification kits to determine whether a patient with melanoma will respond to treatment with an inhibitor of bromodomain and extraterminal domain (BET) proteins. In a further aspect, the invention is related to the use of a BET inhibitor for the treatment of melanoma in a patient by stratifying a sample of body fluid or tumor tissue in vitro and determining whether a patient suffering from melanoma will respond to treatment with a BET inhibitor.

PPARGC1A stands for peroxisome proliferator-activated receptor gamma, co-activator 1-alpha. It is also named PGC1-α, PGC-lalpha, PGC-1(alpha), LEM6, PGC-1v, PGC1, PGC la or PGC1A.

In humans, the protein is encoded by the PPARGC1A gene (Gene ID 10891, (http://www.ncbi.nlm.nih.gov/gene/10891) and has the NCBI reference sequence identifier NM_013261 (www.ncbi.nlm.nih.gov/nuccore/NM_013261.3).

PPARGC1A is a regulator of mitochondrial biogenesis and function, and is involved in energy metabolism (Z. Wu et al., 1999, Cell, 1999, 98: 115-124; C. Liu and J. D. Lin, Acta Biochim. Biophys. Sin., 2011, 43:248-257). It binds to PPARgamma, thus promoting the interaction with different transcription factors. PPARGC1A is a transcriptional co-activator with a central function in mitochondrial biogenesis in cells. It controls oxidative metabolism and the elevated oxidative metabolism associated with increased PPARGC1A activity could be accompanied by an increase in reactive oxygen species that are generated by mitochondria as part of the incomplete reduction of molecular oxygen in the mitochondrial electron chain (S. Austin and J. St-Pierre, J. Cell. Sci. 2012, 125, 4963-4971).

PPARGC1B stands for peroxisome proliferator-activated receptor gamma, co-activator 1 beta. It is also named PERC, ERRL1, PGC1B or PGC-1(beta). In humans the protein is encoded by the PPARGC1B gene (Gene ID 133522; http://www.ncbi.nlm.nih.gov/gene/133522) and has the NCBI sequence identifier NM_0133263 (www.ncbi.nlm.nih.gov/nuccore/NM_133263.3).

PPARGC1B regulates the activity of several transcription factors, including nuclear receptors. It plays an important role in the control of energy expenditure and in non-oxidative glucose metabolism (C. Liu and J. D. Lin, Acta Biochim. Biophys. Sin., 2011, 43:248-257).

MITF stands for microphthalmia-associated transcription factor. It is also named CMM8, MI, WS2, WS2A, bHLHe32. MITF is a protein that in humans is encoded by the MITF gene (Gene ID 4286, http://www.ncbi.nlm.nih.gov/gene/4286) and has the NCBI sequence identifier NM_198159 (www.ncbi.nlm.nih.govinuccore/NM_198159.2).

MITF is a transcription factor with a role in lineage-specific pathway regulation. In melanocytes, it is essential for the synthesis of melanin. It is also involved in the regulation of genes that control invasion, migration and metastasis (M. L. Hartmann and M. Czyz, Cell. Mol. Life Sci., 2015, 72:1249-1260). Its expression is repressed by the Brn-2 transcription factor (J. Goodall, Cancer Res., 2008, 68:7788-7794).

Tumor cells utilize two main pathways for energy production, glycolysis followed by lactate fermentation in the cytosol, and oxidative phosphorylation in mitochondria. Paradoxically, glycolytic rates and lactate production in tumors are often elevated in tumors, even though this process is far less efficient when it comes to energy production compared to oxidative phosphorylation. This phenomenon is called the Warburg effect or aerobic glycolysis (O. Warburg, Science, 1956, 123:309-314). However, recent research shows that mitochondrial metabolism and oxidative phosphorylation are also required for tumor cell survival in many cancers (V. Fogal et al., Mol. Cell. Biol., 2010, 30:1303-1318; F. Weinberg, et al., Proc. Natl Acad. Sci. USA, 2010, 107:8788-8793). Importantly, a subset of melanomas is critically dependent on oxidative phosphorylation whereas other melanomas rely mainly on glycolysis (B. Vazquez et al., Cancer Cell, 2013, 23:287-301). Higher respiratory capacity is usually linked to higher number of mitochondria per cell or higher level of respiratory chain protein complexes (D. C. Wallace, Nat. Rev. Cancer, 2012, 12, 685-698; L. M. Phan et al., Cancer Biol. Med., 2014, 11, 1-19). Dependence on elevated oxidative phosphorylation is paralleled by PPARGC1A expression, which itself is driven by MITF. High PPARGC1A expression in melanoma samples is furthermore paralleled by elevated expression of ZNF749, DYNC1, C1ORF115, VEPH1, KRTAP19-3, QPCT, C9ORF93, SLC11A2, GHR, HOXA13, PPP1R1A, PRKD3, HPS4, PPM1H, TRIM63, RAB27A, EFHD1, MITF and LOC₂₈₄₈₃₇ (B. Vazquez et al., Cancer Cell, 2013, 23:287-301). Dependence on elevated oxidative phosphorylation can furthermore be evidenced by an increased basal oxygen consumption rate (OCR) in these melanoma cell lines. The dependence on oxidative phosphorylation or glycolysis is not related to BRAF, which is frequently activated by mutations in melanoma (B. Vazquez et al., Cancer Cell, 2013, 23:287-301). Importantly, treatment with an inhibitor of mutated BRAF leads to a switch from the glycolytic to the oxidative metabolism program via induction of PPARGC1A and MITF expression (R. Haq et al., Cancer Cell, 2013, 23 :302-315).

In addition, it has also been shown that diffuse large B cell lymphomas (DLBCL) can be classified in two different groups, depending on their oxidative phosphorylation or glycolysis phenotype (P. Caro et al., Cancer Cell, 2012, 22:547-560).

Targeted cancer drugs have a direct or indirect effect on one or more relevant biochemical pathways. On the other hand, it is well known that when treating patients suffering from cancer, only some of them will respond to the treatment whereas others will not. Prescribing a treatment to a patient who is unlikely to respond to it is not desirable. Thus, it would be very useful to predict whether a patient is likely or not to respond to such treatment before a drug is administered, so that non-responders would not be unnecessarily treated and that those with the best chance of benefiting from the drug are properly treated and monitored. Further, there may be varying degrees of response in patients who respond to treatment.

Stratification in the sense of the invention also means the identification of a patient or a group of patients with shared biological characteristics by using molecular, biochemical and diagnostic testing to select the optimal treatment for the patients and achieve the best possible outcome.

For example, in WO2014/026997 inhibitors are described that have an inhibitory effect on the function of the human BET family.

The human BET protein family has four members (BRD2, BRD3, BRD4 and BRDT) and each member contains two related bromodomains and one extraterminal domain (P. Filippakopoulos and S. Knapp, Nat. Rev. Drug Discov., 2014, 13:337-356; D. Gallenkamp et al., ChemMedChem, 2014, 9:438-464). The bromodomains arc protein regions that recognize acetylated lysinc residues. These acetylatcd lysincs arc often found in the N-terminal tail of histones (e.g. histone 3 or histone 4) and are characteristic features of an open chromatin structure and active gene transcription (M. H. Kuo and C. D. Allis, Bioessays, 1998, 20:615-626).

Mechanistically BET proteins play an important role in controlling transcription elongation of genes involved in cell growth and cell cycle progression (J. Shi and C. R. Vakoc, Mol. Cell., 2014, 54:728-736). They are associated with mitotic chromosomes, suggesting a role in epigenetic memory (A. Dey et al., Mol. Biol. Cell, 2009, 20:4899-4909; Z. Yang et al., Mol. Cell. Biol., 2008, 28:967-976). Further, BET proteins play an important role in various types of tumors, both hematological and solid tumors, including lymphoma and melanoma (P. Filippakopoulos and S. Knapp, Nat. Rev. Drug Discov., 2014, 13:337-356; D. Gallenkamp et al., ChemMedChem, 2014, 9:438-464).

In WO2014/026997 it is described that BET bromodomain inhibitors inhibit the proliferation of different tumor cell lines.

Recently it was described by J. Meloche et al. (American Heart Association's 2014 Scientific Sessions and Resuscitation Science Symposium, Chicago Ill., Circulation 130(-): Conference Abstract 19163 (2014), ISSN: 0009-7322 (EMBASE 2014/PUI71712520) that BRD4 signalling and metabolic disorder play an important role in coronary diseases. The authors showed that cultured human coronary artery smooth muscle cells from patients with stenosis have signs of mitochondrial dysfunction, including down-regulation of PPARGC1A, as seen in Western blot analysis. These mitochondria/metabolic abnormalities increase DNA damage signalling in human coronary arteries with stenosis, as well as Poly(ADP)ribose-polymerase-1 and BRD4 expression, in comparison to control arteries. Importantly, this is reversed following treatment with a BRD4 inhibitor, such as JQ1.

However, nothing is presently disclosed that describes PPARGC1A, PPARGC1B or MITF as stratification markers in tumors. Moreover nothing is disclosed that PPARGC1A, PPARGC1B or MITF can be used as stratification markers in melanomas, with respect to response to a BET inhibitor. Further, nothing is disclosed that monitoring oxidative phosphorylation or glycolysis in tumors or more specifically in melanomas can be used for stratifying patients with respect to an expected response upon treatment with a BET inhibitor.

The term PPARGC1A, is used in the present invention for the PPARGC1A gene (Gene ID 10891, http://www.ncbi.nlm.nih.gov/gene/10891), respectively the human protein encoded by the PPARGC1A gene (Seq. ID No. 1), as shown in FIG. 5.

The term PPARGC1B, is used in the present invention for the PPARGC1B gene (Gene ID 133522, http://www.ncbi.nlm.nih.gov/gene/133522), respectively the human protein encoded by the PPARGC1B gene (Seq. ID No. 2), as shown in FIG. 6.

The term MITF, is used in the present invention for the MITF gene (Gene ID 4286, http://www.ncbi.nlm.nih.gov/gene/4286), respectively the human protein encoded by the MITF gene (Seq. ID No. 3), as shown in FIG. 7.

It is thus an object of the present invention to find a method for the stratification of tumors, more specifically of melanomas with respect to response of a patient following treatment with a BET inhibitor.

A clear stratification marker for melanoma with respect to sensitivity or resistance to BET inhibitors has not yl)t been identified.

There is however a high need for solid and convincing data allowing a reliable stratification with regard to clinical decisions whether to treat or not to treat a cancer patient, especially a melanoma patient with a given drug.

It is thus the object of the present invention to provide a method for safe and reliable stratification that can be used to decide whether a treatment with an active pharmaceutical compound is likely to show efficacy in cancer, more specifically in melanoma patients.

It has now been found that a safe stratification is possible with the described inventive in vitro method for selecting cancer patients, more specifically melanoma patients that arc eligible for treatment with a BET inhibitor.

The invention is an in vitro stratification method for determining whether a patient suffering from melanoma will respond to treatment with a BET inhibitor by:
i) determining the expression level of the stratification markers PPARGC1A, PPARGC1B, and/or MITF by measurement of the respective mRNA or derived cDNA expression levels in a sample of body fluid or tumor tissue of said patient, and comparing the expression level with that of normal human melanocytes,
and/or
ii) determining the protein level of the stratification markers PPARGC1A, PPARGC1B and/or MITF in a melanoma patient in a sample of body fluid or tumor tissue of said patient, and comparing it with that of normal human melanocytes,
and/or
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated mRNA or derived cDNA and/or protein expression level of PPARGC1A, PPARGC1B and/or MITF and/or a lowered OCR following treatment with a BET inhibitor in comparison with the untreated sample is suggestive of a better response to the treatment of melanoma in said patient.

The determination of the expression level of the mRNA or derived cDNA and the determination of the protein level, as well as the determination of the basal OCR can either be done combined, or separately. All combinations are possible to get a valuable result for stratification.

Thus, a further object of the invention is an in vitro stratification method for determining whether a patient suffering from melanoma will respond to treatment with a BET inhibitor by:
i) determining the expression level of the stratification markers PPARGC1A, PPARGC1B, and/or MITF by measurement of the respective mRNA or derived cDNA expression levels in a sample of body fluid or tumor tissue of said patient, and comparing the expression level with that of normal human melanocytes,
or
ii) determining the protein level of the stratification markers PPARGC1A, PPARGC1B and/or MITF in a melanoma patient in a sample of body fluid or tumor tissue of said patient, and comparing it with that of normal human melanocytes,
or
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated mRNA or derived cDNA, or protein expression level of PPARGC1A, PPARGC1B and/or MITF or a lowered OCR following treatment with a BET inhibitor in comparison with the untreated sample is suggestive of a better response to the treatment of melanoma in said patient.

The invention further is an in vitro stratification method for determining whether a patient suffering from melanoma will respond to treatment with a BET inhibitor by:
i) determining the expression level of the stratification markers PPARGC1A, PPARGC1B, and/or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B and/or MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated mRNA or derived cDNA and protein expression level of PPARGC1A, PPARGC1B and/or MITF and a lowered OCR following treatment with a BET inhibitor in comparison with the untreated sample is suggestive of a better response to the treatment of melanoma in said patient.

Further, the invention is an in vitro stratification method for determining whether a patient suffering from melanoma will respond to treatment with a BET inhibitor by:
i) determining the expression level of the stratification markers PPARGC1A, PPARGC1B, and/or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B and/or MITF in a melanoma patient in a sample of body fluid or tumor tissue of said patient, and comparing it with that of normal human melanocytes,

and wherein the presence in said in vitro sample of an elevated mRNA or derived cDNA and protein expression level of PPARGC1A, PPARGC1B and/or MITF following treatment with a BET inhibitor in comparison with the untreated sample is suggestive of a better response to the treatment of melanoma in said patient.

Further, the invention is an in vitro stratification method for determining whether a patient suffering from melanoma will respond to treatment with a BET inhibitor by:
i) determining the expression level of the stratification markers PPARGC1A, PPARGC1B, and/or MITF by measurement of the respective mRNA or derived cDNA expression levels in a sample of body fluid or tumor tissue of said patient, and comparing the expression level with that of normal human melanocytes,
and
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated mRNA or derived cDNA and a lowered OCR following treatment with a BET inhibitor in comparison with the untreated sample is suggestive of a better response to the treatment of melanoma in said patient.

Further, the invention is an in vitro stratification method for determining whether a patient suffering from melanoma will respond to treatment with a BET inhibitor by:
ii) determining the protein level of the stratification markers PPARGC1A, PPARGC1B and/or MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated protein expression level of PPARGC1A, PPARGC1B and/or MITF and a lowered OCR following treatment with a BET inhibitor in comparison with the untreated sample is suggestive of a better response to the treatment of melanoma in said patient.

The determination of the mRNA or derived cDNA, or protein expression level can be done with all of the stratification markers PPARGC1A, PPARGC1B and MITF, or can be done with only the stratification markers PPARGC1A and PPARGC1B, or with only the stratification markers PPARGC1A and MITF, or with only the stratification markers PPARGC1B and MITF, or can separately be done by measurement of the single stratification marker PPARGC1A or PPARGC1B or MITF alone. All combinations are possible to get a valuable result for stratification.

Thus, a further object of the invention is an in vitro stratification method for determining whether a patient suffering from melanoma will respond to treatment with a BET inhibitor by:
i) determining the expression level of the stratification markers PPARGC1A, PPARGC1B or MITF by measurement of the respective mRNA or derived cDNA expression levels in a sample of body fluid or tumor tissue of said patient, and comparing the expression level with that of normal human melanocytes,
or
ii) determining the protein level of the stratification markers PPARGC1A, PPARGC1B or MITF in a melanoma patient in a sample of body fluid or tumor tissue of said patient, and comparing it with that of normal human melanocytes,
or
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated mRNA or derived cDNA, or protein expression level of PPARGC1A, PPARGC1B or MITF or a lowered OCR following treatment with a BET inhibitor in comparison with the untreated sample is suggestive of a better response to the treatment of melanoma in said patient.

Thus, a further object of the invention is an in vitro stratification method for determining whether a patient suffering from melanoma will respond to treatment with a BET inhibitor by:
i) determining the expression level of the stratification markers PPARGC1A, PPARGC1B and MITF by measurement of the respective mRNA or derived cDNA expression levels in a sample of body fluid or tumor tissue of said patient, and comparing the expression level with that of normal human melanocytes,
or
ii) determining the protein level of the stratification markers PPARGC1A, PPARGC1B and MITF in a melanoma patient in a sample of body fluid or tumor tissue of said patient, and comparing it with that of normal human melanocytes,
or
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated mRNA or derived cDNA, or protein expression level of PPARGC1A, PPARGC1B and MITF or a lowered OCR following treatment with a BET inhibitor in comparison with the untreated sample is suggestive of a better response to the treatment of melanoma in said patient.

Thus, a further object of the invention is an in vitro stratification method for determining whether a patient suffering from melanoma will respond to treatment with a BET inhibitor by:
i) determining the expression level of the stratification markers PPARGC1A and PPARGC1B by measurement of the respective mRNA or derived cDNA expression levels in a sample of body fluid or tumor tissue of said patient, and comparing the expression level with that of normal human melanocytes,
or
ii) determining the protein level of the stratification markers PPARGC1A and PPARGC1B in a melanoma patient in a sample of body fluid or tumor tissue of said patient, and comparing it with that of normal human melanocytes,
or
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated mRNA or derived cDNA, or protein expression level of PPARGC1A and PPARGC1B or a lowered OCR following treatment with a BET inhibitor in comparison with the untreated sample is suggestive of a better response to the treatment of melanoma in said patient.

Thus, a further object of the invention is an in vitro stratification method for determining whether a patient suffering from melanoma will respond to treatment with a BET inhibitor by:
i) determining the expression level of the stratification markers PPARGC1A and MITF by measurement of the respective mRNA or derived cDNA expression levels in a sample of body fluid or tumor tissue of said patient, and comparing the expression level with that of normal human melanocytes,
or
ii) determining the protein level of the stratification markers PPARGC1A and MITF in a melanoma patient in a sample of body fluid or tumor tissue of said patient, and comparing it with that of normal human melanocytes,
or
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated mRNA or derived cDNA, or protein expression level of PPARGC1A and MITF or a lowered OCR following treatment with a BET inhibitor in comparison with the untreated sample is suggestive of a better response to the treatment of melanoma in said patient.

Thus, a further object of the invention is an in vitro stratification method for determining whether a patient suffering from melanoma will respond to treatment with a BET inhibitor by:
i) determining the expression level of the stratification markers PPARGC1B and MITE by measurement of the respective mRNA or derived cDNA expression levels in a sample of body fluid or tumor tissue of said patient, and comparing the expression level with that of normal human melanocytes,
or
ii) determining the protein level of the stratification markers PPARGC1B and MITF in a melanoma patient in a sample of body fluid or tumor tissue of said patient, and comparing it with that of normal human melanocytes,
or
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated mRNA or derived cDNA, or protein expression level of PPARGC1B and MITF or a lowered OCR following treatment with a BET inhibitor in comparison with the untreated sample is suggestive of a better response to the treatment of melanoma in said patient.

Thus, a further object of the invention is an in vitro stratification method for determining whether a patient suffering from melanoma will respond to treatment with a BET inhibitor by:
i) determining the expression level of the stratification markers PPARGC1A, PPARGC1B or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B or MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated mRNA or derived cDNA and protein expression level of PPARGC1A, PPARGC1B or MITF and a lowered OCR following treatment with a BET inhibitor in comparison with the untreated sample is suggestive of a better response to the treatment of melanoma in said patient.

Thus, a further object of the invention is an in vitro stratification method for determining whether a patient suffering from melanoma will respond to treatment with a BET inhibitor by:
i) determining the expression level of the stratification markers PPARGC1A, PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated mRNA or derived cDNA, and protein expression level of PPARGC1A, PPARGC1B and MITF and a lowered OCR following treatment with a BET inhibitor in comparison with the untreated sample is suggestive of a better response to the treatment of melanoma in said patient.

Thus, a further object of the invention is an in vitro stratification method for determining whether a patient suffering from melanoma will respond to treatment with a BET inhibitor by:
i) determining the expression level of the stratification markers PPARGC1A and PPARGC1B by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A and PPARGC1B in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated mRNA or derived cDNA, and protein expression level of PPARGC1A and PPARGC1B and a lowered OCR following treatment with a BET inhibitor in comparison with the untreated sample is suggestive of a better response to the treatment of melanoma in said patient.

Thus, a further object of the invention is an in vitro stratification method for determining whether a patient suffering from melanoma will respond to treatment with a BET inhibitor by:
i) determining the expression level of the stratification markers PPARGC1A and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated mRNA or derived cDNA, and protein expression level of PPARGC1A and MITF and a lowered OCR following treatment with a BET inhibitor in comparison with the untreated sample is suggestive of a better response to the treatment of melanoma in said patient.

Thus, a further object of the invention is an in vitro stratification method for determining whether a patient suffering from melanoma will respond to treatment with a BET inhibitor by:
i) determining the expression level of the stratification markers PPARGC1B and M1TF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1B and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated mRNA or derived cDNA, and protein expression level of PPARGC1B and MITF and a lowered OCR following treatment with a BET inhibitor in comparison with the untreated sample is suggestive of a better response to the treatment of melanoma in said patient.

Thus, a further object of the invention is an in vitro stratification method for determining whether a patient suffering from melanoma will respond to treatment with a BET inhibitor by:
i) determining the expression level of the stratification markers PPARGC1A, PPARGC1B or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B or MITF in a melanoma patient in a sample of body fluid or tumor tissue of said patient, and comparing it with that of normal human melanocytes,

and wherein the presence in said in vitro sample of an elevated mRNA or derived cDNA and protein expression level of PPARGC1A, PPARG C1B or MITF following treatment with a BET inhibitor in comparison with the untreated sample is suggestive of a better response to the treatment of melanoma in said patient.

Thus, a further object of the invention is an in vitro stratification method for determining whether a patient suffering from melanoma will respond to treatment with a BET inhibitor by:
i) determining the expression level of the stratification markers PPARGC1A, PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B and MITF in a melanoma patient in a sample of body fluid or tumor tissue of said patient, and comparing it with that of normal human melanocytes,

and wherein the presence in said in vitro sample of an elevated mRNA or derived cDNA, and protein expression level of PPARGC1A, PPARGC1B and MITF following treatment with a BET inhibitor in comparison with the untreated sample is suggestive of a better response to the treatment of melanoma in said patient.

Thus, a further object of the invention is an in vitro stratification method for determining whether a patient suffering from melanoma will respond to treatment with a BET inhibitor by:
i) determining the expression level of the stratification markers PPARGC1A and PPARGC1B by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A and PPARGC1B in a melanoma patient in a sample of body fluid or tumor tissue of said patient, and comparing it with that of normal human melanocytes,

and wherein the presence in said in vitro sample of an elevated mRNA or derived cDNA and protein expression level of PPARGC1A and PPARGC1B following treatment with a BET inhibitor in comparison with the untreated sample is suggestive of a better response to the treatment of melanoma in said patient.

Thus, a further object of the invention is an in vitro stratification method for determining whether a patient suffering from melanoma will respond to treatment with a BET inhibitor by:
i) determining the expression level of the stratification markers PPARGC1A and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A and MITF in a melanoma patient in a sample of body fluid or tumor tissue of said patient, and comparing it with that of normal human melanocytes,

and wherein the presence in said in vitro sample of an elevated mRNA or derived cDNA and protein expression level of PPARGC1A and MITF following treatment with a BET inhibitor in comparison with the untreated sample is suggestive of a better response to the treatment of melanoma in said patient.

Thus, a further object of the invention is an in vitro stratification method for determining whether a patient suffering from melanoma will respond to treatment with a BET inhibitor by:
i) determining the expression level of the stratification markers PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1B and MITF in a melanoma patient in a sample of body fluid or tumor tissue of said patient, and comparing it with that of normal human melanocytes,

and wherein the presence in said in vitro sample of an elevated mRNA or cDNA, and protein expression level of PPARGC1B and MITF following treatment with a BET inhibitor in comparison with the untreated sample is suggestive of a better response to the treatment of melanoma in said patient.

Thus, a further object of the invention is an in vitro stratification method for determining whether a patient suffering from melanoma will respond to treatment with a BET inhibitor by:
i) determining the expression level of the stratification markers PPARGC1A, PPARGC1B or MITF by measurement of the respective mRNA or derived cDNA expression levels in a sample of body fluid or tumor tissue of said patient, and comparing the expression level with that of normal human melanocytes,
and
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated mRNA or derived cDNA expression level of PPARGC1A, PPARGC1B or MITF and a lowered OCR following treatment with a BET inhibitor in comparison with the untreated sample is suggestive of a better response to the treatment of melanoma in said patient.

Thus, a further object of the invention is an in vitro stratification method for determining whether a patient suffering from melanoma will respond to treatment with a BET inhibitor by:
i) determining the expression level of the stratification markers PPARGC1A, PPARGC1B and MITF by measurement of the respective mRNA or derived cDNA expression levels in a sample of body fluid or tumor tissue of said patient, and comparing the expression level with that of normal human melanocytes,
and
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated mRNA or derived cDNA expression level of PPARGC1A, PPARGC1B and MITF and a lowered OCR following treatment with a BET inhibitor in comparison with the untreated sample is suggestive of a better response to the treatment of melanoma in said patient.

Thus, a further object of the invention is an in vitro stratification method for determining whether a patient suffering from melanoma will respond to treatment with a BET inhibitor by:
i) determining the expression level of the stratification markers PPARGC1A and PPARGC1B by measurement of the respective mRNA or derived cDNA expression levels in a sample of body fluid or tumor tissue of said patient, and comparing the expression level with that of normal human melanocytes,
and
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated mRNA or derived cDNA expression level of PPARGC1A and PPARGC1B and a lowered OCR following treatment with a BET inhibitor in comparison with the untreated sample is suggestive of a better response to the treatment of melanoma in said patient.

Thus, a further object of the invention is an in vitro stratification method for determining whether a patient suffering from melanoma will respond to treatment with a BET inhibitor by:
i) determining the expression level of the stratification markers PPARGC1A and MITF by measurement of the respective mRNA or derived cDNA expression levels in a sample of body fluid or tumor tissue of said patient, and comparing the expression level with that of normal human melanocytes,
and
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated mRNA or derived cDNA expression level of PPARGC1A and MITF and a lowered OCR following treatment with a BET inhibitor in comparison with the untreated sample is suggestive of a better response to the treatment of melanoma in said patient.

Thus, a further object of the invention is an in vitro stratification method for determining whether a patient suffering from melanoma will respond to treatment with a BET inhibitor by:
i) determining the expression level of the stratification markers PPARGC1B and MITF by measurement of the respective mRNA or derived cDNA expression levels in a sample of body fluid or tumor tissue of said patient, and comparing the expression level with that of normal human melanocytes,
and
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated mRNA or derived cDNA expression level of PPARGC1B and MITF and a lowered OCR following treatment with a BET inhibitor in comparison with the untreated sample is suggestive of a better response to the treatment of melanoma in said patient.

Thus, a further object of the invention is an in vitro stratification method for determining whether a patient suffering from melanoma will respond to treatment with a BET inhibitor by:
i) determining the protein level of the stratification markers PPARGC1A, PPARGC1B or MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated protein expression level of PPARGC1A, PPARGC1B or MITF and a lowered OCR following treatment with a BET inhibitor in comparison with the untreated sample is suggestive of a better response to the treatment of melanoma in said patient.

Thus, a further object of the invention is an in vitro stratification method for determining whether a patient suffering from melanoma will respond to treatment with a BET inhibitor by:
ii) determining the protein level of the stratification markers PPARGC1A, PPARGC1B and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated protein expression level of PPARGC1A, PPARGC1B and MITF and a lowered OCR following treatment with a BET inhibitor in comparison with the untreated sample is suggestive of a better response to the treatment of melanoma in said patient.

Thus, a further object of the invention is an in vitro stratification method for determining whether a patient suffering from melanoma will respond to treatment with a BET inhibitor by:
ii) determining the protein level of the stratification markers PPARGC1A and PPARGC1B in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated protein expression level of PPARGC1A and PPARGC1B and a lowered OCR following treatment with a BET inhibitor in comparison with the untreated sample is suggestive of a better response to the treatment of melanoma in said patient.

Thus, a further object of the invention is an in vitro stratification method for determining whether a patient suffering from melanoma will respond to treatment with a BET inhibitor by:
ii) determining the protein level of the stratification markers PPARGC1A and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated protein expression level of PPARGC1A and MITF and a lowered OCR following treatment with a BET inhibitor in comparison with the untreated sample is suggestive of a better response to the treatment of melanoma in said patient.

Thus, a further object of the invention is an in vitro stratification method for determining whether a patient suffering from melanoma will respond to treatment with a BET inhibitor by:
ii) determining the protein level of the stratification markers PPARGC1B and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated protein expression level of PPARGC1B and MITF and a lowered OCR following treatment with a BET inhibitor in comparison with the untreated sample is suggestive of a better response to the treatment of melanoma in said patient.

The respective mRNA or derived cDNA measurements of the PPARGC1A, PPARGC1B and MITF markers can be done separately or combined with the measurements of the protein expression level of the PPARGC1A, PPARGC1B and MITF markers.

For Example the Following Measurements are Possible:
The respective mRNA or derived cDNA levels of the PPARGC1A, PPARGC1B and MITF markers combined with the protein expression level of the PPARGC1A, PPARGC1B or MITF marker.
The respective mRNA or derived cDNA levels of the PPARGC1A, PPARGC1B or MITF marker combined with the protein expression level of the PPARGC1A, PPARGC1B and MITF markers.
The respective mRNA or derived cDNA levels of the PPARGC1A and PPARGC1B markers combined with the protein expression level of the PPARGC1A or PPARGC1B marker.
The respective mRNA or derived cDNA levels of the PPARGC1A or PPARGC1B marker combined with the protein expression level of the PPARGC1A and PPARGC1B markers.
The respective mRNA or derived cDNA levels of the PPARGC1A and MITF markers combined with the protein expression level of the PPARGC1A or MITF marker.
The respective mRNA or derived cDNA levels of the PPARGC1A or MITF marker combined with the protein expression level of the PPARGC1A and MITF markers.
The respective mRNA or derived cDNA levels of the PPARGC1B and MITF markers combined with the protein expression level of the PPARGC1B or MITF marker.
The respective mRNA or derived cDNA levels of the PPARGC1B or MITF marker combined with the protein expression level of the PPARGC1B and MITF markers.

Thus, a further object of the present invention is an in vitro stratification method for determining whether a patient suffering from melanoma will respond to treatment with a BET inhibitor by:
i) determining the expression level of the stratification markers PPARGC1A, PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B or MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated mRNA or derived cDNA and protein expression level of PPARGC1A, PPARGC1B and MITF and a lowered OCR following treatment with a BET inhibitor in comparison with the untreated sample is suggestive of a better response to the treatment of melanoma in said patient.

Thus, a further object of the present invention is an in vitro stratification method for determining whether a patient suffering from melanoma will respond to treatment with a BET inhibitor by:
i) determining the expression level of the stratification markers PPARGC1A, PPARGC1B or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated mRNA or derived cDNA and protein expression level of PPARGC1A, PPARGC1B and MITF and a lowered OCR following treatment with a BET inhibitor in comparison with the untreated sample is suggestive of a better response to the treatment of melanoma in said patient.

Thus, a further object of the present invention is an in vitro stratification method for determining whether a patient suffering from melanoma will respond to treatment with a BET inhibitor by:
i) determining the expression level of the stratification markers PPARGC1A, PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B or MITF in a melanoma patient in a sample of body fluid or tumor tissue of said patient, and comparing it with that of normal human melanocytes,

and wherein the presence in said in vitro sample of an elevated mRNA or derived cDNA and protein expression level of PPARGC1A, PPARGC1B and MITF following treatment with a BET inhibitor in comparison with the untreated sample is suggestive of a better response to the treatment of melanoma in said patient.

Thus, a further object of the present invention is an in vitro stratification method for determining whether a patient suffering from melanoma will respond to treatment with a BET inhibitor by:
i) determining the expression level of the stratification markers PPARGC1A, PPARGC1B or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B and MITF in a melanoma patient in a sample of body fluid or tumor tissue of said patient, and comparing it with that of normal human melanocytes,

and wherein the presence in said in vitro sample of an elevated mRNA or derived cDNA and protein expression level of PPARGC1A, PPARGC1B and MITF following treatment with a BET inhibitor in comparison with the untreated sample is suggestive of a better response to the treatment of melanoma in said patient.

Thus, a further object of the present invention is an in vitro stratification method for determining whether a patient suffering from melanoma will respond to treatment with a BET inhibitor by:
i) determining the expression level of the stratification markers PPARGC1A and PPARGC1B by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A or PPARGC1B in a melanoma patient in a sample of body fluid or tumor tissue of said patient, and comparing it with that of normal human melanocytes,

and wherein the presence in said in vitro sample of an elevated mRNA or derived cDNA and protein expression level of PPARGC1A, PPARGC1B and MITF following treatment with a BET inhibitor in comparison with the untreated sample is suggestive of a better response to the treatment of melanoma in said patient.

Thus, a further object of the present invention is an in vitro stratification method for determining whether a patient suffering from melanoma will respond to treatment with a BET inhibitor by:
i) determining the expression level of the stratification markers PPARGC1A or PPARGC1B by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A and PPARGC1B in a melanoma patient in a sample of body fluid or tumor tissue of said patient, and comparing it with that of normal human melanocytes,

and wherein the presence in said in vitro sample of an elevated mRNA or derived cDNA and protein expression level of PPARGC1A, PPARGC1B and MITF following treatment with a BET inhibitor in comparison with the untreated sample is suggestive of a better response to the treatment of melanoma in said patient.

Thus, a further object of the present invention is an in vitro stratification method for determining whether a patient suffering from melanoma will respond to treatment with a BET inhibitor by:
i) determining the expression level of the stratification markers PPARGC1A and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A or MITF in a melanoma patient in a sample of body fluid or tumor tissue of said patient, and comparing it with that of normal human melanocytes,

and wherein the presence in said in vitro sample of an elevated mRNA or derived cDNA and protein expression level of PPARGC1A, PPARGC1B and MITF following treatment with a BET inhibitor in comparison with the untreated sample is suggestive of a better response to the treatment of melanoma in said patient.

Thus, a further object of the present invention is an in vitro stratification method for determining whether a patient suffering from melanoma will respond to treatment with a BET inhibitor by:
i) determining the expression level of the stratification markers PPARGC1A or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A and MITF in a melanoma patient in a sample of body fluid or tumor tissue of said patient, and comparing it with that of normal human melanocytes,

and wherein the presence in said in vitro sample of an elevated mRNA or derived cDNA and protein expression level of PPARGC1A, PPARGC1B and MITF following treatment with a BET inhibitor in comparison with the untreated sample is suggestive of a better response to the treatment of melanoma in said patient.

Thus, a further object of the present invention is an in vitro stratification method for determining whether a patient suffering from melanoma will respond to treatment with a BET inhibitor by:
i) determining the expression level of the stratification markers PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1B or MITF in a melanoma patient in a sample of body fluid or tumor tissue of said patient, and comparing it with that of normal human melanocytes,

and wherein the presence in said in vitro sample of an elevated mRNA or derived cDNA and protein expression level of PPARGC1A, PPARGC1B and MITF following treatment with a BET inhibitor in comparison with the untreated sample is suggestive of a better response to the treatment of melanoma in said patient.

Thus, a further object of the present invention is an in vitro stratification method for determining whether a patient suffering from melanoma will respond to treatment with a BET inhibitor by:
i) determining the expression level of the stratification markers PPARGC1B or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1B and MITF in a melanoma patient in a sample of body fluid or tumor tissue of said patient, and comparing it with that of normal human melanocytes,

and wherein the presence in said in vitro sample of an elevated mRNA or derived cDNA and protein expression level of PPARGC1A, PPARGC1B and MITF following treatment with a BET inhibitor in comparison with the untreated sample is suggestive of a better response to the treatment of melanoma in said patient.

Of selected interest is an in vitro stratification method for determining whether a patient suffering from melanoma will respond to treatment with a BET inhibitor by:
i) determining the expression level of the stratification markers PPARGC1A or MITF by measurement of the respective mRNA or derived cDNA expression levels in a sample of body fluid or tumor tissue of said patient, and comparing the expression level with that of normal human melanocytes,
and/or
ii) determining the protein level of the stratification markers PPARGC1A or MITF in a melanoma patient in a sample of body fluid or tumor tissue of said patient, and comparing it with that of normal human melanocytes,
and/or
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated mRNA or derived cDNA and/or protein expression level of PPARGC1A or MITF and/or a lowered OCR following treatment with a BET inhibitor in comparison with the untreated sample is suggestive of a better response to the treatment of melanoma in said patient.

More preferred is an in vitro stratification method for determining whether a patient suffering from melanoma will respond to treatment with a BET inhibitor by:
i) determining the expression level of the stratification markers PPARGC1A measurement of the respective mRNA or derived cDNA expression levels in a sample of body fluid or tumor tissue of said patient, and comparing the expression level with that of normal human melanocytes,
or
ii) determining the protein level of the stratification markers PPARGC1A in a melanoma patient in a sample of body fluid or tumor tissue of said patient, and comparing it with that of normal human melanocytes,
and/or
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated mRNA or derived cDNA or protein expression level of PPARGC1A and/or a lowered OCR following treatment with a BET inhibitor in comparison with the untreated sample is suggestive of a better response to the treatment of melanoma in said patient.

Much more preferred is an in vitro stratification method for determining whether a patient suffering from melanoma will respond to treatment with a BET inhibitor by:
i) determining the expression level of the stratification markers PPARGC1A by measurement of the respective mRNA or derived cDNA expression levels in a sample of body fluid or tumor tissue of said patient, and comparing the expression level with that of normal human melanocytes,
or
ii) determining the protein level of the stratification markers PPARGC1A in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

and wherein the presence in said in vitro sample of an elevated mRNA or derived cDNA or protein expression level of PPARGC1A and a lowered OCR following treatment with a BET inhibitor in comparison with the untreated sample is suggestive of a better response to the treatment of melanoma in said patient.

The present invention concerns a stratification method, as defined above and following a stratification kit and the use of a BET inhibitor for the treatment of melanoma in a patient.

In this regard the features are defined as follows:

Body fluid in the present invention means for example blood, plasma, serum, lymph saliva, sweat, teardrops, urine or feces of a patient.

Tumor tissue in the present invention means for example primary tumor, metastases or circulating tumor cells.

Normal human melanocytes for example in the present invention means PCS-200-013 (www. atcc. org/Products/All/P C S-200-013 . aspx), PCS-200-012 (www. atcc. org/P roducts/All/P C S -200-012.aspx) and CRL-4004 (www.atcc.org/Search_Results.aspx?dsNav=Ntk:PrimarySearch%7ccrl%2f-4004%7c3 %7c,Ny :True,Ro: 0,N:1000552& s earchTerms=cr1-4004&redir=1) cells, can be obtained from ATCC® (www.atcc.org; Manassas, VA, USA), or normal human epidermal melanocytes NHEM.f-c M2 (C-12402, www.promocell.comiproducts/human-primary-cells/melanocytes/#C-12402) or NHEM-c M2 (C-12403, www.promocell.comiproducts/human-primary-cells/melanocytes/#C-12403), which can be obtained from PromoCell (Heidelberg, Germany).

An elevated RNA or protein expression level of PPARGC1A, PPARGC1B or MITF in a sample is suggestive of a better response to the treatment of melanoma in the patient, if the mRNA, cDNA or protein expression level is at least 2-fold higher than in melanocytes.

More preferred is an expression level that is of at least 3-fold to 5-fold higher than in melanocytes. It is also possible that an expression level is more than 5-fold higher than in melanocytes.

A further aspect of the invention is the use of the method for in vitro stratification of a melanoma disease in a patient. The patient is a mammal, especially a human.

Gene expression levels are assessed by determining the amount of RNA, for example mRNA or derived cDNA that is transcribed from a gene or gene sequence and coding for a peptide or protein. Today, the gene expression analysis can be done according to well-established and known processes. Methods for gene expression analysis include, but are not limited to, reverse transcription quantitative PCR, differential display PCR, hybridization-based microarrays and next-generation sequencing, including RNA-Seq (F. Ozsolak and P. M. Milos, Nat. Rev. Genet. 2011, 12:87-98).

For the measurement of gene expression, it is an advantage to amplify RNA, respectively cDNA. Today, well established processes are available for the generation of cDNA from an RNA template, using a reverse transcriptase (S. Hahn et al., Cell. Mol. Life Sci., 2000, 57:96-105).

Gene expression profiles indicative of BET responders are preferably those which show at least a 1.5-, 1.7-, or 2-fold difference relative to BET non-responders with regard to the expression of the respective mRNA or derived cDNA of PPARGC1A, PPARGC1B or MITF.

An expression difference of 1.5- fold in responders versus non-responder cell lines or tumors is clearly predictive of the influence of the BET inhibitor on the diseased cells or tumors. More preferred is a difference of 1.7-fold and much more preferred is a difference of 2-fold, which more clearly indicates that the BET inhibitor will inhibit the proliferation of the diseased cells or tumors.

Protein extracts can be prepared by methods including, but not limited to, ion exchange column, size exclusion chromatography, SDS polyacrylamide gel electrophoresis, high performance liquid chromatography or reversed-phase chromatography (N. E. Labrou, Methods Mol. Biol., 2014, 1129:3-10). Protein levels can be measured by methods including, but not limited to, protein immunostaining and microscopy, immunoprecipitation, immunoelectrophoresis, Western blot, spectrophotometry, mass spectrometry, radioimmunoassay and enzyme-linked immunosorbent assay, immuno-PCR, stable isotope labeling by amino acids, tissue microarrays, protein biochips, proteomics and nanoproteomics (K. K. Jain, J BUON, 2007, Suppl. 1:S31-S38; A. Brewis and P. Brennan, Adv. Protein Chem. Struct. Biol., 2010, 80:1-44; T. C. Collier and D. C. Muddiman, Amino Acids, 2012, 43:1109-1117; S. E. Ong, Anal. Bioanal. Chem, 2012, 404:967-976; E. Rodriguez-Suarez and A. D. Whetton, Mass Spectrom. Rev., 2013, 32:1-26).

Protein levels indicative of BET responders are preferably those which show at least a 1.5-, 1.7-, or 2-fold difference relative to BET non-responders with regard to expression of the respective protein of PPARGC1A, PPARGC1B or MITF.

A protein level difference of 1.5- fold in responders versus non-responder cell lines or tumors clearly indicates that the level of the protein is predictive of the influence of the BET inhibitor on the diseased cells or tumors. More preferred is a difference of 1.7-fold and much more preferred is a difference of 2-fold, which more clearly indicates that the BET inhibitor will inhibit the proliferation of the diseased cells or tumors.

OCR can be measured in tumors using methods including, but not limited to, electron paramagnetic resonance oximetry, the Clark oxygen electrode, the MitoXpress fluorescent assay and the SeaHorse extracellular flux analyzer (C. Diepart et al., Anal. Biochem., 2010, 396:250-256; W. Qian and B. Van Houten, Methods, 2010, 51:452-457).

Before the basal OCR of the samples that are untreated or treated with an inhibitor are determined with a suitable device, the melanoma cells are incubated for 10 to 50 hours, preferably for 20 to 30 hours, most preferred for 24 hours.

A suitable device that can be used for the determination of the basal OCR in the melanoma cell line from the sample of body fluid or tumor tissue of said patient is the Seahorse XF96 instrument [Seahorse Bioscience] under standard conditions.

The use of Seahorse XF96 instruments [Seahorse Bioscience] whereby the detection of the OCR is measured with oxygen-sensing fluorophores and extracellular acidification with a pH sensor simultaneously in the same population of intact cells is preferred, but the basal OCR can also be determined using a Clark-type oxygen electrode (e.g. Hansatech Instruments), whereby the oxygen which is dissolved in the liquid or gas phase in the sample chamber is detected by polarography, or with a Oroboros Oxygraph-2k (Oroboros Instruments), whereby the oxygen which is dissolved in the liquid or the gas phase in the sample chamber is detected by polarography using a Clark-type oxygen electrode with high-resolution respirometry, or with fiber optic oxygen sensors (e.g. Ocean Optics Sensors), whereby a fluorescence method is used to measure the partial pressure of dissolved or gaseous oxygen in a sample.

For example, melanoma cells that are untreated (control) or treated with 1 μM with the inhibitor JQ1 or BAY 123 are incubated for 24 hours. The applied inhibitor concentration is achieved by diluting 10 mM stock solutions. The basal OCR are determined with the Seahorse XF96 instrument.

OCR indicative of BET responders are preferably those which show at least a 1.5-, 1.7-, or 2-fold difference relative to BET non-responders.

An OCR of 1.5- fold in responders vs. non-responder tumors or tumor biopsies clearly indicates that the level of OCR is predictive of the influence of the BET inhibitor on the diseased cells or tumors. More preferred is a difference of 1.7-fold and much more preferred is a difference of 2-fold, which more clearly indicates that the BET inhibitor will inhibit the proliferation of the diseased cells or tumors.

Within the scope of the present invention, melanoma is understood as a disease of mammals, especially as a disease of the human and non-human mammal body, more specifically of the human body.

Melanoma in this regard means lentigo maligna (lentiginous melanoma), lentigo maligna melanoma (a melanoma that has evolved from a Lentigo maligna), superficial spreading melanoma (superficially spreading melanoma), acral lentiginous melanoma, mucosal melanoma, nodular melanoma, polypoid melanoma (a virulent variant of nodular melanoma), desmoplastic melanoma (neurotropic melanoma, or spindled melanoma), amelanotic melanoma, soft-tissue melanoma (clear-cell sarcoma), small-cell melanoma (melanoma with small nevus-like cells), Spitzoid melanoma (melanoma with features of a Spitz nevus) and uveal melanoma.

In a further preferred embodiment of the method for stratification according to the invention, body fluid or body tissue, preferably blood, alternatively whole blood, serum or available plasma, is taken from the patient to be examined, and the analysis is made in vitro, respectively ex vivo, which means outside the mammalian, respectively human or animal body.

Due to the determination of the RNA expression of PPARGC1A, PPARGC1B and MITF or of the corresponding protein or of partial peptide fragments thereof, and its overexpression in at least one patient sample, the stratification can be made.

Within the scope of the invention PPARGC1A is to be understood as a free human protein or polypeptide consisting of 798 amino acids and having the amino acid sequence SEQ ID No. 4: Q9UBK2 (www.uniprot.org/uniprot/Q9UBK2) (see FIG. 8), or a fragment of the PPARGC1A protein sequence of at least 15 amino acids.

Within the scope of the invention PPARGC1B is to be understood as a free human protein or polypeptide consisting of 984 amino acids and having the amino acid sequence SEQ ID No 5: AAI44252 (www.uniprot.org/uniprot/B7ZM40) (see FIG. 9), or a fragment of the PPARGC1B protein sequence of at least 15 amino acids.

Within the scope of the invention MITF is to be understood as a free human protein or polypeptide consisting of 520 amino acids and having the amino acid sequence SEQ ID No 6: NP-937802 (www.uniprot.org/uniprot/O75030) (see FIG. 10), or a fragment of the MITF protein sequence of at least 15 amino acids.

A patient suffering from melanoma can be treated with a therapeutically effective amount of a BET inhibitor if the stratification marker PPARGC1A, PPARGC1B and/or MITF show an elevated mRNA, cDNA or protein expression level, and/or a lowered OCR can be determined following treatment with a BET inhibitor. As already mentioned above, the expression level of the mRNA or derived cDNA and the determination of the protein level, as well as the determination of the basal OCR can either be determined combined, or separately.

All combinations are possible to get a valuable result for stratification and safe information for the administration of an effective amount of a BET inhibitor to a patient suffering from melanoma.

Melanoma that can be treated with a therapeutically effective amount of a BET inhibitor after stratification is selected from the group consisting of lentigo maligna (lentiginous melanoma), lentigo maligna melanoma (a melanoma that has evolved from a Lentigo maligna), superficial spreading melanoma (superficially spreading melanoma), acral lentiginous melanoma, mucosal melanoma, nodular melanoma, polypoid melanoma (a virulent variant of nodular melanoma), desmoplastic melanoma (neurotropic melanoma, or spindled melanoma), amelanotic melanoma, soft-tissue melanoma (clear-cell sarcoma), small-cell melanoma (melanoma with small nevus-like cells), Spitzoid melanoma (melanoma with features of a Spitz nevus) and uveal melanoma.

Thus, a further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B, and/or MITF by measurement of the respective mRNA or derived cDNA expression levels in a sample of body fluid or tumor tissue of said patient, and comparing the expression level with that of normal human melanocytes,
and/or
ii) determining the protein level of the stratification markers PPARGC1A, PPARGC1B and/or MITF in a melanoma patient in a sample of body fluid or tumor tissue of said patient, and comparing it with that of normal human melanocytes,
and/or
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA or protein expression level of PPARGC1A, PPARGC1B and/or MITF and/or a lowered OCR following treatment with a BET inhibitor is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B, and/or MITE by measurement of the respective mRNA or derived cDNA expression levels in a sample of body fluid or tumor tissue of said patient, and comparing the expression level with that of normal human melanocytes,
and/or
ii) determining the protein level of the stratification markers PPARGC1A, PPARGC1B and/or MITF in a melanoma patient in a sample of body fluid or tumor tissue of said patient, and comparing it with that of normal human melanocytes,
and/or
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA or protein expression level of PPARGC1A, PPARGC1B and/or MITF and/or a lowered OCR following treatment with a BET inhibitor is detected, a therapeutically effective amount of a BET inhibitor is administered to the melanoma patient.

A further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B, and/or MITF by measurement of the respective mRNA or derived cDNA expression levels in a sample of body fluid or tumor tissue of said patient, and comparing the expression level with that of normal human melanocytes,
or
ii) determining the protein level of the stratification markers PPARGC1A, PPARGC1B and/or MITF in a melanoma patient in a sample of body fluid or tumor tissue of said patient, and comparing it with that of normal human melanocytes,
or
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA or protein expression level of PPARGC1A, PPARGC1B and/or MITF or a lowered OCR following treatment with a BET inhibitor is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B, and/or MITF by measurement of the respective mRNA or derived cDNA expression levels in a sample of body fluid or tumor tissue of said patient, and comparing the expression level with that of normal human melanocytes,
or
ii) determining the protein level of the stratification markers PPARGC1A, PPARGC1B and/or MITF in a melanoma patient in a sample of body fluid or tumor tissue of said patient, and comparing it with that of normal human melanocytes,
or
determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA or protein expression level of PPARGC1A, PPARGC1B and/or MITF or a lowered OCR following treatment with a BET inhibitor is detected, a therapeutically effective amount of a BET inhibitor is administered to the melanoma patient.

A further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B, and/or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B and/or MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA or protein expression level of PPARGC1A, PPARGC1B and/or MITF and a lowered OCR following treatment with a BET inhibitor is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B, and/or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B and/or MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA or protein expression level of PPARGC1A, PPARGC1B and/or MITF and a lowered OCR following treatment with a BET inhibitor is detected, a therapeutically effective amount of a BET inhibitor is administered to the melanoma patient.

A further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B, and/or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B and/or MITF in a melanoma patient in a sample of body fluid or tumor tissue of said patient, and comparing it with that of normal human melanocytes,

wherein the presence in said in vitro sample of an elevated mRNA or derived cDNA or protein expression level of PPARGC1A, PPARGC1B and/or MITF following treatment with a BET inhibitor is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B, and/or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B and/or MITF in a melanoma patient in a sample of body fluid or tumor tissue of said patient, and comparing it with that of normal human melanocytes,

wherein the presence in said in vitro sample of an elevated mRNA or derived cDNA or protein expression level of PPARGC1A, PPARGC1B and/or MITF is detected, a therapeutically effective amount of a BET inhibitor is administered to the melanoma patient.

A further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B, and/or MITF by measurement of the respective mRNA or derived cDNA expression levels in a sample of body fluid or tumor tissue of said patient, and comparing the expression level with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA expression level of PPARGC1A, PPARGC1B and/or MITF and a lowered OCR following treatment with a BET inhibitor is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B, and/or MITF by measurement of the respective mRNA or derived eDNA expression levels in a sample of body fluid or tumor tissue of said patient, and comparing the expression level with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA expression level of PPARGC1A, PPARGC1B and/or MITF and a lowered OCR following treatment with a BET inhibitor is detected, a therapeutically effective amount of a BET inhibitor is administered to the melanoma patient.

A further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
ii) determining the protein level of the stratification markers PPARGC1A, PPARGC1B and/or MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated protein expression level of PPARGC1A, PPARGC1B and/or MITF and a lowered OCR following treatment with a BET inhibitor is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
ii) determining the protein level of the stratification markers PPARG C IA, PPARG C1B and/or MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated protein expression level of PPARGC1A, PPARGC1B and/or MITF and a lowered OCR following treatment with a BET inhibitor is detected, a therapeutically effective amount of a BET inhibitor is administered to the melanoma patient.

The determination of the mRNA or derived cDNA, or protein expression level can be done with all of the stratification markers PPARGC1A, PPARGC1B and MITF, or can together be done with the stratification markers PPARGC1A and PPARGC1B, or with the stratification markers PPARGC1A and MITF, or can together be done with the stratification markers PPARGC1B and MITF, or can separately be done by measurement of the single stratification marker of PPARGC1A, PPARGC1B or MITF alone. All combinations are possible to get a valuable result for stratification and thus for the treatment of melanoma patients with an effective amount of a BET inhibitor.

Thus, a further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B or MITF by measurement of the respective mRNA or derived cDNA expression levels in a sample of body fluid or tumor tissue of said patient, and comparing the expression level with that of normal human melanocytes,
or
determining the protein level of the stratification markers PPARGC1A, PPARGC1B or MITF in a melanoma patient in a sample of body fluid or tumor tissue of said patient, and comparing it with that of normal human melanocytes,
or
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA, or protein expression level of PPARGC1A, PPARGC1B or MITF or a lowered OCR following treatment with a BET inhibitor is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B or MITF by measurement of the respective mRNA or derived cDNA expression levels in a sample of body fluid or tumor tissue of said patient, and comparing the expression level with that of normal human melanocytes,
or
ii) determining the protein level of the stratification markers PPARGC1A, PPARGC1B or MITF in a melanoma patient in a sample of body fluid or tumor tissue of said patient, and comparing it with that of normal human melanocytes,
or
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA, or protein expression level of PPARGC1A, PPARGC1B or MITF or a lowered OCR following treatment with a BET inhibitor is detected, a therapeutically effective amount of a BET inhibitor is administered to the melanoma patient.

Thus, a further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B and MITF by measurement of the respective mRNA or derived cDNA expression levels in a sample of body fluid or tumor tissue of said patient, and comparing the expression level with that of normal human melanocytes,
or
ii) determining the protein level of the stratification markers PPARGC1A, PPARGC1B and MITF in a melanoma patient in a sample of body fluid or tumor tissue of said patient, and comparing it with that of normal human melanocytes,
or
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA, or protein expression level of PPARGC1A, PPARGC1B and MITF or a lowered OCR following treatment with a BET inhibitor is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B and MITF by measurement of the respective mRNA or derived cDNA expression levels in a sample of body fluid or tumor tissue of said patient, and comparing the expression level with that of normal human melanocytes,
or
ii) determining the protein level of the stratification markers PPARGC1A, PPARGC1B and MITF in a melanoma patient in a sample of body fluid or tumor tissue of said patient, and comparing it with that of normal human melanocytes,
or
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA, or protein expression level of PPARGC1A, PPARGC1B and MITF or a lowered OCR following treatment with a BET inhibitor is detected, a therapeutically effective amount of a BET inhibitor is administered to the melanoma patient.

Thus, a further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A and PPARGC1B by measurement of the respective mRNA or derived cDNA expression levels in a sample of body fluid or tumor tissue of said patient, and comparing the expression level with that of normal human melanocytes,
or
ii) determining the protein level of the stratification markers PPARGC1A and PPARGC1B in a melanoma patient in a sample of body fluid or tumor tissue of said patient, and comparing it with that of normal human melanocytes,
or
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA, or protein expression level of PPARGC1A and PPARGC1B or a lowered OCR following treatment with a BET inhibitor is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A and PPARGC1B by measurement of the respective mRNA or derived cDNA expression levels in a sample of body fluid or tumor tissue of said patient, and comparing the expression level with that of normal human melanocytes,
or
ii) determining the protein level of the stratification markers PPARGC1A and PPARGC1B in a melanoma patient in a sample of body fluid or tumor tissue of said patient, and comparing it with that of normal human melanocytes,
or
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA, or protein expression level of PPARGC1A and PPARGC1B or a lowered OCR following treatment with a BET inhibitor is detected, a therapeutically effective amount of a BET inhibitor is administered to the melanoma patient.

Thus, a further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A and MITF by measurement of the respective mRNA or derived cDNA expression levels in a sample of body fluid or tumor tissue of said patient, and comparing the expression level with that of normal human melanocytes,
or
ii) determining the protein level of the stratification markers PPARGC1A and MITF in a melanoma patient in a sample of body fluid or tumor tissue of said patient, and comparing it with that of normal human melanocytes,
or
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA, or protein expression level of PPARGC1A and MTIF or a lowered OCR following treatment with a BET inhibitor is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A and MITF by measurement of the respective mRNA or derived cDNA expression levels in a sample of body fluid or tumor tissue of said patient, and comparing the expression level with that of normal human melanocytes,
or
ii) determining the protein level of the stratification markers PPARGC1A and MITF in a melanoma patient in a sample of body fluid or tumor tissue of said patient, and comparing it with that of normal human melanocytes,
or
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA, or protein expression level of PPARGC1A and MTIF or a lowered OCR following treatment with a BET inhibitor is detected, a therapeutically effective amount of a BET inhibitor is administered to the melanoma patient.

Thus, a further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1B and MITF by measurement of the respective mRNA or derived cDNA expression levels in a sample of body fluid or tumor tissue of said patient, and comparing the expression level with that of normal human melanocytes,
or
ii) determining the protein level of the stratification markers PPARGC1B and MITF in a melanoma patient in a sample of body fluid or tumor tissue of said patient, and comparing it with that of normal human melanocytes,
or
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA, or protein expression level of PPARGC1B and MT1F or a lowered OCR following treatment with a BET inhibitor is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1B and MITF by measurement of the respective mRNA or derived cDNA expression levels in a sample of body fluid or tumor tissue of said patient, and comparing the expression level with that of normal human melanocytes,
or
ii) determining the protein level of the stratification markers PPARGC1B and MITF in a melanoma patient in a sample of body fluid or tumor tissue of said patient, and comparing it with that of normal human melanocytes,
or
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA, or protein expression level of PPARGC1B and MTIF or a lowered OCR following treatment with a BET inhibitor is detected, a therapeutically effective amount of a BET inhibitor is administered to the melanoma patient.

Thus, a further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B or MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA and protein expression level of PPARGC1A, PPARGC1B or MTIF and a lowered OCR following treatment with a BET inhibitor is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B or MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA and protein expression level of PPARGC1A, PPARGC1B or MTIF and a lowered OCR following treatment with a BET inhibitor is detected, a therapeutically effective amount of a BET inhibitor is administered to the melanoma patient.

Thus, a further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA and protein expression level of PPARGC1A, PPARGC1B and MTIF and a lowered OCR following treatment with a BET inhibitor is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA and protein expression level of PPARGC1A, PPARGC1B and MTIF and a lowered OCR following treatment with a BET inhibitor is detected, a therapeutically effective amount of a BET inhibitor is administered to the melanoma patient.

Thus, a further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A and PPARGC1B by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A and PPARGC1B in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA and protein expression level of PPARGC1A and PPARGC1B and a lowered OCR following treatment with a BET inhibitor is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A and PPARGC1B by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A and PPARGC1B in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA and protein expression level of PPARGC1A and PPARGC1B and a lowered OCR following treatment with a BET inhibitor is detected, a therapeutically effective amount of a BET inhibitor is administered to the melanoma patient.

Thus, a further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA and protein expression level of PPARGC1A and MITF and a lowered OCR following treatment with a BET inhibitor is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA and protein expression level of PPARGC1A and MITF and a lowered OCR following treatment with a BET inhibitor is detected, a therapeutically effective amount of a BET inhibitor is administered to the melanoma patient.

Thus, a further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1B and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA and protein expression level of PPARGC1B and MITF and a lowered OCR following treatment with a BET inhibitor is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1B and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA and protein expression level of PPARGC1B and MITF and a lowered OCR following treatment with a BET inhibitor is detected, a therapeutically effective amount of a BET inhibitor is administered to the melanoma patient.

Thus, a further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B or MITF in a melanoma patient in a sample of body fluid or tumor tissue of said patient, and comparing it with that of normal human melanocytes,

wherein the presence in said in vitro sample of an elevated mRNA or derived cDNA and protein expression level of PPARGC1A, PPARGC1B or MITF is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B or MITF in a melanoma patient in a sample of body fluid or tumor tissue of said patient, and comparing it with that of normal human melanocytes,

wherein the presence in said in vitro sample of an elevated mRNA or derived cDNA and protein expression level of PPARGC1A, PPARGC1B or MITF is detected, a therapeutically effective amount of a BET inhibitor is administered to the melanoma patient.

Thus, a further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B and MITF in a melanoma patient in a sample of body fluid or tumor tissue of said patient, and comparing it with that of normal human melanocytes,

wherein the presence in said in vitro sample of an elevated mRNA or derived cDNA and protein expression level of PPARGC1A, PPARGC1B and MITF is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B and MITF in a melanoma patient in a sample of body fluid or tumor tissue of said patient, and comparing it with that of normal human melanocytes,

wherein the presence in said in vitro sample of an elevated mRNA or derived cDNA and protein expression level of PPARGC1A, PPARGC1B and MITF is detected, a therapeutically effective amount of a BET inhibitor is administered to the melanoma patient.

Thus, a further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A and PPARGC1B by measurement of the respective mRNA or derived cDNA expression levels in a sample of body fluid or tumor tissue of said patient, and comparing the expression level with that of normal human melanocytes,
and
ii) determining the protein level of the stratification markers PPARC;C lA and PPARGC1B in a melanoma patient in a sample of body fluid or tumor tissue of said patient, and comparing it with that of normal human melanocytes,

wherein the presence in said in vitro sample of an elevated mRNA or derived cDNA and protein expression level of PPARGC1A and PPARGC1B is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A and PPARGC1B by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A and PPARGC1B in a melanoma patient in a sample of body fluid or tumor tissue of said patient, and comparing it with that of normal human melanocytes,

wherein the presence in said in vitro sample of an elevated mRNA or derived cDNA and protein expression level of PPARGC1A and PPARGC1B is detected, a therapeutically effective amount of a BET inhibitor is administered to the melanoma patient.

Thus, a further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A and MITF in a melanoma patient in a sample of body fluid or tumor tissue of said patient, and comparing it with that of non⁻nal human melanocytes,

wherein the presence in said in vitro sample of an elevated mRNA or derived cDNA and protein expression level of PPARGC1A and M1TF is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A and M1TF in a melanoma patient in a sample of body fluid or tumor tissue of said patient, and comparing it with that of normal human melanocytes,

wherein the presence in said in vitro sample of an elevated mRNA or derived cDNA and protein expression level of PPARGC1A and MITF is detected, a therapeutically effective amount of a BET inhibitor is administered to the melanoma patient.

Thus, a further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1B and MITF in a melanoma patient in the sample of body fluid or tumor tissue of said patient, and comparing it with that of normal human melanocytes,

wherein the presence in said in vitro sample of an elevated mRNA or derived cDNA and protein expression level of PPARGC1B and MITF is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1B and MITF in a melanoma patient in a sample of body fluid or tumor tissue of said patient, and comparing it with that of normal human melanocytes,

wherein the presence in said in vitro sample of an elevated mRNA or derived cDNA and protein expression level of PPARGC1B and MITF is detected, a therapeutically effective amount of a BET inhibitor is administered to the melanoma patient.

Thus, a further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B or MITF by measurement of the respective mRNA or derived cDNA expression levels in a sample of body fluid or tumor tissue of said patient, and comparing the expression level with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA expression level of PPARGC1A, PPARGC1B or MITF, and a lowered OCR following treatment with a BET inhibitor is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B or MITF by measurement of the respective mRNA or derived cDNA expression levels in a sample of body fluid or tumor tissue of said patient, and comparing the expression level with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA expression level of PPARGC1A, PPARGC1B or MITF, and a lowered OCR following treatment with a BET inhibitor is detected, a therapeutically effective amount of a BET inhibitor is administered to the melanoma patient.

Thus, a further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B and MITF by measurement of the respective mRNA or derived cDNA expression levels in a sample of body fluid or tumor tissue of said patient, and comparing the expression level with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA expression level of PPARGC1A, PPARGC1B and MITF and a lowered OCR following treatment with a BET inhibitor is detected, a therapeutically effective amount of a BET inhibitor is administered to the melanoma patient.

Respectively, a further aspect of the invention is the use of a BET inhibitor for the production of a medicament for treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B and MITF by measurement of the respective mRNA or derived cDNA expression levels in a sample of body fluid or tumor tissue of said patient, and comparing the expression level with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA expression level of PPARGC1A, PPARGC1B and MITF and a lowered OCR following treatment with a BET inhibitor is detected, a therapeutically effective amount of a BET inhibitor is administered to the melanoma patient.

Thus, a further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A and PPARGC1B by measurement of the respective mRNA or derived cDNA expression levels in a sample of body fluid or tumor tissue of said patient, and comparing the expression level with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA expression level of PPARGC1A and PPARGC1B and a lowered OCR following treatment with a BET inhibitor is detected, a therapeutically effective amount of a BET inhibitor is administered to the melanoma patient.

Respectively, a further aspect of the invention is the use of a BET inhibitor for production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A and PPARGC1B by measurement of the respective mRNA or derived cDNA expression levels in a sample of body fluid or tumor tissue of said patient, and comparing the expression level with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA expression level of PPARGC1A and PPARGC1B and a lowered OCR following treatment with a BET inhibitor is detected, a therapeutically effective amount of a BET inhibitor is administered to the melanoma patient.

Thus, a further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A and MITF by measurement of the respective mRNA or derived cDNA expression levels in a sample of body fluid or tumor tissue of said patient, and comparing the expression level with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA expression level of PPARGC1A and M1TF and a lowered OCR following treatment with a BET inhibitor is detected, a therapeutically effective amount of a BET inhibitor is administered to the melanoma patient.

Respectively, a further aspect of the invention is the use of a BET inhibitor for production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A and MITF by measurement of the respective mRNA or derived cDNA expression levels in a sample of body fluid or tumor tissue of said patient, and comparing the expression level with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA expression level of PPARGC1A and MITF and a lowered OCR following treatment with a BET inhibitor is detected, a therapeutically effective amount of a BET inhibitor is administered to the melanoma patient.

Thus, a further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1B and MITF by measurement of the respective mRNA or derived cDNA expression levels in a sample of body fluid or tumor tissue of said patient, and comparing the expression level with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA expression level of PPARGC1B and MITF and a lowered OCR following treatment with a BET inhibitor is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1B and MITF by measurement of the respective mRNA or derived cDNA expression levels in a sample of body fluid or tumor tissue of said patient, and comparing the expression level with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA expression level of PPARGC1B and MITF and a lowered OCR following treatment with a BET inhibitor is detected, a therapeutically effective amount of a BET inhibitor is administered to the melanoma patient.

Thus, a further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
ii) determining the protein level of the stratification markers PPARGC1A, PPARGC1B or MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA expression level of PPARGC1A, PPARGC1B or MITF and a lowered OCR following treatment with a BET inhibitor is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
ii) determining the protein level of the stratification markers PPARGC1A, PPARGC1B or MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA expression level of PPARGC1A, PPARGC1B or MITF and a lowered OCR following treatment with a BET inhibitor is detected, a therapeutically effective amount of a BET inhibitor is administered to the melanoma patient.

Thus, a further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
ii) determining the protein level of the stratification markers PPARGC1A, PPARGC1B and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA expression level of PPARGC1A, PPARGC1B and MITF and a lowered OCR following treatment with a BET inhibitor is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
ii) determining the protein level of the stratification markers PPARGC1A, PPARGC1B and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA expression level of PPARGC1A, PPARGC1B and MITF and a lowered OCR following treatment with a BET inhibitor is detected, a therapeutically effective amount of a BET inhibitor is administered to the melanoma patient.

Thus, a further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
ii) determining the protein level of the stratification markers PPARGC1A and PPARGC1B in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated protein expression level of PPARGC1A and PPARGC1B and a lowered OCR following treatment with a BET inhibitor is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
ii) determining the protein level of the stratification markers PPARGC1A and PPARGC1B in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated protein expression level of PPARGC1A and PPARGC1B and a lowered OCR following treatment with a BET inhibitor is detected, a therapeutically effective amount of a BET inhibitor is administered to the melanoma patient.

Thus, a further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
ii) determining the protein level of the stratification markers PPARGC1A and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated protein expression level of PPARGC1A and MTIF and a lowered OCR following treatment with a BET inhibitor is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
ii) determining the protein level of the stratification markers PPARGC1A and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated protein expression level of PPARGC1A and MTIF and a lowered OCR following treatment with a BET inhibitor is detected, a therapeutically effective amount of a BET inhibitor is administered to the melanoma patient.

Thus, a further aspect of the invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
ii) determining the protein level of the stratification markers PPARGC1B and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated protein expression level of PPARGC1B and MTIF and a lowered OCR following treatment with a BET inhibitor is detected, a therapeutically effective amount of a BET inhibitor is administered to the melanoma patient.

Respectively, a further aspect of the invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
ii) determining the protein level of the stratification markers PPARGC1B and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated protein expression level of PPARGC1B and MTIF and a lowered OCR following treatment with a BET inhibitor is detected, a therapeutically effective amount of a BET inhibitor is administered to the melanoma patient.

When using the BET inhibitor for the treatment of melanoma in a patient, respectively when using the BET inhibitor for the production of a medicament for the treatment of melanoma in a patient, the respective mRNA or derived cDNA measurements of the PPARGC1A, PPARGC1B and MITF markers can be done separately or combined with the measurements of the protein expression level of the PPARGC1A, PPARGC1B and MITF markers.

For example, when using the BET inhibitor for the treatment of melanoma in a patient, respectively when using the BET inhibitor for the production of a medicament for the treatment of melanoma in a patient the following measurements are possible:
The respective mRNA or derived cDNA levels of the PPARGC1A, PPARGC1B and MITF markers combined with the protein expression level of the PPARGC1A, PPARGC1B or MITF marker.
The respective mRNA or derived cDNA levels of the PPARGC1A, PPARGC1B or MITF marker combined with the protein expression level of the PPARGC1A, PPARGC1B and MITF markers.
The respective mRNA or derived cDNA levels of the PPARGC1A and PPARGC1B markers combined with the protein expression level of the PPARGC1A or PPARGC1B marker.
The respective mRNA or derived cDNA levels of the PPARGC1A or PPARGC1B marker combined with the protein expression level of the PPARGC1A and PPARGC1B markers.
The respective mRNA or derived cDNA levels of the PPARGC1A and MITF markers combined with the protein expression level of the PPARGC1A or MITF marker.
The respective mRNA or derived cDNA levels of the PPARGC1A or MITF marker combined with the protein expression level of the PPARGC1A and MITF markers.
The respective mRNA or derived cDNA levels of the PPARGC1B and MITF markers combined with the protein expression level of the PPARGC1B or MITF marker.
The respective mRNA or derived cDNA levels of the PPARGC1B or MITF marker combined with the protein expression level of the PPARGC1B and MITF markers.

Thus, a further object of the present invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B or MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA level of the stratification markers PPARGC1A, PPARGC1B and MITF, and a protein expression level of PPARGC1A, PPARGC1B or MITF, and a lowered OCR following treatment with a BET inhibitor is detected, a therapeutically effective amount of a BET inhibitor is administered to the melanoma patient.

Respectively, a further object of the present invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B or MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA level of the stratification markers PPARGC1A, PPARGC1B and MITF, and a protein expression level of PPARGC1A, PPARGC1B or MITF, and a lowered OCR following treatment with a BET inhibitor is detected, a therapeutically effective amount of a BET inhibitor is administered to the melanoma patient.

Thus, a further object of the present invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA level of the stratification markers PPARGC1A, PPARGC1B or MITF, and a protein expression level of PPARGC1A, PPARGC1B and MITF, and a lowered OCR following treatment with a BET inhibitor is detected, a therapeutically effective amount of a BET inhibitor is administered to the melanoma patient.

Respectively, a further object of the present invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA level of the stratification markers PPARGC1A, PPARGC1B or MITF, and a protein expression level of PPARGC1A, PPARGC1B and MITF, and a lowered OCR following treatment with a BET inhibitor is detected, a therapeutically effective amount of a BET inhibitor is administered to the melanoma patient.

Thus, a further object of the present invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B or MITF in a melanoma patient in a sample of body fluid or tumor tissue of said patient, and comparing it with that of normal human melanocytes,

wherein the presence in said in vitro sample of an elevated mRNA or derived cDNA level of the stratification markers PPARGC1A, PPARGC1B and MITF, and a protein expression level of PPARGC1A, PPARGC1B or MITF, a therapeutically effective amount of a BET inhibitor is administered to the melanoma patient.

Respectively, a further object of the present invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC 1A, PPARGC1B or MITF in a melanoma patient in a sample of body fluid or tumor tissue of said patient, and comparing it with that of normal human melanocytes,

wherein the presence in said in vitro sample of an elevated mRNA or derived cDNA level of the stratification markers PPARGC1A, PPARGC1B and MITF, and a protein expression level of PPARGC1A, PPARGC1B or MITF, a therapeutically effective amount of a BET inhibitor is administered to the melanoma patient.

Thus, a further object of the present invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC 1A, PPARGC1B and MITF in a melanoma patient in a sample of body fluid or tumor tissue of said patient, and comparing it with that of normal human melanocytes,

wherein the presence in said in vitro sample of an elevated mRNA or derived cDNA level of the stratification markers PPARGC1A, PPARGC1B or MITF, and a protein expression level of PPARGC1A, PPARGC1B or MITF, a therapeutically effective amount of a BET inhibitor is administered to the melanoma patient.

Respectively, a further object of the present invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A, PPARGC1B or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B and MITF in a melanoma patient in a sample of body fluid or tumor tissue of said patient, and comparing it with that of normal human melanocytes,

wherein the presence in said in vitro sample of an elevated mRNA or derived cDNA level of the stratification markers PPARGC1A, PPARGC1B or MITF, and a protein expression level of PPARGC1A, PPARGC1B or MITF, a therapeutically effective amount of a BET inhibitor is administered to the melanoma patient.

Thus, a further object of the present invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A and PPARGC1B by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A or PPARGC1B in a melanoma patient in a sample of body fluid or tumor tissue of said patient, and comparing it with that of normal human melanocytes,

wherein the presence in said in vitro sample of an elevated mRNA or derived cDNA level of the stratification markers PPARGC1A and PPARGC1B, and a protein expression level of PPARGC1A or PPARGC1B, a therapeutically effective amount of a BET inhibitor is administered to the melanoma patient.

Respectively, a further object of the present invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A and PPARGC1B by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A or PPARGC1B in a melanoma patient in a sample of body fluid or tumor tissue of said patient, and comparing it with that of normal human melanocytes,

wherein the presence in said in vitro sample of an elevated mRNA or derived cDNA level of the stratification markers PPARGC1A and PPARGC113, and a protein expression level of PPARGC1A or PPARGC1B, a therapeutically effective amount of a BET inhibitor is administered to the melanoma patient.

Thus, a further object of the present invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A or PPARGC1B by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A and PPARGC113 in a melanoma patient in a sample of body fluid or tumor tissue of said patient, and comparing it with that of normal human melanocytes,

wherein the presence in said in vitro sample of an elevated mRNA or derived cDNA level of the stratification markers PPARGC1A or PPARGC1B, and a protein expression level of PPARGC1A and PPARGC1B, a therapeutically effective amount of a BET inhibitor is administered to the melanoma patient.

Respectively, a further object of the present invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A or PPARGC1B by measurement of the respective mRNA or derived cDNA expression levels in a sample of body fluid or tumor tissue of said patient, and comparing the expression level with that of normal human melanocytes,
and
ii) determining the protein level of the stratification markers PPARGC 1A and PPARGC1B in a melanoma patient in a sample of body fluid or tumor tissue of said patient, and comparing it with that of normal human melanocytes,

wherein the presence in said in vitro sample of an elevated mRNA or derived cDNA level of the stratification markers PPARGC1A or PPARGC1B, and a protein expression level of PPARGC1A and PPARGC1B, a therapeutically effective amount of a BET inhibitor is administered to the melanoma patient.

Thus, a further object of the present invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A or MITF in a melanoma patient in a sample of body fluid or tumor tissue of said patient, and comparing it with that of normal human melanocytes,

wherein the presence in said in vitro sample of an elevated mRNA or derived cDNA level of the stratification markers PPARGC1A and MITF, and a protein expression level of PPARGC1A or MITF, a therapeutically effective amount of a BET inhibitor is administered to the melanoma patient.

Respectively, a further object of the present invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A or MITF in a melanoma patient in a sample of body fluid or tumor tissue of said patient, and comparing it with that of normal human melanocytes,

wherein the presence in said in vitro sample of an elevated mRNA or derived cDNA level of the stratification markers PPARGC1A and MITF, and a protein expression level of PPARGC1A or MITF, a therapeutically effective amount of a BET inhibitor is administered to the melanoma patient.

Thus, a further object of the present invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A and MITF in a melanoma patient in a sample of body fluid or tumor tissue of said patient, and comparing it with that of normal human melanocytes,

wherein the presence in said in vitro sample of an elevated mRNA or derived cDNA level of the stratification markers PPARGC1A or MITF, and a protein expression level of PPARGC1A and MITF, a therapeutically effective amount of a BET inhibitor is administered to the melanoma patient.

Respectively, a further object of the present invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A and MITF in a melanoma patient in a sample of body fluid or tumor tissue of said patient, and comparing it with that of normal human melanocytes,

wherein the presence in said in vitro sample of an elevated mRNA or derived cDNA level of the stratification markers PPARGC1A or MITF, and a protein expression level of PPARGC1A and MITF, a therapeutically effective amount of a BET inhibitor is administered to the melanoma patient.

Thus, a further object of the present invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1B or MITF in a melanoma patient in a sample of body fluid or tumor tissue of said patient, and comparing it with that of normal human melanocytes,

wherein the presence in said in vitro sample of an elevated mRNA or derived cDNA level of the stratification markers PPARGC1A and MITF, and a protein expression level of PPARGC1B or MITF, a therapeutically effective amount of a BET inhibitor is administered to the melanoma patient.

Respectively, a further object of the present invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1B or MITF in a melanoma patient in a sample of body fluid or tumor tissue of said patient, and comparing it with that of normal human melanocytes,

wherein the presence in said in vitro sample of an elevated mRNA or derived cDNA level of the stratification markers PPARGC1 A and MITF, and a protein expression level of PPARGCI B or MITF, a therapeutically effective amount of a BET inhibitor is administered to the melanoma patient.

Thus, a further object of the present invention is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1B or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1B and MITF in a melanoma patient in a sample of body fluid or tumor tissue of said patient, and comparing it with that of normal human melanocytes,

wherein the presence in said in vitro sample of an elevated mRNA or derived cDNA level of the stratification markers PPARGC1B or MITF, and a protein expression level of PPARGC1B and MITF, a therapeutically effective amount of a BET inhibitor is administered to the melanoma patient.

Respectively, a further object of the present invention is the use of a BET inhibitor for the production of a medicament for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1B or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1B and MITF in a melanoma patient in a sample of body fluid or tumor tissue of said patient, and comparing it with that of normal human melanocytes,

wherein the presence in said in vitro sample of an elevated mRNA or derived cDNA level of the stratification markers PPARGC1B or MITF, and a protein expression level of PPARGC1B and MITF, a therapeutically effective amount of a BET inhibitor is administered to the melanoma patient

Of selected interest is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A or MITF by measurement of the respective mRNA or derived cDNA expression levels in a sample of body fluid or tumor tissue of said patient, and comparing the expression level with that of normal human melanocytes,
and/or
ii) determining the protein level of the stratification markers PPARGC1A or MITF in a melanoma patient in a sample of body fluid or tumor tissue of said patient, and comparing it with that of normal human melanocytes,
and/or
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA, or derived cDNA, and/or protein expression level of PPARGC1A, PPARGC1B and/or MITF, and/or a lowered OCR following treatment with a BET inhibitor is detected, a therapeutically effective amount of a BET inhibitor is administered to the melanoma patient.

More preferred is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A measurement of the respective mRNA or derived cDNA expression levels in a sample of body fluid or tumor tissue of said patient, and comparing the expression level with that of normal human melanocytes,
or
ii) determining the protein level of the stratification markers PPARGC1A in a melanoma patient in a sample of body fluid or tumor tissue of said patient, and comparing it with that of normal human melanocytes,
and/or
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA, or derived cDNA, or protein expression level of PPARGC1A and/or a lowered OCR following treatment with a BET inhibitor is detected, a therapeutically effective amount of a BET inhibitor is administered to the melanoma patient.

Much more preferred is the use of a BET inhibitor for the treatment of melanoma in a patient by stratifying a sample of body fluid or tumor tissue of said patient in vitro and determining whether a patient suffering from melanoma will respond to treatment with a BET inhibitor, by
i) determining the expression level of the stratification markers PPARGC1A by measurement of the respective mRNA or derived cDNA expression levels in a sample of body fluid or tumor tissue of said patient, and comparing the expression level with that of normal human melanocytes,
or
ii) determining the protein level of the stratification markers PPARGC1A in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA, or derived cDNA, or protein expression level of PPARGC1A, and a lowered OCR following treatment with a BET inhibitor is detected, a therapeutically effective amount of a BET inhibitor is administered to the melanoma patient.

An active amount of the inhibitor means an inhibitor concentration within the range of 0.05 to 5 μM, preferred within the range of 0.2 to 2 μM, more preferred in the range of 0.8 to 1.5 ?AM and most preferred is the amount of 1 μM. Said inhibitor concentrations can be achieved from concentrated stock solutions which are diluted with a suitable solvent. The concentration of such concentrated solutions vary from 5 mM to 100 mM, preferably a suitable inhibitor concentration is 10 mM.

Suitable solvents that can be used are for example dimethyl sulfoxide (DMSO), tetrahydrofuran, ethyl acetate, acetone, acetonitrile, isopropanol, ethanol, methanol, water.

A preferred suitable solvent is for example DMSO.

All compounds that are found to be active as bromodomain inhibitors can be used in the inventive in vitro test with the PPARGC1A, PPARGC1B or M1TF stratification marker, or the OCR stratification marker to determine whether a melanoma patient is a responder or non-responder to BET inhibition.

Today, several BET bromodomain inhibitors are known. Thus, it is a further object of the instant invention to use the known BET bromodomain inhibitors for stratification with the inventive in vivo method to predict their activity as cancer active compounds in patients, especially in patients suffering from melanoma.

BET bromodomain inhibitors that are known are for example those compounds that are disclosed in:

WO 2013/097052 concerning heterocyclic bromodomain inhibitors,

WO 2013/0158952 concerning isoindolone BET inhibitors,

WO 2013/024104 concerning 4-(8-methoxy-1-((l-methoxypropan-2-yl)-2-(tetrahydro-2H-pyran-4-yl)-1 H-imidazo[4,5-C]quinolin-7-yl)-3,5-dimethylisoxazoles as BET inhibitors,

WO 2013/097601 concerning bromodomain inhibitors,

WO 2013/184876 concerning benzo isoxazoloazepine bromodomain inhibitors,

WO 2013/184878 concerning benzo isoxazoloazepine as bromodomain inhibitors,

WO 2013/185284 concerning pyridinone and pyridazinone derivatives as BET inhibitors,

WO 2013/188381 concerning pyridinone and pyridazinone derivatives as BET inhibitors,

WO 2013/184876 concerning benzo-C-isoxazoloazepine bromodomain inhibitors,

WO 2014/001356 concerning thenotriazolodiazepines,

WO 2014/015175 concerning modulators of BRD4 bioactivity,

WO 2014/076146 concerning triazolopyridaz nes,

WO 2014/076237 concerning triazolopyrazines as BRD4 inhibitors,

WO 2014/076703 concerning BET inhibitors,

WO 2014/078257 concerning thieno[3,2-C]pyridin-4(5H)-ones as BET bromodomain inhibitors,

WO 2014/080290 concerning cyclic amines as bromodomain inhibitors,

WO 2014/080291 concerning byaryl derivatives as bromodomain inhibitors,

WO 2014/095774 concerning BET protein inhibiting dihydroquinoxalinones,

WO 2014/095775 concerning BET protein inhibiting dihydropyridopyrazinones,

WO 2014/0179648 concerning heterocyclic compounds as BET inhibitors,

WO 2014/128655 concerning substituted im dazo[4,5-C]quinoline derivatives,

WO 2014/134232 concerning carbazole compounds as bromodomain inhibitors,

WO 2014/134267 concerning carbazole compounds as bromodomain inhibitors,

WO 2014/139324 concerning tetracyclic bromodomain inhibitors,

WO 2014/140077 concerning furopyr dines as BET inhibitors,

WO 2014/140076 concerning 2,3-disubstituted 1-acyl-4-amino-1,2,3,4-tetrahydroquinoline,

WO 2014/143768 concerning tricyclic heterocycles as BET bromodomain inhibitors,

WO 2014/145051 concerning heterocyclic compounds,

WO 2014/152029 concerning oxazolo[5,4-C]quinolino-2-one,

WO 2014/154760 concerning indolinone analogues as BRD4 inhibitors,

WO 2014/154762 concerning dihydroquinazolinone analogues as BRD4 inhibitors,

WO 2014/159837 concerning methods and compositions for inhibition of bromodomain-containing proteins,

WO 2014/159392 concerning bromodomain binding reagents,

WO 2014/160873 concerning benzimidazolone derivatives as bromodomain inhibitors,

WO 2014/164596 concerning BET bromodomain inhibitors,

WO 2014/165143 concerning dihydro-pyrrolopyridinone bromodomain inhibitors,

WO 2014/170350 concerning compounds for use as bromodomain inhibitors,

WO 2014/173241 concerning substituted 5-(3,5-dimethyli soxazol-4-yl)indoline-2-ones,

WO 2014/182929 concerning benzimidazole derivatives as bromodomain inhibitors,

WO 2014/191894 concerning imidazopyrrolidinone derivatives as BET inhibitors,

WO 2014/191906 concerning pyrazolo-pyrrolidin-4-one derivatives as BET inhibitors,

WO 2014/191911 concerning pyrazolo-pyrrolidin-4-one derivatives as BET inhibitors,

WO 2014/193951 concerning pyrazolo-pyrrolidin-4-one derivatives as BET inhibitors,

WO 2014/202578 concerning substituted phenyl-2,3-benzodiazepines as BET inhibitors,

WO 2014/206150 concerning heterocyclic bromodomain inhibitors,

WO 2014/206345 concerning bromodomain inhibitors,

WO 2015/002754 concerning bicyclic bromodomain inhibitors,

WO 2015/013635 concerning inhibitors of bromodomain-containing proteins,

WO 2015/015318 concerning quinazolinones as bromodomain inhibitors,

WO 2015/018520 concerning BET/BRD4 inhibitors,

WO 2015/018523 concerning BET/BRD4 inhibitors,

WO 2015/022332 concerning pyridinones as BRD4 inhibitors,

WO 2015/031741 concerning deuterated thienotriazolodiazepine,

WO 2015/031824 concerning cyclic vinylogous amide BET inhibitors,

WO 2015/049629 concerning imidazoquinolines as bromodomain inhibitors,

WO 2015/058100 concerning bromodomain inhibitors,

WO 2015/067770 concerning triazolopyraz ne BET inhibitors.

Especially the therapeutic active compounds of interest exist in a multitude of forms but share the essential inhibitory function of interfering with the RNA or protein expression of PPARGC1A, PPARGC1B or MITF, or the OCR in proliferative diseases, such as melanoma.

There arc several selected BET bromodomain inhibitors available that arc tested in cancer cells.

Selected compounds of general interest for stratification are as follows:

JQ-1

[(RR,S)-4-(4-Chlorophenyl)-2,3,9-trimethyl-6H-1-thia-5,7,8,9a-tetraaza-cyclopenta[c]azulen-6-yl]-acetic acid tert-butyl ester

I-BET 762

(S)-2-(6-(4-chlorophenyl)-8-methoxy-1-methyl-4H-benzo[f][1,2,4]triazol o [4,3-a][1,4]diazepin-4-yl)-N-ethylacetamide

1-BET 151

7-(3,5-dimethylisoxazol-4-yl)-8-methoxy-1-((R)-1-(pyridin-2-yl)ethyl)-1H-imidazo [4,5-c]quinolin-2 (3H)-one

1-BET 726

2-[(4S)-6-(4-chlorophenyl)-8-methoxy-1-methyl-4H-[1,2,4] azolo[4,3-a][1.4]benzodiazepin-4-yl]N-ethylacctamide

OTX-015

(S)-2-[4-(4-chlorophenyl)-2,3,9-trimethyl-6H-thieno [3,2-f][1,2,4]triazolo-[4,3-a][1 ,4]diazepin-6-yl]-N-(4-hydroxyphenypacetamide,

CPI-203

(S)-2-(4-(4-chlorophenyl)-2,3 ,9-trimethyl-6 H-thieno [3,2-f][1,2 ,4]triazolo [4,3-a][1,4]diazepin-6-yl)acetamide,

CPI-0610

2-[(4S)-6-(4-chlorophenyl)-1-methyl-4H-[1,2]oxazolo[5,4-d][2]benzazepin-4-yl]acctamide

PFI-1

2-Methoxy-N-(3-methyl-2-oxo-1,2,3,4-tetrahydro-quinazolin-6-yl)-bezenesulfonamide

RVX-208

2-[4-(2-hydroxyl)thoxy)-3,5-dimethylphenyl]-5-dimethoxy-1H-quinazolin-4-one-bromosporine

MS-436

4-[(2Z)-2-(2-amino-5-methyl-4-oxocyclohexa-2,5-dien-1-ylidene)hydrazinyl]-N-pyridin-2-ylbenzenesulfonamide

For example, bromodomain inhibitors such as JQ1 (WO2011/143660), I-BET762 (WO2011/054553), OTX015 (EP0989131; US 5,712,274), CPI-0610 (WO 2012/075383), I-BET151 (WO2011/054846), PFI1 (presented at SCl/RSC Med. Chem. Symposium in 09/2011) and RVX-208 (WO2008/092231) can be used as inhibitor in the inventive method for treatment of stratified melanoma.

Preferably, [(R,S)-4-(4-Chlorophenyl)-2,3,9-trimethyl-6H-1-thia-5,7,8,9a-tetraaza-cyclopenta[e]azulen-6-yl]-acetic acid tert-butyl ester (JQ-1) can be used as inhibitor in the inventive method for treatment of stratified melanoma.

A further object of the invention is that compounds that are disclosed in WO2013/030150 (6H-Thieno[3,2-f][1,2,4]triazolo-[4,3-a][4,3-a][1,4] diazepines), WO2014/128111 (4-substituted Pyrrolo and Pyrazolo-Diazepines), WO2014/128070 (Pyrrolo- and Pyrazolo Diazepines) , WO2014/048945 (5-Aryl-Triazoloazepines), WO 2014/095774 (Dihydropyridopyrazinones), WO2014/202578 (2,3-Benzodiazepines), WO2014/12867 (Bicyclo- and spirocyclic substituted 2,3-Benzodeazepines), WO2015/004075 (Dihydrochinoxalinones and Dihydropyridopyrazinones) and WO2014/095775 (Dihydrochinoxalinones) can be used as inhibitors in the inventive method for treatment of stratified melanoma.

More especially those compounds have been found active within the inventive in vitro test that are disclosed in WO2014/026997 and which are of general formula (I)

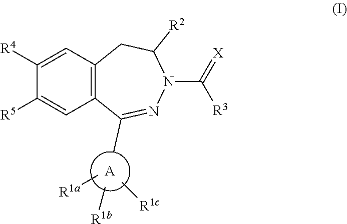

in which
X represents an oxygen or sulphur atom
A represents a monocyclic heteroaryl ring which has 5 or 6 ring atoms or represents a phenyl ring, and
R^{1a} represents hydrogen, halogen, cyano, carboxyl, amino or aminosulphonyl,
   or
   represents a C₁-C₆-alkoxy, C₁-C₃-alkoxy-C₂-C₃-alkoxy, C₁-C₆-alkylamino, C₁-C₆-alkylcarbonylamino, C₁-C₆-alkylamino-C₁-C₆-alkyl, N-(heterocyclyl)-C₁-C₆-alkyl, N-(heterocyclyl)-C₁-C₆-alkoxy, hydroxy-C₁-C₆-alkyl, hydroxy-C₁-C₆-alkoxy, halo-C₁-C₆-alkyl, halo-C₁-C₆-alkoxy, C₁-C₆-alkylcarbonyl or C₁-C₆-alkoxycarbonyl radical,
   or
   represents a monocyclic heterocyclyl radical having 3 to 8 ring atoms, which may optionally be mono- or polysubstituted by identical or different substituents from the group consisting of halogen, cyano, nitro, hydroxy, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, pyridinyl, —NR⁶C(═O)—R⁹, C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, and/or a monocyclic heterocyclyl radical having 3 to 8 ring atoms,
   or
   represents a monocyclic heteroaryl radical having 5 or 6 ring atoms, which may optionally be mono- or polysubstituted by identical or different substituents from the group consisting of halogen, hydroxy, amino, cyano, nitro, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, and/or by a monocyclic heterocyclyl radical having 3 to 8 ring atoms, and/or by a monocyclic heteroaryl radical having 5 or 6 ring atoms, and/or by a phenyl radical which for its part may optionally be mono- or polysubstituted by halogen, C₁-C₃-alkyl and/or C₁-C₃-alkoxy,
   or
   represents a phenyl radical, which may optionally be mono- or polysubstituted by identical or different substituents from the group consisting of halogen, amino, hydroxy, cyano, nitro, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino, C₁-C₆-alkyl, —C(═O)NR⁶R⁷, —C(═O)R⁸, C₁-C₃-alkylsulphinyl, C₁-C₃-alkylsulphonyl, —S(═O)₂NH₂, C₁-C₃-alkylsulphonylamino, C₁-C₃-alkylaminosulphonyl, C₃-C₆-cycloalkylaminosulphonyl, halo-C₁-C₆-alkyl, halo-C₁-C₆-alkoxy, hydroxy-C₁-C₆-alkyl, C₃-C₁₀-cycloalkyl, and/or a monocyclic heterocyclyl radical having 3 to 8 ring atoms, and/or a monocyclic heteroaryl radical having 5 or 6 ring atoms and which for its part may optionally be mono- or polysubstituted by identical or different substituents from the group consisting of halogen, C₁-C₃-alkyl and C₁-C₃-alkoxy, and
R^{1b} and R^{1c} independently of one another being able to represent hydrogen, halogen, hydroxy, cyano, nitro and/or a C₁-C₆-alkyl, C₁-C₆-alkoxy, C₁-C₆-alkoxy-C₁-C₆-alkyl, halo-C₁-C₆-alkyl, halo-C₁-C₆-alkoxy, C₃-C₁₀-cycloalkyl radical and/or a monocyclic heterocyclyl radical having 3 to 8 ring atoms, and
R² represents a C₁-C₃-alkyl or trifluoromethyl or a C₃- or C₄-cycloalkyl radical, and
R³ represents cyclopropyl, C₁-C₃ alkyl, C₁-C₃-alkoxy, amino, cyclopropylamino or C₁-C₃-alkylamino, and
R⁴ and R⁵ independently of one another represent hydrogen, hydroxy, cyano, nitro, amino, aminocarbonyl, fluorine, chlorine, bromine, C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl,
   or
   represent C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl which may optionally be mono- or polysubstituted by identical or different substituents from the group consisting of halogen, amino, hydroxy, carboxyl, hydroxy-C₁-C₆-alkyl, C₁-C₆-alkoxy, C₁-C₆-alkylamino, amino-C₁-C₆-alkyl, monocyclic heterocyclyl having 3 to 8 ring atoms and/or monocyclic heteroaryl having 5 or 6 ring atoms, where the monocyclic heterocyclyl and heteroaryl radicals mentioned for their part may optionally be monosubstituted by C₁-C₃-alkyl,
   or
   represent C₃-C₁₀-cycloalkyl which may optionally be mono- or polysubstituted by identical or different substituents from the group consisting of halogen, amino, hydroxy, carboxyl, C₁-C₆-alkyl, C₁-C₆-alkoxy, C₁-C₆-alkylamino, amino C₁-C₆-alkyl, C₁-C₆-alkylamino-C₁-C₆-alkyl, halo-C₁-C₆-alkyl, halo-C₁-C₆-alkoxy, and/or a monocyclic heterocyclyl radical having 3 to 8 ring atoms,
   or
   represent monocyclic heteroaryl which has 5 or 6 ring atoms and which may optionally be mono- or polysubstituted by identical or different substituents from the group consisting of halogen, amino, hydroxy, cyano, nitro, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆alkyl, halo-C₁-C₆-alkyl, halo-C₁-C₆-alkoxy, C₃-C₁₀-cycloalkyl, —C(═O)R⁸, —S(═O)₂R⁹, —NR⁶R⁷, and/or a monocyclic heterocyclyl radical having 3 to 8 ring atoms,
   or
   represent monocyclic heterocyclyl having 3 to 8 ring atoms and which may optionally be mono- or polysubstituted by identical or different substituents from the group consisting of halogen, amino, hydroxy, cyano, oxo, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, hydroxy-C₁-C₆-alkyl, halo-C₁-C₆-alkyl, halo-C₁-C₆-alkoxy, C₃-C₁₀-cycloalkyl, —C(═O)R⁸, —S(═O)₂R⁹, —NR⁶R⁷, and/or a monocyclic heterocyclyl radical having 3 to 8 ring atoms,
   or
   represent phenyl which may optionally be mono- or polysubstituted by identical or different substituents from the group consisting of halogen, amino, hydroxy, cyano, nitro, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylaminocarbonyl, C₁-C₆-alkylaminosulphonyl, C₁-C₆-alkylamino-C₁-C₆-alkyl, hydroxy-C₁-C₆-alkyl, halo-C₁-C₆-alkyl, halo-C₁-C₆-alkoxy, C₃-C₁₀-cycloalkyl and/or a monocyclic heterocyclyl radical having 3 to 8 ring atoms, and
R⁶ and R⁷ independently of one another represent hydrogen, C₁-C₃-alkyl, cyclopropyl or di-C₁-C₃-alkyl-amino-C₁-C₃-alkyl or fluoropyridyl, and
R⁸ represents hydroxy, C₁-C₆-alkyl, halo-C₁-C₃-alkyl, hydroxy-C₁-C₃-alkoxy-C₁-C₃-alkyl, C₃-C₈-cycloalkyl, phenyl, monocyclic heterocyclyl having 3 to 8 ring atoms or monocyclic heteroaryl having 5 or 6 ring atoms, where phenyl, heteroaryl and heterocyclyl may optionally be mono- or disubstituted by halogen, C₁-C₃-alkoxy or C₁-C₃-alkyl, and
R⁹ represents hydrogen, C₁-C₆-alkyl or C₁-C₄-alkoxy, and their polymorphs, enantiomers, diastereomers, racemates, tautomers, solvates, physiologically acceptable salts and solvates of these salts.

Preference is given to those compounds of the general formula 1 in which
X represents an oxygen atom, and
A represents a phenyl or pyridyl ring, and
R^{1a} represents hydrogen, halogen, cyano, carboxyl, amino or amino sulphonyl, or represents a C₁-C₆-alkoxy, C₁-C₃-alkoxy-C₁-C₃-alkyl, C₁-C₃-alkoxy-C₂-C₃-alkoxy, C₁-C₆-alkylamino, C₁-C₆-alkylcarbonylamino, C₁-C₆-alkylamino-C₁-C₆-alkyl, N-(heterocyclyl)-C₁-C₆-alkyl, N-(heterocyclyl)-C₁-C₆-alkoxy, hydroxy-C₁-C₆-alkyl, hydroxy-C₁-C₆-alkoxy, halo C₁-C₆ alkyl, halo-C₁-C₆-alkoxy, C₁-C₆-alkylcarbonyl or C₁-C₆-alkoxycarbonyl radical,
   or
   represents a monocyclic heterocyclyl radical having 4 to 7 ring atoms, which may optionally be mono- or polysubstituted by identical or different substituents from the group consisting of halogen, cyano, nitro, hydroxy, amino, oxo, carboxyl, C₁-C₆-alkyl, C₁-C₆-alkoxy, C₁-C₆-alkoxy-C₁-C₆alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, pyridinyl, —NR⁶C(═O)R⁹,
   —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, and/or by a monocyclic heterocyclyl radical having 4 to 7 ring atoms
   or
   represents a monocyclic heteroaryl radical having 5 or 6 ring atoms, which may optionally be mono- or polysubstituted by identical or different substituents from the group consisting of halogen, hydroxy, amino, cyano, nitro, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, and/or by a monocyclic heterocyclyl radical having 4 to 7 ring atoms and/or by a monocyclic heteroaryl radical having 5 or 6 ring atoms and/or by a phenyl radical which for its part may optionally be mono- or polysubstituted by identical or different substituents from the group consisting of halogen, C₁-C₃-alkyl and/or C₁-C₃-alkoxy,
   or
   represents a phenyl radical, which may optionally be mono- or polysubstituted by identical or different substituents from the group consisting of halogen, amino, hydroxy, cyano, nitro, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, —C(═O)NR⁶R⁷, C(═O)R⁸,
   C₁-C₃-alkylsulphinyl, C₁-C₃-alkylsulphonyl, —S(═O)₂NH₂, C₁-C₃-alkylsulphonylamino, C₁-C₃-alkylaminosulphonyl, C₃-C₆-cycloalkylaminosulphonyl, halo-C₁-C₆-alkyl, halo-C₁-C₆-alkoxy, hydroxy-C₁-C₆-alkyl, C₃-C₁₀-cycloalkyl, and/or a monocyclic heterocyclyl radical having 4 to 7 ring atoms and/or a monocyclic heteroaryl radical having 5 or 6 ring atoms and which for its part may optionally be mono- or polysubstituted by identical or different substituents from the group consisting of halogen, C₁-C₃-alkyl and C₁-C₃-alkoxy, and
R^{1b} and R^{1c} independently of one another represent hydrogen, halogen, hydroxy, cyano, nitro or a C₁-C₆-alkyl, C₁-C₆-alkoxy, C₁-C₆-alkoxy-C₁-C₆-alkyl, halo-C₁-C₆-alkyl, halo-C₁-C₆-alkoxy, C₃-C₁₀-cycloalkyl radical and/or a monocyclic heterocyclyl radical having 4 to 7 ring atoms, and
R² represents methyl, ethyl or isopropyl, and
R³ represents cyclopropyl, C₁-C₃-alkoxy, amino, cyclopropylamino or C₁-C₃-alkylamino, and
R⁴ and R⁵ independently of one another represent hydrogen, hydroxy, cyano, nitro, amino, aminocarbonyl, fluorine, chlorine, bromine, C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl,
   or
   represent C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl which may optionally be mono- or polysubstituted by identical or different substituents from the group consisting of halogen, amino, hydroxy, carboxyl, hydroxy-C₁-C₆-alkyl, C₁-C₆-alkoxy, C₁-C₆-alkoxy-C₁-C₆-alkyl, C₁-C₆-alkylamino, amino-C₁-C₆-alkyl, a monocyclic heterocyclyl having 4 to 7 ring atoms and/or a monocyclic heteroaryl having 5 or 6 ring atoms, where the monocyclic hetcrocyclyl and heteroaryl radicals mentioned for their part may optionally be monosubstituted by C₁-C₃-alkyl,
   or
   represent a C₃-C₁₀-cycloalkyl radical, which may optionally be mono- or polysubstituted by identical or different substituents from the group consisting of halogen, amino, hydroxy, carboxyl, C₁-C₆-alkyl, C₁-C₆-alkoxy, C₁-C₆-alkoxy-C₁-C₆-alkyl, C₁-C₆-alkylamino, amino C₁-C₆-alkyl, C₁-C₆-alkylamino C₁-C₆-alkyl, halo-C₁-C₆-alkyl, halo-C₁-C₆-alkoxy, and/or a monocyclic heterocyclyl radical having 4 to 7 ring atoms,
   or
   represent monocyclic heteroaryl having 5 or 6 ring atoms and which may optionally be mono- or polysubstituted by identical or different substituents from the group consisting of halogen, amino, hydroxy, cyano, nitro, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, —C(═O)R⁸, —S(═O)₂R⁹, —NR⁶R⁷, and/or a monocyclic heterocyclyl radical having 4 to 7 ring atoms,
   or
   represent monocyclic heterocyclyl having 4 to 7 ring atoms and which may optionally be mono- or polysubstituted by identical or different substituents from the group consisting of halogen, amino, hydroxy, cyano, oxo, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, hydroxy-C₁-C₆-alkyl, halo C₁-C₆-alkyl, halo-C₁-C₆-alkoxy, C₃-C₁₀-cycloalkyl, —C(═O)R⁸, —S(═O)₂R⁹, —NR⁶R⁷, and/or a monocyclic heterocyclyl radical having 4 to 7 ring atoms,
   or
   represent a phenyl radical, which may optionally be mono- or polysubstituted by identical or different substituents from the group consisting of halogen, amino, hydroxy, cyano, nitro, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylaminocarbonyl, C₁-C₆-alkylaminosulphonyl, C₁-C₆-alkylamino-C₁-C₆-alkyl, hydroxy-C₁-C₆-alkyl, halo-C₁-C₆-alkyl, halo-C₁-C₆-alkoxy, C₃-C₁₀-cycloalkyl, and/or a monocyclic heterocyclyl radical having 4 to 7 ring atoms, and
R⁶ and R⁷ independently of one another represent hydrogen, C₁-C₃-alkyl, cyclopropyl, di-C₁-C₃-alkyl-amino-C₁-C₃-alkyl or fluoropyridyl, and
R⁸ represents hydroxy, C₁-C₆-alkyl, halo-C₁-C₃-alkyl, hydroxy-C₁-C₃-alkyl, C₁-C₃-alkoxy-C₁-C₃-alkyl, C₃-C₈-cycloalkyl, phenyl, monocyclic heterocyclyl having 5 or 6 ring atoms, and
R⁹ represents hydrogen, C₁-C₆-alkyl or C₁-C₄-alkoxy, and their polymorphs, enantiomers, diastereomers, racemates, tautomers, solvates, physiologically acceptable salts and solvates of these salts.

Particular preference is given to those compounds of the general formula (I) in which
X represents an oxygen atom, and
A represents a phenyl or pyridyl ring, and
R^{1a} represents hydrogen, halogen, cyano, carboxyl, amino or aminosulphonyl, or represents a C₁-C₆-alkoxy, C₁-C₆-alkoxy-C₁-C₃-alkyl, C₁-C₃-alkoxy-C₂-C₃-alkoxy, C₁-C₃-alkylamino, C₁-C₃-alkylcarbonylamino, C₁-C₃-alkylamino-C₁-C₃-alkyl, hydroxy-C₁-C₃-alkyl, fluoro-C₁-C₃-alkyl, fluoro-C₁-C₃-alkoxy, C₁-C₃-alkylcarbonyl or C₁-C₄-alkoxycarbonyl radical,
   or
   represents a monocyclic heterocyclyl radical having 4 to 7 ring atoms, which may optionally be mono- or polysubstituted by identical or different substituents from the group consisting of halogen, cyano, nitro, hydroxy, amino, oxo, carboxyl, C₁-C₃-alkyl, C₁-C₃-alkoxy, C₁-C₂-alkoxy-C₁-C₂-alkyl, hydroxy-C₁-C₃-alkyl, C₁-C₃-alkylamino, amino-C₁-C₃-alkyl, fluoro-C₁-C₃-alkyl, fluoro-C₁-C₃-alkoxy, C₃-C₆-cycloalkyl, phenyl, halophenyl, phenyl-C₁-C₃-alkyl, pyridinyl, —NR⁶C(═O)—R⁹, —C(═O)—NR⁶R⁷, —C(═O)—R⁸,
   —S(═O)₂—NR⁶R⁷, —S(═O)—R⁹, —S(═O)₂—R⁹, —NH—S(═O)₂—R⁹, and/or by a monocyclic heterocyclyl radical having 4 to 7 ring atoms,
   or
   represents a monocyclic heteroaryl radical having 5 or 6 ring atoms, which may optionally be mono- or polysubstituted by identical or different substituents from the group consisting of halogen, hydroxy, amino, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, fluoro-C₁-C₃-alkyl, fluoro-C₁-C₃-alkoxy, C₃-C₆-cycloalkyl, —C(═O)—NR⁶R², —C(═O)—R⁸, —S(═O)₂—NR⁶R⁷, —S(═O)—R⁹, —S(═O)₂—R⁹, —NH—S(═O)₂—R⁹, and/or by a monocyclic heterocyclyl radical having 4 to 7 ring atoms and/or by a monocyclic heteroaryl radical having 5 or 6 ring atoms and/or by a phenyl radical which for its part may optionally be mono- or polysubstituted by identical or different substituents from the group consisting of fluorine, chlorine, bromine, methyl and/or methoxy,
   or
   represents a phenyl radical, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, —C(═O)NR⁶R⁷, —C(═O)R⁸, C₁-C₃-alkylsulphinyl, C₁-C₃-alkylsulphonyl, —S(═O)₂NH₂, C₁-C₃-alkylsulphonylamino, C₁-C₃-alkylaminosulphonyl, C₃-C₆-cycloalkylaminosulphonyl, fluoro-C₁-C₃-alkyl, fluoro-C₁-C₃-alkoxy, hydroxy-C₁-C₃-alkyl, C₃-C₆-cycloalkyl and/or a monocyclic heterocyclyl radical having 4 to 7 ring atoms and/or a monocyclic heteroaryl radical having 5 or 6 ring atoms and which for its part may optionally be mono- or polysubstituted by identical or different substituents from the group consisting of fluorine, chlorine, bromine, methyl and/or methoxy, and
R^{1b} represents hydrogen, halogen, hydroxy, cyano, nitro or represents a C₁-C₃-alkyl, C₁-C₃-alkoxy, fluoro-C₁-C₃-alkyl or fluoro-C₁-C₃-alkoxy radical, and
R^{1c} represents hydrogen, fluorine, chlorine, bromine or cyano, and
R² represents methyl, ethyl or isopropyl, and
R³ represents cyclopropyl, C₁-C₃ alkyl, C₁-C₃-alkoxy, cyclopropylamino or C₁-C₃-alkylamino, and
R⁴ and R⁵ independently of one another represent hydrogen, hydroxy, cyano, nitro, amino, aminocarbonyl, fluorine, chlorine, bromine, C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl,
   or
   represent C₁-C₃-alkyl, C₁-C₃-alkoxy, C₁-C₃-alkylamino, which may be mono- or polysubstituted by identical or different substituents from the group consisting of halogen, amino, hydroxy, carboxyl, hydroxy-C₁-C₃-alkyl, C₁-C₃-alkoxy, C₁-C₃-alkylamino, amino-C₁-C₃-alkyl, monocyclic heterocyclyl having 4 to 7 ring atoms, and/or monocyclic heteroaryl having 5 or 6 ring atoms, where the monocyclic heterocyclyl and heteroaryl radicals mentioned for their part may optionally be monosubstituted by C₁-C₃-alkyl,
   or
   represent a C₃-C₇-cycloalkyl radical, which may optionally be mono- or polysubstituted by identical or different substituents from the group consisting of halogen, amino, hydroxy, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, fluoro-C₁-C₃-alkyl, fluoro-C₁-C₃-alkoxy, and/or a monocyclic heterocyclyl radical having 4 to 7 ring atoms,
   or
   represent monocyclic heterocyclyl having 5 or 6 ring atoms and which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, hydroxy-C₁-C₃-alkyl, amino-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷, and/or a monocyclic heterocyclyl radical having 4 to 7 ring atoms,
   or
   represent monocyclic heterocyclyl having 4 to 7 ring atoms and which may optionally be mono- or polysubstituted by identical or different substituents from the group consisting of halogen, amino, hydroxy, cyano, oxo, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, hydroxy-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷ and/or a monocyclic heterocyclyl radical having 4 to 7 ring atoms,
   or
   represent a phenyl radical, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, C₁-C₃-alkylaminocarbonyl, C₁-C₃-alkylaminosulphonyl, hydroxy-C₁-C₃-alkyl, fluoro-C₁-C₃-alkyl, fluoro-C₁-C₃-alkoxy, C₃-C₆-cycloalkyl and/or a monocyclic heterocyclyl radical having 4 to 7 ring atoms, and
R⁶ and R⁷ independently of one another represent hydrogen, C₁-C₃-alkyl, cyclopropyl, di-C₁-C₃-alkylamino-C₁-C₃-alkyl or fluoropyridyl, and
R⁸ represents hydroxy, C₁-C₆-alkyl, hydroxy-C₁-C₃-alkyl, fluoro C₁-C₃ alkyl, C₁-C₃-alkoxy-C₁-C₃-alkyl, C₃-C₈-cycloalkyl or monocyclic heterocyclyl which has 5 or 6 ring atoms, and
R⁹ represents hydrogen, C₁-C₆-alkyl or C₁-C₄-alkoxy, and their polymorphs, enantiomers, diastereomers, racemates, tautomers, solvates, physiologically acceptable salts and solvates of these salts.

Particular preference is further given to those compounds of the general formula (1) in which
X represents an oxygen atom, and
A represents a phenyl or pyridyl ring, and
R^{1a} represents a monocyclic hcterocycyl radical having 4 to 7 ring atoms, which may optionally be mono- or polysubstituted by identical or different substituents from the group consisting of halogen, cyano, nitro, hydroxy, amino, oxo, carboxyl, C₁-C₃-alkyl, C₁-C₃-alkoxy, C₁-C₂-alkoxy-C₁-C₂-alkyl, hydroxy-C₁-C₃-alkyl, C₁-C₃-alkylamino, amino-C₁-C₃-alkyl, fluoro C₁-C₃ alkyl, fluoro-C₁-C₃-alkoxy, C₃-C₆-cycloalkyl, phenyl, halophenyl, phenyl-C₁-C₃-alkyl, pyridinyl, —NR⁶C(═O)—R⁹, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, and/or by a monocyclic heterocyclyl radical having 4 to 7 ring atoms,
   or
   represents a monocyclic heteroaryl radical having 5 or 6 ring atoms, which may optionally be mono- or polysubstituted by identical or different substituents from the group consisting of halogen, hydroxy, amino, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, fluoro-C₁-C₃-alkyl, fluoro-C₁-C₃-alkoxy, C₃-C₆-cycloalkyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, and/or by a monocyclic heterocyclyl radical having 4 to 7 ring atoms, and/or by a monocyclic heteroaryl radical having 5 or 6 ring atoms, and/or by a phenyl radical which for its part may be mono- or polysubstituted by identical or different substituents from the group consisting of fluorine, chlorine, bromine, methyl or methoxy,
   or
   represents a phenyl radical, which may optionally be mono- or polysubstituted by identical or different substituents from the group consisting of halogen, amino, hydroxy, cyano, nitro, carboxyl, C₁-C₃-alkyl, C₁-C₃ alkoxy, C₁-C₂-alkoxy-C₁-C₂-alkyl, C₁-C₃-alkylamino, amino-C₁-C₃-alkyl, —C(═O)NR⁶R⁷, —C(═O)R⁸, C₁-C₃-alkylsulphinyl, C₁-C₃-alkylsulphonyl, —S(═O)₂NH₂, C₁-C₃-alkylsulphonylamino, C₁-C₃-alkylaminosulphonyl, C₃-C₆-cycloalkylaminosulphonyl, fluoro-C₁-C₃-alkyl, fluoro-C₁-C₃-alkoxy, hydroxy-C₁-C₃-alkyl, C₃-C₆-cycloalkyl and/or a monocyclic heterocyclyl radical having 4 to 7 ring atoms, and/or a monocyclic heteroaryl radical having 5 or 6 ring atoms which for its part may optionally be mono- or polysubstituted by identical or different substituents from the group consisting of fluorine, chlorine, bromine, methyl or methoxy, and
R^{1b} represents hydrogen, halogen, hydroxy, cyano, nitro or represents a C₁-C₃-alkyl, C₁-C₃-alkoxy, fluoro-C₁-C₃-alkyl or fluoro-C₁-C₃-alkoxy radical, and
R^{1c} represents hydrogen, fluorine, chlorine, bromine or cyano, and
R² represents methyl, ethyl or isopropyl, and
R³ represents cyclopropyl, C₁-C₃-alkoxy, cyclopropylamino or C₁-C₃-alkylamino, and
R⁴ and R⁵ independently of one another represent hydrogen, hydroxy, cyano, nitro, amino, aminocarbonyl, fluorine, chlorine, bromine, C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl,
   or
   represent C₁-C₃-alkyl, C₁-C₃-alkoxy, C₁-C₃-alkylamino, which may be mono- or polysubstituted by identical or different substituents from the group consisting of halogen, amino, hydroxy, carboxyl, hydroxy-C₁-C₃-alkyl, C₁-C₃-alkoxy, C₁-C₃-alkylamino, amino-C₁-C₃-alkyl, monocyclic heterocyclyl having 4 to 7 ring atoms, and/or monocyclic heteroaryl having 5 or 6 ring atoms, where the monocyclic heterocyclyl and heteroaryl radicals mentioned for their part may optionally be monosubstituted by C₁-C₃-alkyl,
   or
   represent a C₃-C₇-cycloalkyl radical, which may optionally be mono- or polysubstituted by identical or different substituents from the group consisting of halogen, amino, hydroxy, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, fluoro-C₁-C₃-alkyl, fluoro-C₁-C₃-alkoxy, and/or a monocyclic heterocyclyl radical having 4 to 7 ring atoms,
   Or
   represent monocyclic heteroaryl having 5 or 6 ring atoms, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, hydroxy-C₁-C₃-alkyl, C₁-C₃-alkylamino, amino-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷, and/or a monocyclic heterocyclyl radical having 4 to 7 ring atoms,
   or
   represent monocyclic heterocyclyl having 4 to 7 ring atoms, which may optionally be mono- or polysubstituted by identical or different substituents from the group consisting of halogen, amino, hydroxy, cyano, oxo, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, hydroxy-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷, and/or a monocyclic heterocyclyl radical having 4 to 7 ring atoms,
   or
   represent a phenyl radical, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, C₁-C₃-alkylaminocarbonyl, C₁-C₃-alkylaminosulphonyl, hydroxy-C₁-C₃-alkyl, fluoro-C₁-C₃-alkyl, fluoro-C₁-C₃-alkoxy, C₃-C₆-cycloalkyl, and/or a monocyclic heterocyclyl radical having 4 to 7 ring atoms, and
R⁶ and R⁷ independently of one another represent hydrogen, C₁-C₃-alkyl, cyclopropyl, di-C₁-C₃-alkylamino-C₁-C₃-alkyl or fluoropyridyl, and
R⁸ represents hydroxy, C₁-C₆-alkyl, hydroxy-C₁-C₃-alkyl, fluoro-C₁-C₃-alkoxy-C₁-C₃alkyl, C₃-C₈-cycloalkyl or monocyclic heterocyclyl which has 5 or 6 ring atoms, and
R⁹ represents hydrogen, C₁-C₆-alkyl or C₁-C₄-alkoxy, and their polymorphs, enantiomers, diastereomers, racemates, tautomers, solvates, physiologically acceptable salts and solvates of these salts.

Particular preference is further given to those compounds of the general formula (I) in which
X represents an oxygen atom, and
A represents a phenyl or pyridyl ring, and
R^{1a} represents hydrogen, halogen, cyano, carboxyl, amino or aminosulphonyl, or represents a C₁-C₆-alkoxy, C₁-C₃-alkoxy-C₁-C₃-alkyl, C₁-C₃-alkoxy-C₂-C₃-alkoxy, C₁-C₃-alkylamino, C₁-C₃-alkylcarbonylamino, C -C₃-alkylamino-C₁-C₃-alkyl, hydroxy-C₁-C₃-alkyl, fluoro-C₁-C₃-alkyl, fluoro-C₁-C₃-alkoxy, C₁-C₃-alkylcarbonyl or C₁-C₄-alkoxycarbonyl radical,
   or
   represents a monocyclic heterocyclyl radical having 4 to 7 ring atoms, which may optionally be mono- or polysubstituted by identical or different substituents from the group consisting of halogen, cyano, nitro, hydroxy, amino, oxo, carboxyl, C₁-C₃-alkyl, C₁-C₃-alkoxy, C₁-C₂-alkoxy-C₁-C₂-alkyl , hydroxy-C₁-C₃-alkyl, C₁-C₃-alkylamino, amino-C₁-C₃-alkyl, fluoro-C₁-C₃-alkyl, fluoro-C₁-C₃-alkoxy, C₃-C₆-cycloalkyl, phenyl, halophenyl, phenyl-C₁-C₃-alkyl, pyridinyl, —NR⁶C(═O)—R⁹, —C(═O)—NR⁶R⁷, —C(═O)—R⁸,
   —S(═O)₂—NR⁶R⁷, —S(═O)—R⁹, —S(═O)₂—R⁹, —NH—S(═O)₂—R⁹, and/or by a monocyclic heterocyclyl radical having 4 to 7 ring atoms,
   or
   represents a monocyclic heteroaryl radical having 5 or 6 ring atoms, which may optionally be mono- or polysubstituted by identical or different substituents from the group consisting of halogen, hydroxy, amino, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, fluoro-C₁-C₃-alkyl, fluoro-C₁-C₃-alkoxy, C₃-C₆-cycloalkyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, and/or by a monocyclic heterocyclyl radical having 4 to 7 ring atoms, and/or by a monocyclic heteroaryl radical having 5 or 6 ring atoms, and/or by a phenyl radical which for its part may be mono- or polysubstituted by identical or different substituents from the group consisting of fluorine, chlorine, bromine, methyl and/or methoxy,
   or
   represents a phenyl radical, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, —C(—O)NR⁶R⁷, C(—O)R⁸, C₁-C₃-alkylsulphinyl, C₁-C₃-alkylsulphonyl, —S(═O)₂NH₂, C₁-C₃-alkylsulphonylamino, C₁-C₃-alkylaminosulphonyl, C₃-C₆-cycloalkylaminosulphonyl, fluoro-C₁-C₃-alkyl, fluoro-C₁-C₃-alkoxy, hydroxy-C₁-C₃-alkyl, C₃-C₆-cycloalkyl and/or a monocyclic heterocyclyl radical having 4 to 7 ring atoms, and/or a monocyclic heteroaryl radical having 5 or 6 ring atoms which for its part may optionally be mono- or polysubstituted by identical or different substituents from the group consisting of fluorine, chlorine, bromine, methyl and/or methoxy, and
R^{1b} represents hydrogen, halogen, hydroxy, cyano, nitro or represents a C₁-C₃-alkyl, C₁-C₃-alkoxy, fluoro-C₁-C₃-alkyl or fluoro-C₁-C₃-alkoxy radical, and
R^{1c} represents hydrogen, fluorine, chlorine, bromine or cyano, and
R² represents methyl, ethyl or isopropyl, and
R³ represents cyclopropyl, C₁-C₃-alkyl, C₁-C₃-alkoxy, cyclopropylamino or C₁-C₃-alkylamino, and
R⁴ represents a C₃-C₇-cycloalkyl radical, which may optionally be mono- or polysubstituted by identical or different substituents from the group consisting of halogen, amino, hydroxy, carboxyl, C₁-C₃-alkyl, C₁-C₃-alkoxy, C₁-C₂-alkoxy-C₁-C₂-alkyl, C₁-C₃-alkylamino, amino-C₁-C₃ alkyl, fluoro-C₁-C₃-alkyl, tluoro-C₁-C₃-alkoxy, and/or a monocyclic heterocyclyl radical having 4 to 7 ring atoms,
   or
   represents monocyclic heteroaryl having 5 or 6 ring atoms, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, hydroxy-C₁-C₃-alkyl, C₁-C₃-alkylamino, amino-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷, and/or a monocyclic heterocyclyl radical having 4 to 7 ring atoms,
   or
   represents monocyclic heterocyclyl having 4 to 7 ring atoms, which may optionally be mono- or polysubstituted by identical or different substituents from the group consisting of halogen, amino, hydroxy, cyano, oxo, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, hydroxy-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷, and/or a monocyclic heterocyclyl radical having 4 to 7 ring atoms,
   or
   represents a phenyl radical, which may optionally be mono- or polysubstituted by identical or different substituents from the group consisting of halogen, amino, hydroxy, cyano, nitro, carboxyl, C₁-C₃-alkoxy, C₁-C₃-alkoxy-C₁-C₂-alkyl, C₁-C₃-alkylamino, amino-C₁-C₃-alkyl, C₁-C₃-alkylaminocarbonyl, C₁-C₃-alkylaminosulphonyl, hydroxy-C₁-C₃-alkyl, fluoro-C₁-C₃-alkyl, fluoro-C₁-C₃-alkoxy, C₃-C₆-cycloalkyl, and/or a monocyclic heterocyclyl radical having 4 to 7 ring atoms, and
R⁵ represents hydrogen, hydroxy, cyano, nitro, amino, aminocarbonyl, fluorine, chlorine, bromine, C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl, and
R⁶ and R⁷ independently of one another represent hydrogen, C₁-C₃-alkyl, cyclopropyl, di-C₁-C₃-alkylamino-C₁-C₃-alkyl or fluoropyridyl, and
R⁸ represents hydroxy, C₁-C₆-alkyl, hydroxy-C₁-C₃-alkyl, fluoro-C₁-C₃-alkoxy-C₁-C₃-alkyl, C₃-C₈-cycloalkyl or monocyclic heterocyclyl which has 5 or 6 ring atoms, and
R⁹ represents hydrogen, C₁-C₆-alkyl or C₁-C₄-alkoxy, and their polymorphs, cnantiomers, diastereomers, racemates, tautomers, solvates, physiologically acceptable salts and solvates of these salts.

Particular preference is further given to those compounds of the general formula (1) in which
X represents an oxygen atom, and
A represents a phenyl or pyridyl ring, and
R^{1a} represents hydrogen, halogen, cyano, carboxyl, amino or aminosulphonyl, or represents a C₁-C₆-alkoxy, C₁-C₃-alkoxy-C₁-C₃-alkyl, C₁-C₃-alkoxy-C₂-C₃-alkoxy, C₁-C₃-alkylamino, C₁-C₃-alkylcarbonylamino, C₁-C₃-alkylamino-C₁-C₃-alkyl, hydroxy-C₁-C₃-alkyl, fluoro-C₁-C₃-alkyl, fluoro-C₁-C₃-alkoxy, C₁-C₃-alkylcarbonyl or C₁-C₄-alkoxycarbonyl radical,
   or
   represents a monocyclic heterocyclyl radical having 4 to 7 ring atoms, which may optionally be mono- or polysubstituted by identical or different substituents from the group consisting of halogen, cyano, nitro, hydroxy, amino, oxo, carboxyl, C₁-C₃-alkyl, C₁-C₃-alkoxy, C₁-C₂-alkoxy-C₁-C₂-alkyl, hydroxy-C₁-C₃-alkyl, C₁-C₃-alkylamino, amino-C₁-C₃-alkyl, fluoro-C₁-C₃-alkyl, fluoro-C₁-C₃-alkoxy, C₃-C₆-cycloalkyl, phenyl, halophenyl, phenyl-C₁-C₃-alkyl, pyridinyl, —NR⁶C(═O)—R⁹, —C(═O)—NR⁶R⁷, —C(═O)—R⁸,
   —S(═O)₂—NR⁶R⁷, —S(═O)—R⁹, —S(═O)₂—R⁹, —NH—S(═O)₂—R⁹, and/or by a monocyclic heterocyclyl radical having 4 to 7 ring atoms,
   or
   represents a monocyclic heteroaryl radical having 5 or 6 ring atoms, which may optionally be mono- or polysubstituted by identical or different substituents from the group consisting of halogen, hydroxy, amino, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, fluoro-C₁-C₃-alkyl, fluoro-C₁-C₃-alkoxy, C₃-C₆-cycloalkyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, and/or by a monocyclic heterocyclyl radical having 4 to 7 ring atoms, and/or by a monocyclic heteroaryl radical having 5 or 6 ring atoms, and/or by a phenyl radical which for its part may be mono- or polysubstituted by identical or different substituents from the group consisting of fluorine, chlorine, bromine, methyl and/or methoxy,
   or
   represents a phenyl radical, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, —C(═O)NR⁶R⁷, —C(═O)R⁸, C₁-C₃-alkylsulphinyl, C₁-C₃-alkylsulphonyl, —S(═O)₂NH₂, C₁-C₃-alkylsulphonylamino, C₁-C₃-alkylaminosulphonyl, C₃-C₆-cycloalkylaminosulphonyl, fluoro-C₁-C₃-alkyl, fluoro-C₁-C₃-alkoxy, hydroxy-C₁-C₃-alkyl, C₃-C₆-cycloalkyl and/or a monocyclic heterocyclyl radical having 4 to 7 ring atoms, and/or a monocyclic heteroaryl radical having 5 or 6 ring atoms which for its part may optionally be mono- or polysubstituted by identical or different substituents from the group consisting of fluorine, chlorine, bromine, methyl and/or methoxy, and
R^{1b} represents hydrogen, halogen, hydroxy, cyano, nitro or represents a C₁-C₃-alkyl, C₁-C₃-alkoxy, fluoro-C₁-C₃-alkyl or fluoro-C₁-C₃-alkoxy radical, and
R^{1c} represents hydrogen, fluorine, chlorine, bromine or cyano, and
R² represents methyl, ethyl or isopropyl, and
R³ represents cyclopropyl, C₁-C₃-alkoxy, cyclopropylamino or C₁-C₃-alkylamino, and
R⁴ represents hydrogen, hydroxy, cyano, nitro, amino, aminocarbonyl, fluorine, chlorine, bromine, C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl, and
R⁵ represents a C₃-C₇-cycloalkyl radical, which may optionally be mono- or polysubstituted by identical or different substituents from the group consisting of halogen, amino, hydroxy, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, fluoro-C₁-C₃-alkyl, fluoro-C₁-C₃-alkoxy, and/or a monocyclic heterocyclyl radical having 4 to 7 ring atoms,
   or
   represents monocyclic heteroaryl having 5 or 6 ring atoms, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, hydroxy-C₁-C₃-alkyl, C₁-C₃-alkylamino, amino-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷, and/or a monocyclic heterocyclyl radical having 4 to 7 ring atoms,
   or
   represents monocyclic heterocyclyl having 4 to 7 ring atoms, which may optionally be mono- or polysubstituted by identical or different substituents from the group consisting of halogen, amino, hydroxy, cyano, oxo, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, hydroxy-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷, and/or a monocyclic heterocyclyl radical having 4 to 7 ring atoms,
   or
   represents a phenyl radical, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, C₁-C₃-alkylaminocarbonyl, C₁-C₃-alkylaminosulphonyl, hydroxy-C₁-C₃-alkyl, fluoro-C₁-C₃-alkyl, fluoro-C₁-C₃-alkoxy, C₃-C₆-cycloalkyl, and/or a monocyclic heterocyclyl radical having 4 to 7 ring atoms, and
R⁶ and R⁷ independently of one another represent hydrogen, C₁-C₃-alkyl, cyclopropyl, di-C₁-C₃-alkylamino-C₁-C₃-alkyl or fluoropyridyl, and
R⁸ represents hydroxy, C₁-C₆-alkyl, hydroxy-C₁-C₃-alkyl, fluoro-C₁-C₃-alkoxy-C₁-C₃-alkyl, C₃-C₈-cycloalkyl or monocyclic heterocyclyl which has 5 or 6 ring atoms, and
R⁹ represents hydrogen, C₁-C₆-alkyl or C₁-C₄-alkoxy, and their polymorphs, enantiomers, diastereomers, racemates, tautomers, solvates, physiologically acceptable salts and solvates of these salts.

Particular preference is further given to those compounds of the general formula (I) in which
X represents an oxygen atom, and
A represents a phenyl or 3-pyridyl ring, and
R^{1a} represents hydrogen or chlorine,
   or
   represents piperazinyl, pyrrolidinyl, piperidinyl, diazepanyl, oxazinanyl, oxazolidinyl, morpholinyl, thiomorpholinyl, thiazolidinyl or azetidinyl, which may optionally be mono- or polysubstituted by identical or different substituents from the group consisting of fluorine, chlorine, cyano, nitro, hydroxy, oxo, C₁-C₃-alkyl, C₁-C₃-alkoxy, C₁-C₂-alkoxy-C₁-C₂-alkyl , hydroxy-C₁-C₃-alkyl, dimethylamino, trifluoromethyl, difluoroethyl, trifluoroethyl, trifluoromethoxy, cyclopropyl, phenyl, fluorophenyl, phenyl, C₁-C₃-alkyl, pyridinyl, —NR⁶C(═O)—R⁹, —C(═O)—NR⁶R⁷, —C(═O)—R⁸,
   —S(═O)₂—NR⁶12⁷, —S(═O)—R⁹, —S(═O)₂—R⁹, and/or —NH—S(═O)₂—R⁹,
   or
   represents tetrazolyl,
   or
   represents isoxazolyl, pyrazolyl, thienyl, thiazolyl, imidazolyl, triazolyl, pyrrolyl, oxadiazolyl, pyridinyl or pyrimidinyl, which may optionally be mono- or polysubstituted by identical or different substituents from the group consisting of fluorine, chlorine, hydroxy, amino, cyano, nitro, C₁-C₃-alkyl, C₁-C₃-alkoxy, C₁-C₂-alkoxy-C₁-C₂-alkyl, dimethylamino, trifluoromethyl, difluoroethyl, trifluoroethyl, trifluoromethoxy, cyclopropyl, pyridinyl, phenyl, fluorophenyl, —C(═O)—NR⁶R⁷, —C(═O)—R⁸, —S(═O)₂—NR⁶R⁷, —S(═O)—R⁹, —S(═O)₂—R⁹, and/or —NH—S(═O)₂—R⁹,
   or
   represents a phenyl radical, which may optionally be mono- or polysubstituted by identical or different substituents from the group consisting of fluorine, chlorine, amino, hydroxy, cyano, nitro, carboxyl, C₁-C₃-alkyl, C₁-C₃-alkoxy, C₁-C₂-alkoxy-C₁-C₂-alkyl, dimethylamino, —C(═O)NR⁶R⁷, —C(═O)R⁸, C₁-C₃-alkylsulphinyl, C₁-C₃-alkylsulphonyl, —S(═O)₂NH₂, C₁-C₃-alkylsulphonylamino, C₁-C₃-alkylaminosulphonyl, C₃-C₆-cycloalkylaminosulphonyl, trifluoromethyl, difluoroethyl, trifluoroethyl, trifluoromethoxy, hydroxy-C₁-C₃-alkyl, cyclopropyl, chlorothienyl, morpholino and/or pyridinyl, and
R^{1b} represents hydrogen, fluorine, bromine or cyano,
R^{1c} represents hydrogen or bromine, and
R² represents methyl, ethyl or isopropyl, and
R³ represents cyclopropyl, methyl, ethyl, methoxy, ethoxy, cyclopropylamino, methylamino or ethylamino, and
R⁴ and R⁵ independently of one another represent hydrogen, hydroxy, cyano, amino, chlorine, C₁-C₆-alkyl, methoxy, ethoxy or C₁-C₃-alkylcarbonylamino,
   or
   represent difluoromethoxy or trifluoromethoxy, or represent C₁-C₃-alkoxy, which may be substituted by pyridinyl, morpholinyl, pyrrolidinyl or piperazinyl,
   in which pyridinyl and piperazinyl in turn may be optionally substituted by C alkyl,
   or
   represent cyclopropyl,
   or
   represent pyridinyl, pyrazolyl, triazolyl or isoxazolyl, which may be optionally mono- or polysubstituted by identical or different substituents from the group consisting of hydroxy and/or methyl,
   or
   represent pyrrolidinyl, morpholinyl, piperidinyl, piperazinyl, oxazolidinyl, thiomorpholinyl, which may optionally be mono- or polysubstituted by oxo, methyl and/or —S(═O)₂R⁹,
   or
   represent phenyl optionally substituted by C₁-C₃-alkylaminosulphonyl or fluorine,
   and
R⁶ and R⁷ independently of one another represent hydrogen, C₁-C₃-alkyl, cyclopropyl, di-C₁-C₃-alkylamino-C₁-C₃-alkyl or fluoropyridyl, and
R⁸ represents hydroxy, C₁-C₃-alkyl, hydroxy-C₁-C₃-alkyl, trifluoromethyl, pyrrolidinyl, morpholinyl or piperidinyl, and
R⁹ represents C₁-C₄-alkyl or C₁-C₄-alkoxy, and their polymorphs, enantiomers, diastereomers, racemates, tautomers, solvates, physiologically acceptable salts and solvates of these salts.

Of very particular interest, furthermore, are those compounds of the general formula (I) in which
X represents an oxygen atom, and
A represents a phenyl or 3-pyridyl ring, and
R^{1a} represents hydrogen or chlorine,
   or
   represents piperazinyl, pyrrolidinyl, piperidinyl, diazepanyl, oxazinanyl, oxazolidinyl, morpholinyl, thiomorpholinyl, thiazolidinyl or azetidinyl, which may optionally be mono- or polysubstituted by identical or different substituents from the group consisting of fluorine, hydroxy, oxo, C₁-C₃-alkyl, methoxy, hydroxy-C₁-C₃-alkyl, dimethylamino, difluoroethyl, trifluoroethyl, benzyl, —NR⁶C(═O)—R⁹, —C(═O)—NR⁶R⁷, —C(═O)—R⁸, and/or
   —S(═O)₂—R⁹,
   or
   represents tetrazolyl,
   or
   represents isoxazolyl, pyrazolyl, thienyl, thiazolyl, imidazolyl, triazolyl, pyrrolyl, oxadiazolyl, pyridinyl or pyrimidinyl, which may optionally be mono- or polysubstituted by identical or different substituents from the group consisting of fluorine, chlorine, hydroxy, cyano, C₁-C₂ alkyl, methoxy, methoxymethyl, trifluoromethyl, cyclopropyl, pyridinyl, phenyl, fluorophenyl and/or —C(═O)—R⁸,
   or
   represents a phenyl radical, which may optionally be mono- or polysubstituted by identical or different substituents from the group consisting of fluorine, chlorine, hydroxy, cyano, nitro, carboxyl, C₁-C₃-alkyl, methoxy, —C(═O)NR⁶R⁷, —C(═O)R⁸, C₁-C₃ alkylsulphinyl, C₁-C₃-alkylsulphonyl, —S(═O)₂NH₂, C₁-C₃-alkylsulphonylamino, C₁-C₃-alkylaminosulphonyl, C₃-C₆-cyclo-alkylaminosulphonyl, trifluoromethyl, trifluoromethoxy, hydroxy-C₁-C₃-alkyl, cyclopropyl, chlorothienyl and/or morpholino, and
R^{1b} represents hydrogen, fluorine, bromine or cyano, and
R^{1c} represents hydrogen or bromine, and
R² represents methyl, ethyl or isopropyl, and
R³ represents cyclopropyl, methyl, ethyl, methoxy, ethoxy, cyclopropylamino, methylamino or ethylamino, and
R⁴ and R⁵ independently of one another represent hydrogen, hydroxy, cyano, amino, chlorine, C₁-C₆-alkyl, methoxy, ethoxy or C₁-C₃-alkylcarbonylamino,
   or
   represent difluoromethoxy or trifluoromethoxy,
   or represent C₁-C₃-alkoxy, which may be substituted by pyridinyl, morpholinyl, pyrrolidinyl or piperazinyl,
   in which pyridinyl and piperazinyl in turn may be optionally substituted by C₁-C₃-alkyl,
   or
   represent cyclopropyl,
   or
   represent pyridinyl, pyrazolyl, triazolyl or isoxazolyl, optionally mono- or polysubstituted by identical or different substituents from the group consisting of hydroxy and/or methyl,
   or
   represent pyrrolidinyl, morpholinyl, piperidinyl, piperazinyl, oxazolidinyl, thiomorpholinyl, which may optionally be mono- or polysubstituted by methyl, oxo and/or —S(═O)₂R⁹,
   or
   represent phenyl optionally substituted by C₁-C₃-alkylaminosulphonyl or fluorine,
   and
R⁶ and R⁷ independently of one another represent hydrogen, C₁-C₃-alkyl, cyclopropyl, di-C₁-C₃-alkylamino-C₁-C₃-alkyl or fluoropyridyl, and
R⁸ represents hydroxy, C₁-C₃-alkyl, hydroxy-C₁-C₃-alkyl, trifluoromethyl, pyrrolidinyl, morpholinyl or piperidinyl, and
R⁹ represents C₁-C₄-alkyl or C₁-C₄-alkoxy, and their polymorphs, enantiomers, diastereomers, racemates, tautomers, solvates, physiologically acceptable salts and solvates of these salts.

Of exceptional interest are those compounds of the general formula (I) in which
X represents an oxygen atom, and
A represents a phenyl ring, and
R^{1a} represents piperazinyl, pyrrolidinyl, piperidinyl, diazepanyl, oxazinanyl, oxazolidinyl, morpholinyl, thiomorpholinyl, thiazolidinyl or azetidinyl, which may optionally be mono- or polysubstituted by identical or different substituents from the group consisting of fluorine, hydroxy, oxo, C₁-C₃-alkyl, methoxy, hydroxy-C₁-C₃-alkyl, dimethylamino, difluoroethyl, trifluoroethyl, benzyl, —NR⁶C(═O)—R⁹, —C(═O)—NR⁶R⁷, —C(═O)—R⁸, and/or —S(═O)₂—R⁹,
   or
   represents tetrazolyl,
   or
   represents isoxazolyl, pyrazolyl, thienyl, thiazolyl, imidazolyl, triazolyl, pyrrolyl, oxadiazolyl, pyridinyl or pyrimidinyl, which may optionally be mono- or polysubstituted by identical or different substituents from the group consisting of fluorine, chlorine, hydroxy, cyano, C₁-C₂-alkyl, methoxy, methoxymethyl, trifluoromethyl, cyclopropyl, pyridinyl, phenyl, fluorophenyl and/or —C(═O)—R⁸,
   or
   represents a phenyl radical, which may optionally be mono- or polysubstituted by identical or different substituents from the group consisting of fluorine, chlorine, hydroxy, cyano, nitro, carboxyl, C₁-C₃-alkyl, methoxy, —C(═O)NR⁶R⁷, C(═O)R⁸, C₁-C₃-alkylsulphinyl, C₁-C₃-alkylsulphonyl, —S(═O)₂NH₂, C₁-C₃-alkylsulphonylamino, C₁-C₃-alkylaminosulphonyl, C₃-C₆-cycloalkylaminosulphonyl, trifluoromethyl, trifluoromethoxy, hydroxy-C₁-C₃-alkyl, cyclopropyl, chlorothienyl and/or morpholino,
R^{1b} represents hydrogen, fluorine, bromine or cyano, and
R^{1c} represents hydrogen, and
R² represents methyl or ethyl, and
R³ represents mcthylamino, and
R⁴ and R⁵ independently of one another represent hydrogen, hydroxy, cyano, chlorine, C₁-C₆-alkyl, methoxy, ethoxy or C₁-C₃-alkylcarbonylamino,
   or
   represent difluoromethoxy or trifluoromethoxy, and
R⁶ and R⁷ independently of one another represent hydrogen, C₁-C₃-alkyl, cyclopropyl or di-C₁-C₃-alkylamino-C₁-C₃-alkyl,and
R⁸ represents hydroxy, C₁-C₃-alkyl, hydroxy-C₁-C₃-alkyl, trifluoromethyl, pyrrolidinyl, morpholinyl or piperidinyl, and
R⁹ represents C₁-C₄-alkyl or C₁-C₄-alkoxy, and their polymorphs, tautomers, solvates, physiologically acceptable salts and solvates of these salts, and
where the stereocentre, which is represented by the carbon atom of the benzodiazepine skeleton which is bound to R², is present either in racemic form or predominantly or completely in the (S) configuration.

Of exceptional interest, furthermore, are those compounds of the general formula (I) in which
X represents an oxygen atom, and
A represents a phenyl ring, and
R^{1a} represents hydrogen or chlorine, and
R^{1b} represents hydrogen, fluorine, bromine or cyano, and
R^{1c} represents hydrogen, and
R² represents methyl or ethyl, and
R³ represents methylamino, and
R⁴ represents cyclopropyl,
   or
   represents pyridinyl, pyrazolyl, triazolyl or isoxazolyl, which may optionally be mono- or polysubstituted by identical or different substituents from the group consisting of hydroxy and/or methyl,
   or
   represents pyrrolidinyl, morpholinyl, piperidinyl, piperazinyl, oxazolidinyl or thiomorpholinyl, which may optionally be mono- or polysubstituted by methyl, oxo and/or —S(═O)₂R⁹,
   or
   represents phenyl optionally substituted by C₁-C₁-alkylaminosulphonyl or fluorine,
   and
R⁵ represents hydrogen, hydroxy, cyano, chlorine, C₁-C₆-alkyl, methoxy, ethoxy or C₁-C₃-alkylcarbonylamino,
   or
   represents difluoromethoxy or trifluoromethoxy, and
R⁶ and R⁷ independently of one another represent hydrogen, C₁-C₃-alkyl, cyclopropyl or di-C₁-C₃-alkylamino-C₁-C₃-alkyl,and
R⁸ represents hydroxy, C₁-C₃-alkyl, hydroxy-C₁-C₃-alkyl, trifluoromethyl, pyrrolidinyl, morpholinyl or piperidinyl, and
R⁹ represents C₁-C₄-alkyl or C₁-C₄-alkoxy, and their polymorphs, tautomers, solvates, physiologically acceptable salts and solvates of these salts, and
where the stereocentre, which is represented by the carbon atom of the benzodiazepine skeleton which is bound to R², is present either in racemic form or predominantly or completely in the (S) configuration.

Of exceptional interest, furthermore, are those compounds of the general formula (I) in which
X represents an oxygen atom, and
A represents a phenyl ring, and
R^{1a} represents hydrogen or chlorine, and
R^{1b} represents hydrogen, fluorine, bromine or cyano, and
R^{1c} represents hydrogen, and
R² represents methyl or ethyl, and
R³ represents methylamino, and
R⁴ represents hydrogen, chlorine, methoxy or ethoxy,
   or
   represents ditluoromethoxy or trifluoromethoxy, and
R⁵ represents cyclopropyl,
   or
   represents pyridinyl or pyrazolyl, which may optionally be substituted one or more times by methyl,
   or
   represents morpholinyl, piperidinyl, piperazinyl or thiomorpholinyl, which may optionally be mono- or polysubstituted by methyl, oxo and/or —S(═O)₂R⁹,
   or
   represents phenyl which is substituted by C₁-C₃-alkylaminosulphonyl, and
R⁶ and R⁷ independently of one another represent hydrogen, C₁-C₃-alkyl, cyclopropyl or di-C₁-C₃-alkylamino-C₁-C₃-alkyl, and
R⁸ represents hydroxy, C₁-C₃-alkyl, hydroxy-C₁-C₃-alkyk trifluoromethyl, pyrrolidinyl, morpholinyl or piperidinyl, and
R⁹ represents C₁-C₄-alkyl or C₁-C₄-alkoxy, and their polymorphs, tautomers, solvates, physiologically acceptable salts and solvates of these salts, and
where the stereocentre, which is represented by the carbon atom of the benzodiazepine skeleton which is bound to R², is present either in racemic form or predominantly or completely in the (S) configuration.

Of exceptional interest, furthermore, are those compounds of the general formula (I) in which
X represents an oxygen atom, and
A represents a phenyl ring, and
R^{1a} represents piperazinyl, pyrrolidinyl, piperidinyl, diazepanyl, oxazinanyl, oxazolidinyl, morpholinyl, thiomorpholinyl, thiazolidinyl or azetidinyl, which may optionally be mono- or polysubstituted by identical or different substituents from the group consisting of fluorine, hydroxy, oxo, C₁-C₃-alkyl, methoxy, hydroxy-C₁-C₃-alkyl, dimethylamino, difluoroethyl, trifluoroethyl, benzyl, —NR⁶C(═O)—R⁹, —C(═O)—NR⁶R⁷, —C(═O)—R⁸ and/or
   —S(═O)₂—R⁹,
   or
   represents isoxazolyl or pyrazolyl, which may optionally be substituted one or more times by identical or different C₁-C₂-alkyls, and
R^{1b} represents hydrogen, fluorine, bromine or cyano, and
R^{1c} represents hydrogen, and
R² represents methyl, and
R³ represents methylamino, and
R⁴ and R⁵ independently of one another represent hydrogen, hydroxy, cyano, chlorine, C₁-C₆-alkyl, methoxy, ethoxy or C₁-C₃-alkylcarbonylamino,
   or
   represent difluoromethoxy or trifluoromethoxy, and
R⁶ and R⁷ independently of one another represent hydrogen, C₁-C₃-alkyl, cyclopropyl or di-C₁-C₃-alkylamino-C₁-C₃-alkyl,and
R⁸ represents hydroxy, C₁-C₃-alkyl, hydroxy-C₁-C₃-alkyl, trifluoromethyl, pyrrolidinyl, morpholinyl or piperidinyl, and
R⁹ represents C₁-C₄-alkyl or C₁-C₄-alkoxy,
and their polymorphs, tautomers, solvates, physiologically acceptable salts and solvates of these salts, and
where the stereocentre, which is represented by the carbon atom of the benzodiazepine skeleton which is bound to R², is present either in racemic form or predominantly or completely in the (S) configuration.

Of exceptional interest, furthermore, are those compounds of the general formula (I) in which
X represents an oxygen atom, and
A represents a phenyl ring, and
R^{1a} represents piperazinyl, pyrrolidinyl, piperidinyl, morpholinyl, thiomorpholinyl or azetidinyl, which may optionally be mono- or polysubstituted by identical or different substituents from the group consisting of hydroxy, oxo, methoxy, dimethylamino, difluoroethyl, trifluoroethyl, —NR⁶C(═O)—R⁹, —C(═O)—NR⁶R⁷ and/or
   —C(═O)—R⁸, and
R^{1b} represents hydrogen, fluorine, bromine or cyano, and
R^{1c} represents hydrogen, and
R² represents methyl, and
R³ represents methylamino, and
R⁴ and R⁵ independently of one another represent hydrogen, chlorine, methoxy or ethoxy, or represent difluoromethoxy or trifluoromethoxy, and
R⁶ and R⁷ independently of one another represent hydrogen or C₁-C₃-alkyl, and represents methyl, and
R⁹ represents methyl,
and their polymorphs, tautomers, solvates, physiologically acceptable salts and solvates of these salts, and
where the stereocentre, which is represented by the carbon atom of the benzodiazepine skeleton which is bound to R², is present either in racemic form or predominantly or completely in the (S) configuration.

The invention is based on the following definitions:

Alkyl:

Alkyl represents a straight-chain or branched saturated monovalent hydrocarbon radical having generally 1 to 6 (C₁-C₆-alkyl), preferably 1 to 3 carbon atoms (C₁-C₃-alkyl). The following may be mentioned by way of example: methyl, ethyl, propyl, butyl, pentyl, hexyl, isopropyl, isobutyl, sec-butyl, tert-butyl, isopentyl, 2-methylbutyl, methylbutyl, 1-methylbutyl, 1-ethylpropyl, 1,2-dimethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, 1,2-dimethylbutyl. Preference is given to a methyl, ethyl, propyl, isopropyl or tert-butyl radical.

Cycloalkyl:

Cycloalkyl represents a mono- or bicyclic saturated monovalent hydrocarbon radical having generally 3 to 10 (C₃-C₁₀-cycloalkyl), preferably 3 to 8 (C₃-C₈-cycloalkyl), and particularly preferably 3 to 7 (C₃-C₇-cycloalkyl) carbon atoms.

The following may be mentioned by way of example and by way of preference for monocyclic cycloalkyl radicals:

cyclopropyl, cyclobutyl, cyclopentyl, cyclohexyl and cycloheptyl. Particular preference is given to a cyclopropyl, cylopentyl or a cyclohexyl radical. The following may be mentioned by way of example for bicyclic cycloalkyl radicals: perhydropentalenyl, decalinyl.

Phenylalkyl:

Phenyl-C₁-C₆-alkyl is to be understood as meaning a group composed of an optionally substituted phenyl radical and a C₁-C₆-alkyl group, which is attached via the C₁-C₆-alkyl group to the remainder of the molecule. Here, the alkyl radical has the meanings given above under alkyl. Preference is given to phenyl-C₁-C₃-alkyl. The following may be mentioned by way of example: benzyl, phenethyl, phenylpropyl, phenylpentyl, with benzyl being particularly preferred.

Alkoxy:

Alkoxy represents a straight-chain or branched saturated alkyl ether radical of the formula —O-alkyl having generally 1 to 6 (C₁-C₆-alkoxy), preferably 1 to 3 (C₁-C₃-alkoxy) carbon atoms. The following may be mentioned by way of example and by way of preference: methoxy, ethoxy, n-propoxy, isopropoxy, tert-butoxy, n-pentyloxy and n-hexyloxy.

Alkoxyalkyl

Alkoxyalkyl represents an alkoxy-substituted alkyl radical. Here, C₁-C₃-alkoxy-C₁-C₃-alkyl means that the binding to the rest of the molecule is via the alkyl moiety.

Alkoxyalkoxy

Alkoxyalkoxy represents an alkoxy-substituted alkoxy radical. Here, C₁-C₃-alkoxy-C₂-C₃-alkoxy means that the binding to the rest of the molecule is via the inner C₂-C₃-alkoxy moiety.

Oxo

Oxo, an oxo group or an oxo substituent is to be understood as meaning a doubly attached oxygen atom ═O. Oxo may be attached to atoms of suitable valency, for example to a saturated carbon atom or to sulphur.

Preference is given to binding to carbon with formation of a carbonyl group —C(═O)—. Preference is furthermore given to binding two doubly attached oxygen atoms to a sulphur atom with formation of a sulphonyl group S(═O)₂—.

Alkylamino

Alkylamino represents an amino radical having one or two alkyl substituents (chosen independently of one another) having generally 1 to 6 (C₁-C₆-alkylamino), preferably 1 to 3 carbon atoms (C₁-C₃-alkylamino). (C₁-C₃)-alkylamino represents, for example, a monoalkylamino radical having 1 to 3 carbon atoms or represents a dialkylamino radical having in each case 1 to 3 carbon atoms per alkyl substituent. The following may be mentioned by way of example: methylamino, ethylamino, n-propylamino, isopropylamino, tert-butylamino, n-pentylamino, n-hexylamino, N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino.

Alkylaminocarbonyl

Alkylaminocarbonyl represents the group alkylamino-C(═O)— having one or two alkyl substituents (chosen independently of one another) having generally 1 to 6 (C₁-C₆-alkylaminocarbonyl), preferably 1 to 3 carbon atoms (C₁-C₃-alkylaminocarbonyl).

Cycloalkylaminocarbonyl

Cycloalkylaminocarbonyl represents the group cycloalkyl-NH—C(═O)— having a cycloalkyl substituent, generally consisting of 3 to 6 (C₃-C₆-cycloalkylaminocarbonyl) carbon atoms. The following may be mentioned by way of example and by way of preference: cyclopropylaminocarbonyl and cyclopentylaminocarbonyl.

Alkylcarbonyl

Alkylcarbonyl represents the group —C(═O)-alkyl having generally 1 to 6 (C₁-C₆-alkylcarbonyl), preferably 1 to 4, and particularly preferably 1 to 3 carbon atoms in the alkyl moiety. The following are mentioned by way of example: acetyl and propanoyl.

Alkylcarbonylamino

Alkylcarbonylamino represents the group alkyl-C(═O)—NH— having generally 1 to 6 (C₁-C₆-alkylcarbonylamino), preferably 1 to 4, and particularly preferably 1 to 3 carbon atoms in the alkyl moiety.

Alkoxycarbonyl

Alkoxycarbonyl represents the group —C(═O)-0-alkyl having generally 1 to 6 (C₁-C₆-alkoxycarbonyl), preferably 1 to 4, and particularly preferably 1 to 3 carbon atoms in the alkyl moiety. The following may be mentioned by way of example: methoxycarbonyl, ethoxycarbonyl, propoxycarbonyl, isopropoxycarbonyl, tert-butoxycarbonyl, n-pentyloxycarbonyl and n-hexyloxycarbonyl.

Alkylsulphonyl

Alkylsulphonyl represents a straight-chain or branched saturated radical of the formula —S(═O)₂-alkyl having generally 1 to 6 (C₁-C₆-alkylsulphonyl), preferably 1 to 3 (C₁-C₃-alkylsulphonyl) carbon atoms. The following may be mentioned by way of example and by way of preference: methylsulphonyl, ethylsulphonyl, propylsulphonyl.

Alkylsulphinyl

Alkylsulphinyl represents a straight-chain or branched saturated radical of the formula —S(═O)-alkyl having generally 1 to 6 (C₁-C₆-alkylsulphinyl), preferably 1 to 3 (C₁-C₁-alkylsulphinyl) carbon atoms. The following may be mentioned by way of example and by way of preference: methylsulphinyl, ethylsulphinyl, propylsulphinyl.

Alkylsulphonylamino

Alkylsulphonylamino represents a straight-chain or branched saturated radical of the formula —NH—S(═O)₂-alkyl having 1 to 3 (C₁-C₃-alkylsulphonyl) carbon atoms in the alkyl group. The following may be mentioned by way of example and by way of preference: methylsulphonylamino, ethylsulphonylamino, propylsulphonylamino.

Alkylaminosulphonyl

Alkylaminosulphonyl represents the group alkylamino-S(═O)₂— having one or two alkyl substituents (chosen independently of one another) having generally 1 to 6 (C₁-C₆-alkylaminosulphonyl), preferably 1 to 3 carbon atoms.

The following may be mentioned by way of example and by way of preference: methylaminosulphonyl, ethylaminosulphonyl, dimethylaminosulphonyl.

Cycloalkylaminosulphonyl

Cycloalkylaminosulphonyl represents the group cycloalkyl-NH—S(═O)₂— having a cycloalkyl substituent, generally consisting of 3 to 6 (C₃-C₆-cycloalkylaminosulphonyl) carbon atoms. The following may be mentioned by way of example and by way of preference: cyclopropylaminosulphonyl.

Heteroatoms

Heteroatoms are to be understood as meaning oxygen, nitrogen and sulphur atoms.

Heteroaryl

Heteroaryl denotes a monovalent monocyclic aromatic ring system having 5 or 6 ring atoms, of which at least one is a heteroatom. Heteroatoms present may be nitrogen atoms, oxygen atoms and/or sulphur atoms. The binding valency may be located at any aromatic carbon atom or at an oxygen atom.

A monocyclischer heteroaryl radical in accordance with the present invention has 5 or 6 ring atoms.

Heteroaryl radicals having 5 ring atoms include, for example, the following rings: thienyl, thiazolyl, fu yl, pyrrolyl, oxazolyl, imidazolyl, pyrazolyl, isoxazolyl, isothiazolyl, oxadiazolyl, triazolyl, tetrazolyl and thiadiazolyl.

Heteroaryl radicals having 6 ring atoms include, for example, the following rings: pyridyl, pyridazinyl, pyrimidinyl, pyrazinyl and triazinyl.

Heterocyclyl

Heterocyclyl means a non-aromatic monocyclic ring system having at least one heteroatom or a heterogroup. Heteroatoms which may be present are nitrogen atoms, oxygen atoms and/or sulphur atoms. Heterogroups which may be present are —S(═O), —S(═O)2— or —N⁺(O⁻)—.

A monocyclic heterocyclyl ring in accordance with the present invention may have 3 to 8, preferably 5 to 8 or 4 to 7, particularly preferably 5 or 6, ring atoms.

The following may be mentioned in an exemplary and preferred manner for monocyclic heterocyclyl radicals having 3 ring atoms: aziridinyl.

The following may be mentioned in an exemplary and preferred manner for monocyclic heterocyclyl radicals having 4 ring atoms: azetidinyl, oxetanyl.

The following may be mentioned in an exemplary and preferred manner for monocyclic heterocyclyl radicals having 5 ring atoms: pyrrolidinyl, imidazolidinyl, pyrazolidinyl, pyrrolinyl, dioxolanyl and tetrahydrofuranyl.

The following may be mentioned in an exemplary and preferred manner for monocyclic heterocyclyl radicals having 6 ring atoms: piperidinyl, piperazinyl, morpholinyl, dioxanyl, tetrahydropyranyl and thiomorpholinyl.

The following may be mentioned in an exemplary and preferred manner for monocyclic heterocyclyl radicals having 7 ring atoms: azepanyl, oxepanyl, 1,3-diazepanyl, 1,4-diazepanyl.

The following may be mentioned in an exemplary and preferred manner for monocyclic heterocyclyl radicals having 8 ring atoms: oxocanyl, azocanyl.

Preference is given to 5- to 8- and 4 to 7-membered monocyclic saturated heterocyclyl radicals having up to two heteroatoms from the group consisting of 0, N and S. Particular preference is given to morpholinyl, piperidinyl, piperazinyl and pyrrolidinyl.

N-Heterocyclyl

N-Heterocyclyl means a non-aromatic cyclic ring system having at least one nitrogen atom as heteroatom, which is attached to the remainder of the molecule via the nitrogen atom.

Halogen

The term halogen comprises fluorine, chlorine, bromine and iodine. Preference is given to fluorine and chlorine.

Halo

Halo represents halogen and comprises fluorine, chlorine and bromine and refers to a radical substituted by fluorine, chlorine or bromine such as, for example, halophenyl, which is a phenyl radical which is mono- or polysubstituted by identical or different fluorine, chlorine and/or bromine substituents.

Haloalkyl

Haloalkyl represents an alkyl radical having at least one halogen substituent. A halo-C₁-C₆-alkyl radical is an alkyl radical having 1-6 carbon atoms and at least one halogen substituent. If a plurality of halogen substituents are present, these may also be different from one another. Preference is given to fluoro-C₁-C₃-alkyl radicals. The following may be mentioned by way of example and by way of further preference: the trifluoromethyl, 2,2,2-trifluoroethyl, pentafluoroethyl, 4,4,5,5,5-pentafluoropentyl or 3,3,4,4,5,5,5-heptafluoropentyl group. Particular preference is given to trifluoromethyl, 2,2-difluoroethyl and 2,2,2-trifluoroethyl.

Haloalkoxy

Haloalkoxy represents an alkoxy radical having at least one halogen substituent. A halo-C₁-C₆-alkoxy radical is an alkoxy radical having 1-6 carbon atoms and at least one halogen substituent. If a plurality of halogen substituents are present, these may also be different from one another. Preference is given to fluoro-C₁-C₃-alkoxy radicals. The following may be mentioned by way of example and by way of particular preference: difluoromethoxy, trifluoromethoxy or 2,2,2-trifluoroethoxy.

Hydroxyalkyl

Hydroxyalkyl represents an alkyl radical having at least one hydroxy substituent. A hydroxy-C₁-C₆-alkyl radical is an alkyl radical having 1-6 carbon atoms and at least one hydroxy substituent. Preference is given to hydroxy-C₁-C₃-alkyl.

In the general formula (I) it is possible that X represents an oxygen or sulphur atom.

In the general formula (I) X preferably represents an oxygen atom.

In the general formula (I) it is possible that A represents a monocyclic heteroaryl ring having 5 or 6 ring atoms or represents a phenyl ring.

In the general formula (I) A preferably represents a monocyclic heteroaryl ring having 6 ring atoms or represents a phenyl ring.

In the general formula (I) A more preferably represents a pyridyl ring or a phenyl ring.

In the general formula (I) A very preferably represents pyrid-3-yl.

In the general formula (I) A very preferably represents a phenyl ring.

In the general formula (I) it is possible that R¹a^{.} represents hydrogen, halogen, cyano, carboxyl, amino or aminosulphonyl,

or

represents a C₁-C₆-alkoxy, C₁-C₃-alkoxy-C₂-C₃-alkoxy, C₁-C₆-alkylamino, C₁-C₆-alkylcarbonylamino, C₁-C₆-alkylamino-C₁-C₆-alkyl, N-(heterocyclyl)-C₁-C₆-alkyl, N-(heterocyclyl)-C₁-C₆-alkoxy, hydroxy-C₁-C₆-alkyl, hydroxy-C₁-C₆-alkoxy, halo-C₁-C₆-alkyl, halo-C₁-C₆-alkoxy, C₁-C₆-alkylcarbonyl or C₁-C₆-alkoxycarbonyl radical,

or

represents a monocyclic heterocyclyl radical having 3 to 8 ring atoms, which may optionally be mono- or polysubstituted by identical or different substituents from the group consisting of halogen, cyano, nitro, hydroxy, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, pyridinyl, —NR⁶C(═O)—R⁹, —C(═O)—NR⁶1e, —C(═O)—R⁸, —S(═O)₂-Nlele, —S(═O)—R⁹,

—S(═O)₂—R⁹, —NH—S(═O)₂—R⁹, and/or a monocyclic heterocyclyl radical having 3 to 8 ring atoms,

or

represents a monocyclic heteroaryl radical having 5 or 6 ring atoms, which may optionally be mono- or polysubstituted by identical or different substituents from the group consisting of halogen, hydroxy, amino, cyano, nitro, carboxyl, C₁-C₆-alkyl, C₁-C₆-alkoxy, C₁-C₆-alkoxy-C₁-C₆-alkyl, C1 C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, and/or by a monocyclic heterocyclyl radical having 3 to 8 ring atoms, and/or by a monocyclic heteroaryl radical having 5 or 6 ring atoms, and/or by a phenyl radical which for its part may optionally be mono- or polysubstituted by identical or different substituents from the group consisting of halogen, C₁-C₃-alkyl and/or C₁-C₃-alkoxy,

or

represents a phenyl radical, which may optionally be mono- or polysubstituted by identical or different substituents from the group consisting of halogen, amino, hydroxy, cyano, nitro, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, —C(═O)NR⁶R⁷, —C(═O)R⁸, C₁-C₃-alkylsulphinyl, C₁-C₃-alkylsulphonyl, —S(═O)₂NH₂, C₁-C₃-alkylsulphonylamino, C₁-C₃-alkylaminosulphonyl, C₃-C₆-cycloalkylaminosulphonyl, halo-C₁-C₆-alkyl, halo-C₁-C₆-alkoxy, hydroxy-C₁-C₆-alkyl, C₃-C₁₀-cycloalkyl and/or a monocyclic heterocyclyl radical having 3 to 8 ring atoms, and/or a monocyclic heteroaryl radical having 5 or 6 ring atoms and which for its part may optionally be mono- or polysubstituted by identical or different substituents from the group consisting of halogen, C₁-C₃-alkyl and/or C₁-C₃-alkoxy.

In the general formula (I) R¹a preferably represents hydrogen, halogen, cyano, carboxyl, amino or aminosulphonyl,

or

represents a C₁-C₆-alkoxy, C₁-C₃-alkoxy-C₁-C₃-alkyl, C₁-C₃-alkoxy-C₂-C₃-alkoxy, C₁-C₆-alkylamino, C₁-C₆-alkylcarbonylamino, C₁-C₆-alkylamino-C₁-C₆-alkyl, N-(heterocyclyl)-C₁-C₆-alkyl, N-(heterocyclyl)-C₁-C₆-alkoxy, hydroxy-C₁-C₆-alkyl, hydroxy-C₁-C₆-alkoxy, halo-C₁-C₆-alkyl, halo-C₁-C₆-alkoxy, C₁-C₆-alkylcarbonyl or C₁-C₆-alkoxycarbonyl radical,

or

represents a monocyclic heterocyclyl radical having 4 to 7 ring atoms, which may optionally be mono- or polysubstituted by identical or different substituents from the group consisting of halogen, cyano, nitro, hydroxy, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₁-C₁₀-cycloalkyl, phenyl, halophenyl, phenyl-C₁-C₆-alkyl, pyridinyl, —NR⁶C(═O)R⁹, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, and by a monocyclic heterocyclyl radical having 4 to 7 ring atoms,

or

represents a monocyclic heteroaryl radical having 5 or 6 ring atoms, which may optionally be mono- or polysubstituted by identical or different substituents from the group consisting of halogen, hydroxy, amino, cyano, nitro, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, and by a monocyclic heterocyclyl radical having 4 to 7 ring atoms and/or by a monocyclic heteroaryl radical having 5 or 6 ring atoms and/or by a phenyl radical which for its part may optionally be mono- or polysubstituted by identical or different substituents from the group consisting of halogen, C₁-C₃-alkyl and/or C₁-C₃-alkoxy,

or

represents a phenyl radical, which may optionally be mono- or polysubstituted by identical or different substituents from the group consisting of halogen, amino, hydroxy, cyano, nitro, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, Cl-C6-alkylamino-C₁-C₆-alkyl, —C(═O)NR⁶R⁷, C(═O)R⁸, C₁-C₃-alkylsulphinyl, C₁-C₃-alkylsulphonyl, —S(═O)₂NH₂, C₁-C₃-alkylsulphonylamino, C₁-C₃-alkylaminosulphonyl, C₃-C₆-cycloalkylaminosulphonyl, halo-C₁-C₆-alkyl, halo-C₁-C₆-alkoxy, hydroxy-C₁-C₆-alkyl, C₃-C₁₀-cycloalkyl and/or a monocyclic heterocyclyl radical having 4 to 7 ring atoms and/or a monocyclic heteroaryl radical having 5 or 6 ring atoms and which for its part may optionally be mono- or polysubstituted by identical or different substituents from the group consisting of halogen, C₁-C₃-alkyl and/or C₁-C₃-alkoxy.

In the general formula (I) R^{1a} very preferably represents hydrogen, halogen, cyano, carboxyl, amino or aminosulphonyl,

or

represents a C₁-C₆-alkoxy, C₁-C₃-alkoxy-C₁-C₃-alkyl, C₁-C₃-alkoxy-C₂-C₃-alkoxy, C₁-C₃-alkylamino, C₁-C₃-alkylcarbonylamino, C₁-C₃-alkylamino-C₁-C₃-alkyl, hydroxy-C₁-C₃-alkyl, fluoro-C₁-C₃-alkyl, fluoro-C₁-C₃-alkoxy, C₁-C₃-alkylcarbonyl or C₁-C₄-alkoxycarbonyl radical,

or

represents a monocyclic heterocyclyl radical having 4 to 7 ring atoms, which may optionally be mono- or polysubstituted by identical or different substituents from the group consisting of halogen, cyano, nitro, hydroxy, amino, oxo, carboxyl, C₁-C₃-alkyl, C₁-C₃-alkoxy, C₁-C₂-alkoxy-C₁-C₂-alkyl, hydroxy-C₁-C₃-alkyl, C₁-C₃-alkylamino, amino-C₁-C₃-alkyl, fluoro-C₁-C₃-alkyl, fluoro-C₁-C₃-alkoxy, C₃-C₆-cycloalkyl, phenyl, halophenyl, phenyl-C_{I} pyridinyl,

—NR⁶C(═O)—R⁹, —C(═O)—NR⁶R⁷, —C(═O)—R⁸, - S(═O)₂—NR⁶R⁷, —S(═O)—R⁹, —S(═O)₂—R⁹, —NH—S(═O)₂—R⁹, and/or by a monocyclic heterocyclyl radical having 4 to 7 ring atoms,

or

represents a monocyclic heteroaryl radical having 5 or 6 ring atoms, which may optionally be mono- or polysubstituted by identical or different substituents from the group consisting of halogen, hydroxy, amino, cyano, nitro, carboxyl, C₁-C₃-alkyl, C₁-C₃-alkoxy, C₁-C₂-alkoxy-C₁-C₂-alkyl, C₁-C₃ alkylamino, amino-C₁-C₃-alkyl, fluoro-C₁-C₃-alkyl, fluoro-C₁-C₃-alkoxy, C₃-C₆-cycloalkyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, and/or by a monocyclic heterocyclyl radical having 4 to 7 ring atoms, and/or by a monocyclic heteroaryl radical having 5 or 6 ring atoms, and/or by a phenyl radical which for its part may optionally be mono- or polysubstituted by identical or different substituents from the group consisting of fluorine, chlorine, bromine, methyl or methoxy,

or

represents a phenyl radical, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, —C(═O)NR⁶R⁷, C(═O)R⁸, C₁-C₃-alkylsulphinyl, C₁-C₃-alkylsulphonyl, —S(═O)₂NH₂, C₁-C₃-alkylsulphonylamino, C₁-C₃-alkylaminosulphonyl, C₃-C₆-cycloalkylaminosulphonyl, fluoro-C₁-C₃-alkyl, fluoro-C₁-C₃-alkoxy, hydroxy-C₁-C₃-alkyl, C₃-C₆-cycloalkyl and/or a monocyclic heterocyclyl radical having 4 to 7 ring atoms, and/or a monocyclic heteroaryl radical having 5 or 6 ring atoms and which for its part may optionally be mono- or polysubstituted by identical or different substituents from the group consisting of fluorine, chlorine, bromine, methyl or methoxy.

In the general formula (I) R^{1a} particularly preferably represents a monocyclic heterocyclyl radical having 4 to 7 ring atoms, which may optionally be mono- or polysubstituted by identical or different substituents from the group consisting of halogen, cyano, nitro, hydroxy, amino, oxo, carboxyl, C₁-C₃-alkyl, C₁-C₃-alkoxy, C₁-C₂-alkoxy-C₁-C₂-alkyl, hydroxy-C₁-C₃-alkyl, C₁-C₃-alkylamino, amino-C₁-C₃-a1kyl, fluoro-C₁-C₃-alkyl, fluoro-C₁-C₃-alkoxy, C₃-C₆-cycloalkyl, phenyl, halophenyl, phenyl-C₁-C₃-alkyl, pyridinyl, —NR⁶C(═O)—R⁹, —C(═O)-NIVIC, —C(═O)—R⁸,

—S(═O)₂—NR⁶R⁷, —S(═O)—R⁹, —S(═O)₂—R⁹, —NH—S(═O)₂—R⁹, and/or by a monocyclic heterocyclyl radical having 4 to 7 ring atoms,

or

represents a monocyclic heteroaryl radical having 5 or 6 ring atoms, which may optionally be mono- or polysubstituted by identical or different substituents from the group consisting of halogen, hydroxy, amino, cyano, nitro, carboxyl, C₁-C₃-alkyl, C₁-C₃-alkoxy, C₁-C₂-alkoxy-C₁-C₂-alkyl, C₁-C₃ alkylamino, amino-C,-C₃-alkyl, fluoro-C₁-C₃-alkoxy, C₃-C₆-cycloalkyl, —C(═O)-Nlele, —C(═O)—R⁸, —S(═O)₂—NR⁶R⁷, —S(═O)—R⁹, —S(═O)₂—R⁹, —NH—S(═O)₂—R⁹, and/or by a monocyclic heterocyclyl radical having 4 to 7 ring atoms, and/or by a monocyclic heteroaryl radical having 5 or 6 ring atoms, and/or by a phenyl radical which for its part may optionally be mono- or polysubstituted by identical or different substituents from the group consisting of fluorine, chlorine, bromine, methyl or methoxy,

or

represents a phenyl radical, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, —C(═O)NR⁶R⁷, C(═O)R⁸, C₁-C₃-alkylsulphinyl, C₁-C₃-alkylsulphonyl, —S(═O)₂NH₂, C₁-C₃-alkylsulphonylamino, C₁-C₃-alkylaminosulphonyl, C₃-C₆-cycloalkylaminosulphonyl, fluoro-C₁-C₃-alkyl, fluoro-C₁-C₃-alkoxy, hydroxy-C₁-C₃-alkyl, C₃-C₆-cycloalkyl and/or a monocyclic heterocyclyl radical having 4 to 7 ring atoms, and/or a monocyclic heteroaryl radical having 5 or 6 ring atoms and which for its part may optionally be mono- or polysubstituted by identical or different substituents from the group consisting of fluorine, chlorine, bromine, methyl or methoxy.

In the general formula (I) R^{1a} further particularly preferably represents hydrogen, halogen, cyano, carboxyl, amino or aminosulphonyl,

or

represents a C₁-C₆-alkoxy, C₁-C₃ alkoxy C₁-C₃ alkyl, C₁-C₃-alkoxy-C₂-C₃-alkoxy, C₁-C₃ alkylamino, C₁-C₃-alkylcarbonylamino, C₁-C₃-alkylamino-C₁-C₃-alkyl, hydroxy-C₁-C₃-alkyl, fluoro-C₁-C₃-alkyl, fluoro-C₁-C₃-alkoxy, C₁-C₃-alkylcarbonyl or C₁-C₄-alkoxycarbonyl radical.

In the general formula (I) R^{1a} further particularly preferably represents a monocyclic heterocyclyl radical having 4 to 7 ring atoms, which may optionally be mono- or polysubstituted by identical or different substituents from the group consisting of halogen, cyano, nitro, hydroxy, amino, oxo, carboxyl, C₁-C₃-alkyl, C₁-C₃-alkoxy, C₁-C₂-alkoxy-C₁-C₂-alkyl, hydroxy-C₁-C₃-alkyl, C₁-C₃-alkylamino, amino-C₁-C₃-alkyl, fluoro-C₁-C₃-alkyl, fluoro-C₁-C₃-alkoxy, C₃-C₆-cycloalkyl, phenyl, halophenyl, phenyl-C₁-C₃-alkyl, pyridinyl, —NR⁶C(═O)—R⁹, —C(═O)—NR⁶R⁷, —C(═O)—R⁸,

—S(═O)₂—NR⁶R⁷, —S(═O)—R⁹, —S(═O)₂—R⁹, —NH—S(═O)₂—R⁹, and/or by a monocyclic heterocyclyl radical having 4 to 7 ring atoms.

In the general formula (1) R ^{1a} further particularly preferably represents a monocyclic heteroaryl radical having 5 or 6 ring atoms, which may optionally be mono- or polysubstituted by identical or different substituents from the group consisting of halogen, hydroxy, amino, cyano, nitro, carboxyl, C₁-C₃-alkyl, C₁-C i-alkoxy, C₁-C₂-alkoxy-C₁-C₂-alkyl, C₁-C₃-alkylamino, amino-C,-C₃-alkyl, fluoro-C₁-C₃-alkyl, fluoro-C₁-C₃-alkoxy, C₃-C₆-cycloalkyl, —C(═O)—NR⁶R⁷, —C(═O)-le, —S(═O)₂—NR⁶R⁷, —S(═O)—R⁹, —S(═O)₂—R⁹, —NH—S(═O)₂R⁹, and/or by a monocyclic heterocyclyl radical having 4 to 7 ring atoms, and/or by a monocyclic heteroaryl radical having 5 or 6 ring atoms, and/or by a phenyl radical which for its part may optionally be mono- or polysubstituted by identical or different substituents from the group consisting of fluorine, chlorine, bromine, methyl or methoxy.

In the general formula (I) R^{1a} further particularly preferably represents a phenyl radical, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, —C(═O)NR⁶R⁷, C(═O)R⁸, C₁-C₃-alkylsulphinyl, C₁-C₃-alkylsulphonyl, —S(═O)₂NH₂, C₁-C₃-alkylsulphonylamino, C₁-C₃-alkylaminosulphonyl, C₃-C₆-cycloalkylaminosulphonyl, fluoro-C₁-C₃-alkyl, fluoro-C₁-C₃-alkoxy, hydroxy-C₁-C₃-alkyl, C₃-C₆-cycloalkyl and/or a monocyclic heterocyclyl radical having 4 to 7 ring atoms, and/or a monocyclic hetcroaryl radical having 5 or 6 ring atoms and which for its part may optionally be mono- or polysubstituted by identical or different substituents from the group consisting of fluorine, chlorine, bromine, methyl or methoxy.

In the general formula (I) Rⁱa very preferably represents hydrogen or chlorine,

or

represents piperazinyl, pyrrolidinyl, piperidinyl, diazepanyl, oxazinanyl, oxazolidinyl, morpholinyl, thiomorpholinyl, thiazolidinyl or azetidinyl, which may optionally be mono- or polysubstituted by identical or different substituents from the group consisting of fluorine, chlorine, cyano, nitro, hydroxy, oxo, C₁-C₃-alkyl, C₁-C₃-alkoxy, C₁-C₂-alkoxy-C₁-C₂-alkyl, hydroxy-C₁-C₃-alkyl, dimethylamino, trifluoromethyl, difluoroethyl, trifluoroethyl, trifluoromethoxy, cyclopropyl, phenyl, fluorophenyl, phenyl-C₁-C₃-alkyl, pyridinyl,

—NR⁶C(═O)—R⁹, —C(═O)—NR⁶R⁷, —C(═O)—R⁸, —S(═O)₂—NR⁶R⁷, —S(═O)—R⁹, —S(═O)₂—R⁹, and/or —NH—S(═O)₂—R⁹,

or

represents tetrazolyl,

or

represents isoxazolyl, pyrazolyl, thienyl, thiazolyl, imidazolyl, triazolyl, pyrrolyl, oxadiazolyl, pyridinyl or pyrimidinyl, which may optionally be mono- or polysubstituted by identical or different substituents from the group consisting of fluorine, chlorine, hydroxy, amino, cyano, nitro, C₁-C₃-alkyl, C₁-C₃-alkoxy, dimethylamino, trifluoromethyl, difluoroethyl, trifluoroethyl, trifluoromethoxy, cyclopropyl, —C(═O)—NR⁶R⁷, —C(═O)—R⁸,

—S(═O)₂—NR⁶R⁷, —S(═O)—R⁹, —S(═O)₂—R⁹, —NH—S(═O)₂—R⁹, pyridinyl, phenyl, and/or fluorophenyl,

or

represents phenyl, which may optionally be mono- or polysubstituted by identical or different substituents from the group consisting of fluorine, chlorine, amino, hydroxy, cyano, nitro, carboxyl, C₁-C₃-alkyl, C₁-C₃-alkoxy, C₁-C₂-alkoxy-C₁-C₂-alkyl, dimethylamino, —C(═O)NR⁶R⁷, C(═O)R⁸, C₁-C₃-alkylsulphinyl, C₁-C₃-alkylsulphonyl, —S(═O)₂NH₂, C₁-C₃-alkylsulphonylamino, C₁-C₃-alkylaminosulphonyl, C₃-C₆-cycloalkylaminosulphonyl, trifluoromethyl, difluoroethyl, trifluoroethyl, trifluoromethoxy, hydroxy-C₁-C₃-alkyl, cyclopropyl, morpholino and/or pyridinyl.

In the general formula (I) R^{1a} further very particularly preferably represents piperazinyl, pyrrolidinyl, piperidinyl, diazepanyl, oxazinanyl, oxazolidinyl, morpholinyl, thiomorpholinyl, thiazolidinyl or azetidinyl, which may optionally be mono- or polysubstituted by identical or different substituents from the group consisting of fluorine, chlorine, cyano, nitro, hydroxy, oxo, C₁-C₃-alkoxy, C₁-C₂-alkoxy-C₁-C₂-alkyl, hydroxy-C₁-C₃-alkyl, dimethylamino, trifluoromethyl, difluoroethyl, trifluoroethyl, trifluoromethoxy, cyclopropyl, phenyl, fluorophenyl, pyridinyl, —NR⁶C(═O)—R⁹, —C(═O)—NR⁶R⁷, —C(═O)—R⁸, —S(═O)₂—NR⁶R⁷,

—S(═O)—R⁹, —S(═O)₂—R⁹, and/or —NH—S(═O)₂—R⁹,

or

represents tetrazolyl,

or

represents isoxazolyl, pyrazolyl, thienyl, thiazolyl, imidazolyl, triazolyl, pyrrolyl, oxadiazolyl, pyridinyl or pyrimidinyl, which may optionally be mono- or polysubstituted by identical or different substituents from the group consisting of fluorine, chlorine, hydroxy, amino, cyano, nitro, C₁-C₃-alkyl, C₁-C₃-alkoxy-C₁-C₂-alkyl, dimethylamino, trifluoromethyl, difluoroethyl, trifluoroethyl, trifluoromethoxy, cyclopropyl, —C(═O)-12⁸,

—S(═O)₂—NR⁶R⁷, —S(═O)—R⁹, —S(═O)₂—R⁹, —NH—S(═O)₂—R⁹, pyridinyl, phenyl, and/or fluorophenyl,

or

represents phenyl which may optionally be mono- or polysubstituted by identical or different substituents from the group consisting of fluorine, chlorine, amino, hydroxy, cyano, nitro, carboxyl, C₁-C₃-alkyl, C₁-C₃-alkoxy, C₁-C₂-alkoxy-C₁-C₂-alkyl, dimethylamino, —C(═O)NR⁶R⁷, C(═O)R⁸, C₁-C₃-alkylsulphinyl, C₁-C₃-alkylsulphonyl, —S(═O)₂NH₂, C₁-C₃-alkylsulphonylamino, C₁-C₃-alkylaminosulphonyl, C₃-C₆-cycloalkylaminosulphonyl, trifluoromethyl, difluoroethyl, trifluoroethyl, trifluoromethoxy, hydroxy-C₁-C₃-alkyl, cyclopropyl, morpholino and/or pyridinyl.

In the general formula (1) R^{1a} further very particularly preferably represents hydrogen or chlorine.

In the general formula (I) R^{1a} further very particularly preferably represents piperazinyl, pyrrolidinyl, piperidinyl, diazepanyl, oxazinanyl, oxazolidinyl, morpholinyl, thiomorpholinyl, thiazolidinyl or azetidinyl, which may optionally be mono- or polysubstituted by identical or different substituents from the group consisting of fluorine, chlorine, cyano, nitro, hydroxy, oxo, C₁-C₃-alkoxy, hydroxy-C₁-C₃-alkyl, dimethylamino, trifluoromethyl, difluoroethyl, trifluoroethyl, trifluoromethoxy, cyclopropyl, phenyl, fluorophenyl, phenyl C₁-C₃ alkyl, pyridinyl, —NR⁶C(═O)—R⁹, —C(═O)—NR⁶R⁷, —C(═O)—R⁸, —S(═O)₂—NR⁶R⁷, —S(═O)—R⁹, —S(═O)₂—R⁹, and/or —NH—S(═O)₂—R₉.

In the general formula (I) R^{1a} further very particularly preferably represents isoxazolyl, pyrazolyl, thienyl, thiazolyl, imidazolyl, triazolyl, pyrrolyl, oxadiazolyl, pyridinyl or pyrimidinyl, which may optionally be mono- or polysubstituted by identical or different substituents from the group consisting of fluorine, chlorine, hydroxy, amino, cyano, nitro, C₁-C₃-alkyl, C₁-C₃-alkoxy, C₁-C₂ alkoxy-C₁-C₂ alkyl dimethylamino, trifluoromethyl, difluoroethyl, trifluoroethyl, trifluoromethoxy, cyclopropyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, pyridinyl, phenyl, and/or fluorophenyl.

In the general formula (I) R^{1a} further very particularly preferably represents phenyl, which may optionally be mono- or polysubstituted by identical or different substituents from the group consisting of fluorine, chlorine, amino, hydroxy, cyano, nitro, carboxyl, C₁-C₃-alkyl, C₁-C₃-alkoxy, C₁-C₂-alkoxy-C₁-C₂-alkyl, dimethylamino, —C(═O)NR⁶R⁷, C(═O)R⁸, C₁-C₃-alkylsulphinyl, C₁-C₃-alkylsulphonyl, —S(═O)₂NH₂, C₁-C₃-alkylsulphonylamino, C₁-C₃-alkylaminosulphonyl, C₃-C₆-cycloalkylaminosulphonyl, trifluoromethyl, difluoroethyl, trifluoroethyl, trifluoromethoxy, hydroxy-C₁-C₃-alkyl, cyclopropyl, morpholino and/or pyridinyl.

In the general formula (I) R^{1a} very particularly preferably represents hydrogen or chlorine,

or

represents piperazinyl, pyrrolidinyl, piperidinyl, diazepanyl, oxazinanyl, oxazolidinyl, morpholinyl, thiomorpholinyl, thiazolidinyl or azetidinyl, which may optionally be mono- or polysubstituted by identical or different substituents from the group consisting of fluorine, hydroxy, oxo, C₁-C₃-alkyl, methoxy, hydroxy-C₁-C₃-alkyl, dimethylamino, difluoroethyl, trifluoroethyl, benzyl, —NR⁶C(═O)—R⁹, —C(═O)—NR⁶R⁷, —C(═O)—R⁸, and/or —S(═O)₂—R⁹,

or

represents tetrazolyl,

or

represents isoxazolyl, pyrazolyl, thienyl, thiazolyl, imidazolyl, triazolyl, pyrrolyl, oxadiazolyl, pyridinyl or pyrimidinyl, which may optionally be mono- or polysubstituted by identical or different substituents from the group consisting of fluorine, chlorine, hydroxy, cyano, C₁-C₂-alkyl, methoxy, methoxymethyl, trifluoromethyl, cyclopropyl, —C(═O)—R⁸, pyridinyl, phenyl, and/or fluorophenyl,

or

represents phenyl which may optionally be mono- or polysubstituted by identical or different substituents from the group consisting of fluorine, chlorine, hydroxy, cyano, nitro, carboxyl, C₁-C₃-alkyl, methoxy, —C(═O)NR⁶R⁷, C(═O)R⁸, C₁-C₃-alkylsulphinyl, C₁-C₃-alkylsulphonyl, —S(═O)₂NH₂, C₁-C₃-alkylsulphonylamino, C₁-C₃-alkylaminosulphonyl, C₃-C₆-cycloalkylaminosulphonyl, trifluoromethyl, trifluoromethoxy, hydroxy-C₁-C₃-alkyl, cyclopropyl, and/or morpholino.

In the general formula (I) R^{1a} further very particularly preferably represents piperazinyl, pyrrolidinyl, piperidinyl, diazepanyl, oxazinanyl, oxazolidinyl, morpholinyl, thiomorpholinyl, thiazolidinyl or azetidinyl, which may optionally be mono- or polysubstituted by identical or different substituents from the group consisting of fluorine, hydroxy, oxo, C₁-C₃-alkyl, methoxy, hydroxy-C₁-C₃-alkyl, dimethylamino, difluoroethyl, trifluoroethyl, benzyl, —NR⁶C(═O)—R⁹,

—C(═O)—NR⁶R⁷, —C(═O)—R⁸, and/or —S(═O)₂—R⁹,

or

represents tetrazolyl,

or

represents isoxazolyl, pyrazolyl, thienyl, thiazolyl, imidazolyl, triazolyl, pyrrolyl, oxadiazolyl, pyridinyl or pyrimidinyl, which may optionally be mono- or polysubstituted by identical or different substituents from the group consisting of fluorine, chlorine, hydroxy, cyano, C₁-C₂-alkyl, methoxy, methoxymethyl, trifluoromethyl, cyclopropyl, —C(═O)—R⁸, pyridinyl, phenyl, and/or fluorophenyl,

or

represents phenyl, which may optionally be mono- or polysubstituted by identical or different substituents from the group consisting of fluorine, chlorine, hydroxy, cyano, nitro, carboxyl, C₁-C₃-alkyl, methoxy, —C(═O)NR⁶R⁷, C(═O)R⁸, C₁-C₃ alkylsulphinyl, C₁-C₃-alkylsulphonyl, —S(═O)₂NH₂, C₁-C₃ alkylsulphonylamino, C₁-C₃-alkylaminosulphonyl, C₃-C₆-cycloalkylaminosulphonyl, trifluoromethyl, trifluoromethoxy, hydroxy-C₁-C₃-alkyl, cyclopropyl, and/or morpholino.

In the general formula (1) R^{1a} further very particularly preferably represents piperazinyl, piperidinyl, diazepanyl, oxazinanyl, oxazolidinyl, morpholinyl, thiomorpholinyl, thiazolidinyl or azetidinyl, which may optionally be mono- or polysubstituted by identical or different substituents from the group consisting of fluorine, hydroxy, oxo, methoxy, hydroxy-C₁-C₃-alkyl, dimethylamino, difluoroethyl, trifluoroethyl, benzyl, —NR⁶C(═O)—R⁹, —C(═O)—NR⁶R⁷, —C(═O)—R⁸, and/or —S(═O)₂—R⁹,

or

represents tetrazolyl,

or

represents isoxazolyl, pyrazolyl, thienyl, thiazolyl, imidazolyl, triazolyl, pyrrolyl, oxadiazolyl, pyridinyl or pyrimidinyl, which may optionally be mono- or polysubstituted by identical or different substituents from the group consisting of fluorine, chlorine, hydroxy, cyano, C₁-C₂-alkyl, methoxy, methoxymethyl, trifluoromethyl, cyclopropyl, —C(═O)—R⁸, pyridinyl, phenyl, and/or fluorophenyl,

or

represents phenyl, which may optionally be mono- or polysubstituted by identical or different substituents from the group consisting of fluorine, chlorine, hydroxy, cyano, nitro, carboxyl, C₁-C₃-alkyl, C₁-C₃-methoxy, —C(═O)NR⁶R⁷, C(═O)R⁸, C₁-C₃-alkylsulphinyl, C₁-C₃-alkylsulphonyl, —S(═O)₂NH₂, C₁-C₃-alkylsulphonylamino, C₁-C₃-alkylaminosulphonyl, C₃-C₆-cycloalkylaminosulphonyl, trifluoromethyl, trifluoromethoxy, hydroxy-C₁-C₃-alkyl, cyclopropyl, and/or morpholino.

In the general formula (I) R^{1a} further exceptionally preferably represents piperazinyl, pyrrolidinyl, piperidinyl, diazepanyl, oxazinanyl, oxazolidinyl, morpholinyl, thiomorpholinyl, thiazolidinyl or azetidinyl, which may optionally be mono- or polysubstituted by identical or different substituents from the group consisting of fluorine, hydroxy, oxo, C₁-C₃-alkyl, methoxy, hydroxy-C₁-C₃-alkyl, dimethylamino, difluoroethyl, trifluoroethyl, benzyl, —NR⁶C(═O)—R⁹, —C(═O)—NR⁶R⁷, —C(═O)—R⁸, and/or —S(═O)₂—R⁹.

In the general formula (I) R^{1a} further exceptionally preferably represents isoxazolyl, pyrazolyl, thienyl, thiazolyl, imidazolyl, triazolyl, pyrrolyl, oxadiazolyl, pyridinyl or pyrimidinyl, which may optionally be mono- or polysubstituted by identical or different substituents from the group consisting of fluorine, chlorine, hydroxy, cyano, C₁-C₂-alkyl, methoxy, methoxymethyl, trifluoromethyl, cyclopropyl, —C(═O)—R⁸, pyridinyl, phenyl, and/or fluorophenyl.

In the general formula (I) R^{1a} further exceptionally preferably represents phenyl, which may optionally be mono- or polysubstituted by identical or different substituents from the group consisting of fluorine, chlorine, hydroxy, cyano, nitro, carboxyl, C₁-C₃-alkyl, C₁-C₃-methoxy, —C(═O)N12⁶R⁷, C(═O)R⁸, C₁-C₃-alkylsulphinyl, C₁-C₃-alkylsulphonyl, —S(═O)₂NH₂, C₁-C₃-alkylsulphonylamino, C₁-C₃-alkylaminosulphonyl, C₃-C₆-cycloalkylaminosulphonyl, trifluoromethyl, trifluoromethoxy, hydroxy-C₁-C₃-alkyl, cyclopropyl, and/or morpholino.

In the general formula (I) R^{1a} further exceptionally preferably represents piperazinyl, pyrrolidinyl, piperidinyl, diazepanyl, oxazinanyl, oxazolidinyl, morpholinyl, thiomorpholinyl, thiazolidinyl or azetidinyl, which may optionally be mono- or polysubstituted by identical or different substituents from the group consisting of fluorine, hydroxy, oxo, C₁-C₃-alkyl, methoxy, hydroxy-C₁-C₃-alkyl, dimethylamino, difluoroethyl, trifluoroethyl, benzyl, —NR⁶C(═O)—R⁹, —C(═O)-Nlele, —C(═O)—R⁸, and/or —S(═O)₂—R⁹,

or

represents isoxazolyl or pyrazolyl, which may optionally be mono- or polysubstituted by identical or different C₁-C₂-alkyl substituents.

In the general formula (I) R^{1a} further exceptionally preferably represents piperazinyl, pyrrolidinyl, piperidinyl, diazepanyl, oxazinanyl, oxazolidinyl, morpholinyl, thiomorpholinyl, thiazolidinyl or azetidinyl, which may optionally be mono- or polysubstituted by identical or different substituents from the group consisting of fluorine, hydroxy, oxo, C₁-C₃-alkyl, methoxy, hydroxy-C₁-C₃-alkyl, dimethylamino, difluoroethyl, trifluoroethyl, benzyl, —NR⁶C(═O)—R⁹, —C(═O)—NR⁶R⁷, —C(═O)—R⁸, and/or —S(═O)₂—R₉.

In the general formula (I) R^{1a} further exceptionally preferably represents isoxazolyl or pyrazolyl, which may optionally be mono- or polysubstituted by identical or different C₁-C₂-alkyl substituents.

In the general formula (I) R^{1a} further exceptionally preferably represents piperazinyl, pyrrolidinyl, piperidinyl, morpholinyl, thiomorpholinyl or azetidinyl, which may optionally be mono- or polysubstituted by identical or different substituents from the group consisting of hydroxy, oxo, C₁-C₃-alkyl, methoxy, dimethylamino, difluoroethyl, trifluoroethyl, —NR⁶C(═O)—R⁹, —C(═O)—NR(R⁷, and/or —C(═O)—R⁸.

In the general formula (1) R^{1a} further exceptionally preferably represents piperazinyl, pyrrolidinyl, piperidinyl, morpholinyl, thiomorpholinyl or azetidinyl, which may optionally be monosubstituted by C₁-C₃-alkyl.

In the general formula (1) R ^{l}a further exceptionally preferably represents piperazinyl, piperidinyl, morpholinyl, thiomorpholinyl or azetidinyl, which may optionally be monosubstituted by methyl.

In the general formula (I) R^{1a} further exceptionally preferably represents piperazinyl which may optionally be mono- or polysubstituted by identical or different substituents from the group consisting of hydroxy, oxo, C₁-C₃-alkyl, methoxy, dimethylamino, difluoroethyl, trifluoroethyl, —NR⁶C(═O)—R⁹, —C(═O)—NR⁶R⁷, and/or —C(═O)—R⁸.

In the general formula (I) R^{1b} and R^{1c} preferably and independently of one another represent hydrogen, halogen, hydroxy, cyano, nitro or represent a C₁-C₆-alkyl, C₁-C₆-alkoxy, C₁-C₆-alkoxy-C₁-C₆-alkyl, halo-C₁-C₆-alkyl, halo-C₁-C₆-alkoxy, C₃-C₁₀-cycloalkyl radical, and/or a monocyclic heterocycyl radical having 4 to 7 ring atoms.

In the general formula (I) R^{1b} preferably represents hydrogen, halogen, hydroxy, cyano, nitro or represents a C₁-C₆-alkyl, C₁-C₆-alkoxy-C₁-C₆-alkoxy-C₁-C₆-alkyl, halo-C₁-C₆-alkyl, halo-C₁-C₆-alkoxy, C₃-C₁₀-cycloalkyl radical, or a monocyclic heterocycyl radical having 4 to 7 ring atoms.

In the general formula (I) R^{1b} very preferably represents hydrogen, halogen, hydroxy, cyano, nitro or represents a C₁-C₃-alkyl, C₁-C₃-alkoxy, fluoro-C₁-C₃-alkyl or fluoro-C₁-C₃-alkoxy radical.

In the general formula (I) R^{1c} very preferably represents hydrogen, fluorine, chlorine, bromine or cyano.

In the general formula (I) R^{1b} very preferably represents hydrogen, fluorine, bromine or cyano.

In the general formula (I) R^{1c} very preferably represents hydrogen or bromine.

In the general formula (I) R^{1c} very preferably represents hydrogen.

In the general formula (I) R^{1b} very preferably represents hydrogen, fluorine, bromine or cyano and R^{1c} represents hydrogen.

In the general formula (I) it is possible that R² represents a C₁-C₃-alkyl or trifluoromethyl or a C₃- or C₄-cycloalkyl radical.

In the general formula (I) R² preferably represents methyl, ethyl or isopropyl.

In the general formula (I) R² very preferably represents methyl or ethyl.

In the general formula (I) R² exceptionally preferably represents methyl.

In the general formula (I) R³ preferably represents cyclopropyl, C₁-C₃-alkoxy, amino, cyclopropylamino or C₁-C₃-alkylamino.

In the general formula (I) R³ very preferably represents cyclopropyl, C₁-C₃-alkyl, C₁-C₃-alkoxy, cyclopropylamino or C₁-C₃-alkylamino.

In the general formula (I) R³ very preferably represents cyclopropyl, methyl, ethyl, methoxy, ethoxy, cyclopropylamino, methylamino or ethylamino.

In the general formula (I) R³ exceptionally preferably represents methylamino.

In the general formula (I) it is possible that R⁴ and R⁵ independently of one another represent hydrogen, hydroxy, cyano, nitro, amino, aminocarbonyl, fluorine, chlorine, bromine, C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl,

or

represent C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl, which may optionally be mono- or polysubstituted by identical or different substituents from the group consisting of halogen, amino, hydroxy, carboxyl, hydroxy-C₁-C₆-alkyl, C₁-C₆-alkoxy, C₁-C₆-alkoxy-C₁-C₆-alkyl, C₁-C₆-alkylamino, amino-C₁-C₆-alkyl, monocyclic heterocyclyl having 3 to 8 ring atoms and/or monocyclic heteroaryl having 5 or 6 ring atoms, where the monocyclic heterocyclyl and heteroaryl radicals mentioned for their part may optionally be monosubstituted by C₁-C₃-alkyl,

or

represent C₃-C₁₀-cycloalkyl which may optionally be mono- or polysubstituted by identical or different substituents from the group consisting of halogen, amino, hydroxy, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, and/or a monocyclic heterocyclyl radical having 3 to 8 ring atoms,

or

represent monocyclic heteroaryl which has 5 or 6 ring atoms which may optionally be mono- or polysubstituted by identical or different substituents from the group consisting of halogen, amino, hydroxy, cyano, nitro, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₁-C₁₀-cycloalkyl, —C(═O)R⁸, —S(═O)₂R⁹, —NR⁶R⁷, and/or a monocyclic heterocyclyl radical having 3 to 8 ring atoms,

or

represent monocyclic heterocyclyl having 3 to 8 ring atoms which may optionally be mono- or polysubstituted by identical or different substituents from the group consisting of halogen, amino, hydroxy, cyano, oxo, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-a lkylamino-C₁-C₆-alkyl, hydroxy-C₁-C₆-alkyl, halo-C₁-C₆-alkyl, halo-C₁-C₆-alkoxy, C₃-C₁₀-cycloalkyl, —C(═O)R⁸, —S(═O)₂R⁹, —NR⁶R⁷, and/or a monocyclic heterocyclyl radical having 3 to 8 ring atoms,

or

represent phenyl which may optionally be mono- or polysubstituted by identical or different substituents from the group consisting of halogen, amino, hydroxy, cyano, nitro, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylaminocarbonyl, C₁-C₆-alkylaminosulphonyl, C₁-C₆-alkylamino-C₁-C₆-alkyl, hydroxy-C₁-C₆-alkyl, halo-C₁-C₆-alkyl, halo-C₁-C₆-alkoxy, C₃-C₁₀-cycloalkyl and/or a monocyclic heterocyclyl radical having 3 to 8 ring atoms.

In the general formula (I) R⁴ and R⁵ preferably and independently of one another represent hydrogen, hydroxy, cyano, nitro, amino, aminocarbonyl, fluorine, chlorine, bromine, C₁-C₆-alkyl, C₁-C₆-alkoxy, C₆-alkylamino, C₁-C₆-alkylcarbonylamino, C₁-C₆-alkylaminocarbonyl or C₁-C₆-alkylaminosulphonyl,

or

represent C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl, which may optionally be mono- or polysubstituted by identical or different substituents from the group consisting of halogen, amino, hydroxy, carboxyl, hydroxy-C₁-C₆-alkyl, C₁-C₆-alkoxy, C₁-C₆-alkoxy-C₁-C₆-alkyl, C₁-C₆-alkylamino, amino-C₁-C₆-alkyl, a monocyclic heterocyclyl radical having 4 to 7 ring atoms, and/or a monocyclic heteroaryl radical having 5 or 6 ring atoms, where the monocyclic heterocyclyl and heteroaryl radicals mentioned for their part may optionally be monosubstituted by C₁-C₃-alkyl,

or

represents a C₃-C₁₀-cycloalkyl radical, which may optionally be mono- or polysubstituted by identical or different substituents from the group consisting of halogen, amino, hydroxy, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, and/or a monocyclic heterocyclyl radical having 4 to 7 ring atoms,

or

represents a monocyclic heteroaryl radical having 5 or 6 ring atoms, which may optionally be mono- or polysubstituted by identical or different substituents from the group consisting of halogen, amino, hydroxy, cyano, nitro, carboxyl, CG-C6-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, —C(═O)R⁸, —S(═O)₂R⁹, —NR⁶R⁷, and/or a monocyclic heterocyclyl radical having 4 to 7 ring atoms,

or

represents a monocyclic heterocyclyl radical having 4 to 7 ring atoms, which may optionally be mono- or polysubstituted by identical or different substituents from the group consisting of halogen, amino, hydroxy, cyano, oxo, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, hydroxy-C₁-C₆-alkyl, halo-C₁-C₆-alkyl, halo-C₁-C₆-alkoxy, C₃-C₁₀-cycloalkyl, —C(═O)R⁸, —S(═O)₂R⁹, —NR⁶R⁷, and/or a monocyclic heterocyclyl radical having 4 to 7 ring atoms.

In the general formula (I) R⁴ preferably represents a C₃-Cio-cycloalkyl radical, which may optionally be mono- or polysubstituted by identical or different substituents from the group consisting of halogen, amino, hydroxy, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, and/or a monocyclic heterocyclyl radical having 4 to 7 ring atoms,

or

represents a monocyclic heteraryl radical having 5 or 6 ring atoms, which may optionally be mono- or polysubstituted by identical or different substituents from the group consisting of halogen, amino, hydroxy, cyano, nitro, carboxyl, C₁-C₆-alkyl, C₁-C₆-alkoxy, C₁-C₆-alkoxy-C₁-C₆-alkyl, hydroxy-C₁-C₆-alkyl, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, —C(═O)R⁸, —S(═O)₂R⁹, —NR⁶R⁷, and/or a monocyclic heterocyclyl radical having 4 to 7 ring atoms,

or

represents a monocyclic heterocyclyl radical having 4 to 7 ring atoms, which may optionally be mono- or polysubstituted by identical or different substituents from the group consisting of halogen, amino, hydroxy, cyano, oxo, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, hydroxy-C₁-C₆-alkyl, halo-C₁-C₆-alkyl, halo-C₁-C₆-alkoxy, C₃-C₁₀-cycloalkyl, —C(═O)R⁸, —S(═O)₂R⁹, —NR⁶R⁷, and/or a monocyclic heterocyclyl radical having 4 to 7 ring atoms,

and R⁵ preferably represents hydrogen, hydroxy, cyano, nitro, amino, aminocarbonyl, fluorine, chlorine, bromine, C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl,

or

represents C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl, which may optionally be mono- or polysubstituted by identical or different substituents from the group consisting of halogen, amino, hydroxy, carboxyl, hydroxy-C₁-C₆-alkyl, C₁-C₆-alkoxy, C₁-C₆-alkoxy-C₁-C₆-alkyl, C₁-C₆-alkylamino, amino-C₁-C₆-alkyl, a monocyclic heterocyclyl radical having 4 to 7 ring atoms, and/or a monocyclic heteroaryl radical having 5 or 6 ring atoms, where the monocyclic heterocyclyl and heteroaryl radicals mentioned for their part may optionally be monosubstituted by C₁-C₃-alkyl.

In the general formula (I) R⁴ preferably represents hydrogen, hydroxy, cyano, nitro, amino, aminocarbonyl, fluorine, chlorine, bromine, C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl,

or

represents C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl, which may optionally be mono- or polysubstituted by identical or different substituents from the group consisting of halogen, amino, hydroxy, carboxyl, hydroxy-C₁-C₆-alkyl, C₁-C₆-alkoxy, C₁-C₆-alkoxy-C₁-C₆-alkyl, C₁-C₆-alkylamino, amino-C₁-C₆-alkyl, a monocyclic heterocyclyl radical having 4 to 7 ring atoms, and/or a monocyclic heteroaryl radical having 5 or 6 ring atoms, where the monocyclic heterocyclyl and heteroaryl radicals mentioned for their part may optionally be monosubstituted by C₁-C₃-alkyl,

and R⁵ preferably represents a C₃-C₁₀-cycloalkyl radical, which may optionally be mono- or polysubstituted by identical or different substituents from the group consisting of halogen, amino, hydroxy, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, and/or a monocyclic heterocyclyl radical having 4 to 7 ring atoms,

or

represents a monocyclic heteroaryl radical having 5 or 6 ring atoms, which may optionally be mono- or polysubstituted by identical or different substituents from the group consisting of halogen, amino, hydroxy, cyano, nitro, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, —C(═O)R⁸, —S(═O)₂R⁹, —NR⁶R⁷, and/or a monocyclic heterocyclyl radical having 4 to 7 ring atoms,

or

represents a monocyclic heterocyclyl radical having 4 to 7 ring atoms, which may optionally be mono- or polysubstituted by identical or different substituents from the group consisting of halogen, amino, hydroxy, cyano, oxo, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, hydroxy-C₁-C₆-alkyl, halo-C₁-C₆-alkyl, halo-C₁-C₆-alkoxy, C₃-C₁₀-cycloalkyl, —C(═O)R⁸, —S(═O)₂R⁹, —NR⁶R⁷, and/or a monocyclic heterocyclyl radical having 4 to 7 ring atoms.

In the general formula (I) Wand R⁵ particularly preferably and independently of one another represent hydrogen, hydroxy, cyano, nitro, amino, aminocarbonyl, fluorine, chlorine, bromine, C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl,

or

represent C₁-C₃-alkyl, C₁-C₃-alkoxy, C₁-C₃-alkylamino, which may optionally be mono- or polysubstituted by identical or different substituents from the group consisting of halogen, amino, hydroxy, carboxyl, hydroxy-C₁-C₃-alkyl, C₁-C₃-alkoxy, C₁-C₃-alkylamino, amino-C₁-C₃-alkyl, a monocyclic heterocyclyl radical having 4 to 7 ring atoms, and/or a monocyclic heteroaryl radical having 5 or 6 ring atoms, where the monocyclic heterocyclyl and heteroaryl radicals mentioned for their part may optionally be monosubstituted by C₁-C₃-alkyl,

or

represents a C₃-C₇-cycloalkyl radical, which may optionally be mono- or polysubstituted by identical or different substituents from the group consisting of halogen, amino, hydroxy, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, fluoro-C₁-C₃-alkyl, fluoro-C₁-C₃-alkoxy, and/or a monocyclic heterocyclyl radical having 4 to 7 ring atoms,

or

represents a monocyclic heteroaryl radical having 5 or 6 ring atoms, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, hydroxy-C₁-C₃-alkyl, C₁-C₃-alkylamino, amino-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷, and/or a monocyclic heterocyclyl radical having 4 to 7 ring atoms,

or

represents a monocyclic heterocyclyl radical having 4 to 7 ring atoms, which may optionally be mono- or polysubstituted by identical or different substituents from the group consisting of halogen, amino, hydroxy, cyano, oxo, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, hydroxy-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷, and/or a monocyclic heterocyclyl radical having 4 to 7 ring atoms,

or

represents a phenyl radical, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, C,-C₃-alkylamino, amino-C,-C₃-alkyl, C₁-C₃-alkylaminocarbonyl, C₁-C₃-alkylaminosulphonyl, hydroxy-C₁-C₃-alkyl, fluoro-C₁-C₃-alkyl, fluoro-C₁-C₃-alkoxy, C₃-C₆-cycloalkyl, and/or a monocyclic heterocyclyl radical having 4 to 7 ring atoms.

In the general formula (I) R⁴ very preferably represents hydrogen, hydroxy, cyano, nitro, amino, aminocarbonyl, fluorine, chlorine, bromine, C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl,

or

represents C₁-C₃-alkyl, C₁-C₃-alkoxy, C₁-C₃-alkylamino, which may optionally be mono- or polysubstituted by identical or different substituents from the group consisting of halogen, amino, hydroxy, carboxyl, hydroxy-C₁-C₃-alkyl, C₁-C₃-alkoxy, C₁-C₃-alkylamino, amino-C₁-C₃-alkyl, a monocyclic heterocyclyl radical having 4 to 7 ring atoms, and/or a monocyclic heteroaryl radical having 5 or 6 ring atoms, where the monocyclic heterocyclyl and heteroaryl radicals mentioned for their part may optionally be monosubstituted by C₁-C₃-alkyl,

and R⁵ very preferably represents a C₃-C₇-cycloalkyl radical, which may optionally be mono- or polysubstituted by identical or different substituents from the group consisting of halogen, amino, hydroxy, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, fluoro-C₁-C₃-alkyl, fluoro-C₁-C₃-alkoxy, and/or a monocyclic heterocyclyl radical having 4 to 7 ring atoms,

or

represents a monocyclic heteroaryl radical having 5 or 6 ring atoms, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, hydroxy-C₁-C₃-alkyl, C₁-C₃-alkylamino, amino-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷, and/or a monocyclic heterocyclyl radical having 4 to 7 ring atoms,

or

represents a monocyclic heterocyclyl radical having 4 to 7 ring atoms, which may optionally be mono- or polysubstituted by identical or different substituents from the group consisting of halogen, amino, hydroxy, cyano, oxo, carboxyl, C₁-C₃-alkyl, C₁-C₃-alkoxy, C₁-C₂ alkoxy C₁-C₂ alkyl, C₁-C₃-alkylamino, amino-C₁-C₃-alkyl, hydroxy-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷, and/or a monocyclic heterocyclyl radical having 4 to 7 ring atoms,

or

represents a phenyl radical, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, C₁-C₃-alkylaminocarbonyl, C₁-C₁-alkylaminosulphonyl, hydroxy-C₁-C₃-alkyl, fluoro-C₁-C₃-alkyl, fluoro-C₁-C₃-alkoxy, C₃-C₆-cycloalkyl, and/or a monocyclic heterocyclyl radical having 4 to 7 ring atoms.

In the general formula (I) R⁴ very preferably represents a C₃-C₇-cycloalkyl radical, which may optionally be mono- or polysubstituted by identical or different substituents from the group consisting of halogen, amino, hydroxy, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, fluoro-C₁-C₃-alkyl, fluoro-C₁-C₃-alkoxy, and/or a monocyclic heterocyclyl radical having 4 to 7 ring atoms,

or

represents a monocyclic heteroaryl radical having 5 or 6 ring atoms, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, hydroxy-C₁-C₃-alkyl, C₁-C₃-alkylamino, amino-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷, and/or a monocyclic heterocyclyl radical having 4 to 7 ring atoms,

or

represents a monocyclic heterocyclyl radical having 4 to 7 ring atoms, which may optionally be mono- or polysubstituted by identical or different substituents from the group consisting of halogen, amino, hydroxy, cyano, oxo, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, hydroxy-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷, and/or a monocyclic heterocyclyl radical having 4 to 7 ring atoms,

or

represents a phenyl radical, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, C₁-C₃-alkylaminocarbonyl, C₁-C₃-alkylaminosulphonyl, hydroxy-C₁-C₃-alkyl, fluoro-C₁-C₃-alkyl, fluoro-C₁-C₃-alkoxy, C₃-C₆-cycloalkyl, and/or a monocyclic heterocyclyl radical having 4 to 7 ring atoms, and R⁵ very preferably represents hydrogen, hydroxy, cyano, nitro, amino, aminocarbonyl, fluorine, chlorine, bromine, C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl,

or

represents Cl-C₃-alkyl, C₁-C₃-alkoxy, C₁-C₃-alkylamino, which may optionally be mono- or polysubstituted by identical or different substituents from the group consisting of halogen, amino, hydroxy, carboxyl, hydroxy-C₁-C₃-alkyl, C₁-C₃-alkoxy, C₁-C₃-alkylamino, amino-C₁-C₃-alkyl, a monocyclic heterocyclyl radical having 4 to 7 ring atoms, and/or a monocyclic heteroaryl radical having 5 or 6 ring atoms, where the monocyclic heterocyclyl and heteroaryl radicals mentioned for their part may optionally be monosubstituted by C₁-C₃-alkyl.

In the general formula (I) R⁴ very preferably represents a C₃-C₂-cycloalkyl radical, which may optionally be mono- or polysubstituted by identical or different substituents from the group consisting of halogen, amino, hydroxy, carboxyl, C₁-C₃-alkyl, C₁-C₃ alkoxy, C₁-C₂ alkoxy C₁-C₂ alkyl, C₁-C₃-alkylamino, amino-C₁-C₃-alkyl, fluoro-C₁-C₃-alkyl, fluoro-C₁-C₃-alkoxy, and/or a monocyclic heterocyclyl radical having 4 to 7 ring atoms,

or

represents a monocyclic heteroaryl radical having 5 or 6 ring atoms, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, hydroxy-C₁-C₃-alkyl, C₁-C₃-alkylamino, amino-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷, and/or a monocyclic heterocyclyl radical having 4 to 7 ring atoms,

or

represents a monocyclic heterocyclyl radical having 4 to 7 ring atoms, which may optionally be mono- or polysubstituted by identical or different substituents from the group consisting of halogen, amino, hydroxy, cyano, oxo, carboxyl, C₁-C₃-alkyl, C₁-C₃-alkoxy, C₁-C₂ alkoxy C₁-C₂ alkyl, C₁-C₃ alkylamino, amino-C₁-C₃-alkyl, hydroxy-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷, and/or a monocyclic heterocyclyl radical having 4 to 7 ring atoms,

or

represents a phenyl radical, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, C₁-C₃ alkylamino, amino-C₁-C₃-alkyl, C₁-C₃-alkylaminocarbonyl, C₁-C₃-alkylaminosulphonyl, hydroxy-C₁-C₃-alkyl, fluoro-C₁-C₃-alkyl, fluoro-C₁-C₃-alkoxy, C₃-C₆-cycloalkyl, and/or a monocyclic heterocyclyl radical having 4 to 7 ring atoms.

In the general formula (I) R⁴ very preferably represents a C₃-C₇-cycloalkyl radical, which may optionally be mono- or polysubstituted by identical or different substituents from the group consisting of halogen, amino, hydroxy, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, fluoro-C₁-C₃-alkyl, fluoro-C₁-C₃-alkoxy, and/or a monocyclic heterocyclyl radical having 4 to 7 ring atoms.

In the general formula (I) R⁴ very preferably represents a monocyclic heteroaryl radical having 5 or 6 ring atoms, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, hydroxy-C₁-C₃-alkyl, C₁-C₃-alkylamino, amino-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷, and/or a monocyclic heterocyclyl radical having 4 to 7 ring atoms.

In the general formula (I) R⁴ very preferably represents a monocyclic heterocyclyl radical having 4 to 7 ring atoms, which may optionally be mono- or polysubstituted by identical or different substituents from the group consisting of halogen, amino, hydroxy, cyano, oxo, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, hydroxy-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷, and/or a monocyclic heterocyclyl radical having 4 to 7 ring atoms.

In the general formula (I) R⁴ very preferably represents a phenyl radical, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, C₁-C₃-alkylamino, amino-C I -C₃-alkyl, C₁-C₃-alkylaminocarbonyl, C -C₃-alkylaminosulphonyl, hydroxy-C₁-C₃-alkyl, fluoro-C₁-C₃-alkyl, fluoro-C₁-C₃-alkoxy, C₃-C₆-cycloalkyl, and/or a monocyclic heterocyclyl radical having 4 to 7 ring atoms.

In the general formula (I) R⁵ very preferably represents a C₃-C₇-cycloalkyl radical, which may optionally be mono- or polysubstituted by identical or different substituents from the group consisting of halogen, amino, hydroxy, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, fluoro-C₁-C₃-alkyl, fluoro-C₁-C₃-alkoxy, and/or a monocyclic heterocyclyl radical having 4 to 7 ring atoms,

or

represents a monocyclic heteroaryl radical having 5 or 6 ring atoms, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, hydroxy-C₁-C₃-alkyl, C₁-C₃-alkylamino, amino-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷, and/or a monocyclic heterocyclyl radical having 4 to 7 ring atoms,

or

represents a monocyclic heterocyclyl radical having 4 to 7 ring atoms, which may optionally be mono- or polysubstituted by identical or different substituents from the group consisting of halogen, amino, hydroxy, cyano, oxo, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, hydroxy-C₁-C₃-alkyl, fluoro-C₁-C₃-alkyl, fluoro-C₁-C₃-alkoxy, C₃-C₆-cycloalkyl, —C(═O)1e, —S(═O)₂R⁹, —NR⁶R⁷, and/or a monocyclic heterocyclyl radical having 4 to 7 ring atoms,

or

represents a phenyl radical, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, C₁-C₃-alkylaminocarbonyl, C₁-C₃-alkylaminosulphonyl, hydroxy-C₁-C₃-alkyl, fluoro-C₁-C₃-alkyl, fluoro-C₁-C₃-alkoxy, C₃-C₆-cycloalkyl, and/or a monocyclic heterocyclyl radical having 4 to 7 ring atoms.

In the general formula (I) R⁵ very preferably represents a C₃-C₇-cycloalkyl radical, which may optionally be mono- or polysubstituted by identical or different substituents from the group consisting of halogen, amino, hydroxy, carboxyl, C₁-C₃-alkyl, C₁-C₃ alkoxy, C₁-C₂-alkoxy-C₁-C₂-alkyl. C₁-C₃-alkylamino, amino-C₁-C₃-alkyl, fluoro-C₁-C₃-alkyl, fluoro-C₁-C₃-alkoxy, and/or a monocyclic heterocyclyl radical having 4 to 7 ring atoms.

In the general formula (I) R⁵ very preferably represents a monocyclic heteroaryl radical having 5 or 6 ring atoms, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, hydroxy-C₁-C₃-alkyl, C₁-C₃-alkylamino, amino-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, -NleR⁷, and/or a monocyclic heterocyclyl radical having 4 to 7 ring atoms.

In the general formula (I) R⁵ very preferably represents a monocyclic heterocyclyl radical having 4 to 7 ring atoms, which may optionally be mono- or polysubstituted by identical or different substituents from the group consisting of halogen, amino, hydroxy, cyano, oxo, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, hydroxy-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷, and/or a monocyclic heterocyclyl radical having 4 to 7 ring atoms.

In the general formula (I) R⁵ very preferably represents a phenyl radical, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₃-alkylamino, amino-C₁-C₃-alkyl, C₁-C₃-alkylaminocarbonyl, C₁-C₃-alkylaminosulphonyl, hydroxy-C₁-C₃-alkyl, fluoro-C₁-C₃-alkyl, fluoro-C₁-C₃-alkoxy, C₃-C₆-cycloalkyl, and/or a monocyclic heterocyclyl radical having 4 to 7 ring atoms.

In the general formula (I) R⁴ and R⁵ very preferably and independently of one another represent hydrogen, hydroxy, cyano, amino, chlorine, C₁-C₆-alkyl, methoxy, ethoxy or C₁-C₃-alkylcarbonylamino,

or

represent difluoromethoxy or trifluoromethoxy,

or

represent C₁-C₃-alkoxy, which may be substituted by pyridinyl, morpholinyl, pyrrolidinyl or piperazinyl, in which the pyridinyl and piperazinyl may in turn optionally be substituted by C₁-C₃-alkyl,

or

represent cyclopropyl,

or

represent pyridinyl, pyrazolyl, triazolyl or isoxazolyl, which may optionally be mono- or polysubstituted by identical or different substituents from the group consisting of hydroxy and methyl,

or

represent pyrrolidinyl, morpholinyl, piperidinyl, piperazinyl, oxazolidinyl, thiomorpholinyl, which may optionally be mono- or polysubstituted by methyl, oxo, —S(═O)₂R⁹,

or

represent phenyl optionally substituted by C₁-C₃-alkylaminosulphonyl or fluorine.

In the general formula (I) R⁴ very preferably represents hydrogen, hydroxy, cyano, amino, chlorine, C₁-C₆-alkyl, methoxy, ethoxy or C₁-C₃-alkylcarbonylamino,

or

represents difluoromethoxy or trifluoromethoxy,

or

represents C₁-C₃-alkoxy, which may be substituted by pyridinyl, morpholinyl, pyrrolidinyl or piperazinyl, in which the pyridinyl and piperazinyl may in turn optionally be substituted by C₁-C₃-alkyl,

or

represents cyclopropyl,

or

represents pyridinyl, pyrazolyl, triazolyl or isoxazolyl, which may optionally be mono- or polysubstituted by identical or different substituents from the group consisting of hydroxy and methyl,

or

represents pyrrolidinyl, morpholinyl, piperidinyl, piperazinyl, oxazolidinyl, thiomorpholinyl, which may optionally be mono- or polysubstituted by methyl, oxo, —S(═O)₂R⁹,

or

represents phenyl optionally substituted by C₁-C₃-alkylaminosulphonyl or fluorine.

In the general formula (1) R⁵ very preferably represents hydrogen, hydroxy, cyano, amino, chlorine, C₁-C₆-alkyl, methoxy, ethoxy or C₁-C₃-alkylcarbonylamino,

or

represents difluoromethoxy or trifluoromethoxy,

or

represents C₁-C₃-alkoxy, which may be substituted by pyridinyl, morpholinyl, pyrrolidinyl or piperazinyl, in which the pyridinyl and piperazinyl may in turn optionally be substituted by C₁-C₃-alkyl,

or

represents cyclopropyl,

or

represents pyridinyl, pyrazolyl, triazolyl or isoxazolyl, which may optionally be mono- or polysubstituted by identical or different substituents from the group consisting of hydroxy and methyl,

or

represents pyrrolidinyl, morpholinyl, piperidinyl, piperazinyl, oxazolidinyl, thiomorpholinyl, which may optionally be mono- or polysubstituted by methyl, oxo, —S(═O)₂R⁹,

or

represents phenyl optionally substituted by C₁-C₃-alkylaminosulphonyl or fluorine.

In the general formula (I) R⁴ very preferably represents cyclopropyl,

or

represents pyridinyl, pyrazolyl, triazolyl or isoxazolyl, which may optionally be mono- or polysubstituted by identical or different substituents from the group consisting of hydroxy and methyl,

or

represents pyrrolidinyl, morpholinyl, piperidinyl, piperazinyl, oxazolidinyl, thiomorpholinyl, which may optionally be mono- or polysubstituted by methyl, oxo, —S(═O)₂R⁹,

or

represents phenyl optionally substituted by C₁-C₃-alkylaminosulphonyl or fluorine.

In the general formula (1) R⁵ very preferably represents cyclopropyl,

or

represents pyridinyl, pyrazolyl, triazolyl or isoxazolyl, which may optionally be mono- or polysubstituted by identical or different substituents from the group consisting of hydroxy and methyl,

or

represents pyrrolidinyl, morpholinyl, piperidinyl, piperazinyl, oxazolidinyl, thiomorpholinyl, which may optionally be mono- or polysubstituted by methyl, oxo, —S(═O)₂R⁹,

or

represents phenyl optionally substituted by C₁-C₃-alkylaminosulphonyl or fluorine.

In the general formula (I) R⁴ very particularly preferably represents C₁-C₃-alkoxy, which may be substituted by pyridinyl, morpholinyl, pyrrolidinyl or piperazinyl,

in which the pyridinyl and piperazinyl may in turn optionally be substituted by C₁-C₃-alkyl.

In the general formula (I) R⁵ very particularly preferably represents C₁-C₃-alkoxy, which may be substituted by pyridinyl, morpholinyl, pyrrolidinyl or piperazinyl,

in which the pyridinyl and piperazinyl may in turn optionally be substituted by C₁-C₃-alkyl.

In the general formula (I) R⁴ very particularly preferably represents difluoromethoxy or trifluoromethoxy.

In the general formula (I) R⁵ very particularly preferably represents difluoromethoxy or trifluoromethoxy.

In the general formula (I) R⁵ exceptionally preferably represents trifluoromethoxy.

In the general formula (I) R⁴ very preferably represents cyclopropyl,

or

represents pyridinyl, pyrazolyl, triazolyl or isoxazolyl, which may optionally be mono- or polysubstituted by identical or different substituents from the group consisting of hydroxy and methyl,

or

represents pyrrolidinyl, morpholinyl, piperidinyl, piperazinyl, oxazolidinyl or thiomorpholinyl, which may optionally be mono- or polysubstituted by oxo, methyl, —S(═O)₂R⁹,

or

represents phenyl optionally substituted by C₁-C₃-alkylaminosulphonyl or fluorine, and R⁵ exceptionally preferably represents hydrogen, hydroxy, cyano, chlorine, C₁-C₆-alkyl, methoxy, ethoxy, C₁-C₃-alkylcarbonylamino, difluoromethoxy or trifluoromethoxy.

In the general formula (I) R⁴ exceptionally preferably represents hydrogen, chlorine, methoxy, ethoxy, difluoromethoxy or trifluoromethoxy, and R⁵ exceptionally preferably represents cyclopropyl,

or

represents pyridinyl or pyrazolyl, which may optionally be mono- or polysubstituted by methyl,

or

represents morpholinyl, piperidinyl, piperazinyl, or thiomorpholinyl, which may optionally be mono- or polysubstituted by oxo, methyl, —S(═O)₂R⁹,

or

represents phenyl substituted by C₁-C₃-alkylaminosulphonyl.

In the general formula (I) R⁴ and R⁵ exceptionally preferably and independently of one another represent hydrogen, hydroxy, cyano, chlorine, C₁-C₆-alkyl, methoxy, ethoxy, C₁-C₃-alkylcarbonylamino, difluoromethoxy or trifluoromethoxy.

In the general formula (I) R⁴ and R⁵ exceptionally preferably and independently of one another represent hydrogen, chlorine, methoxy, ethoxy, difluoromethoxy or trifluoromethoxy.

In the general formula (I) R⁶ and R⁷ preferably and independently of one another represent hydrogen, C₁-C₃-alkyl, cyclopropyl, di-C₁-C₃-alkyl-amino-C₁-C₃-alkyl or fluoropyridyl.

In the general formula (I) R⁶ and R⁷ particularly preferably and independently of one another represent hydrogen, C₁-C₃-alkyl, cyclopropyl, di-C₁-C₃-alkyl-amino-C₁-C₃-alkyl.

In the general formula (I) R⁶ and R⁷ very preferably and independently of one another represent hydrogen or C₁-C₃-alkyl.

In the general formula (I) it is possible that IV represents hydroxy, C₁-C₆-alkyl, halo-C₁-C₃-alkyl, hydroxy-C₁-C₃-alkyl, C₁-C₃-alkoxy-C₁-C₃-alkyl, C₃-C₈-cycloalkyl, phenyl, monocyclic heterocyclyl having 3 to 8 ring atoms or monocyclic heteroaryl having 5 or 6 ring atoms, in which phenyl, heteroaryl and heterocyclyl may optionally be mono- or disubstituted by halogen, C₁-C₃-alkoxy- or C₁-C₃-alkyl.

In the general formula (I) R⁸ preferably represents hydroxy, C_{I}-C₆-alkyl, halo-C₁-C₃-alkyl, hydroxy-C₁-C₃-alkyl, C₁-C₃-alkoxy-C₁-C₃-alkyl, C₃-C₈-cycloalkyl, phenyl, monocyclic heterocyclyl having 5 or 6 ring atoms.

In the general formula (I) R⁸ very preferably represents hydroxy, hydroxy-C₁-C₃-alkyl, trifluoromethyl, pyrrolidinyl, morpholinyl or piperidinyl.

In the general formula (I) R⁸ very preferably represents C₁-C₃-alkyl.

In the general formula (I) R⁸ very preferably represents methyl.

In the general formula (I) R⁹ preferably represents hydrogen, C₁-C₆-alkyl or C₁-C₄-alkoxy.

In the general formula (I) R⁹ very preferably represents C₁-C₄-alkyl or C₁-C₄-alkoxy.

In the general formula (I) R⁹ very preferably represents methyl.

In the general formula (I) R⁹ very preferably represents tert-butoxy.

In the general formula (I) the stereocentre, which is represented by the carbon atom of the benzodiazepine skeleton which is bound to R², is preferably present either in racemic form or predominantly or completely in the (S) configuration.

In the general formula (I) the stereocentre, which is represented by the carbon atom of the benzodiazepine skeleton which is bound to R², is preferably present in racemic form.

In the general formula (I) the stereocentre, which is represented by the carbon atom of the benzodiazepine skeleton which is bound to R², is more preferably present predominantly or completely in the (S) configuration.

In the general formula (1) the stereocentre, which is represented by the carbon atom of the benzodiazepine skeleton which is bound to R², is more preferably present predominantly in the (S) configuration.

In the general formula (I) the stereocentre, which is represented by the carbon atom of the benzodiazepine skeleton which is bound to R², is more preferably present completely in the (S) configuration.

The invention additionally relates to compounds of the general formula (I) in which A represents phenyl and R⁴ represents hydrogen, fluorine, chlorine or bromine and R⁵ represents C₁-C₆-alkoxy which is mono- or polysubstituted by identical or different halogen substituents, and R¹ represents halogen.

The invention additionally relates to compounds of the general formula (I) in which A represents phenyl and R⁴ represents hydrogen and R⁵ represents C₁-C₆-alkoxy which is mono- or polysubstituted by identical or different substituents from the group consisting of a monocyclic heterocyclyl radical having 3 to 8 ring atoms, and/or a monocyclic heteroaryl radical having 5 or 6 ring atoms, in which the stated monocyclic heterocyclyl and heteroaryl radicals may in turn optionally be monosubstituted by C₁-C₃-alkyl, and R¹′ represents halogen.

The invention additionally relates to compounds of the general formula (I) in which A represents phenyl and R⁴ represents hydrogen and R⁵ represents C₁-C₆-alkoxy which is mono- or polysubstituted by identical or different substituents from the group consisting of a monocyclic heterocyclyl radical having 4 to 7 ring atoms, and/or a monocyclic heteroaryl radical having 5 or 6 ring atoms, in which the stated monocyclic heterocyclyl and heteroaryl radicals may in turn optionally be monosubstituted by C₁-C₃-alkyl, and R1′ represents halogen.

Preferred compounds of the general formula (I) are those in which A represents phenyl and R⁴ represents hydrogen, fluorine, chlorine or bromine and R⁵ represents C₁-C₃-alkoxy which is mono- or polysubstituted by identical or different halogen substituents, and R^{1a} represents halogen.

Preferred compounds of the general formula (I), furthermore, are those in which A represents phenyl and R⁴ represents hydrogen and R⁵ represents C₁-C₃-alkoxy which is mono- or polysubstituted by identical or different substituents from the group consisting of a monocyclic heterocyclyl radical having 4 to 7 ring atoms, and/or a monocyclic heteroaryl radical having 5 or 6 ring atoms, in which the stated monocyclic heterocyclyl and heteroaryl radicals may in turn optionally be monosubstituted by C₁-C₃-alkyl, and R^{1a} represents halogen.

Of very particular interest are compounds of the general formula (I) in which A represents a phenyl ring and R⁴ represents hydrogen or chlorine and R⁵ represents trifluoromethoxy, and R^{1a} represents chlorine.

Of very particular interest, furthermore, are compounds of the general formula (1) in which A represents a phenyl ring and R⁴ represents hydrogen and R⁵ represents C₁-C₃-alkoxy which is substituted by morpholinyl, pyrrolidinyl, piperazinyl or pyridyl, it being possible for the piperazinyl and pyridinyl itself to be substituted by C₁-C₃-alkyl, and R¹a represents chlorine.

Also of interest are those compounds of the general formula (I) in which A represents phenyl and R^{1a} represents a phenyl radical which may optionally be mono- or polysubstituted by identical or different substituents from the group consisting of fluorine, chlorine, amino, hydroxy, cyano, nitro, carboxyl, C₁-C₃-alkyl, C₁-C₃-alkoxy, C₁-C₂-alkoxy-C₁-C₂-alkyl, dimethylamino, —C(═O)NR⁶R⁷, —C(═O)R⁸, C₁-C₃-alkylsulphinyl, C₁-C₃-alkylsulphonyl, —S(═O)₂NH₂, C₁-C₃-alkylsulphonylamino, C₁-C₃-alkylaminosulphonyl, C₃-C₆-cycloalkylaminosulphonyl, trifluoromethyl, difluoroethyl, trifluoroethyl, trifluoromethoxy, hydroxy-C₁-C₃-alkyl, cyclopropyl, chlorothienyl, morpholino and/or pyridinyl.

Most preference is given to the following compounds:
(±)-1-(4-chlorophenyl)-N,4-dimethyl-8-(tri fluoromethoxy)-4,5-dihydro-3H-2,3-benzodiazepine-3-carboxamide;
(4S)-1-(4-chlorophenyl)-N,4-dimethyl-8-(trifluoromethoxy)-4,5-dihydro-3H-2,3-benzodiazepine-3-carboxamide;
(4R)-1-(4-chlorophenyl)-N,4-dimethyl-8-(tri fluoromethoxy)-4,5-dihydro-3H-2,3-benzodiazepine-3-carboxamide;
(±)-1-[4-(3,5-dimethyl-4-isoxazolyl)phenyl]-7,8-dimethoxy-N,4-dimethyl-4,5-dihydro-3H-2,3-benzodiazepine-3-carboxamide;
(4R)-1-[4-(3,5-dimethyl-4-isoxazolyl)phenyl]-7,8-dimethoxy-N,4-dimethyl-4,5-dihydro-3H-2,3-benzodiazepine-3-carboxamide;
(4S)-1-[4-(3,5-dimethyl-4-isoxazolyl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[4-(1H-pyrazol-3-yl)phenyl]-4,5-dihydro-3H-2,3-benzodiazepine-3-carboxamide;
(±)-1-[4-(2-chloropyridin-3-yl)phenyl]-7,8-dimethoxy-N,4-dimethyl-4,5-dihydro-3H-2,3-benzodiazepine-3-carboxamide;
(±)-5-(4-{7,8-dimethoxy-4-methyl-3-[(methylamino)carbonyl]-4,5-dihydro-3H-2,3-benzodiazepin-1-yl}phenyl)thiophene-2-carboxylic acid;
(±)-4-{7,8-dimethoxy-4-methyl-3-[(methylamino)carbonyl]-4,5-dihydro-3H-2,3-benzodiazepin-1-yl}biphenyl-2-carboxylic acid;
(±)-7,8-dimethoxy-N,4-dimethyl-1-[4-(morpholin-4-yl)phenyl]-4,5-dihydro-3H-2,3-benzodiazepine-3-carboxamide;
(±)-1-(4-chlorophenyl)-N,4-dimethyl-8-(pyridin-4-yl)-4,5-dihydro-3H-2,3-benzodiazepine-3-carboxamide;
(±)-1-(4-chlorophenyl)-8-cyclopropyl-N,4-dimethyl-4,5-dihydro-3H-2,3-benzodiazepine-3-carboxamide;
(±)-1-(4-chlorophenyl)-N,4-dimethyl-8-{4-[(methylamino)sulphonyl]phenyl}-4,5-dihydro-3H-2,3-benzodiazepine-3-carboxamide;
(±)-1-(4-chlorophenyl)-N,4-dimethyl-8-(morpholin-4-yl)-4,5-dihydro-3H-2,3-benzodiazepine-3-carboxamide;
(4S)-1-(4-chlorophenyl)-N,4-dimethyl-8-(morpholin-4-yl)-4,5-dihydro-3H-2,3-benzodiazepine-3-carboxamide;
(4R)-1-(4-chlorophenyl)-N,4-dimethyl-8-(morpholin-4-yl)-4,5-dihydro-3H-2,3-benzodiazepine-3-carboxamide;
(±)-1-(4-chlorophenyl)-N,4-dimethyl-8-(4-methylpiperazin-1-yl)-4,5-dihydro-3H-2,3-benzodiazepine-3-carboxamide;
(±)-1-(4-chlorophenyl)-N,4-dimethyl-8-(piperidin-1-yl)-4,5-dihydro-3H-2,3-benzodiazepine-3-carboxamide;
(±)-8-methoxy-N,4-dimethyl-1-(pyridin-3-yl)-4,5-dihydro-3H-2,3-benzodiazepine-3-carboxamide;
(±)-7-chloro-1-(4-chlorophenyl)-N,4-dimethyl-8-(trifluoromethoxy)-4,5-dihydro-3H-2,3-benzodiazepine-3-carboxamide;
7-chloro-1-(4-chlorophenyl)-N,4-dimethyl-8-(trifluoromethoxy)-4,5-dihydro-3H-2,3-benzodiazepine-3-carboxamide, enantiomer 1;
(4S)-1-[4-(3,5-dimethyl-1H-pyrazol-1-yl)phenyl]-8-methoxy-N,4-dimethyl-4,5-dihydro-3H-2,3-benzodiazepine-3-carboxamide;
(4S)-8-methoxy-N,4-dimethyl-1-[4-(morpholin-4-yl)phenyl]-4,5-dihydro-3H-2,3-benzodiazepine-3-carboxamide;
(4S)-1-[4-(4-isoxazolyl)phenyl]-8-methoxy-N,4-dimethyl-4,5-dihydro-3H-2,3-benzodiazepine-3-carboxamide;
(4S)-8-methoxy-N,4-dimethyl-1-[4-(1-methyl-1H-pyrazol-5-yl)phenyl]-4,5-dihydro-3H-2,3-benzodiazepine-3-carboxamide;
(4S)-1-[4-(3,5-dimethyl-4-isoxazolyl)phenyl]-8-methoxy-N,4-dimethyl-4,5-dihydro-3H-2,3-benzodiazepine-3-carboxamide;
(4S)-8-methoxy-N,4-dimethyl-1-[4-(1-methyl-1H-pyrazol-4-yl)phenyl]-4,5-dihydro-3H-2,3-benzodiazepine-3-carboxamide;
(4S)-8-methoxy-N,4-dimethyl-1-[4-(1,3,5-tr methyl-1H-pyrazol-4-yl)phenyl]-4,5-dihydro-3H-2,3-benzodiazepine-3-carboxamide;
(4S)-8-methoxy-N,4-dimethyl-1-[4-(1H-pyrazol-5-yl)phenyl]-4,5-dihydro-3H-2,3-benzodiazepine-3-carboxamide;
(4S)-1-[4-(3-cyclopropyl-5-ethyl-1H-pyrazol-1-yl)phenyl]-8-methoxy-N,4-dimethyl-4,5-dihydro-3H-2,3-benzodiazepine-3-carboxamide;
(4S)-1-[4-(5-cyclopropyl-3-ethyl-1H-pyrazol-1-yl)phenyl]-8-methoxy-N,4-dimethyl-4,5-dihydro-3H-2,3-benzodiazepine-3-carboxamide;
(4S)-8-methoxy-1-{4-[3-(methoxymethyl)-5-methyl-1H-p yrazol-1-yl]phenyl}-N,4-dimethyl-4,5-dihydro-3H-2,3-benzodiazepine-3-carboxamide;
(4S)-8-methoxy-1-{4-[5-(methoxymethyl)-3-methyl-1H-pyrazol-1-yl]phenyl}-N,4-dimethyl-4,5-dihydro-3H-2,3-benzodiazepine-3-carboxamide;
(4S)-1-{4-[5-cyclopropyl-3-(pyridin-2-yl)-1H-pyrazol-1-yl]phenyl}-8-methoxy-N,4-dimethyl-4,5-dihydro-3H-2,3-bcnzodiazepinc-3-carboxamide;
(4S)-1-{4-[3-cyclopropyl-5-(pyridin-2-yl)-1H-pyrazol-1-yl]phenyl}-8-methoxy-N,4-dimethyl-4,5-dihydro-3H-2,3-bcnzodiazepinc-3-carboxamide;
(4S)-8-methoxy-N,4-dimethyl-1-[4-(1H-tetrazol-1-yl)phenyl]-4,5-dihydro-3H-2,3-benzodiazepine-3-carboxamide;
(±)-1-[3-(3,5-dimethylisoxazol-4-yl)phenyl]-7,8-dimethoxy-N,4-dimethyl-4,5-dihydro-3H-2,3-benzodiazepine-3-carboxamide;
(±)-1-(4-chlorophenyl)-N,4-dimethyl-8-[2-(morpholin-4-yl)ethoxy]-4,5-dihydro-3H-2,3-benzodiazepine-3-carboxamide;
(±)-1-(4-chlorophenyl)-N,4-dimethyl-8-[2-(pyrrolidin-1-yl)ethoxy]-4,5-dihydro-3H-2,3-benzodiazepine-3-carboxamide;
(±)-7,8-dimethoxy-N,4-dimethyl-1-[4-(2-oxooxazolidin-3-yl)phenyl]-4,5-dihydro-3H-2,3-benzodiazepine-3-carboxamide;
(4S)-7,8-dimethoxy-N,4-dimethyl-1-[4-(2-oxooxazolidin-3-yl)phenyl]-4,5-dihydro-3H-2,3-benzodiazepine-3-carboxamide;
(±)-7,8-dimethoxy-N,4-dimethyl-1-[4-(2-oxopiperidin-1-yl)phenyl]-4,5-dihydro-3H-2,3-benzodiazepine-3-carboxamide;
(±)-1-[4-(4-benzyl-2-oxopiperazin-1-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[4-(4-methyl-2-oxo-1,4-diazepan-1-yl)phenyl]-4,5-dihydro-3H-2,3-benzodiazepine-3-carboxamide;
(±)-7,8-dimethoxy-N,4-dimethyl-1-[4-(2-oxo-1,3-oxazinan-3-yl)phenyl]-4,5-dihydro-3H-2,3-benzodiazepine-3-carboxamide;
(±)-7,8-dimethoxy-N,4-dimethyl-1-[4-(2-oxopyrrolidin-1-yl)phenyl]-4,5-dihydro-3H-2,3-benzodiazepine-3-carboxamide;
(±)-7,8-dimethoxy-N,4-dimethyl-1-[4-(3-oxomolpholin-4-yl)phenyl]-4,5-dihydro-3H-2,3-benzodiazepine-3-carboxamide;
(4S)-7,8-dimethoxy-N,4-dimethyl-1-[4-(3-oxomorpholin-4-yl)phenyl]-4,5-dihydro-3H-2,3-benzodiazepine-3-carboxamide;
(±)-7,8-dimethoxy-N,4-dimethyl-1-[4-(2-methyl-5-oxomorpholin-4-yl)phenyl]-4,5-dihydro-3H-2,3-benzodiazepine-3-carboxamide (stereoisomer mixture);
(±)-7,8-dimethoxy-N,4-dimethyl-1-[4-(2-methyl-3-oxomorpholin-4-yl)phenyl]-4,5-dihydro-3H-2,3-benzodiazepine-3-carboxamide (stereoisomer mixture);
(±)-7,8-dimethoxy-N,4-dimethyl-1-[4-(4-methyl-2-oxopiperazin-1-yl)phenyl]-4,5-dihydro-3H-2,3-benzodiazepine-3-carboxamide;
(4S)-7,8-dimethoxy-N,4-dimethyl-1-[4-(4-methyl-2-oxopiperazin-1-yl)phenyl]-4,5-dihydro-3H-2,3-benzodiazepine-3-carboxamide;
(4R)-7,8-dimethoxy-N,4-dimethyl-1-[4-(4-methyl-2-oxopiperazin-1-yl)phenyl]-4,5-dihydro-3H-2,3-benzodiazepine-3-carboxamide;
(±)-7,8-dimethoxy-N,4-dimethyl-1-[4-(2-oxopiperazin-1-yl)phenyl]-4,5-dihydro-3H-2,3-benzodiazepine-3-carboxamide;
(±)-7,8-dimethoxy-N,4-dimethyl-1-[4-(1-methyl-1H-1,2,3-triazol-4-yl)phenyl]-4,5-dihydro-3H-2,3-benzodiazepine-3-carboxamide;
(4S)-7,8-dimethoxy-N,4-dimethyl-1-[4-(1-methyl-1H-1,2,3-triazol-4-yl)phenyl]-4,5-dihydro-3H-2,3-benzodiazepine-3-carboxamide;
(±)-1-[4-(2,4-dimethylthiazol-5-yl)phenyl]-7,8-dimethoxy-N,4-dimethyl-4,5-dihydro-3H-2,3-benzodiazepine-3-carboxamide;
(4S)-1-[4-(2,4-dimethylthiazol-5-yl)phenyl]-7,8-dimethoxy-N,4-dimethyl-4,5-dihydro-3H-2,3-benzodiazepine-3-carboxamide;
(4R)-1-[4-(2,4-dimethylthiazol-5-yl)phenyl]-7,8-dimethoxy-N,4-dimethyl-4,5-dihydro-3H-2,3-benzodiazepine-3-carboxamide;
(±)-1-[4-(1,2-dimethyl-1H-imidazol-5-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-{4- [2-(trifluoromethyl)pyridin-3-yl]phenyl}-4,5-dihydro-3H-2,3-benzodiazepine-3-carboxamide;
(±)-1-[4-(6-hydroxypyridin-3-yl)phenyl]-7,8-dimethoxy-N,4-dimethyl-4,5-dihydro-3/1-2,3-benzodiazepine-3-carboxamide;
(4S)-1-[4-(6-hydroxypyridin-3-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-{4-[6-(trifluoromethyl)pyridin-3-yl]phenyl}-4,5-dihydro-3H-2,3-benzodiazepine-3-carboxamide;
(±)-7,8-dimethoxy-N,4-dimethyl-1-[4-(1,3,5-trimethyl-1H-pyrazol-4-yl)phenyl]-4,5-dihydro-3H-2,3-benzodiazepine-3-carboxamide;
(4R)-7,8-dimethoxy-N,4-dimethyl-1-[4-(1,3,5-trimethyl-1H-pyrazol-4-yl)phenyl]-4,5-dihydro-3H-2,3-benzodiazepine-3-carboxamide;
(4S)-7,8-dimethoxy-N,4-dimethyl-1-[4-(1,3,5-trimethyl-1H-pyrazol-4-yl)phenyl]-4,5-dihydro-3H-2,3-benzodiazepine-3-carboxamide;
(±)-1-[4-(isoxazol-4-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[3-(1,3,5-trimethyl-1H-pyrazol-4-yl)phenyl]-4,5-dihydro-3H-2,3-benzodiazepine-3-carboxamide;
(±)-7,8-dimethoxy-N,4-dimethyl-1-[3-(1-methyl-1H-pyrazol-4-yl)phenyl]-4,5-dihydro-3H-2,3-benzodiazepine-3-carboxamide;
(±)-7,8-dimethoxy-N,4-dimethyl-1-[3-(1-methyl-1H-pyrazol-5-yl)phenyl]-4,5-dihydro-3H-2,3-benzodiazepine-3-carboxamide;
(±)-1-[4-fluoro-3-(1,3,5-trimethyl-1H-pyrazol-4-yl)phenyl]-7,8-dimethoxy-N,4-dimethyl-4,5-dihydro-3H-2,3-benzodiazepine-3-carboxamide;
(±)-1-[4-fluoro-3-(1-methyl-1H-pyrazol-4-yl)phenyl]-7,8-dimethoxy-N,4-dimethyl-4,5-dihydro-3H-2,3-benzodiazepine-3-carboxamide;
(±)-1-[3-(3,5-dimethylisoxazol-4-yl)-4-fluoroophenyl]-7,8-dimethoxy-N,4-dimethyl-4,5-dihydro-3H-2,3-benzodiazepine-3-carboxamide;
(±)-7,8-dimethoxy-N,4-d methyl-1-(3′-nitrobiphenyl-4-yl)-4,5-dihydro-3H-2,3-benzodiazepine-3-carboxamide;
(±)-1-(biphenyl-4-yl)-7,8-dimethoxy-N,4-dimethyl-4,5-dihydro-3H-2,3-benzodiazepine-3-carboxamide;
(±)-1-(2′,4′-dichlorobiphenyl-4-yl)-7,8-dimethoxy-N,4-dimethyl-4,5-dihydro-3H-2,3-benzodiazepine-3-carboxamide;
(±)-1-(4′-fluorobiphenyl-4-yl)-7,8-dimethoxy-N,4-dimethyl-4,5-dihydro-3H-2,3-benzodiazepine-3-carboxamide;
(±)-1-(4′-chlorobiphenyl-4-yl)-7,8-dimethoxy-N,4-dimethyl-4,5-dihydro-3H-2,3-benzodiazepine-3-carboxamide;
(±)-7,8-dimethoxy-N,4-dimethyl-1-(4′-methylbiphenyl-4-yl)-4,5-dihydro-3H-2,3-benzodiazepine-3-carboxamide;
(±)-7,8-dimethoxy-1-(4′-methoxybiphenyl-4-yl)-N,4-dimethyl-4,5-dihydro-3H-2,3-benzodiazepine-3-carboxamide;
(±)-7,8-dimethoxy-1-[4-(6-methoxypyridin-3-yl)phenyl]-N,4-dimethyl-4,5-dihydro-3H-2,3-benzodiazepine-3-carboxamide;
(±)-7,8-dimethoxy-N,4-dimethyl-1-[4′-(methylsulphinyl)biphenyl-4-yl]-4,5-dihydro-3H-2,3-benzodiazepine-3-carboxamide;
(±)-7,8-dimethoxy-N,4-dimethyl-1-{2′-[(methylsulphonyl)amino]biphenyl-4-yl}-4,5-dihydro-3H-2,3-benzodiazepine-3-carboxamide;
(±)-7,8-dimethoxy-N,4-dimethyl1-[2′-(methylsulphonyl)biphenyl-4-yl]-4,5-dihydro-3H-2,3-benzodiazepine-3-carboxamide;
(±)-7,8-dimethoxy-N,4-dimethyl-1-{4′-[(methylsulphonyl)amino]biphenyl-4-yl}-4,5-dihydro-3H-2,3-benzodiazepine-3-carboxamide;
(±)-7,8-dimethoxy-N,4-dimethyl-1-{3′-[(methylsulphonyl)amino]biphenyl-4-yl}-4,5-dihydro-3H-2,3-benzodiazepine-3-carboxamide;
(±)-7,8-dimethoxy-N,4-dimethyl-1-(2′-methylbiphenyl-4-yl)-4,5-dihydro-3H-2,3-benzodiazepine-3-carboxamide;
(±)-7,8-dimethoxy-N,4-dimethyl-1-[3′-(methylsulphonyl)biphenyl-4-yl]-4,5-dihydro-3H-2,3-benzodiazepine-3-carboxamide;
(±)-7,8-dimethoxy-1-[4-(2-methoxypyrimidin-5-yl)phenyl]-N,4-dimethyl-4,5-dihydro-3H-2,3-benzodiazepine-3-carboxamide;
(±)-1-(3′-cyano-4′-fluorobiphenyl-4-yl)-7,8-dimethoxy-N,4-dimethyl-4,5-dihydro-3H-2,3-benzodiazepine-3-carboxamide;
(±)-7,8-dimethoxy-1-[4-(2-methoxypyridin-3-yl)phenyl]-N,4-dimethyl-4,5-dihydro-3H-2,3-benzodiazepine-3-carboxamide;
(±)-1-(3′-carbamoylbiphenyl-4-yl)-7,8-dimethoxy-N,4-dimethyl-4,5-dihydro-3H-2,3-benzodiazepine-3-carboxamide;
(±)-7,8-dimethoxy-N,4-dimethyl-1-[4′-(pyrrolidin-1-ylcarbonyl)biphenyl-4-yl]-4,5-dihydro-3H-2,3-benzodiazepine-3-carboxamide;
(±)-7,8-dimethoxy-N,4-dimethyl-1-[4′-(morpholin-4-ylcarbonyl)biphenyl-4-yl]-4,5-dihydro-3H-2,3-benzodiazepine-3-carboxamide;
(±)-7,8-dimethoxy-1-[4-(5-methoxypyridin-3-yl)phenyl]-N,4-dimethyl-4,5-dihydro-3H-2,3-benzodiazepine-3-carboxamide;
(±)-7,8-dimethoxy-N,4-dimethyl-1-[4-(5-methylpyridin-3-yl)phenyl]-4,5-dihydro-3H-2,3-benzodiazepine-3-carboxamide;
(±)-7,8-dimethoxy-N,4-dimethyl-1-[4-(4-methylpyridin-3-yl)phenyl]-4,5-dihydro-3H-2,3-benzodiazepine-3-carboxamide;
(±)-1-[4(cyclopropylcarbamoyl)biphenyl-4-yl]-7,8-dimethoxy-N,4-dimethyl-4,5-dihydro-3H-2,3-benzodiazepine-3-carboxamide;
(±)-1-[4-(3-fluoropyridin-4-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[3′-(trifluoromethyl)biphenyl-4-yl]-4,5-dihydro-3H-2,3-benzodiazepine-3-carboxamide;
(±)-7,8-dimethoxy-N,4-dimethyl-1-[4′-(trifluoromethyl)biphenyl-4-yl]-4,5-dihydro-3H-2,3-benzodiazepine-3-carboxamide;
(±)-7,8-dimethoxy-1-(3 ′-methoxybiphenyl-4-yl)-N,4-dimethyl-4,5-dihydro-3H-2,3-benzodiazepine-3-carboxamide;
(±)-1-[4′-(5-chlorothien-2-yl)biphenyl-4-yl]-7,8-dimethoxy-N,4-dimethyl-4,5-dihydro-3H-2,3-benzodiazepine-3-carboxamide;
(±)-1-(3′-fluorobiphenyl-4-yl)-7,8-dimethoxy-N,4-dimethyl-4,5-dihydro-3H-2,3-benzodiazepine-3-carboxamide;
(±)-7,8-dimethoxy-1-(2′-methoxybiphenyl-4-yl)-N,4-40dimethyl-4,5-dihydro-3H-2,3-benzodiazepine-3-carboxamide;
(±)-7,8-dimethoxy-N,4-dimethyl-1-[2′-(trifluoromethyl)biphenyl-4-yl]-dihydro-3H-2,3-benzodiazepine-3-carboxamide;
(±)-1-(2′-chlorobiphenyl-4-yl)-7,8-dimethoxy-N,4-dimethyl-4,5-dihydro-3H-2,3-benzodiazepine-3-carboxamide;
(±)-1-(2′-fluorobiphenyl-4-yl)-7,8-dimethoxy-N,4-dimethyl-4,5-dihydro-3H-2,3-benzodiazepine-3-carboxamide;
(±)-1-[4′-(hydroxymethyl)biphenyl-4-yl]-7,8-dimethoxy-N,4-dimethyl-4,5-dihydro-3H-2,3-benzodiazepine-3-carboxamide;
(±)-7,8-dimethoxy-N,4-dimethyl-1-[4′-(trifluoromethoxy)biphenyl-4-yl]-dihydro-3H-2,3-benzodiazepine-3-carboxamide;
(±)-7,8-dimethoxy-N,4-dimethyl-1-[3′-(pyrrolidin-1-ylcarbonyl)biphenyl-4-yl]-dihydro-3H-2,3-benzodiazepine-3-carboxamide;
(±)-7,8-dimethoxy-N,4-dimethyl-1-[3′-(piperidin-1-ylcarbonyl)biphenyl-4-yl]-4,5-dihydro-3H-2,3-benzodiazepine-3-carboxamide;
(±)-7,8-dimethoxy-N,4-dimethyl-1-[3′-(morpholin-4-ylcarbonyl)biphenyl-4-yl]-dihydro-3H-2,3-benzodiazepine-3-carboxamide;
(±)1-[3′-(cyclopropylcarbamoyl)biphenyl-4-yl]-dimethoxy-N,4-dimethyl-4,5-dihydro-3H-2,3-benzodiazepine-3-carboxamide;
(±)-1-(2′,4′-difluorobiphenyl-4-yl)-7,8-dimethoxy-N,4-dimethyl-4,5-dihydro-3H-2,3-benzodiazepine-3-carboxamide;
(±)-7,8-dimethoxy-N,4-dimethyl-1-[4-(1-methyl-1H-pyrazol-5-yl)phenyl]-4,5-dihydro-3H-2,3-benzodiazepine-3-carboxamide;
(±)-7,8-dimethoxy-N,4-dimethyl-1-(4′-nitrobiphenyl-4-yl)-4,5-dihydro-3H-2,3-benzodiazepine-3-carboxamide;
(±)-7,8-dimethoxy-N,4-dimethyl-1-[4-(pyridin-3-yl)phenyl]-4,5-dihydro-3H-2,3-benzodiazepine-3-carboxamide;
(±)-7,8-dimethoxy-1-[4-(4-methoxypyridin-3-yl)phenyl]-N,4-dimethyl-4,5-dihydro-3H-2,3-benzodiazepine-3-carboxamide;
(±)-1-(3′-cyanobiphenyl-4-yl)-7,8-dimethoxy-N,4-dimethyl-4,5-dihydro-3H-2,3-benzodiazepine-3-carboxamide;
(±)-1-(4′-cyanobiphenyl-4-yl)-7,8-dimethoxy-N,4-dimethyl-4,5-dihydro-3H-2,3-benzodiazepine-3-carboxamide;
(±)-7,8-dimethoxy-N,4-dimethyl-1-[2′-(trifluoromethoxy)biphenyl-4-yl]-4,5-dihydro-3H-2,3-benzodiazepine-3-carboxamide;
(±)-7,8-dimethoxy-N,4-dimethyl-1-[4′-(methylsulphonyl)biphenyl-4-yl]-4,5-dihydro-3H-2,3-benzodiazepine-3-carboxamide;
(±)-1-(2′-cyanobiphenyl-4-yl)-7,8-dimethoxy-N,4-dimethyl-4,5-dihydro-3H-2,3-benzodiazepine-3-carboxamide;
(±)-7,8-dimethoxy-N,4-dimethyl-1-[4′-(morpholin-4-yl)biphenyl-4-yl]-4,5-dihydro-3H-2,3-benzodiazepine-3-carboxamide;
(±)-7,8-dimethoxy-N,4-dimethyl-1-[4-(pyrimidin-5-yl)phenyl]-4,5-dihydro-3H-2,3-benzodiazepine-3-carboxamide;
(±)-1-[2′-hydroxymethyl)biphenyl-4-yl]-7,8-dimethoxy-N,4-dimethyl-4,5-dihydro-3H-2,3-benzodiazepine-3-carboxamide;
(±)-1-(3′-{[2-(dimethylamino)ethyl]carbamoyl}biphenyl-4-yl)-7,8-dimethoxy-N,4-dimethyl-4,5-dihydro-3H-2,3-benzodiazepine-3-carboxamide;
(±)-7,8-dimethoxy-N,4-dimethyl-1-(3′-sulphamoylbiphenyl-4-yl)-4,5-dihydro-3H-2,3-benzodiazepine-3-carboxamide;
(±)-7,8-dimethoxy-N,4-dimethyl-1-[4′-(methylsulphamoyl)biphenyl-4-yl]-4,5-dihydro-3H-2,3-benzodiazepine-3-carboxamide;
(±)-7,8-dimethoxy-N,4-dimethyl-1-[4-(1-methyl-1H-pyrrol-2-yl)phenyl]-4,5-dihydro-3H-2,3-benzodiazepine-3-carboxamide;
(±)-7,8-dimethoxy-N,4-dimethyl-1-[4-(6-methylpyridin-3-yl)phenyl]-4,5-dihydro-3H-2,3-benzodiazepine-3-carboxamide;
(±)-1-[4′-(cyclopropylsulphamoyl)biphenyl-4-yl]-7,8-dimethoxy-N,4-dimethyl-4,5-dihydro-3H-2,3-benzodiazepine-3-carboxamide;
(±)-1-(3′-fluoro-5′-hydroxybiphenyl-4-yl)-7,8-dimethoxy-N,4-dimethyl-4,5-dihydro-3H-2,3-benzodiazepine-3-carboxamide;
(±)-1-(3′-fluoro-5′-methylbiphenyl-4-yl)-7,8-dimethoxy-N,4-dimethyl-4,5-dihydro-3H-2,3-benzodiazepine-3-carboxamide;
(±)-7,8-dimethoxy-N,4-dimethyl-1-[3′-(methylsulphamoyl)biphenyl-4-yl]-4,5-dihydro-3H-2,3-benzodiazepine-3-carboxamide;
(±)-1-[4-(5-fluoropyridin-3-yl)phenyl]-7,8-dimethoxy-N,4-dimethyl-4,5-dihydro-3H-2,3 benzodiazepine-3-carboxamide;
(±)-1-[4-(4-fluoropyridin-3-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[4-(2-methylpyridin-3-yl)phenyl]-dihydro-3H-2,3-benzodiazepine-3-carboxamide;
(±)-7,8-dimethoxy-1-[4-(2-methoxypyridin-4-yl)phenyl]-N,4-dimethyl-4,5-dihydro-3H-2,3-benzodiazepine-3-carboxamide;
(±)-1-[4-(5-cyanopyridin-3-yl)phenyl]-7,8-dimethoxy-N,4-dimethyl-4,5-dihydro-3H-2,3-benzodiazepine-3-carboxamide;
(4R)-7,8-dimethoxy-N,4-dimethyl-1-[4-(moipholin-4-yl)phenyl]-4,5-dihydro-3H-2,3-benzodiazepine-3-carboxamide;
(4S)-7,8-dimethoxy-N,4-dimethyl-1-[4-(molpholin-4-yl)phenyl]-4,5-dihydro-3H-2,3-benzodiazepine-3-carboxamide;
(4S)-7,8-dimethoxy-N,4-dimethyl-1-[4-(4-methylpiperazin-1-yl)phenyl]-4,5-dihydro-3H-2,3-benzodiazepine-3-carboxamide;
(±)-1-[4-(azetidin-1-yl)phenyl]-7,8-dimethoxy-N,4-dimethyl-4,5-dihydro-3H-2,3-benzodiazepine-3-carboxamide;
(4R)-1-[4-(azetidin-1-yl)phenyl]-7,8-dimethoxy-N,4-dimethyl-4,5-dihydro-3H-2,3-benzodiazepine-3-carboxamide;
(4S)-1-[4-(azetidin-1-yl)phenyl]-7,8-dimethoxy-N,4-dimethyl-4,5-dihydro-3H-2,3-benzodiazepine-3-carboxamide;
(±)-1-[4-(3-fluoroazetidin-1-yl)phenyl]-7,8-dimethoxy-N,4-dimethyl-4,5-dihydro-3H-2,3-benzodiazepine-3-carboxamide;
(4R)-1-[4-(3-fluoroazetidin-1-yl)phenyl]-7,8-dimethoxy-N,4-dimethyl-4,5-dihydro-3H-2,3-benzodiazepine-3-carboxamide;
(4S)-1-[4-(3-fluoroazetidin-1-yl)phenyl]-7,8-dimethoxy-N,4-dimethyl-4,5-dihydro-3H-2,3-benzodiazepine-3-carboxamide;
(±)-1-[4-(4-hydroxypiperidin-1-yl)phenyl]-7,8-dimethoxy-N,4-dimethyl-4,5-dihydro-3H-2,3-benzodiazepine-3-carboxamide;
(4S)-1-[4-(4-hydroxypiperidin-1-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[4-(piperazin-1-yl)phenyl]-4,5-dihydro-3H-2,3-benzodiazepine-3-carboxamide;
(4S)-7,8-dimethoxy-N,4-dimethyl-1-[4-(piperazin-1-yl)phenyl]-4,5-dihydro-3H-2,3-benzodiazepine-3-carboxamide;
(±)-7,8-dimethoxy-N,4-dimethyl-1-[4-(4-methylpiperazin-1-yl)phenyl]-4,5-dihydro-3H-2,3-benzodiazepine-3-carboxamide;
(±)-1-[4-(4-acctylpiperazin-1-yl)phenyl]-7,8-dimethoxy-N,4-dimethyl-4,5-dihydro-3H-2,3-benzodiazepine-3-carboxamide;
(4R)-1-[4-(4-acetylpiperazin-1-yl)phenyl]-7,8-dimethoxy-N,4-dimethyl-4,5-dihydro-3H-2,3-benzodiazepine-3-carboxamide;
(4S)-1-[4-(4-acetylpiperazin-1-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-{4-[4-(trifluoroacetyppiperazin-1-yl]phenyl}-4,5-dihydro-3H-2,3-benzodiazepine-3-carboxamide;
(±)-1-{4-[4-(2-hydroxy-2-methylpropanoyl)piperazin-1-yl]phenyl}-7,8-dimethoxy-N,4-dimethyl-4,5-dihydro-3H-2,3-benzodiazepine-3-carboxamide;
(±)-7,8-dimethoxy-N,4-dimethyl-1-{4-[4-(methylsulphonyl)piperazin-1-yl]phenyl}-4,5-dihydro-3H-2,3-benzodiazepine-3-carboxamide;
(4S)-1-[4-(1, 1-dioxidothiomorpholin-4-yl)phenyl]-7,8-dimethoxy-N,4-dimethyl-4,5-dihydro-3H-2,3-b cnzodiazepinc-3-carboxamide;
(±)-7,8-dimethoxy-N,4-dimethyl-1-[4-(3-oxopiperazin-1-yl)phenyl]-4,5-dihydro-3H-2,3-benzodiazepine-3-carboxamide;
(±)-7,8-dimethoxy-N,4-dimethyl-1-[4-(4-methyl-3-oxopiperazin-1-yl)phenyl]-4,5-dihydro-3H-2,3-benzodiazepine-3-carboxamide;
(±)-7,8-dimethoxy-N,4-dimethyl-1-[4-(piperidin-1-yl)phenyl]-4,5-dihydro-3H-2,3-benzodiazepine-3-carboxamide;
(±)-7,8-dimethoxy-N,4-dimethyl-1-[3-(morpholin-4-yl)phenyl]-4,5-dihydro-3H-2,3-benzodiazepine-3-carboxamide;
(±)-1-[3-(3,3-difluoroazetidin-1-yl)phenyl]-7,8-dimethoxy-N,4-dimethyl-4,5-dihydro-3H-2,3-benzodiazepine-3-carboxamide;
(±)-1-[3-(azetidin-1-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[3-(4-methylpiperazin-1-yl)phenyl]-4,5-dihydro-3H-2,3-benzodiazepine-3-carboxamide;
(±)-1-[4-fluoro-3-(morpholin-4-yl)phenyl]-7,8-dimethoxy-N,4-dimethyl-4,5-dihydro-3H-2,3-benzodiazepine-3-carboxamide;
(±)-1-[3-(3,3-difluoroazetidin-1-yl)-4-fluorophenyl]-7,8-dimethoxy-N,4-dimethyl-4,5-dihydro-3H-2,3-benzodiazepine-3-carboxamide;
(±)-1-[4-fluoro-3-(4-hydroxypiperidin-1-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[4-(5-methyl-3-phenyl-1H-pyrazol-1-yl)phenyl]-4,5-dihydro-3H-2,3-benzodiazepine-3-carboxamide;
(4S)-7,8-dimethoxy-N,4-dimethyl-1-[4-(5-methyl-3-phenyl-1H-pyrazol-1-yl)phenyl]-dihydro-3H-2,3-benzodiazepine-3-carboxamide;
(±)-1-[4-(5-cyclopropyl-3-phenyl-1H-pyrazol-1-yl)phenyl]-7,8-dimethoxy-N,4-dimethyl-4,5-dihydro-3H-2,3-benzodiazepine-3-carboxamide;
(4S)-1-[4-(5-cyclopropyl-3-phenyl-1H-pyrazol-1-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-{4-[3-phenyl-5-(trifluoromethyl)-1H-pyrazol-1-yl]phenyl}-4,5-dihydro-3H-2,3-benzodiazepine-3-carboxamide;
(±)-1-{4-[3-(4-fluorophenyl)-1H-pyrazol-1-yl]phenyl}-7,8-dimethoxy-N,4-dimethyl-4,5-dihydro-3H-2,3-benzodiazepine-3-carboxamide;
(4S)-1-{4-[3-(4-fluorophenyl)-1H-pyrazol-1-yl]phenyl}-7,8-dimethoxy-N,4-dimethyl-4,5-dihydro-3H-2,3-benzodiazepine-3-carboxamide;
(4R)-1-{4-[3-(4-fluorophenyl)-1H-pyrazol-1-yl]phenyl}-7,8-dimethoxy-N,4-dimethyl-4,5-dihydro-3H-2,3-benzodiazepine-3-carboxamide;
(±)-7,8-dimethoxy-N,4-dimethyl-1-{4-[5-methyl-3-(trifluoromethyl)-1H-pyrazol-1-₃71] phenyl}-4,5-dihydro-3H-2,3-benzodiazepine-3-carboxamide;
(±)-7,8-dimethoxy-N,4-dimethyl-1-[4-(1H-1,2,4-triazol-1-yl)phenyl]-4,5-dihydro-3H-2,3-benzodiazepine-3-carboxamide;
(±)-7,8-dimethoxy-N,4-dimethyl-1-[4-(5-methyl-1H-1,2,4-triazol-1-yl)phenyl]-4,5-dihydro-3H-2,3-benzodiazepine-3-carboxamide;
(±)-1-[4-(3,5-dimethyl-1H-1,2,4-triazol-1-yl)phenyl]-7,8-dimethoxy-N,4-dimethyl-4,5-dihydro-3 H-2,3-benzodiazepine-3-carboxamide;
(±)-8-tert-butyl-1-[4-(3,5-dimethylisoxazol-4-yl)phenyl]-N,4-dimethyl-4,5-dihydro-3H-2,3-benzodiazepine-3-carboxamide;
(±)-7-chloro-1-[4-(3,5-dimethylisoxazol-4-yl)phenyl]-N,4-dimethyl-8-(trifluoromethoxy)-4,5-dihydro-3H-2,3-benzodiazepine-3-carboxamide;
(4S)-7-chloro-1-[4-(3,5-dimethylisoxazol-4-yl)phenyl]-N,4-dimethyl-8-(trifluoromethoxy)-4,5-dihydro-3H-2,3-benzodiazepine-3-carboxamide;
(4R)-7-chloro-1-[4-(3,5-dimethylisoxazol-4-yl)phenyl]-N,4-dimethyl-8-(tri fluoromethoxy)-4,5-dihydro-3H-2,3-benzodiazepine-3-carboxamide;
(±)-8-chloro-1-[4-(3,5-dimethylisoxazol-4-yl)phenyl]-7-methoxy-N,4-dimethyl-4,5-dihydro-3H-2,3-benzodiazepine-3-carboxamide;
(4S)-8-chloro-1-[4-(3,5-dimethylisoxazol-4-yl)phenyl]-7-methoxy-N,4-dimethyl-4,5-dihydro-3H-2,3-benzodiazepine-3-carboxamide;
(4R)-8-chloro-1-[4-(3,5-dimethylisoxazol-4-yl)phenyl]-7-methoxy-N,4-dimethyl-4,5-dihydro-3H-2,3-benzodiazepine-3-carboxamide;
(±)-1-[4-(3,5-dimethylisoxazol-4-yl)phenyl]-N,4,8- trimethyl-4,5-dihydro-3H-2,3-b enzod iazepine-3-carboxamide;
(±)-7,8-b is(difluoromethoxy)-1-[4-(3,5-dimethylisoxazol-4-yl)phenyl]-N,4-dimethyl-4,5-dihydro-3H-2,3-benzodiazepine-3-carboxamide;
(±)-1-[4-(3,5-dimethylisoxazol-4-yl)phenyl]-7,8-diethoxy-N,4-dimethyl-4,5-dihydro-3H-2,3-benzodiazepine-3-carboxamide;
(±)-7-(difluoromethoxy)-1-[4-(3,5-dimethylisoxazol-4-yl)phenyl]-8-methoxy-N,4-dimethyl-4,5-dihydro-3H-2,3-benzodiazepine-3-carboxamide;
(4S)-7-(difluoromethoxy)-1-[4-(3,5-dimethylisoxazol-4-yl)phenyl]-8-methoxy-N,4-dimethyl-4,5-dihydro-3H-2,3-benzodiazepine-3-carboxamide;
(4R)-7-(difluoromethoxy)-1-[4(3,5-dimethylisoxazol-4-yl)phenyl]-8-methoxy-N,4-dimethyl-4,5-dihydro-3H-2,3-benzodiazepine-3-carboxamide;
(±)-7-(difluoromethoxy)-8-methoxy-N,4-dimethyl-1-[4-(4-methylpiperazin-1-yl)phenyl]-4,5-dihydro-3H-2,3-benzodiazepine-3-carboxamide;
(4R)-7-(difluoromethoxy)-8-methoxy-N,4-dimethyl-1-[4-(4-methylpiperazin-1-yl)phenyl]-4,5-dihydro-3H-2,3-benzodiazepine-3-carboxamide;
(4S)-7-(difluoromethoxy)-8-methoxy-N,4-dimethyl-1-[4-(4-methylpiperazin-1-yl)phenyl]-4,5-dihydro-3H-2,3-benzodiazepine-3-carboxamide;
(±)-1-(4-chlorophenyl)-N,4-dimethyl-8-[3-(4-methylpiperazin-1-yl)propoxy]-4,5-dihydro-3H-2,3-benzodiazepine-3-carboxamide;
(4S)-1-(4-chlorophenyl)-N,4-dimethyl-8-[3-(4-methylpiperazin-1-yl)propoxy]-4,5-dihydro-3H-2,3-benzodiazepine-3-carboxamide;
(±)-1-(4-chlorophenyl)-N,4-dimethyl-8-[3-(morpholin-4-yl)propoxy]-4,5-dihydro-3H-2,3-benzodiazepine-3-carboxamide;
(±)-1-(4-chlorophenyl)-N,4-dimethyl-8-[2-(4-methylpiperazin-1-yl) ethoxy]-4,5-dihydro-3H-2,3-benzodiazepine-3-carboxamide;
(±)-1-(4-chlorophenyl)-N,4-dimethyl-8-[(6-methylpyridin-2-yl)methoxy]-4,5-dihydro-3H-2,3-benzodiazepine-3-carboxamide;
(±)-1-[4-(3,5-dimethylisoxazol-4-yl)phenyl]-8-hydroxy-N,4-dimethyl-4,5-dihydro-3H-2,3-benzodiazepine-3-carboxamide;
(±)-1-[4-(3,5-dimethylisoxazol-4-yl)phenyl]-N,4-dimethyl-8-[3-(molpholin-4-yl)propoxy]-4,5-dihydro-3H-2,3-benzodiazepine-3-carboxamide;
(±)-7-cyano-1-[4-(3,5-dimethylisoxazol-4-yl)phenyl]-8-methoxy-N,4-dimethyl-4,5-dihydro-3H-2,3-benzodiazepine-3-carboxamide;
(±)-8-acetamido-N,4-dimethyl-1-[4-(morpholin-4-yl)phenyl]-4,5-dihydro-3H-2,3-benzodiazepine-3-carboxamide;
(±)-8-acetamido-N,4-dimethyl-1-[4-(4-methylpiperazin-1-yl)phenyl]-4,5-dihydro-3H-2,3-benzodiazepine-3-carboxamide;
(±)-8-acetamido-1-[4-(3,5-dimethylisoxazol-4-yl)phenyl]-N,4-dimethyl-4,5-dihydro-3H-2,3-benzodiazepine-3-carboxamide;
(±)-1-(4-chlorophenyl)-8-(3,5-dimethyl-1H-pyrazol-1-yl)-N,4-dimethyl-4,5-dihydro-3H-2,3-benzodiazepine-3-carboxamide;
(±)-1-[4-(3,5-dimethylisoxazol-4-yl)phenyl]-8-(3,5-dimethyl-1H-pyrazol-1-yl)-N,4-dimethyl-4,5-dihydro-3H-2,3-benzodiazepine-3-carboxamide;
(±)-1-[4-(3,5-dimethylisoxazol-4-yl)phenyl]-N,4-dimethyl-8-(morpholin-4-yl)-4,5-dihydro-3H-2,3-benzodiazepine-3-carboxamide;
(4R)-1-[4-(3,5-dimethylisoxazol-4-yl)phenyl]-N,4-dimethyl-8-(moipholin-4-yl)-4,5-dihydro-3H-2,3-benzodiazepine-3-carboxamide;
(4S)-1-[4-(3,5-dimethylisoxazol-4-yl)phenyl]-N,4-dimethyl-8-(morpholin-4-yl)-4,5-dihydro-3H-2,3-benzodiazepine-3-carboxamide;
(4S)-8-methoxy-N,4-dimethyl-1-[4-(3-oxomorpholin-4-yl)phenyl]-4,5-dihydro-3H-2,3-benzodiazepine-3-carboxamide;
(+)-1-{4-[4-(2-hydroxyethyl)piperazin-1-yl]phenyl}-7,8-dimethoxy-N,4-dimethyl-4,5-dihydro-3H-2,3-benzodiazepine-3-carboxamide;
(±)-7,8-dimethoxy-1-[4-(4-methoxypiperidin-1-yl)phenyl]N,4-dimethyl-4,5-dihydro-3H-2,3-benzodiazepine-3-carboxamide;
(4R)-7,8-dimethoxy-1-[4-(4-methoxypiperidin-1-yl)phenyl]-N,4-dimethyl-4,5-dihydro-31/-2,3-benzodiazepine-3-carboxamide;
(4S)-7,8-dimethoxy-1-[4-(4-methoxypiperidin-1-yl)phenyl]-N,4-dimethyl-4,5-dihydro-3H-2,3-benzodiazepine-3-carboxamide;
(±)-1-{4-[4-(dimethylamino)piperidin-1-yl]phenyl}-7,8-dimethoxy-N,4-dimethyl-4,5-dihydro-3H-2,3-benzodiazepine-3-carboxamide;
(4R)-1-{4-[4-(dimethylamino)piperidin-1-yl]phenyl}-7,8-dimethoxy-N,4-dimethyl-4,5-dihydro-3H-2,3-benzodiazepine-3-carboxamide;
(4S)-1-{4-[4-(dimethylamino)piperidin-1-yl]phenyl}-7,8-dimethoxy-N,4-dimethyl-4,5-dihydro-3H-2,3-benzodiazepine-3-carboxamide;
(±)-1-[4-(3,3-difluoroazetidin-1-yl)phenyl]-7,8-dimethoxy-N,4-dimethyl-4,5-dihydro-3H-2,3-benzodiazepine-3-carboxamide;
(±)-1-[4-(4-acetamidopiperidin-1-yl)phenyl]-7,8-dimethoxy-N,4-dimethyl-4,5-dihydro-3H-2,3-benzodiazepine-3-carboxamide;
(±)-1-{4-[4-(2-hydroxyethyl)piperidin-1-yl]phenyl}-7,8-dimethoxy-N,4-dimethyl-4,5-dihydro-3 H-2,3-benzodiazepine-3-carboxamide;
(±)-1-[4-(3-hydroxyazetidin-1-yl)phenyl]-7,8-dimethoxy-N,4-dimethyl-4,5-dihydro-3H-2,3-benzodiazepine-3-carboxamide;
(±)-1-[4-(3-hydroxy-3-methylazetidin-1-yl)phenyl]-7,8-dimethoxy-N,4-dimethyl-4,5-dihydro-3H-2,3-benzodiazepine-3-carboxamide;
(4R)-1-[4-(3-hydroxy-3-methylazetidin-1-yl)phenyl]-7,8-dimethoxy-N,4-dimethyl-4,5-dihydro-3 H-2,3-benzodiazepine-3-carboxamide;
(4S)-1-[4-(3-hydroxy-3-methylazetidin-1-yl)phenyl]-7,8-dimethoxy-N,4-dimethyl-4,5-dihydro-3 H-2,3-benzodiazepine-3-carboxamide;
(±)-1-[4-(4-Isopropylpiperazin-1-yl)phenyl]-7,8-dimethoxy-N,4-dimethyl-4,5-dihydro-3H-2,3-benzodiazepine-3-carboxamide;
(±)-7,8-dimethoxy-1-[4-(3-methoxyazetidin-1-yl)phenyl]-N,4-dimethyl-4,5-dihydro-3H-2,3-benzodiazepine-3-carboxamide;
(±)-1-[4-(4-hydroxy-4-methylpiperidin-1-yl)phenyl]-7,8-dimethoxy-N,4-dimethyl-4,5-dihydro-3 H-2,3-benzodiazepine-3-carboxamide;
(4R)-1-[4-(4-hydroxy-4-methylpiperidin-1-yl)phenyl]-7,8-dimethoxy-N,4-dimethyl-4,5-dihydro-3 H-2,3-benzodiazepine-3-carboxamide;
(4S)-1-[4-(4-hydroxy-4-methylpiperidin-1-yl)phenyl]-7,8-dimethoxy-N,4-dimethyl-4,5-dihydro-3 H-2,3-benzodiazepine-3-carboxamide;
(±)-1-[4-(4-is opropylpiperazin-1-yl)phenyl]-7,8-dimethoxy-N,4-dimethyl-4,5-dihydro-3H-2,3-benzodiazepine-3-carboxamide;
(±)-7,8-dimethoxy-1-[4-(3-methoxyazetidin-1-yl)phenyl]-N,4-dimethyl-4,5-dihydro-3H-2,3-benzodiazepine-3-carboxamide;
(±)-1-[4-(4-hydroxy-4-methylpiperidin-1-yl)phenyl]-7,8-dimethoxy-N,4-dimethyl-4,5-dihydro-3 H-2,3-benzodiazepine-3-carboxamide;
(4R)-1-[4-(4-hydroxy-4-methylpiperidin-1-yl)phenyl]-7,8-dimethoxy-N,4-dimethyl-4,5-dihydro-3 H-2,3-benzodiazepine-3-carboxamide;
(4S)-1-[4-(4-hydroxy-4-methylpiperidin-1-yl)phenyl]-7,8-dimethoxy-N,4-dimethyl-4,5-dihydro-3 H-2,3-benzodiazepine-3-carboxamide;
(±)-7,8-dimethoxy-N,4-dimethyl-1-{4-[4-(methylcarbamoyl)piperidin-1-yl]phenyl}-4,5-dihydro-3 H-2,3-benzodiazepine-3-carboxamide;
(±)-7,8-dimethoxy-N,4-dimethyl-1-{4-[4-(methylcarbamoyl)piperidin-1-yl]phenyl}-4,5-dihydro-3 H-2,3-benzodiazepine-3-carboxamide;
(4R)-7,8-dimethoxy-N,4-dimethyl-1-{4-[4-(methylcarbamoyl)piperidin-1-yl]phenyl}-4,5-dihydro-3H-2,3-benzodiazepine-3-carboxamide;
(±)-1-{4-[(3S)-3-hydroxypyrrolidin-1-yl]phenyl}-7,8-dimethoxy-N,4-dimethyl-4,5-dihydro-3H-2,3-benzodiazepine-3-carboxamide;
(4S)-1-{4-[(3S)-3-hydroxypyrrolidin-1-yl]phenyl}-7,8-dimethoxy-N,4-dimethyl-4,5-dihydro-3H-2,3-benzodiazepine-3-carboxamide;
(±)-tert-butyl (1-{4-[7,8-dimethoxy-4-methyl-3-(methylcarbamoyl)-4,5-dihydro-3H-2,3-benzodiazepin-1-yl]phenyl}-4-methylpiperidin-4-yl)carbamate;
(±)- -{4-[(2S,5R)-2,5-dimethylpiperazin-1-yl]phenyl}-7,8-dimethoxy-N,4-dimethyl-4,5-dihydro-3 H-2,3-benzodiazepine-3-carboxamide;
(±)-1-{4-[4-(2,2-difluoroethyl)piperazin-1-yl]phenyl}-7,8-thmethoxy-N,4-dimethyl-4,5-dihydro-3H-2,3-benzodiazepine-3-carboxamide;
(4S)-7,8-dimethoxy-N,4-dimethyl-1-[4-(3-oxopiperazin-1-yl)phenyl]-4,5-dihydro-3H-2,3-benzodiazepine-3-carboxamide;
(4S)-1-{4-[(2R,6S)-2,6-dimethylmoipholin-4-yl]phenyl}-7,8-dimethoxy-N,4-dimethyl-4,5-dihydro-3H-2,3-benzodiazepine-3-carboxamide;
(±)-7,8-dimethoxy-N,4-dimethyl-1-[4-(4-oxopiperidin-1-yl)phenyl]-4,5-dihydro-3H-2,3-benzodiazepine-3-carboxamide;
(±)-7,8-dimethoxy-N,4-dimethyl-1-{4-[4-(2,2,2-tritluoroethyl)piperazin- -yl]phenyl}-4,5-dihydro-3H-2,3-benzodiazepine-3-carboxamide;
(4S)-1-{4-[(3 R,5 S)-3,5-dimethylpiperazin-1-yl]phenyl}-7,8-dimethoxy-N,4-dimethyl-4,5-dihydro-3H-2,3-benzodiazepine-3-carboxamide;
(±)-7,8-dihydroxy-N,4-dimethyl-1-[4-(4-methylpiperazin-1-yl)phenyl]-4,5-dihydro-3H-2,3-benzodiazepine-3-carboxamide;
(±)-7,8-diethoxy-N,4-dimethyl-1-[4-(4-methylpiperazin-1-yl)phenyl]-4,5-dihydro-3H-2,3-benzodiazepine-3-carboxamide;
(±)-4-ethyl-7,8-dimethoxy-N-methyl-1-[4-(4-methylpiperazin-1-yl)phenyl]-4,5-dihydro-3H-2,3-benzodiazepine-3-carboxamide;
(4S)-4-ethyl-7,8-dimethoxy-N-methyl-1-[4-(4-methylpiperazin-1-yl)phenyl]-4,5-dihydro-3H-2,3-benzodiazepine-3-carboxamide;
(±)-4-ethyl-7,8-dimethoxy-N-methyl-1-[4-(piperazin-1-yl)phenyl]-4,5-dihydro-3H-2,3-benzodiazepine-3-carboxamide;
(4S)-4-ethyl-7,8-dimethoxy-N-methyl-1-[4-(piperazin-1-yl)phenyl]-4,5-dihydro-3H-2,3-benzodiazepine-3-carboxamide;
(4R)-4-ethyl-7,8-dimethoxy-N-methyl-1-[4-(piperazin-1-yl)phenyl]-4,5-dihydro-3H-2,3-benzodiazepine-3-carboxamide;
(±)-4-ethyl-7,8-dimethoxy-N-methyl-1-{4-[4-(methylsulphonyl)piperazin-1-yl]phenyl}-4,5-dihydro-3H-2,3-benzodiazepine-3-carboxamide;
(4S)-4-ethyl-7,8-dimethoxy-N-methyl-1-{4-[4-(methylsulphonyl)piperazin-1-yl]phenyl}-4,5-dihydro-3H-2,3-benzodiazepine-3-carboxamide;
(4R)-4-ethyl-7,8-dimethoxy-N-methyl-1-{4-[4-(methylsulphonyl)piperazin-1-yl]phenyl}-4,5-dihydro-3H-2,3-benzodiazepine-3-carboxamide;
(±)-4-ethyl-1-[4-(3-fluorazetidin-1-yl)phenyl]-7,8-dimethoxy-N-methyl-4,5-dihydro-3H-2,3-benzodiazepine-3-carboxamide;
(±)-1-[4-(4-acetylpiperazin-1-yl)phenyl]-4-ethyl-7,8-dimethoxy-N-methyl-4,5-dihydro-3H-2,3-benzodiazepine-3-carboxamide;
(±)-1-[4-(1 , 1-dioxidothi omorpholin-4-yl)phenyl]-4-ethyl-7,8-dimethoxy-N-methyl-4,5-dihydro-3 H-2,3-benzodiazepine-3-carboxamide;
(±)-4-ethyl-1-[4-(4-hydroxypiperidin-1-yl)phenyl]-7,8-dimethoxy-N-methyl-4,5-dihydro-3H-2,3-benzodiazepine-3-carboxamide;
(±)-4-ethyl-7,8-dimethoxy-N-methyl-1-[4-(morpholin-4-yl)phenyl]-4,5-dihydro-3H-2,3-benzodiazepine-3-carboxamide;
(4S)-4-ethyl-7,8-dimethoxy-N-methyl-1-[4-(motpholin-4-yl)phenyl]-4,5-dihydro-3H-2,3-benzodiazepine-3-carboxamide;
(±)-8-chloro-1-[4-(1,1-dioxidothiomorpholin-4-yl)phenyl]N,4-dimethyl-7-(tri fluoromethoxy)-4,5-dihydro-3H-2,3-benzodiazepine-3-carboxamide;
(±)-8-chloro-N,4-dimethyl-1-[4-(piperazin-1-yl)phenyl]-7-(tri flu oromethoxy)-4,5-dihydro-3H-2,3-benzodiazepine-3-carboxamide;
(±)-8-chloro-1-[4-(4-hydroxypiperidin-1-yl)phenyl]-N,4-dimethyl-7-(tri fluoromethoxy)-4,5-dihydro-3H-2,3-benzodiazepine-3-carboxamide;
(±)-8-chloro-N,4-dimethyl-1-[4-(4-methylpiperazin-1-yl)phenyl]-7-(trifluoromethoxy)-4,5-dihydro-3H-2,3-benzodiazepine-3-carboxamide;
(±)-8-chloro-N,4-dimethyl-1-{4-[4-(methylsulphonyl)piperazin-1-yl]phenyl}-7-(trifluoromethoxy)-4,5-dihydro-3H-2,3-benzodiazepine-3-carboxamide;
(±)-8-(1,1-dioxidothiomorpholin-4-yl)-1-[4-(1,1-dioxidothiomorpholin-4-yl)phenyl]-N,4-dimethyl-7-(trifluoromethoxy)-4,5-dihydro-3H-2,3-benzodiazepine-3-carboxamide;
(±)-1-(4-chlorophenyl)-8-(1, 1-dioxidothiomorpholin-4-yl)-N,4-dimethyl-7-(tri fluoromethoxy)-4,5-dihydro-3H-2,3-benzodiazepine-3-carboxamide;
(±)-1-[4-(4-acetylpiperazin-1-yl)phenyl]-N,4-dimethyl-7-(trifluoromethoxy)-4,5-dihydro-3H-2,3-benzodiazepine-3-carboxamide;
(±)-1-{4-[4-(2-hydroxyethyl)piperazin-1-yl]phenyl}-N,4-dimethyl-7-(trifluoromethoxy)-4,5-dihydro-3H-2,3-benzodiazepine-3-carboxamide;
(±)-8-methoxy-N,4-dimethyl-1-[4-(4-methylpiperazin-1-yl)phenyl]-7-(trifluoromethoxy)-4,5-dillydro-3H-2,3-benzodiazepine-3-carboxamide;
(±)-4-ethyl-7,8-dimethoxy-N-methyl-1-[4-(1,3,5-trimethyl-1H-pyrazol-4-yl)phenyl]-4,5-dihydro-3H-2,3-benzodiazepine-3-carboxamide;
(±)-4-ethyl-1-(4′-fluorobiphenyl-4-yl)-7,8-dimethoxy-N-methyl-4,5-dihydro-3H-2,3-benzodiazepine-3-carboxamide;
(±)-4-ethyl-7,8-dimethoxy-N-methyl-1-[4-(1-methyl-1H-1,2,3-thazol-4-yl)phenyl]-4,5-dihydro-3 H-2,3-benzodiazepine-3-carboxamide;
(±)-1-[4-(3,5-dimethyl-1,2-oxazol-4-yl)phonyl]-4-ethyl-7,8-dimethoxy-N-methyl-4,5-dihydro-3H-2,3-benzodiazepine-3-carboxamide;
(±)-4-is opropyl-7,8-dimethoxy-N-methyl-1-[4-(1,3,5-trimethyl-1H-pyrazol-4-yl)phenyl]-4,5-dihydro-3H-2,3-benzodiazepine-3-carboxamide;
(±)-4-ethyl-7,8-dimethoxy-N-methyl-1-[4-(2-oxopyrrolidin-1-yl)phenyl]-4,5-dihydro-3H-2,3-benzodiazepine-3-carboxamide;
(±)-4-ethyl-7,8-dimethoxy-N-methyl-1-[4-3-oxomorpholin-4-yl)phenyl]-4,5-dihydro-3H-2,3-benzodiazepine-3-carboxamide;
(±)-4-ethyl-7,8-dimethoxy-N-methyl-1-[4-(2-oxopiperidin-1-yl)phenyl]-4,5-dihydro-3H-2,3-benzodiazepine-3-carboxamide;
(±)-4-ethyl-7,8-dimethoxy-N-methyl-1-[4-(2-oxo-1,3-oxazolidin-3-yl)phenyl]-4,5-dihydro-3H-2,3-benzodiazepine-3-carboxamide;
(4S)-7,8-dimethoxy-N,4-dimethyl-1-[4-(4-methyl-2-oxo-1 ,4-diaz epan-1-yl)phenyl]-4,5-dihydro-3 H-2,3-benzodiazepine-3-carboxamide;
(±)-1-(4-chlorophenyl)-N,4-dimethyl-7-(2-oxo-1,3-oxazolidin-3-yl)-4,5-dihydro-3H-2,3-benzodiazepine-3-carboxamide;
(4S)-1-(4-chlorophenyl)-N,4-dimethyl-7-(2-oxo-1 ,3-oxazolidin-3-yl)-4,5-dihydro-3H-2,3-benzodiazepine-3-carboxamide;
(±)-1-(4-chlorophenyl)-N,4-dimethyl-7-(2-oxopiperidin-1-yl)-4,5-dihydro-3H-2,3-benzodiazepine-3-carboxamide;
(±)-1-(4-chlorophenyl)-N,4-dimethyl-7-(3-oxomorpholin-4-yl)-4,5-dihydro-3H-2,3-benzodiazepine-3-carboxamide;
(±)-1-(4-chlorophenyl)-N,4-dimethyl-7-(morpholin-4-yl)-4,5-dihydro-3H-2,3-benzodiazepine-3-carboxamide;
(±)-1-(4-chlorophenyl)-N,4-dimethyl-7-(pyrrolidin-1-yl)-4,5-dihydro-3H-2,3-benzodiazepine-3-carboxami de;
(±)-1-(4-chlorophenyl)-7-(1, 1-dioxidothiomorpholin-4-yl)-N,4-dimethyl-4,5-dihydro-3H-2,3-benzodiazepine-3-carboxamide;
(±)-1-(4-chlorophenyl)-N,4-dimethyl-7-(4-methylpiperazin-1-yl)-4,5-dihydro-3H-2,3-benzodiazepine-3-carboxamide;
(±)-N,4-dimethyl-7-(4-methylpiperazin-1-yl)-1-[4-(4-methylpiperazin-1-yl)phenyl]-4,5-dihydro-3 H-2,3-benzodiazepine-3-carboxamide;
(±)-N,4-dimethyl-7-(4-methyl-3-oxopiperazin-1-yl)-1-[4-(4-methyl-3-oxopiperazin-1-yl)phenyl]-4,5-dihydro-3H-2,3-benzodiazepine-3-carboxamide;
(±)-1-(4-chlorophenyl)-7-(4-fluorophenyl)-N,4-dimethyl-4,5-dihydro-3H-2,3-benzodiazepine-3-carboxamide;
(±)-1-(4-chlorophenyl)-N,4-dimethyl-7-(pyridin-4-yl)-4,5-dihydro-3H-2,3-benzodiazepinc-3-3 carboxamide;
(±)-1-(4-chlorophenyl)-7-(6-hydroxypyridin-3-yl)-N,4-dimethyl-4,5-dihydro-3H-2,3-benzodiazepine-3-carboxamide;
(±)-1-(4-chlorophenyl)-7-(3,5-dimethyl-1,2-oxazol-4-yl)-N,4-dimethyl-4,5-dihydro-3H-2,3-benzodiazepine-3-carboxamide;
(±)-1-(4-chlorophenyl)-N,4-dimethyl-7-(1-methyl-1H-1,2,3-triazol-4-yl)-4,5-dihydro-3H-2,3-benzodiazepine-3-carboxamide;
(4R)-1-[4-(4-hydroxypiperidin-1-yl)phenyl]-7,8-dimethoxy-N,4-dimethyl-4,5-dihydro-3H-2,3-benzodiazepine-3-carboxamide;
(4R)-7,8-dimethoxy-N,4-dimethyl-1-[4-(4-methylpiperazin-1-yl)phenyl]-4,5-dihydro-3H-2,3-benzodiazepine-3-carboxamide;
(±)-1-[4-(1,1-dioxido-1,2-thiazolidin-2-yl)phenyl]-7,8-dimethoxy-N,4-dimethyl-4,5-dihydro-3H-2,3-benzodiazepine-3-carboxamide;
(±)-1-{7,8-dimethoxy-4-methyl-1-[4-(4-methylpiperazin-1-yl)phenyl]-4,5-dihydro-3H-2,3-benzodiazepin-3-yl}ethanone;
1-{(4S)-7,8-dimethoxy-4-methyl-1-[4-(4-methylpiperazin-1-yl)phenyl]-4,5-dihydro-3H-2,3-benzodiazepin-3-yl}ethanone;
(±)-1-{1-[4-(3,5-dimethyl-1,2-oxazol-4-yl)phenyl]-7,8-dimethoxy-4-methyl-4,5-dihydro-3H-2,3-benzodiazepin-3-yl}ethanone;
(±)-7,8-dimethoxy-N,4-dimethyl-1-[4-(5-methyl-1,3,4-oxadiazol-2-yl)phenyl]-4,5-dihydro-3H-2,3-benzodiazepine-3-carboxamide;
(±)-7,8-dimethoxy-N,4-dimethyl-1-[4-(3-methyl-1,2,4-oxadiazol-5-yl)phenyl]-4,5-dihydro-3H-2,3-benzodiazepine-3-carboxamide;
(±)-N,4-dimethyl-8-(trifluoromethoxy)-1-[4-(1,3,5-trimethyl-1H-pyrazol-4-yl)phenyl]-4,5-dihydro-3H-2,3-benzodiazepine-3-carboxamide;
(4S)-N,4-dimethyl-8-(trifluoromethoxy)-1-[4-(1,3,5-trimethyl-1H-pyrazol-4-yl)phenyl]-4,5-dihydro-3H-2,3-benzodiazepine-3-carboxamide;
(4R)-N,4-dimethyl-8-(trifluoromethoxy)-1-[4-(1,3,5-trimethyl-1H-pyrazol-4-yl)phenyl]-4,5-dihydro-3H-2,3-benzodiazepine-3-carboxamide;
(±)-N,4-dimethyl-1-[4-(4-methyl-1-piperazinyl)phenyl]-8-(trifluoromethoxy)-4,5-dihydro-3H-2,3-benzodiazepine-3-carboxamide;
(4R)-N,4-dimethyl-1-[4-(4-methyl-1-piperazinyl)phenyl]-8-(trifluoromethoxy)-4,5-dihydro-3H-2,3-benzodiazepine-3-carboxamide;
(4S)-N,4-dimethyl-1-[4-(4-methyl-1-piperazinyl)phenyl]-8-(trifluoromethoxy)-4,5-dihydro-3H-2,3-benzodiazepine-3-carboxamide;
(±)-1-[4-(4-hydroxy-1-piperidinyl)phenyl]-N,4-dimethyl-8-(triflu oromethoxy)-4,5-dihydro-3H-2,3-benzodiazepine-3-carboxamide;
(±)-1-[2,4-dibromo-5-(4-methylpiperazin-1-yl)phenyl]-7,8-dimethoxy-N,4-dimethyl-4,5-dihydro-3 H-2,3-benzodiazepine-3-carboxamide;
(4S)-1-[3-bromo-4-(4-methylpiperazin-1-yl)phenyl]-7,8-dimethoxy-N,4-dimethyl-4,5-dihydro-3 H-2,3-benzodiazepine-3-carboxamide;
(4S)-1-[3-cyano-4-(4-methylpiperazin-1-yl)phenyl]-7,8-dimethoxy-N,4-dimethyl-4,5-dihydro-3H-2,3-benzodiazepine-3-carboxamide;
(±)-7,8-dimethoxy-4-methyl-1-[4-(4-methylpiperazin-1-yl)phenyl]-3-(1-oxopropyl)-4,5-dihydro-3H-2,3-benzodiazepine;
(±)-3-(cyclopropylcarbonyl)-7,8-dimethoxy-4-methyl-1-[4-(4-methylpiperazin-1-yl)phenyl]-4,5-dihydro-3H-2,3-benzodiazepine;
(±)-N-cyclopropyl-7,8-dimethoxy-4-methyl-1-[4-(4-methylpiperazin-1-yl)phenyl]-4,5-dihydro-3 H-2,3-benzodiazepine;
(±)-7,8-dimethoxy-N,4-dimethyl-1-[4-(4-methylpiperazin-1-yl)phenyl]-4,5-dihydro-3H-2,3-benzodiazepin-3-carbothioamide;
methyl (±)-7,8-dimethoxy-4-methyl-1-[4-(4-methylpiperazin-1-yl)phenyl]-4,5-dihydro-3H-2,3-benzodiazepine-3-carboxylate;
ethyl (±)-7,8-dimethoxy-4-methyl-1-[4-(4-methylpiperazin-1-yl)phenyl]-4,5-dihydro-3H-2,3-benzodiazepine-3-carboxylate;
(±)-N-ethyl-7,8-dimethoxy-4-methyl-1-[4-(4-methylpiperazin-1-yl)phenyl]-4,5-dihydro-3H-2,3-benzodiazepine-3-carboxamide
and
(4S)-N-ethyl-7,8-dimethoxy-4-methyl-1-[4-(4-methylpiperazin-1-yl)phenyl]-4,5-dihydro-3H-2,3-benzodiazepine-3-carboxamide.

A compound that is tested in the inventive method for stratification by means of melanoma cell line marker PPARGC1A, PPARGC1B or MITF, is the BET inhibitor compound BAY 123, which is one of the examples described in the patent application WO2014/026997, having the following structure

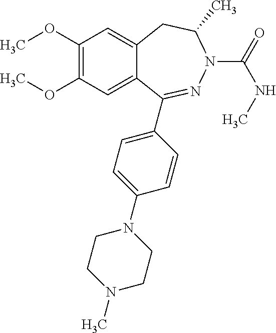 [SMILES: CNC(=O)N1N=C(c2ccc(N3CCN(C)CC3)cc2)c2cc(OC)c(OC)cc2C[C@@H]1C]

((4S)-7,8-dimethoxy-N,4-dimethyl-1-[4-(4-methylpiperazin-1-yl)phenyl]-4,5-dihydro-3H-2,3-benzodiazepine-3-carboxamide)

The invention is, however, not restricted to only those inhibitors according to general formula (I), and thus also inhibitors that are active in melanoma can be stratified in the inventive in vitro method.

For example, a further compound that is tested in the inventive method for stratification by means of melanoma cell line markers PPARGC1A, PPARGC1B or MITF is [(R,S)-4-(4-Chlorophenyl)-2,3,9-trimethyl-6H-1-thia-5,7,8,9a-tetraaza-cyclopenta[e]azulen-6-yl]-acetic acid tert-butyl ester, known as JQ1

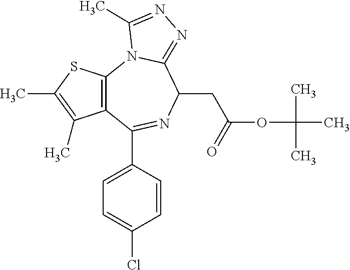 [SMILES: Cc1sc2c(c1C)C(c1ccc(Cl)cc1)=NC(CC(=O)OC(C)(C)C)c1nnc(C)n1-2]

that is disclosed in WO 2011/143669.

Treatment with therapeutics other than compounds of general formula (I), BAY 123 or JQ1, or treatment with therapeutics in addition to compounds of general formula (I), BAY 123 or JQ1 may be beneficial for those patients who would not respond to compounds of general formula (I), BAY 123i or JQ1 or in whom response to compounds of general formula (I), BAY 123 or JQ1 alone is less than desired.

Thus, for those patients it may be beneficial to combine compounds of general formula (I), BAY 123 or JQ1, for example, with one or more compounds selected from

131I-chTNT, abarelix, abiraterone, aclarubicin, ado-trastuzumab emtansine, afatinib, aflibercept, aldesleukin, alemtuzumab, Alendronic acid, alitretinoin, altretamine, amifostine, aminoglutethimide, Hexyl aminolevulinate,amrubicin, amsacrine, anastrozole, ancestim, anethole dithiolethione, angiotensin II, antithrombin III, aprepitant, arcitumomab, arglabin, arsenic trioxide, asparaginase, axitinib, azacitidine, basiliximab, belotecan, bendamustine, belinostat, bevacizumab, bexarotene, bicalutamide, bisantrene, bleomycin, bortezomib, buserelin, bosutinib, brentuximab vedotin, busulfan, cabazitaxel, cabozantinib, calcium folinate, calcium levofolinate, capecitabine, capromab, carboplatin, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribinc, clodronic acid, clofarabine, copanlisib , crisantaspase, cyclophosphamide, cyproterone, cytarabine, dacarbazine, dactinomycin, darbepoetin alfa, dabrafenib, dasatinib, daunorubicin, decitabine, degarelix, denileukin diftitox, denosumab, depreotide, deslorelin, dexrazoxane, dibrospidium chloride, dianhydrogalactitol, diclofenac, docetaxel, dolasetron, doxifluridine, doxorubicin, doxorubicin+estrone, dronabinol, eculizumab, edrecolomab, elliptinium acetate, eltrombopag, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, 1-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, interferon alfa, interferon beta, interferon gamma, iobitridol, iobenguane (123I), iomeprol, ipilimumab, irinotecan, Itraconazole, ixabepilone, lanreotide, lapatinib, Iasocholine, lenalidomide, lenograstim, lentinan, letrozole, leuprorelin, levamisole, levonorgestrel, levothyroxine sodium, lisuride, lobaplatin, lomustine, lonidamine, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, nabilone, nabiximols, nafarelin, naloxone +pentazocine, naltrexone, nartograstim, nedaplatin, nelarabine, neridronic acid, nivolumabpentetreotide, nilotinib, nilutamide, nimorazole, nimotuzumab, nimustine, nitracrine, nivolumab, obinutuzumab, octreotide, ofatumumab, omacetaxine mepesuccinate, omeprazole, ondansetron, oprelvekin, orgotein, orilotimod, oxaliplatin, oxycodone, oxymetholone, ozogamicine, p53 gene therapy, paclitaxel, palifermin, palladium-103 seed, palonosetron, pamidronic acid, panitumumab, pantoprazole, pazopanib, pegaspargase, PEG-epoetin beta (methoxy PEG-epoetin beta), pembrolizumab, pegfilgrastim, peginterferon alfa-2b, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyl)stradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosctron, ramucirumab, ranimustine, rasburicase, razoxane, refametinib , regorafenib, risedronic acid, rhenium-186 etidronate, rituximab, romidepsin, romiplostim, romurtide, roniciclib , samarium (153Sm) lexidronam, sargramostim, satumomab, secretin, sipuleucel-T, sizofiran, sobuzoxane, sodium glycididazole, sorafenib, stanozolol, streptozocin, sunitinib, talaporfin, tamibarotene, tamoxifen, tapentadol, tasonermin, teceleukin, technetium (99mTc) nofetumomab merpentan, 99mTc-HYNIC-[Tyr3]-octreotide, tegafur, tegafur +gimeracil +oteracil, temoporfin, temozolomide, temsirolimus, teniposide, testosterone, tetrofosmin, thalidomide, thiotepa, thymalfasin, thyrotropin alfa, tioguaninc, tocilizumab, topotecan, toremifene, tositumomab, trabectedin, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

The invention also provides the use of a BET inhibitor in the manufacture of a medicament for the treatment of a melanoma patient, wherein the patient has been determined to be a responder by the inventive in vitro method.

The invention further provides the use of a BET inhibitor for the treatment of a melanoma patient, wherein the patient has been determined to be a responder by the inventive in vitro method.

The medicament may comprise a BET inhibitor and a therapeutic composition selected from the group consisting of tyrosine kinase inhibitors, MEK inhibitors, PI3K inhibitors, MAP kinase inhibitors, Alk inhibitors, mTOR inhibitors, P-TEFb inhibitors, apoptosis modulators, hedgehog inhibitors, proteasome inhibitors, HDAC inhibitors, methotrexate, dexamethasone and combinations thereof.

The invention further relates to a kit for an in vitro stratification for determining whether a patient suffering from melanoma will respond to treatment with a BET inhibitor containing the steps
i) determining the expression level of the stratification markers PPARGC1A, PPARGC1B, and/or MITF by measurement of the respective mRNA or derived cDNA expression levels in a sample of body fluid or tumor tissue of said patient, and comparing the expression level with that of normal human melanocytes,
and/or
ii) determining the protein level of the stratification markers PPARGC1A, PPARGC1B and/or MITF in a melanoma patient in a sample of body fluid or tumor tissue of said patient, and comparing it with that of normal human melanocytes,
and/or
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA and/or protein expression level of PPARGC1A, PPARGC1B and/or MITF and/or a lowered OCR following treatment with a BET inhibitor in comparison with the untreated sample is suggestive of a better response to the treatment of melanoma in said patient.

Within the kit, the determination of the expression level of the mRNA or derived cDNA and the determination of the protein level, as well as the determination of the basal OCR can either be done combined, or separately. When using the kit, all combinations are possible to get a valuable result for stratification.

The invention further relates to a kit for an in vitro stratification for determining whether a patient suffering from melanoma will respond to treatment with a BET inhibitor containing the steps
i) determining the expression level of the stratification markers PPARGC1A, PPARGC1B, and/or MITF by measurement of the respective mRNA or derived cDNA expression levels in a sample of body fluid or tumor tissue of said patient, and comparing the expression level with that of normal human melanocytes,
or
ii) determining the protein level of the stratification markers PPARGC1A, PPARGC1B and/or MITF in a melanoma patient in a sample of body fluid or tumor tissue of said patient, and comparing it with that of normal human melanocytes,
or
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA or protein expression level of PPARG C I A, PPARGC1B and/or MITF or a lowered OCR following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
i) determining the expression level of the stratification markers PPARGC1A, PPARGC1B, and/or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B and/or MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA and protein expression level of PPARGC1A, PPARGC1B and/or MITF and a lowered OCR following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
i) determining the expression level of the stratification markers PPARGC1A, PPARGC1B, and/or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B and/or MITF in a melanoma patient in a sample of body fluid or tumor tissue of said patient, and comparing it with that of normal human melanocytes,

wherein the presence in said in vitro sample of an elevated mRNA or derived cDNA and protein expression level of PPARGC1A, PPARGC1B and/or MITF following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
i) determining the expression level of the stratification markers PPARGC1A, PPARGC1B, and/or MITF by measurement of the respective mRNA or derived cDNA expression levels in a sample of body fluid or tumor tissue of said patient, and comparing the expression level with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA expression level of PPARGC1A, PPARGC1B and/or MITF and a lowered OCR following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
ii) determining the protein level of the stratification markers PPARGC1A, PPARGC1B and/or MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated protein expression level of PPARGC1A, PPARGC1B and/or MITF and a lowered OCR following treatment with a BET inhibitor in comparison with the untreated sample is suggestive of a better response to the treatment of melanoma in said patient.

Within the kit, the determination of the mRNA or derived cDNA, or protein expression level can be done with all of the stratification markers PPARGC1A, PPARGC1B and MITF, or can together be done with the stratification markers PPARGC1A and PPARGC1B, or with the stratification markers PPARGC1A and MITF, or can together be done with the stratification markers PPARGC1B and MITF, or can separately be done by measurement of the single stratification marker of PPARGC1A, PPARGC1B or MITF alone. Within the kit, all combinations are possible to get a valuable result for stratification.

The invention further relates to a kit for an in vitro stratification for determining whether a patient suffering from melanoma will respond to treatment with a BET inhibitor containing the steps
i) determining the expression level of the stratification markers PPARGC1A, PPARGC1B or MITF by measurement of the respective mRNA or derived cDNA expression levels in a sample of body fluid or tumor tissue of said patient, and comparing the expression level with that of normal human melanocytes,
or
ii) determining the protein level of the stratification markers PPARGC1A, PPARGC1B or MITF in a melanoma patient in a sample of body fluid or tumor tissue of said patient, and comparing it with that of normal human melanocytes,
or
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA or protein expression level of PPARG C I A, PPARGC1B or MITF or a lowered OCR following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
i) determining the expression level of the stratification markers PPARGC1A, PPARGC1B and MITF by measurement of the respective mRNA or derived cDNA expression levels in a sample of body fluid or tumor tissue of said patient, and comparing the expression level with that of normal human melanocytes,
or
ii) determining the protein level of the stratification markers PPARGC1A, PPARGC1B and MITF in a melanoma patient in a sample of body fluid or tumor tissue of said patient, and comparing it with that of normal human melanocytes,
or
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA or protein expression level of PPARGC1A, PPARGC1B and MITF or a lowered OCR following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
i) determining the expression level of the stratification markers PPARGC1A and PPARGC1B by measurement of the respective mRNA or derived cDNA expression levels in a sample of body fluid or tumor tissue of said patient, and comparing the expression level with that of normal human melanocytes,
or
ii) determining the protein level of the stratification markers PPARGC1A and PPARGC1B in a melanoma patient in a sample of body fluid or tumor tissue of said patient, and comparing it with that of normal human melanocytes,
or
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA or protein expression level of PPARGC1A and PPARGC1B or a lowered OCR following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
i) determining the expression level of the stratification markers PPARGC1A and MITF by measurement of the respective mRNA or derived cDNA expression levels in a sample of body fluid or tumor tissue of said patient, and comparing the expression level with that of normal human melanocytes,
or
ii) determining the protein level of the stratification markers PPARGC1A and MITF in a melanoma patient in a sample of body fluid or tumor tissue of said patient, and comparing it with that of normal human melanocytes,
or
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA or protein expression level of PPARGC1A and MITF or a lowered OCR following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
i) determining the expression level of the stratification markers PPARGC1B and MITF by measurement of the respective mRNA or derived cDNA expression levels in a sample of body fluid or tumor tissue of said patient, and comparing the expression level with that of normal human melanocytes,
or
ii) determining the protein level of the stratification markers PPARGC1B and MITF in a melanoma patient in a sample of body fluid or tumor tissue of said patient, and comparing it with that of normal human melanocytes,
or
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA or protein expression level of PPARGC1B and MITF or a lowered OCR following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
i) determining the expression level of the stratification markers PPARGC1A, PPARGC1B or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B or MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA and protein expression level of PPARGC1A, PPARGC1B or MITF and a lowered OCR following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
i) determining the expression level of the stratification markers PPARGC1A, PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA and protein expression level of PPARGC1A, PPARGC1B and MITF and a lowered OCR following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
i) determining the expression level of the stratification markers PPARGC1A and PPARGC1B by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A and PPARGC1B in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA and protein expression level of PPARGC1A and PPARGC1B and a lowered OCR following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
i) determining the expression level of the stratification markers PPARGC1A and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA and protein expression level of PPARGC1A and MITF and a lowered OCR following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
i) determining the expression level of the stratification markers PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1B and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA and protein expression level of PPARGC1B and MITF and/or a lowered OCR following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
i) determining the expression level of the stratification markers PPARGC1 A, PPARGC1B or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B or MITF in a melanoma patient in a sample of body fluid or tumor tissue of said patient, and comparing it with that of normal human melanocytes,

wherein the presence in said in vitro sample of an elevated mRNA or derived cDNA and protein expression level of PPARGC1A, PPARGC1B or MITF following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
i) determining the expression level of the stratification markers PPARGC1A, PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B and MITF in a melanoma patient in a sample of body fluid or tumor tissue of said patient, and comparing it with that of normal human melanocytes,

wherein the presence in said in vitro sample of an elevated mRNA or derived cDNA and protein expression level of PPARGC1A, PPARGC1B and MITF following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
i) determining the expression level of the stratification markers PPARGC1A and PPARGC1B by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A and PPARGC1B in a melanoma patient in a sample of body fluid or tumor tissue of said patient, and comparing it with that of normal human melanocytes,

wherein the presence in said in vitro sample of an elevated mRNA or derived cDNA and protein expression level of PPARGC1A and PPARGC1B following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
i) determining the expression level of the stratification markers PPARGC1A and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A and MITF in a melanoma patient in a sample of body fluid or tumor tissue of said patient, and comparing it with that of normal human melanocytes,

wherein the presence in said in vitro sample of an elevated mRNA or derived cDNA and protein expression level of PPARGC1A and MITF following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
i) determining the expression level of the stratification markers PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1B and MITF in a melanoma patient in a sample of body fluid or tumor tissue of said patient, and comparing it with that of normal human melanocytes,

wherein the presence in said in vitro sample of an elevated mRNA or derived cDNA and protein expression level of PPARGC1B and MITF following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
i) determining the expression level of the stratification markers PPARGC1A, PPARGC1B or MITF by measurement of the respective mRNA or derived cDNA expression levels in a sample of body fluid or tumor tissue of said patient, and comparing the expression level with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA expression level of PPARGC1A, PPARGC1B or MITF and a lowered OCR following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
i) determining the expression level of the stratification markers PPARGC1A, PPARGC1B and MITF by measurement of the respective mRNA or derived cDNA expression levels in a sample of body fluid or tumor tissue of said patient, and comparing the expression level with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA expression level of PPARGC1A, PPARGC1B and MITF and a lowered OCR following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
i) determining the expression level of the stratification markers PPARGC1A and PPARGC1B by measurement of the respective mRNA or derived cDNA expression levels in a sample of body fluid or tumor tissue of said patient, and comparing the expression level with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA expression level of PPARGC1A and PPARGC1B and a lowered OCR following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
i) determining the expression level of the stratification markers PPARGC1A and MITF by measurement of the respective mRNA or derived cDNA expression levels in a sample of body fluid or tumor tissue of said patient, and comparing the expression level with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA expression level of PPARGC1A and MITF and a lowered OCR following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
i) determining the expression level of the stratification markers PPARGC1B and MITF by measurement of the respective mRNA or derived cDNA expression levels in a sample of body fluid or tumor tissue of said patient, and comparing the expression level with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA expression level of PPARGC1B and MITF and a lowered OCR following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
ii) determining the protein level of the stratification markers PPARGC1A, PPARGC1B or MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated protein expression level of PPARGC1A, PPARGC1B or MITF and a lowered OCR following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
ii) determining the protein level of the stratification markers PPARGC1A, PPARGC1B and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated protein expression level of PPARGC1A, PPARGC1B and MITF and a lowered OCR following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
ii) determining the protein level of the stratification markers PPARGC1A and PPARGC1B in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated protein expression level of PPARGC1A and PPARGC1B and a lowered OCR following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
ii) determining the protein level of the stratification markers PPARGC1A and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated protein expression level of PPARGC1 A and MITF and a lowered OCR following treatment with a BET inhibitor in comparison with the untreated sample is suggestive of a better response to the treatment of melanoma in said patient.

The invention further relates to a kit for an in vitro stratification for determining whether a patient suffering from melanoma will respond to treatment with a BET inhibitor containing the steps
ii) determining the protein level of the stratification markers PPARGC1B and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated protein expression level of PPARGC1B and MITF and a lowered OCR following treatment with a BET inhibitor in comparison with the untreated sample is suggestive of a better response to the treatment of melanoma in said patient.

Within the kit, the respective mRNA or derived cDNA measurements of the PPARGC1A, PPARGC1B and MITF markers can be done separately or combined with the measurements of the protein expression level of the PPARGC1A, PPARGC1B and MITF markers.

For example within the kit the following measurements are possible:
The respective mRNA or derived cDNA levels of the PPARGC1A, PPARGC1B and MITF markers combined with the protein expression level of the PPARGC1A, PPARGC1B or MITF marker.
The respective mRNA or derived cDNA levels of the PPARGC1A, PPARGC1B or MITF marker combined with the protein expression level of the PPARGC1A, PPARGC1B and MITF markers.
The respective mRNA or derived cDNA levels of the PPARGC1A and PPARGC1B markers combined with the protein expression level of the PPARGC1A or PPARGC1B marker.
The respective mRNA or derived cDNA levels of the PPARGC1A or PPARGC1B marker combined with the protein expression level of the PPARGC1A and PPARGC1B markers.
The respective mRNA or derived cDNA levels of the PPARGC1A and MITF markers combined with the protein expression level of the PPARGC1A or MITF marker.
The respective mRNA or derived cDNA levels of the PPARGC1A or MITF marker combined with the protein expression level of the PPARGC1A and MITF markers.
The respective mRNA or derived cDNA levels of the PPARGC1B and MITF markers combined with the protein expression level of the PPARGC1B or MITF marker.
The respective mRNA or derived cDNA levels of the PPARGC1B or MITF marker combined with the protein expression level of the PPARGC1B and MITF markers.

Thus, the invention further relates to a kit for an in vitro stratification for determining whether a patient suffering from melanoma will respond to treatment with a BET inhibitor containing the steps
i) determining the expression level of the stratification markers PPARGC1A, PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B or MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA of PPARGC1A, PPARGC1B and MITF expression level and a protein expression level of PPARGC1A, PPARGC1B or MITF and a lowered OCR following treatment with a BET inhibitor in comparison with the untreated sample is suggestive of a better response to the treatment of melanoma in said patient.

Thus, the invention further relates to a kit for an in vitro stratification for determining whether a patient suffering from melanoma will respond to treatment with a BET inhibitor containing the steps
i) determining the expression level of the stratification markers PPARGC1A, PPARGC1B or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B and MITF in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA expression level of PPARGC1A, PPARGC1B or MITF and protein expression level of PPARGC1A, PPARGC1B and MITF and a lowered OCR following treatment with a BET inhibitor in comparison with the untreated sample is suggestive of a better response to the treatment of melanoma in said patient.

Thus, the invention further relates to a kit for an in vitro stratification for determining whether a patient suffering from melanoma will respond to treatment with a BET inhibitor containing the steps
i) determining the expression level of the stratification markers PPARGC1A, PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B or MITF in a melanoma patient in a sample of body fluid or tumor tissue of said patient, and comparing it with that of normal human melanocytes,

wherein the presence in said in vitro sample of an elevated mRNA or derived cDNA expression level of PPARGC1A, PPARGC1B and MITF and a protein expression level of PPARGC1A, PPARGC1B or MITF following treatment with a BET inhibitor in comparison with the untreated sample is suggestive of a better response to the treatment of melanoma in said patient.

Thus, the invention further relates to a kit for an in vitro stratification for determining whether a patient suffering from melanoma will respond to treatment with a BET inhibitor containing the steps
i) determining the expression level of the stratification markers PPARGC1A, PPARGC1B or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A, PPARGC1B and MITF in a melanoma patient in a sample of body fluid or tumor tissue of said patient, and comparing it with that of normal human melanocytes,

wherein the presence in said in vitro sample of an elevated mRNA or derived cDNA expression level of PPARGC1A, PPARGC1B or MITF and a protein expression level of PPARGC1A, PPARGC1B and MITF following treatment with a BET inhibitor in comparison with the untreated sample is suggestive of a better response to the treatment of melanoma in said patient.

Thus, the invention further relates to a kit for an in vitro stratification for determining whether a patient suffering from melanoma will respond to treatment with a BET inhibitor containing the steps
i) determining the expression level of the stratification markers PPARGC1A and PPARGC1B by measurement of the respective mRNA or derived cDNA expression levels in a sample of body fluid or tumor tissue of said patient, and comparing the expression level with that of normal human melanocytes,
and
determining the protein level of the stratification markers PPARGC1A or PPARGC1B in a melanoma patient in a sample of body fluid or tumor tissue of said patient, and

wherein the presence in said in vitro sample of an elevated mRNA or derived cDNA expression level of PPARGC1A and PPARGC1B and a protein expression level of PPARGC1A or PPARGC1B following treatment with a BET inhibitor in comparison with the untreated sample is suggestive of a better response to the treatment of melanoma in said patient.

Thus, the invention further relates to a kit for an in vitro stratification for determining whether a patient suffering from melanoma will respond to treatment with a BET inhibitor containing the steps
i) determining the expression level of the stratification markers PPARGC1A or PPARGC1B by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A and PPARGC1B in a melanoma patient in a sample of body fluid or tumor tissue of said patient, and comparing it with that of normal human melanocytes,

wherein the presence in said in vitro sample of an elevated mRNA or derived cDNA expression level of PPARGC1A or PPARGC1B and a protein expression level of PPARGC1A and PPARGC1B following treatment with a BET inhibitor in comparison with the untreated sample is suggestive of a better response to the treatment of melanoma in said patient.

Thus, the invention further relates to a kit for an in vitro stratification for determining whether a patient suffering from melanoma will respond to treatment with a BET inhibitor containing the steps
i) determining the expression level of the stratification markers PPARGC1A and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A or MITF in a melanoma patient in a sample of body fluid or tumor tissue of said patient, and comparing

wherein the presence in said in vitro sample of an elevated mRNA or derived cDNA expression level of PPARGC1A and MITF and a protein expression level of PPARGC1A and MITF following treatment with a BET inhibitor in comparison with the untreated sample is suggestive of a better response to the treatment of melanoma in said patient.

Thus, the invention further relates to a kit for an in vitro stratification for determining whether a patient suffering from melanoma will respond to treatment with a BET inhibitor containing the steps
i) determining the expression level of the stratification markers PPARGC1A or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1A and MITF in a melanoma patient in a sample of body fluid or tumor tissue of said patient, and comparing it with that of normal human melanocytes,

wherein the presence in said in vitro sample of an elevated mRNA or derived cDNA expression level of PPARGC1A or MITF and protein expression level of PPARGC1A and MITF following treatment with a BET inhibitor in comparison with the untreated sample is suggestive of a better response to the treatment of melanoma in said patient.

Thus, the invention further relates to a kit for an in vitro stratification for determining whether a patient suffering from melanoma will respond to treatment with a BET inhibitor containing the steps
i) determining the expression level of the stratification markers PPARGC1B and MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1B or MITF in a melanoma patient in a sample of body fluid or tumor tissue of said patient, and comparing it with that of normal human melanocytes,

wherein the presence in said in vitro sample of an elevated mRNA or derived cDNA expression level of PPARGC1B and MITF and a protein expression level of PPARGC1B or MITF following treatment with a BET inhibitor in comparison with the untreated sample is suggestive of a better response to the treatment of melanoma in said patient.

Thus, the invention further relates to a kit for an in vitro stratification for determining whether a patient suffering from melanoma will respond to treatment with a BET inhibitor containing the steps
i) determining the expression level of the stratification markers PPARGC1B or MITF by measurement of the respective mRNA or derived cDNA expression levels in a sample of body fluid or tumor tissue of said patient, and comparing the expression level with that of normal human melanocytes,
and
ii) determining the protein level of the stratification markers PPARGC1B and MITF in a melanoma patient in a sample of body fluid or tumor tissue of said patient, and comparing it with that of normal human melanocytes,

wherein the presence in said in vitro sample of an elevated mRNA or derived cDNA expression level of PPARGC1B or MITF and a protein expression level of PPARGC1B and MITF following treatment with a BET inhibitor in comparison with the untreated sample is suggestive of a better response to the treatment of melanoma in said patient.

Of selected interest is a kit for an in vitro stratification for determining whether a patient suffering from melanoma will respond to treatment with a BET inhibitor containing the steps
i) determining the expression level of the stratification markers PPARGC1A or MITF by measurement of the respective mRNA or derived cDNA expression levels in a sample of body fluid or tumor tissue of said patient, and comparing the expression level with that of normal human melanocytes,
and/or
ii) determining the protein level of the stratification markers PPARGC1A or MITF in a melanoma patient in a sample of body fluid or tumor tissue of said patient, and comparing it with that of normal human melanocytes,
and/or
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA expression level of PPARGC1A or MITF and/or protein expression level of PPARGC1A or MITF and/or a lowered OCRfollowing treatment with a BET inhibitor in comparison with the untreated sample is suggestive of a better response to the treatment of melanoma in said patient.

Of more preferred interest is a kit for an in vitro stratification for determining whether a patient suffering from melanoma will respond to treatment with a BET inhibitor containing the steps
i) determining the expression level of the stratification markers PPARGC1A measurement of the respective mRNA or derived cDNA expression levels in a sample of body fluid or tumor tissue of said patient, and comparing the expression level with that of normal human melanocytes,
or
ii) determining the protein level of the stratification markers PPARGC1A in a melanoma patient in a sample of body fluid or tumor tissue of said patient, and comparing it with that of normal human melanocytes,
and/or
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA expression level of PPARGC1A and/or a lowered OCR following treatment with a BET inhibitor in comparison with the untreated sample is suggestive of a better response to the treatment of melanoma in said patient.

Much more preferred is preferred interest is a kit for an in vitro stratification for determining whether a patient suffering from melanoma will respond to treatment with a BET inhibitor containing the steps
i) determining the expression level of the stratification markers PPARGC1A by measurement of the respective mRNA or derived cDNA expression levels in a sample of body fluid or tumor tissue of said patient, and comparing the expression level with that of normal human melanocytes,
or
ii) determining the protein level of the stratification markers PPARGC1A in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,

wherein the presence in said in vitro sample of an elevated mRNA or derived cDNA expression level of PPARGC1A or a protein expression level of PPARGC1A and a lowered OCR following treatment with a BET inhibitor in comparison with the untreated sample is suggestive of a better response to the treatment of melanoma in said patient.

When using the kit, gene or protein expression profiles indicative of BET responders are preferably those which show at least a 1.5-, 1.7-, or 2-fold difference to melanocytes.

Thus, the present invention also concerns a kit wherein the mRNA, or derived cDNA, or protein expression levels indicative of BET responders show at least a 1.5 fold difference relative to BET non-responders.

Thus, the present invention further concerns a kit wherein the mRNA, or derived cDNA, or protein expression levels indicative of BET responders show at least a 1.7 fold difference relative to BET non-responders.

Further, the present invention concerns a kit wherein the mRNA, or derived cDNA, or protein expression levels indicative of BET responders show at least a 2 fold difference relative to BET non-responders.

The inventive kit can be used for the in vitro stratification of tumors in a patient.

More precisely the kit can be used for the in vitro stratification of melanoma in a patient.

However, the use of the inventive kit is not restricted to melanoma, but can also be used for the in vitro stratification of DLBCL in a patient. That use is also an object of the instant invention.

### BRIEF DESCRIPTION OF THE ABBREVIATIONS

ATCC=American Tissue Culture Collection

BCA=Bicinchon n c acid assay

BET=Bromodomain and extraterminal domain family

DLBCL=Diffuse large B cell lymphoma

DMEM=Dulbecco's modified Eagle's medium

DMSO=Dimethyl sulfoxide

DSMZ=Deutsche Sammlung far Mikroorganismen and Zellkulturen

ECCAC=European Collection of Cell Cultures

FCCP=Carbonyl cyanide-p-trifluoromethoxyphenylhydrazone

FCS=fetal calf serum

HEPES=4-(2-hydroxyethyl)-1-piperazineethanesulfonic acid

hFCS=heat-inactivated and filtered FCS

MEM=Minimum essential medium

OCR=Oxygen consumption rate

PBS=Phosphate-buffered saline

RIPA=Radio-immunoprecipitation assay

RPMI=Roswell Park Memorial Institute

### BRIEF DESCRIPTION OF THE FIGURES

FIG. 1 shows the protein levels of PPARGC1A in melanoma cell lines, as measured by Western blot analysis. PPARGC1A protein is only observed in COLO-792, CHL-1, IPC-298, G-361, MEL-HO and HMCB cell lines.

FIG. 2 shows the relative mRNA expression levels of PPARGC1A, PPARGC1B and MITF in melanoma cell lines. For the calculation, PPARGC1A, PPARGC1B and MITF gene expression levels were first normalized to the housekeeping gene HPRT1 (encoding hypoxanthine phosphoribosyltransferase), whose mRNA expression levels is constant among melanoma cell lines and unrelated to PPARGC1A, PPARGC1B, and MITF. For each gene, the values are reported as the fold-change in expression relative to the respective expression in the RMPI-7951 cell line (whose expression is set as 1.0). Elevated expression of PPARGC1A (more than two-fold higher compared to RPMI-7951) is observed in the COLO-792, CHL-1, G-361, MEL-HO and HMCB cell lines. Elevated expression of PPARGC1B (more than two-fold higher compared to RPMI-7951) is observed in the COLO-792, IPC-298, G-361 and MEL-HO cell lines. Elevated expression of MITF (more than five-fold higher than RPMI-7951) is observed in the COLO-792, CHL-1, IPC-298, G-361, MEL-HO, SK-MEL-28 and SK-MEL-2 cell lines.

FIG. 3 shows the outline of respiration measurements and compound addition with the Seahorse apparatus.

FIG. 4 shows the basal OCR of melanoma cell lines, with and without treatment with BAY 123 or JQ1. Reduction of basal OCR is observed for the COLO-792, CHL-1, G-361 and SK-MEL-2 cell lines.

FIG. 5 shows the 6318 nucleotides long PPARGC1A DNA sequence SEQ ID No. 1 (Gene ID 10891) (NM_013261).

FIG. 6 shows the 10642 nucleotides long PPARGC1B DNA sequence SEQ ID No. 2 (Gene ID 133522) (NM_133263).

FIG. 7 shows the 4815 nucleotides long MITF DNA sequence SEQ ID No. 3 (Gene ID 4286) (Isoform 1, NM_198159).

FIG. 8 shows the 798 amino acids long PPARGC1A protein sequence SEQ ID No. 4(Q9UBK2).

FIG. 9 shows the 984 amino acids long PPARGC1B protein sequence SEQ ID No. 5 (AAI44252).

FIG. 10 shows the 526 amino acids long MITF protein sequence SEQ ID No. 6 (NP-937802).

### BIOLOGICAL EXAMPLES

The following examples describe the feasibility of the present invention. Melanoma cell lines with BRAF wild-type or mutated status were selected. Their response to BET inhibitors was determined following treatment with compound and determination of cell proliferation. Transcript and protein levels of genes involved in mitochondrial function were determined in untreated cell lines to identify stratification biomarkers. Changes in OCR were determined following treatment with a BET inhibitor to identify responsive cell lines.

1. Origin Of Cell Lines

All cell lines are of human origin. The details are given in Table 1.

TABLE 1

| Cell line | Origin | Identifier | Source |
|---|---|---|---|
| COLO-792 | Malignant melanoma, | 93052616 | ECACC |
| | brain metastasis | | |
| CHL-1 | Melanoma | CRL-9446 | ATCC |
| IPC-298 | Cutaneous melanoma | ACC 251 | DSMZ |
| G-361 | Malignant melanoma | CRL-1424 | ATCC |
| MEL-HO | Melanoma | ACC-62 | DSMZ |
| HMCB | Melanoma | CRL-9607 | ATCC |
| A-431 | Epidermoid carcinoma | CRL-1555 | ATCC |
| SK-MEL-28 | Melanoma | HTB-72 | ATCC |
| RPMI-7951 | Malignant melanoma | ACC 66 | DSMZ |
| Hs852.T | Melanoma | CRL-7585 | ATCC |
| SK-MEL-2 | Malignant melanoma | HTB-68 | ATCC |

2. CELL CULTURE

COLO-792, SK-MEL-2, SK-MEL-28, IPC-298 and MEL-HO cells were cultured in RPMI media supplemented with 10% heat-inactivated and filtered FCS (hFCS). CHL-1, Hs852.T, G-361, RPMI-7951 and A-431 cells were cultured in DMEM media supplemented with 10% hFCS. HMCB cells were grown in MEM Earle's media containing 10% hFCS, 1 mM NaPyruvate, non-essential amino acids and 1 mM HEPES. Penicillin (at 50 I.U./mL), streptomycin (at 50 μg/mL), and L-glutamine (at 2 mM) or stable glutamine (supplied with the media) were additional ingredients in all culture media. All cells were grown in an incubator at 37° C. with 5% carbon dioxide.

2. Gi50 Determination

Cells were counted and distributed to 96-well plates, with 1000-5000 cells per well. Each plate also contained wells with media alone for background measurements. Appropriate dilutions of BAY 123 or JQ1 were made in media in a titration series. When adherent cells were fully attached, media was removed and replaced with media containing BAY 123, JQ1, or DMSO as a control. Alternatively, appropriate volumes of BAY 123, JQ1 or DMSO were added to media using a dispenser (HP D300, Tecan).

Cell viability was measured at days 3, 5, and 7, with Alamar Blue (LifeTechnologies) staining and fluorescence detection (excitation 530nm, emission 590nm) in a microplate reader. Alternatively, Cell Titer Glo (Promega) was used, followed by luminescence detection. GI50 values were calculated from triplicate experiments using GraphPad prism software, using curves from plots of fluorescence or luminescence intensity vs. BAY 123 or JQ1 concentration in each cell line. The results show the mean GI50 to vary between 65 nM and >20 000 nM. Cell lines with a GI50 below 600 nM were defined as sensitive to BET inhibition.

The results are shown in Table 2

TABLE 2

| | JQ1 inhibitor | BAY 123 | Mean |
|---|---|---|---|
| Cell line | (GI50, nM) | (GI50, nM) | (GI50, nM) |
| COLO-792 | 50 | 80 | 65 |
| CHL-1 | 60 | 70 | 65 |
| IPC-298 | 60 | 150 | 105 |
| G-361 | 240 | 250 | 245 |
| MEL-HO | 190 | 470 | 330 |
| HMCB | 330 | 570 | 450 |
| A-431 | 720 | 930 | 825 |
| SK-MEL-28 | 1760 | 1260 | 1510 |
| RPMI-7951 | 1460 | 2180 | 1820 |
| Hs 852.T | 7570 | 4440 | 6005 |
| SK-MEL-2 | >20000 | >20000 | >20000 |

3. Western Blot

Adherent cells were grown to 80% confluence. For cell harvest, two methods were used. Cell culture medium was removed, cells were washed once in 37° C. PBS and then scraped from the bottom of the culture dish and transferred into a new tube. Alternatively, adherent cells were detached using trypsinization, followed by trypsin inhibition with cell culture media. Cells were centrifuged for 5 minutes at 150× g, the remaining supernatant was removed, and cell pellets were frozen at −80° C.

Cell pellets were thawed on ice and resuspended in 50-100 μL lysis buffer (RIPA buffer with 1× Roche complete protease inhibitor). The cell lysates were sonicated for 5 minutes (Bioruptor, power M, 30 sec on/30 sec off, 4° C.), then centrifuged for 10 minutes at 4° C., at 13,000 rpm. The supernatant was transferred to a new tube, and protein levels were quantitated using the BCA method. Samples were diluted with lysis buffer to a total protein concentration of 2 mg/mL. 10 μL cell lysate (20 μg total protein) were analyzed using SDS-PAGE (Nu-PAGE 4-12% Bis-Tris protein gels) and Western blotting with the following antibodies: anti-PGC1a (Calbiochem, 4C1.3), anti-MITF (Abcam, ab12039), and anti-β-actin (Sigma), followed by secondary goat-anti-mouse (IRDyl)800CW) or secondary goat-anti-rabbit (IRDyl)680LT) antibodies. Antibody signals were detected and quantitated using a LI-COR instrument.

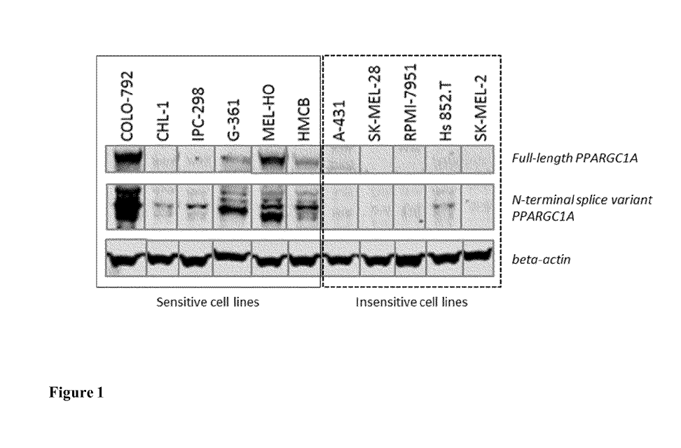

The results are shown in FIG. 1.

They show that cell lines sensitive to BET inhibition express PPARGC1A protein whereas insensitive cell lines do not.

4. mRNA/Gene Expression Analysis

Adherent cells were grown to 80% confluence in an incubator at 37° C. with 5% carbon dioxide. For RNA preparation, cells were grown in a 6-well culture dish, and RNA was isolated from cells in one well per cell line. Total RNA was prepared using the RN easy Plus mini kit (Qiagen), according to the manufacturer's protocol. RNA quantity was determined by UV spectroscopy (Nanodrop).

cDNA was synthesized from RNA using the RT² First Strand kit (SABiosciences/Qiagen), and gene expression was quantitated using RT-PCR (ABI 7900HT 384-well Fast Block). Reagents used were RT² SYBR Green mix with ROX (SABiosciences/Qiagen), and a custom RT² profiler human PCR array (Qiagen; www.qiagen.com/delproducts/cataloglassay-techn ol ogi es/real-tim e-p cr-an d-rt-per-reagents/rt2-profiler-per-arrays/) containing real-time PCR primer assays for the genes PPARGC1A, PPARGC1B, MITF and the HPRT1 housekeeping gene.

Alternatively, cDNA was made using Superscript III (Invitrogen/Life Technologies), followed by qPCR analysis using TaqMan gene expression assays (Applied Biosystems/Life Technologies). The qPCR reactions were set up using TaqMan Fast Advanced Master Mix (Applied Biosystems/Life Technologies) with the following TaqMan probes: MITF (Hs01117294_ml), PPARGC1A (Hs01016719_ml), PPARGC1B (Hs00991677_ml), and HPRT1 (4326321E).

Data were analyzed using the online web tool from SABiosciences/Qiagen, or were analyzed manually, to determine AACt values and fold regulation. All gene expression levels were first normalized to the expression of the HPRT1 gene.

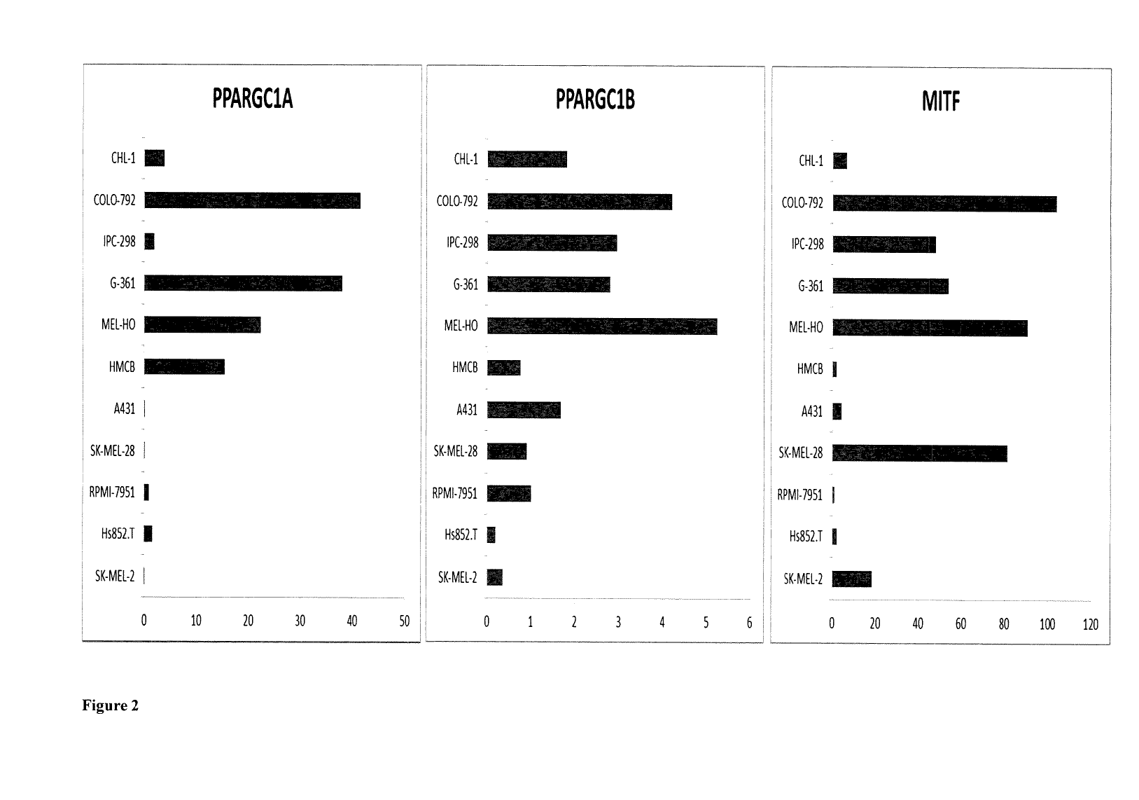

The results are shown in Table 3 and FIG. 2.

TABLE 3
The relative expression levels are shown, determined for PPARGC1A,
PPARGC1B and MITF in each cell line and in comparison to the
RPMI-7951 cell line, which was set to 1, as determined in FIG. 2.

| Cell line | PPARGC1A | PPARGC1B | MITF |
|---|---|---|---|
| COLO-792 | 42 | 4.2 | 104 |
| CHL-1 | 3.9 | 1.8 | 6.6 |
| IPC-298 | 1.9 | 3.0 | 48 |
| G-361 | 38 | 2.8 | 54 |
| MEL-HO | 22 | 5.2 | 90 |
| HMCB | 15 | 0.76 | 1.8 |
| A-431 | 0.003 | 1.7 | 4.2 |
| SK-MEL-28 | 0.11 | 0.91 | 81 |
| RPMI-7951 | 1 | 1 | 1 |
| Hs 852.T | 1.6 | 0.19 | 2.1 |
| SK-MEL-2 | 0.069 | 0.35 | 19 |

5. Measurement of Oxygen Consumption Rates (OCR) using the XF96 Analyzer

5.1 Measurement of OCR using the Mito Stress Kit with Oligomycin, FCCP, Rotenone and Antimycin A as inhibitors

Basal mitochondrial function and mitochondrial stress response were measured by OCR using the XF Mito Stress Test Kit with an XF96 extracellular flux analyzer (Seahorse Bioscience), following the manufacturer's instructions.

The XF Mito Stress Test Kit reveals key parameters of mitochondrial function: basal respiration, ATP production and respiratory capacity. The drug injection ports of the XF96 Assay Cartridge were loaded with assay reagents for a final concentration of 1 μM oligomycin (ATP synthase inhibitor), 0.5 μM FCCP (ionophore and mitochondrial disrupter which disrupts proton gradient and ATP synthesis), 1 μM Rotenone (mitochondrial complex I inhibitor) and 1 μM Antimycin A (mitochondrial complex III inhibitor). Briefly, cells were seeded in quadruplicate at equal densities (20,000-30,000 cells/well) into XF96 tissue culture plates (Seahorse Bioscience). Cell culture medium was changed 12 hours after cell seeding into unbuffered Dulbecco's modified Eagle's medium (DMEM) (8.3 g/l DMEM [Sigma], 2 mM Glutamax [Invitrogen] 5 mM Glucose [Sigma], 1.85 g/l NaCl [Sigma], adjusted to pH 7.4 with NaOH). Real-time measurements of OCR in picomolar per minute in culture medium were conducted. OCR was measured over time at baseline and following consecutive injections of 1 μM Oligomycin, 0.5 μM FCCP and a mix of 1 μM Rotenone+1 μM Antimycin A. The basal mitochondrial OCR was calculated. The OCR values were normalized to cell numbers plated. To this end, the cells were stained using Cyquant (Life technologies) and fluorescence measurements were made using a microplate reader with excitation at 485 nm and emission detection at 530 nm [Tecan].

Basal respiration is predominantly controlled by the parallel re-entry pathways through ATP synthase and uncoupled respiration (proton leak). Addition of Oligomycin blocks ATP synthase and the residual respiration is due to the proton leak. In general, basal respiration shows the energetic demand of the cell under baseline conditions.

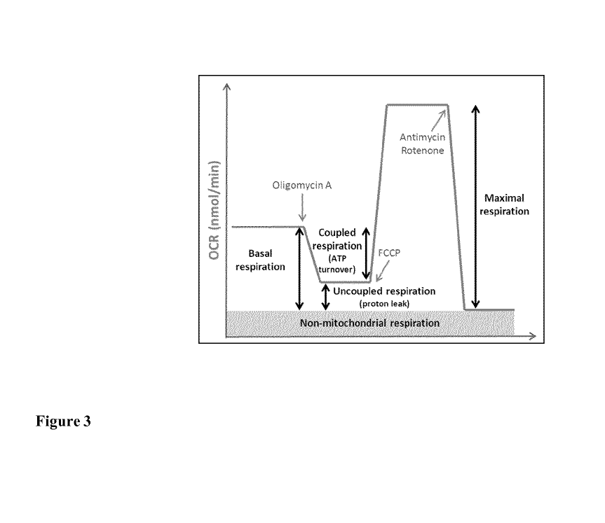

An outline of the experiment is shown in FIG. 3.

5.2 Measurement of OCR using BAY 123 or JQ1 as inhibitors

The OCR in G-361, CHL-1, RPMI-7951 and SK-MEL-2 melanoma cells was measured using the XF96 extracellular flux analyzer (Seahorse Bioscience) under standard conditions and after pre-incubation with 1 μM BAY 123 or JQ1 inhibitor for 20 h. Baseline mitochondrial function and mitochondrial stress response were measured by OCR using the XF Mito Stress Test Kit with an XF96 extracellular flux analyzer (Seahorse Bioscience). The XF Mito Stress Test Kit reveals key parameters of mitochondrial function: basal respiration, ATP production, and respiratory capacity. The drug injection ports of the XF96 Assay Cartridge were loaded with assay reagents at a final concentration of 1 μM oligomycin (ATP synthase inhibitor), 0.5 μM FCCP (ionophore and mitochondrial uncoupler which disrupts proton gradient and ATP synthesis), 1 μM Rotenone (mitochondrial complex I inhibitor) and 1 μM Antimycin A (mitochondrial complex III inhibitor).

Briefly, cells treated with 1 μM BAY 123 or JQ1 inhibitor for 20 h were seeded in quadruplicate at equal densities (20,000-30,000 cells/well) into XF96 tissue culture plates (Seahorse Bioscience). Cell culture medium was changed 12 hours after cell seeding into unbuffered DMEM (8.3 g/l DMEM [Sigma], 2 mM Glutamax [Invitrogen] 5 mM Glucose [Sigma], 1.85 g/l NaCl [Sigma], adjusted to pH 7.4 with NaOH). Real-time measurements of OCR in picomolar per minute in culture medium were conducted. OCR was measured over time at baseline and following consecutive injections of 1 μM Oligomycin, 0.5 μM FCCP and a mix of 1 μM Rotenone+1 μM Antimycin A. The basal mitochondrial OCR was calculated. The OCR values were normalized to cell numbers plated. To this end, the cells were stained using Cyquant (Life technologies) and fluorescence measurements were made using a microplate reader with excitation at 485 nm and emission detection at 530 nm [Tecan].

PPARGC1A, PPARGC1B and MITF positive melanoma cell lines (G-361, CHL-1) had a higher basal OCR compared to those with low PPARGC1A, PPARGC1B and MITF levels (RPM1-7951, SK-MEL-2). After 20 h treatment with BAY 123 or JQ1 [1 μM], basal OCR decreased more dramatically in PPARGC1A, PPARGC1B, MITF positive melanoma compared to negative melanoma cell lines.

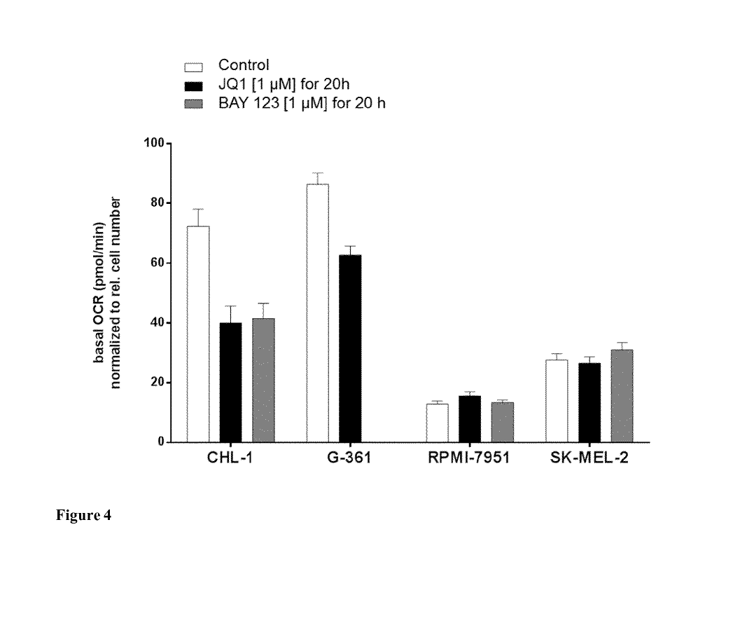

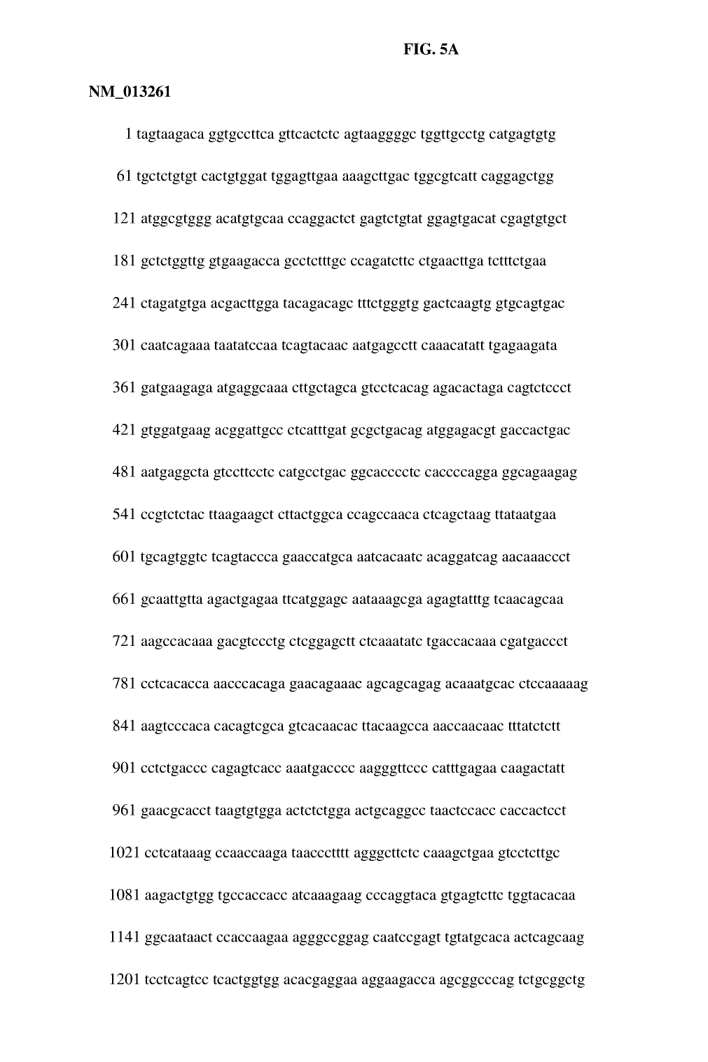

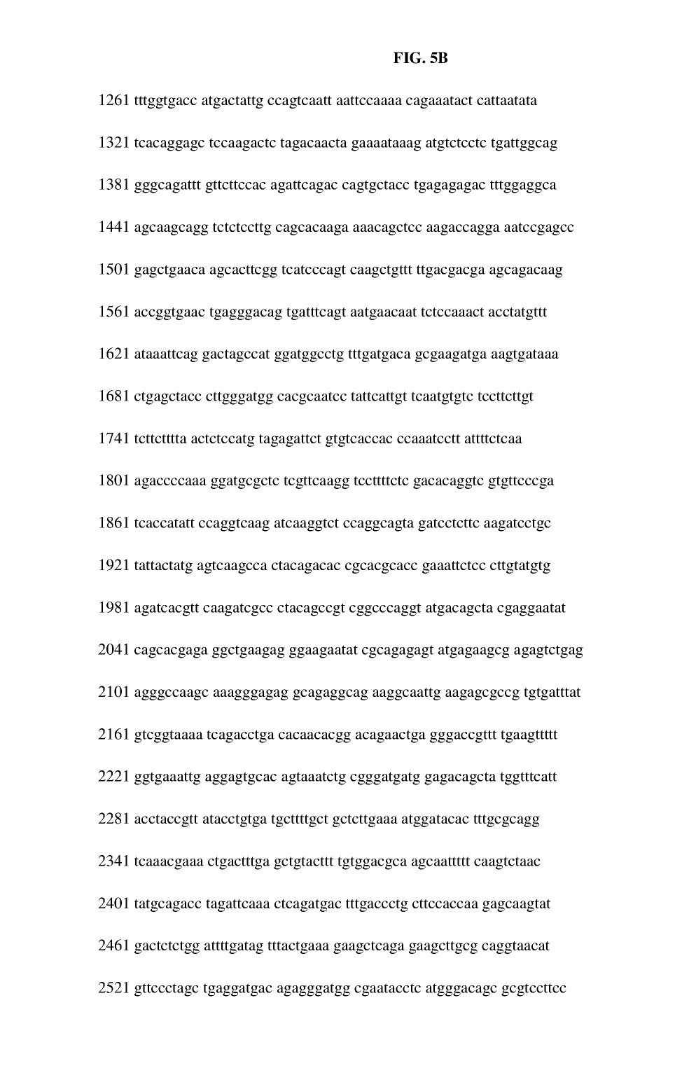

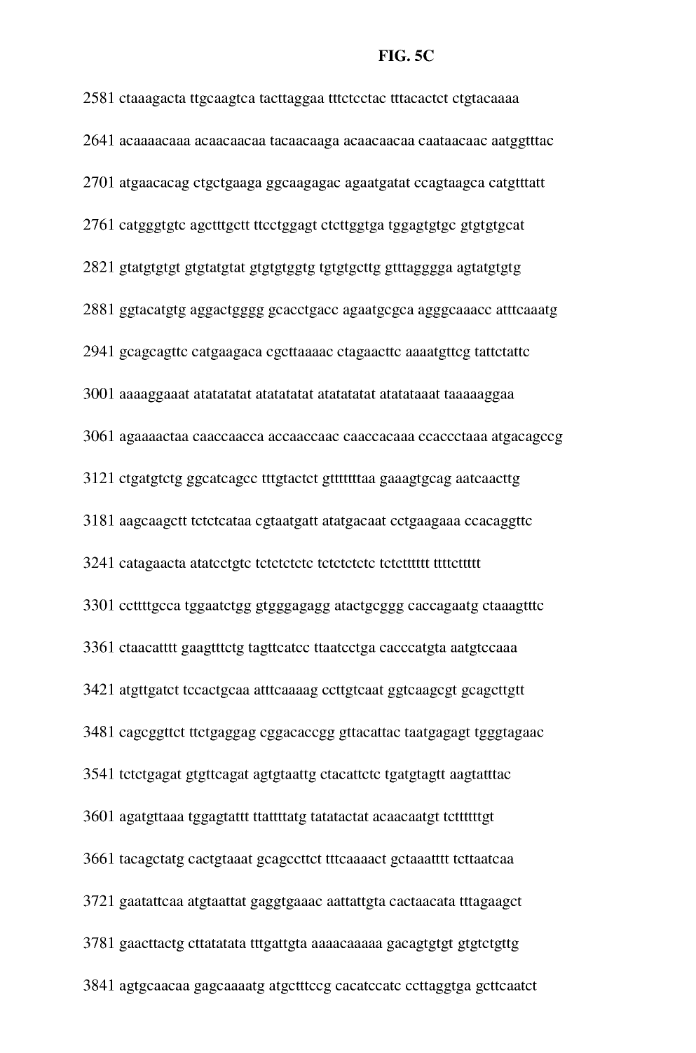

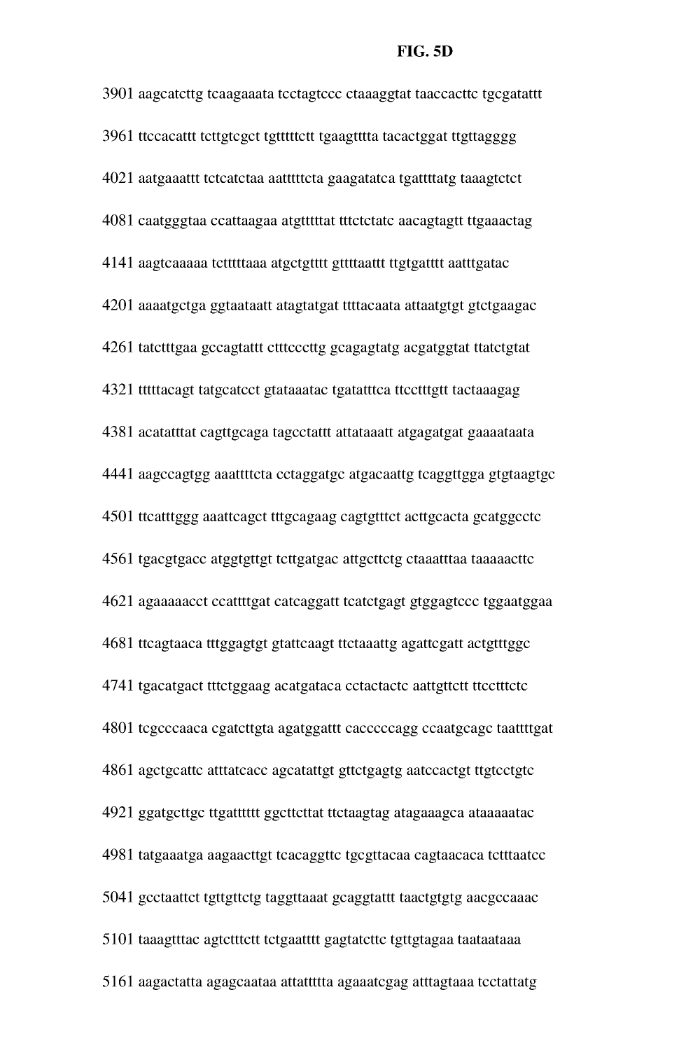

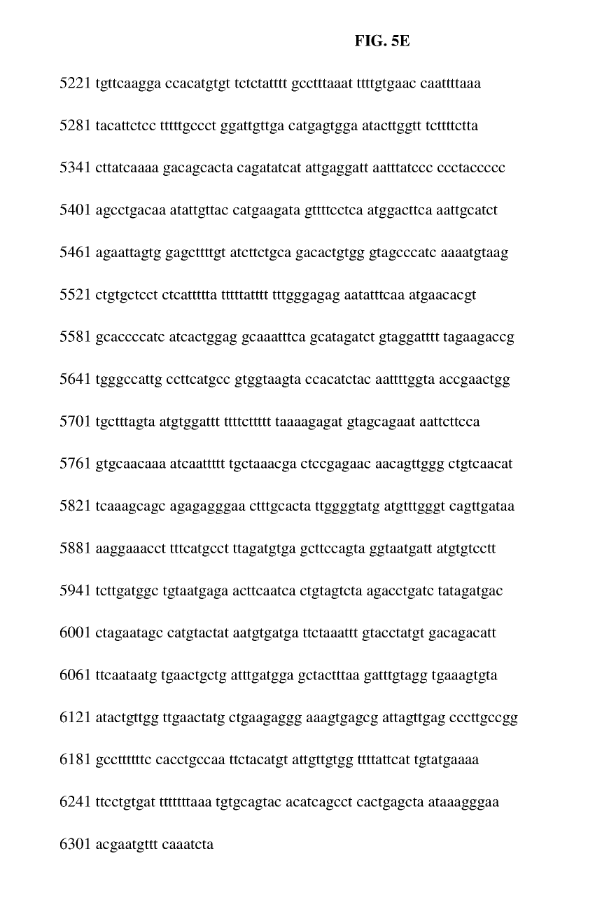

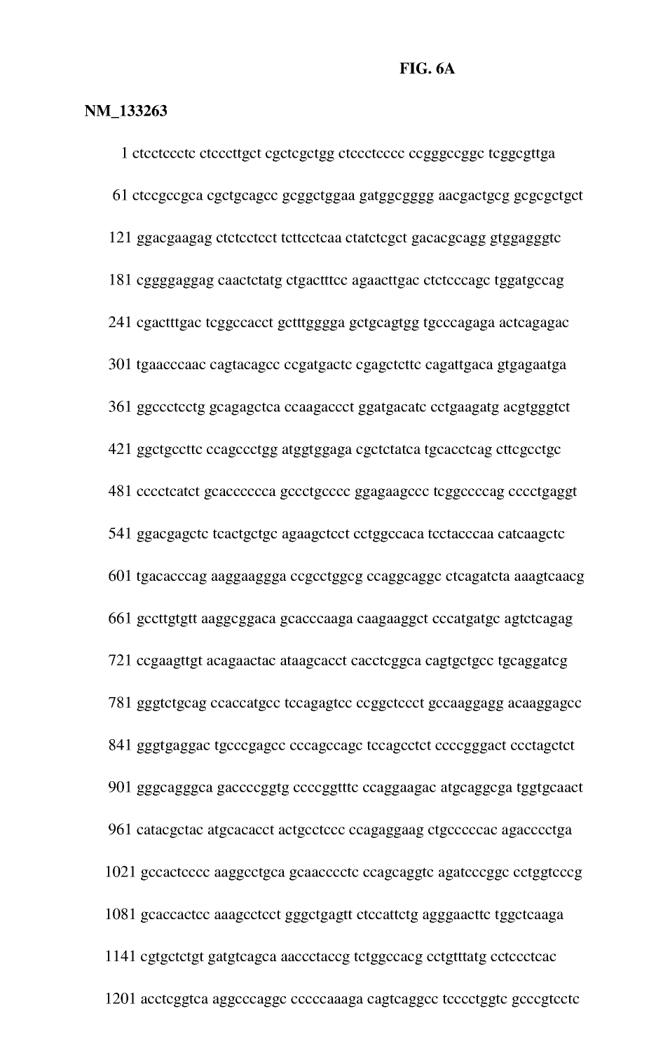

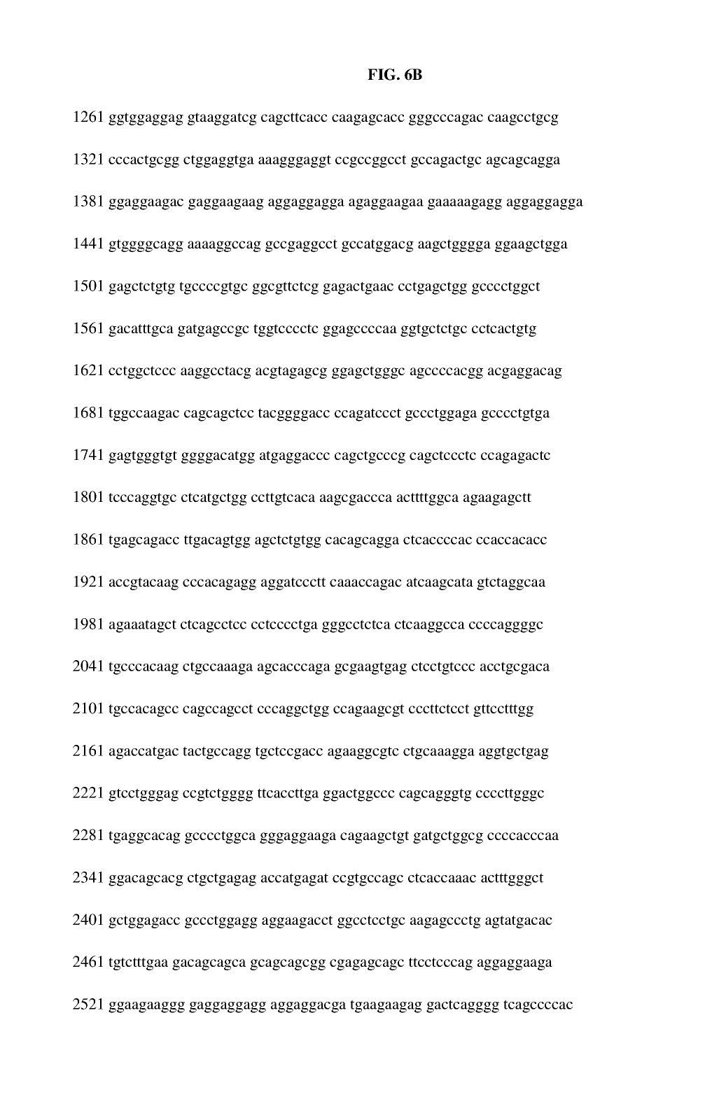

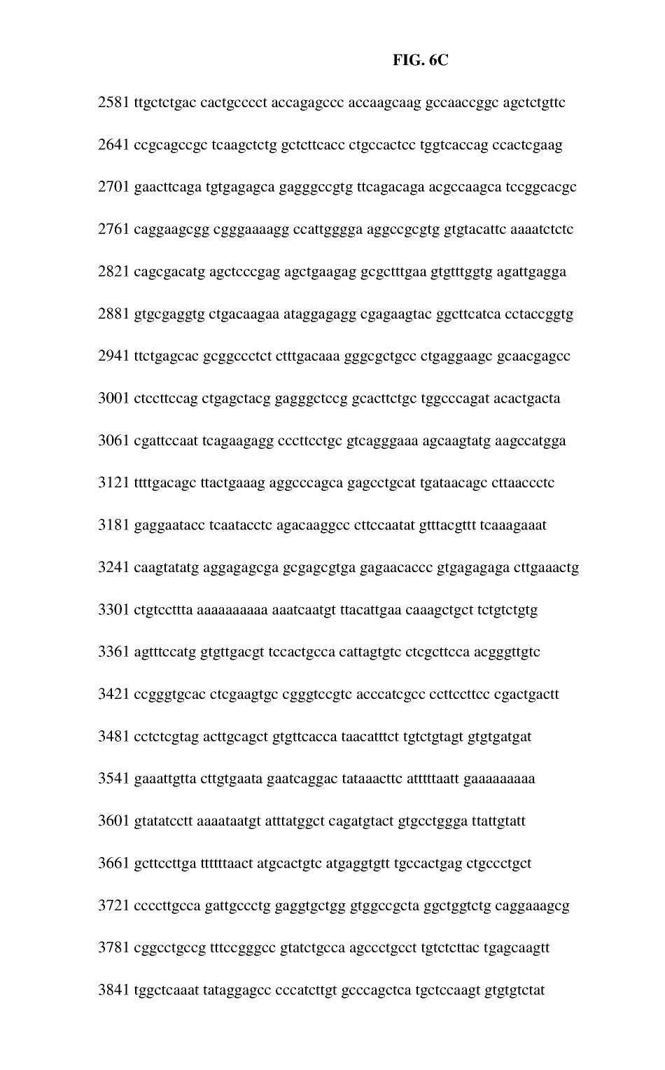

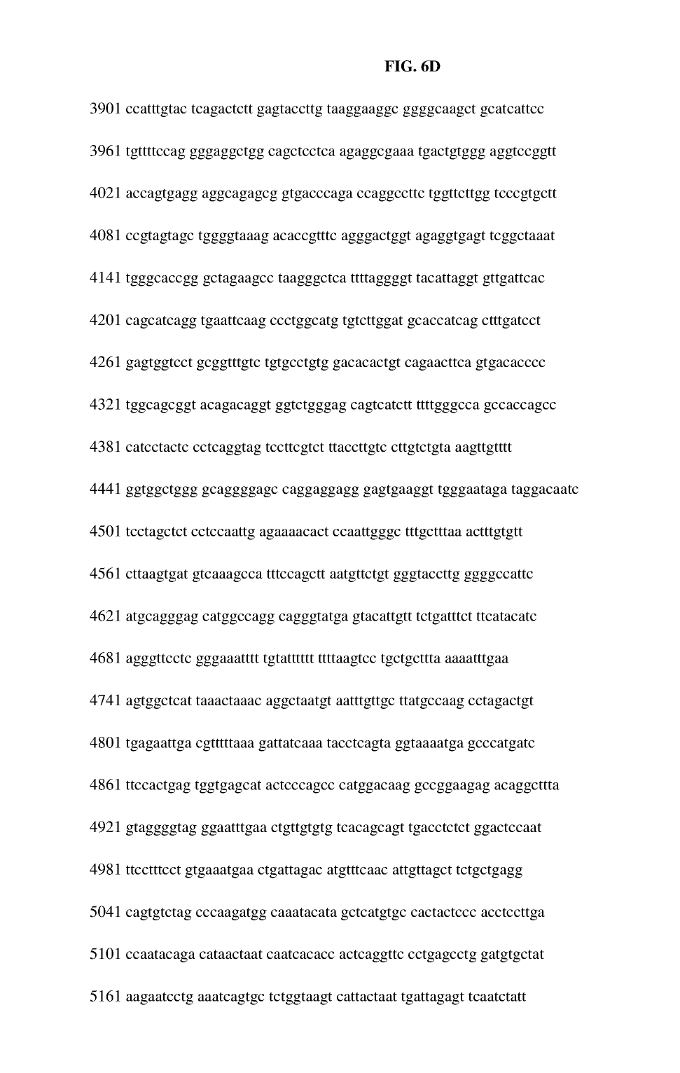

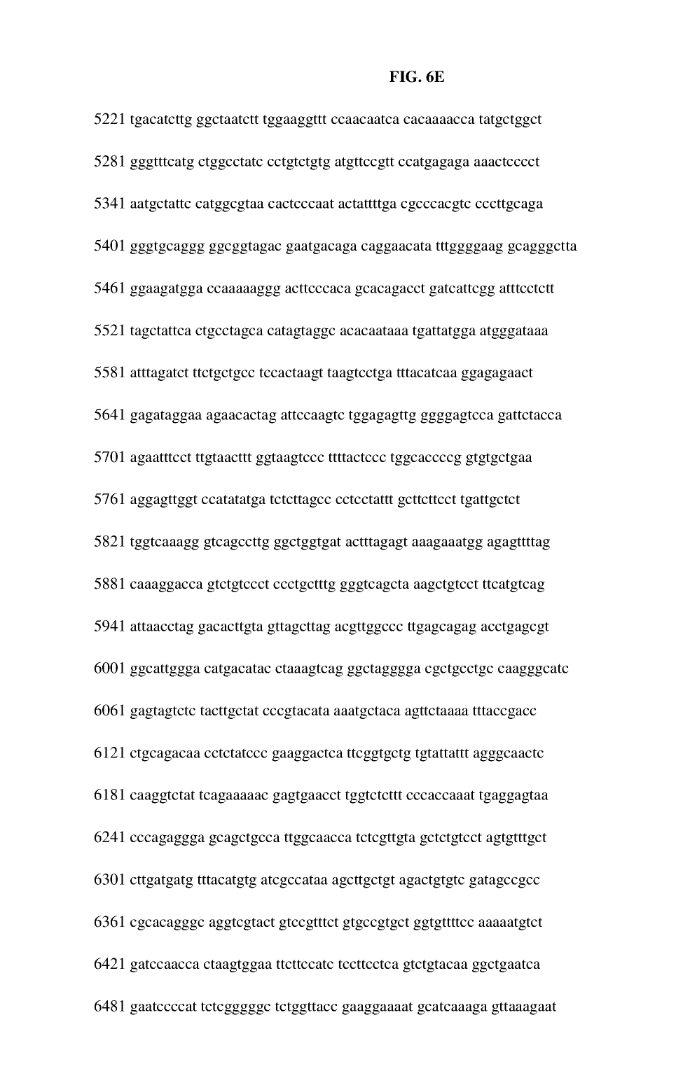

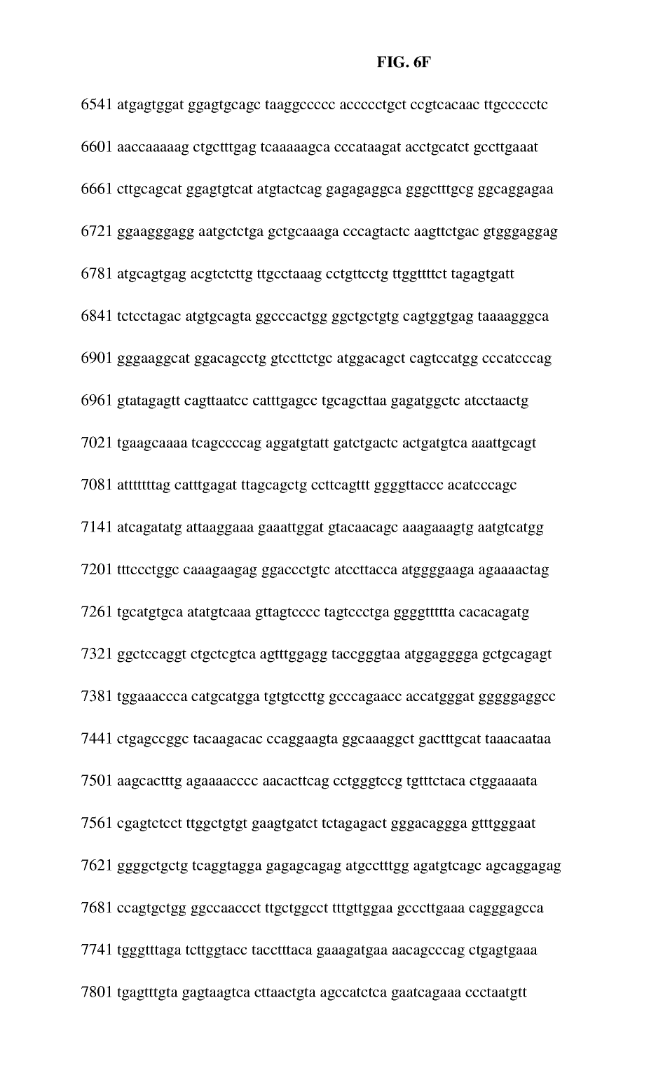

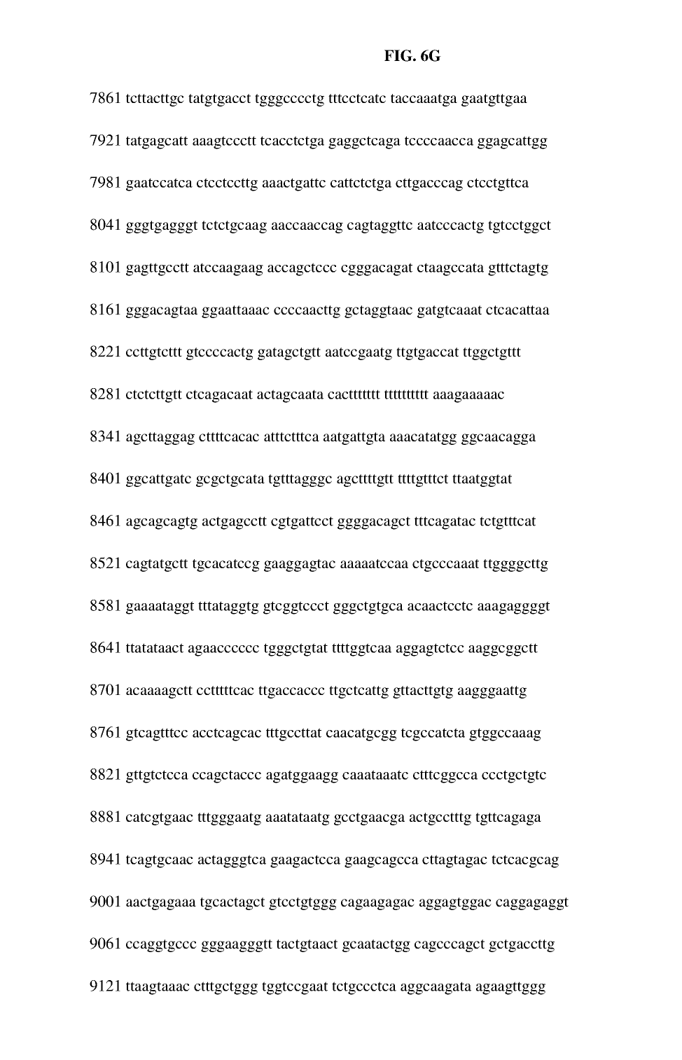

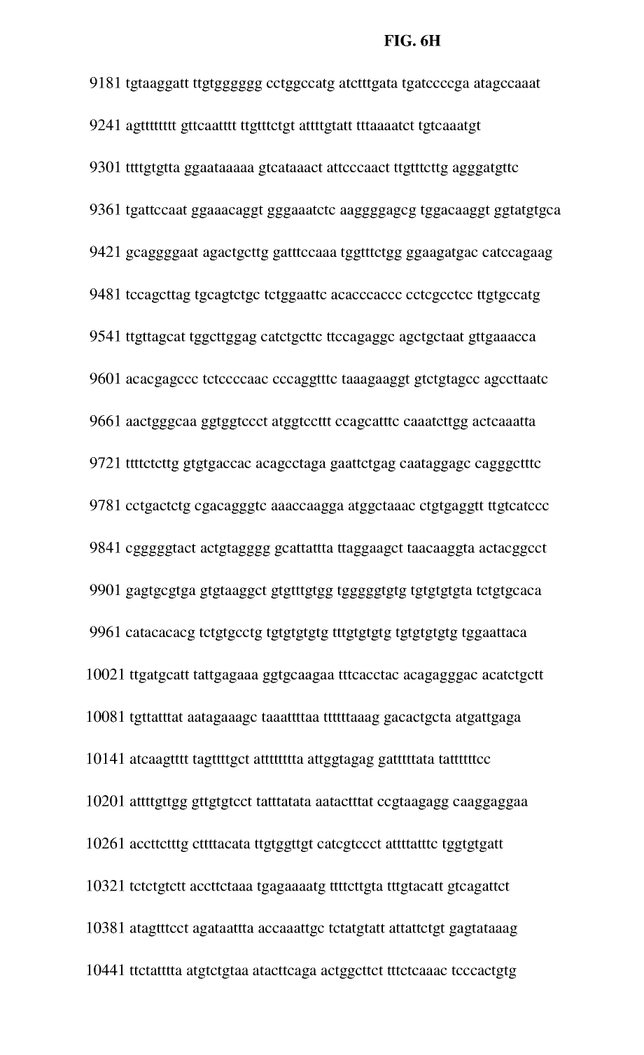

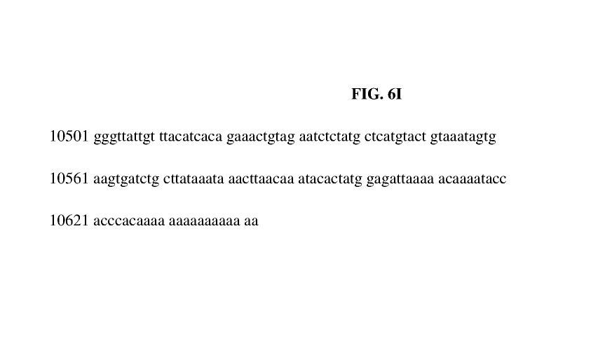

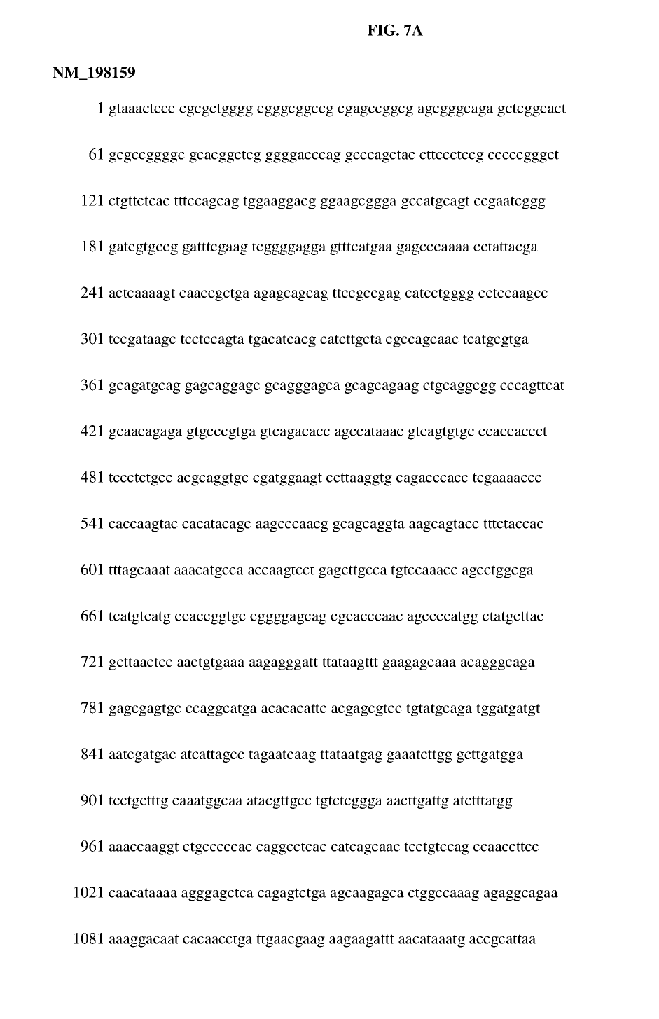

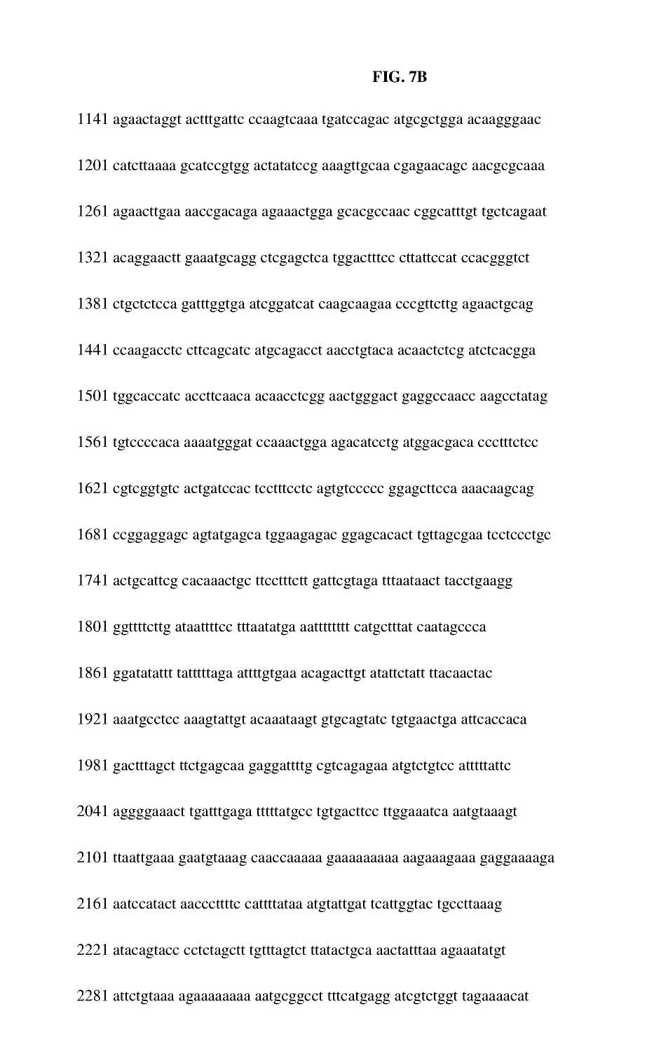

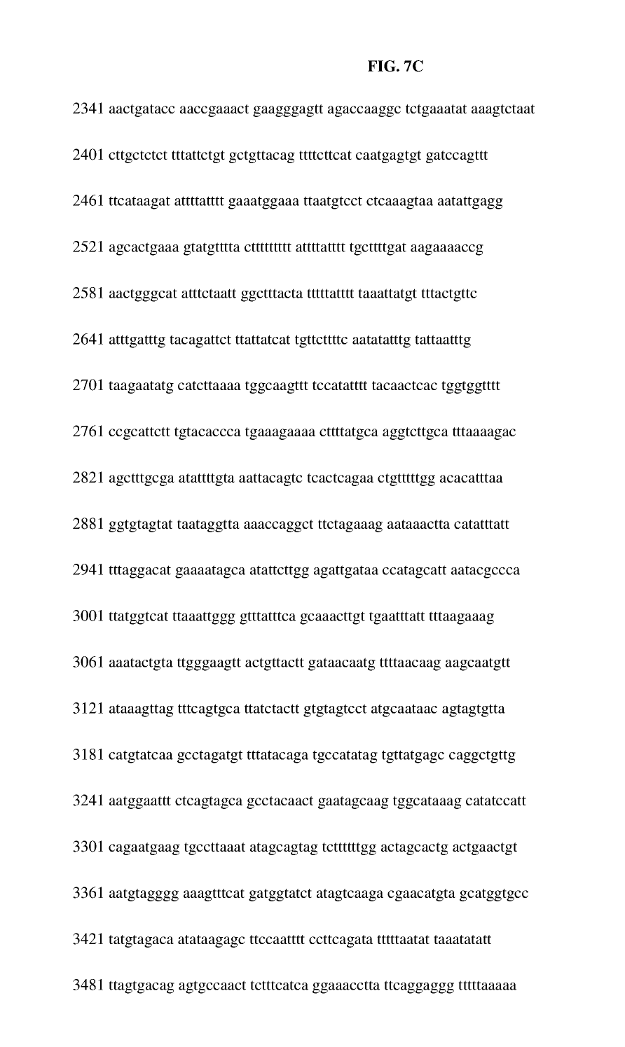

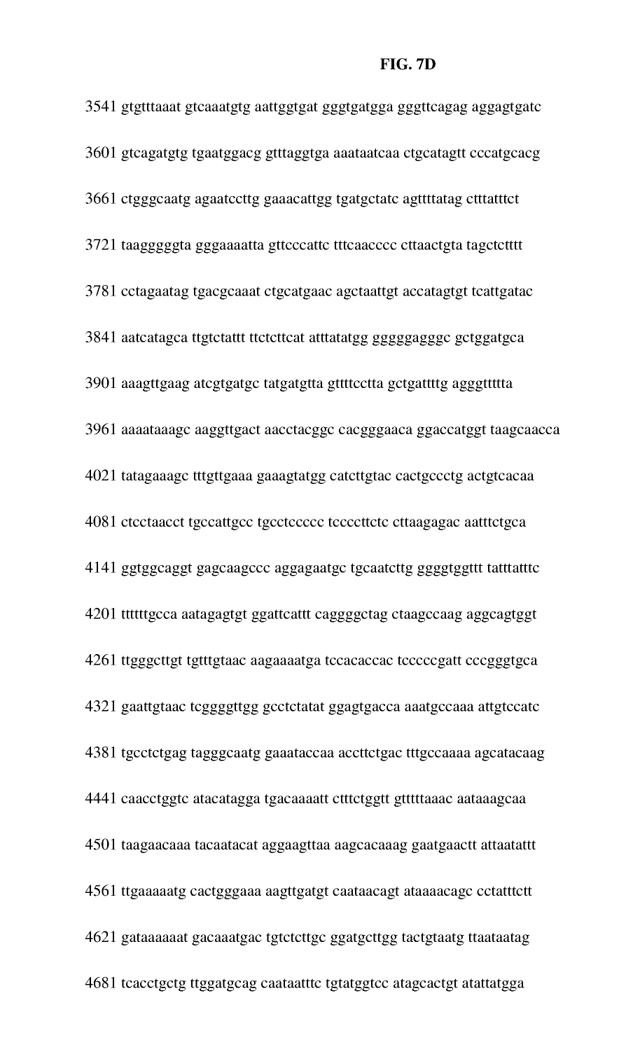

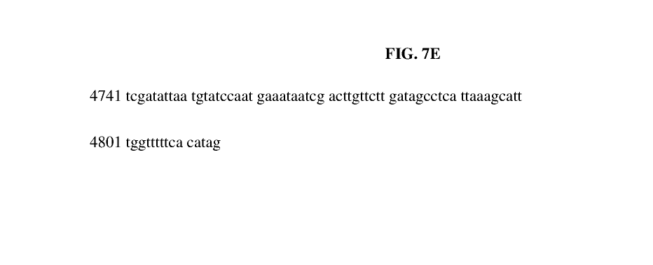

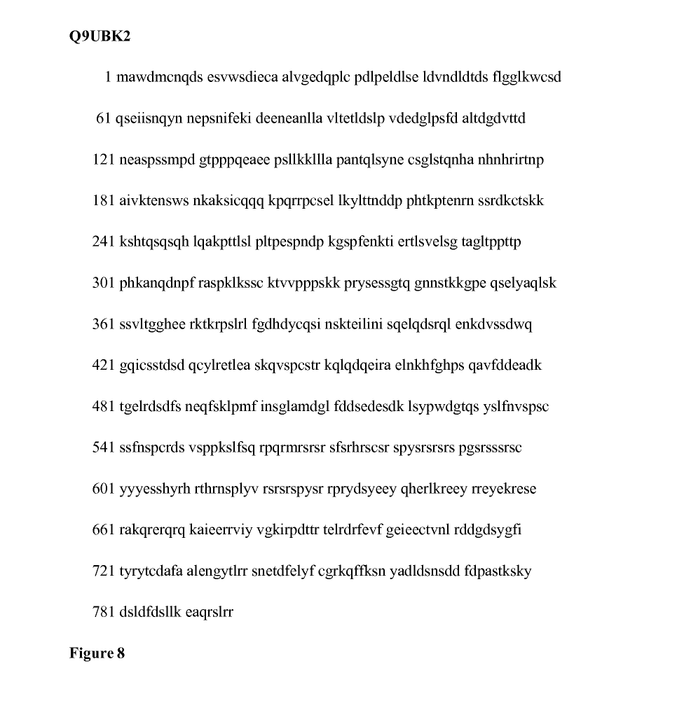

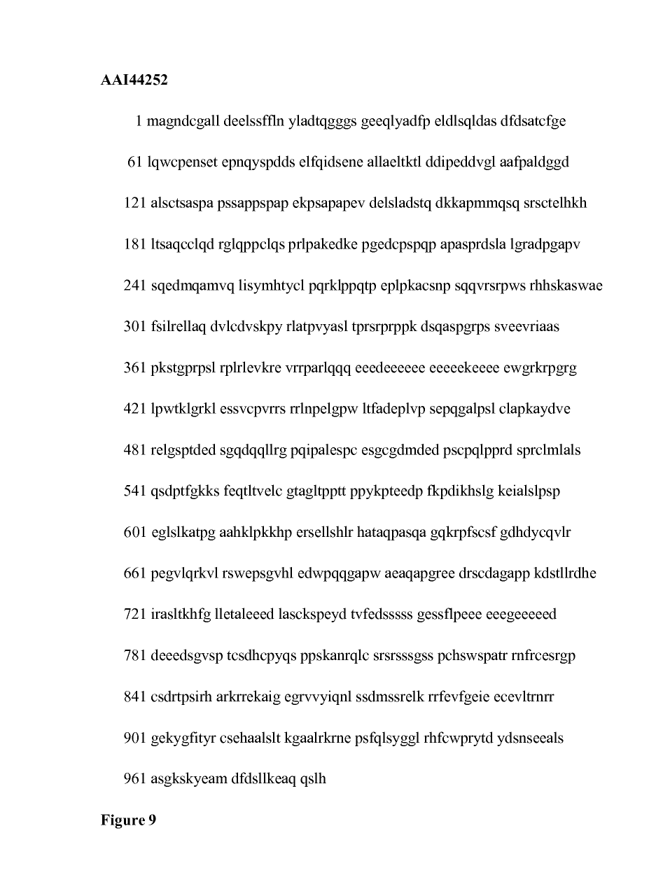

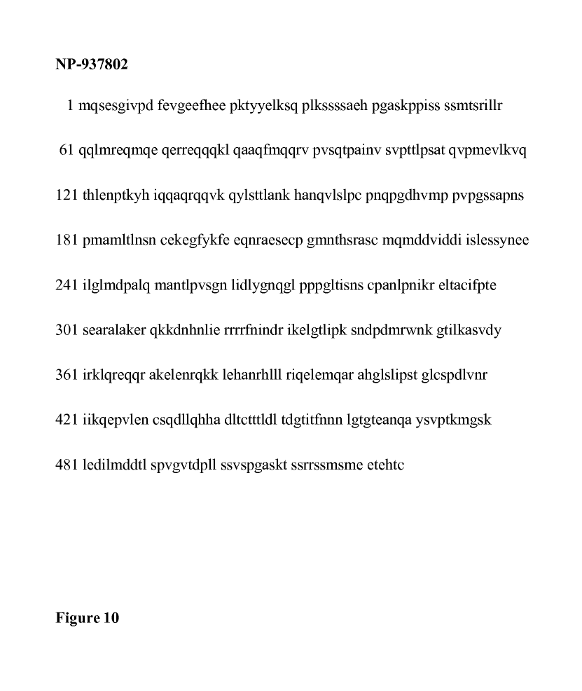

The results are shown in FIG. 4

1. An in vitro stratification method for determining whether a patient suffering from melanoma will respond to treatment with a BET inhibitor by:
i) determining the expression level of the stratification markers PPARGC1A, PPARGC1B, and/or MITF by measurement of the respective mRNA or derived cDNA expression levels in a sample of body fluid or tumor tissue of said patient, and comparing the expression level with that of normal human melanocytes,
and/or
ii) determining the protein level of the stratification markers PPARGC1A, PPARGC1B and/or MITF in a melanoma patient in a sample of body fluid or tumor tissue of said patient, and comparing it with that of normal human melanocytes,
and/or
iii) determining the basal OCR in tumor tissue or circulating tumor cells of a patient before and after treatment with a BET inhibitor, and comparing them with untreated and treated normal human melanocytes,
and wherein the presence in said in vitro sample of an elevated mRNA or derived cDNA and/or protein expression level of PPARGC1A, PPARGC1B and/or MITF and/or a lowered OCR following treatment with a BET inhibitor in comparison with the untreated sample is suggestive of a better response to the treatment of melanoma in said patient.

2. An in vitro stratification method, according to claim 1, for determining whether a patient suffering from melanoma will respond to treatment with a BET inhibitor by:
i) determining the expression level of the stratification markers PPARGC1A or MITF by measurement of the respective mRNA or derived cDNA expression levels in a sample of body fluid or tumor tissue of said patient, and comparing the expression level with that of normal human melanocytes,
and/or
ii) determining the protein level of the stratification markers PPARGC1A or MITF in a melanoma patient in a sample of body fluid or tumor tissue of said patient, and comparing it with that of normal human melanocytes,
and/or
iii) determining the basal OCR in tumor tissue or circulating tumor cells of a patient before and after treatment with a BET inhibitor, and comparing them with untreated and treated normal human melanocytes,
and wherein the presence in said in vitro sample of an elevated mRNA or derived cDNA and/or protein expression level of PPARGC1A or MITF and/or a lowered OCR following treatment with a BET inhibitor in comparison with the untreated sample is suggestive of a better response to the treatment of melanoma in said patient.

3. An in vitro stratification method, according to claim 1, for determining whether a patient suffering from melanoma will respond to treatment with a BET inhibitor by:
i) determining the expression level of the stratification markers PPARGC1A measurement of the respective mRNA or derived cDNA expression levels in a sample of body fluid or tumor tissue of said patient, and comparing the expression level with that of normal human melanocytes,
or
ii) determining the protein level of the stratification markers PPARGC1A in a melanoma patient in a sample of body fluid or tumor tissue of said patient, and comparing it with that of normal human melanocytes,
and/or
iii) determining the basal OCR in tumor tissue or circulating tumor cells of a patient before and after treatment with a BET inhibitor, and comparing them with untreated and treated normal human melanocytes,
and wherein the presence in said in vitro sample of an elevated mRNA or derived cDNA or protein expression level of PPARGC1A and/or a lowered OCR following treatment with a BET inhibitor in comparison with the untreated sample is suggestive of a better response to the treatment of melanoma in said patient.

4. An in vitro stratification method, according to claim 1, for determining whether a patient suffering from melanoma will respond to treatment with a BET inhibitor by:
i) determining the expression level of the stratification markers PPARGC1A by measurement of the respective mRNA or derived cDNA expression levels in a sample of body fluid or tumor tissue of said patient, and comparing the expression level with that of normal human melanocytes,
or
ii) determining the protein level of the stratification markers PPARGC1A in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,
and wherein the presence in said in vitro sample of an elevated mRNA or derived cDNA or protein expression level of PPARGC1A and a lowered OCR following treatment with a BET inhibitor in comparison with the untreated sample is suggestive of a better response to the treatment of melanoma in said patient.

5. An in vitro method according to claim 1, wherein the body fluid is blood, plasma, serum, lymph, circulating free tumor DNA, saliva, sweat, teardrops, urine or feces of a patient.

6. An in vitro method according to claim 1, wherein tissue is tumor tissue or circulating tumor cells.

7. An in vitro method according to claim 1, wherein normal human melanocytes are PCS-200-013, PCS-200-012, CRL-4004, NHEM.f-c M2 or NHEM-c M2 cells.

8. An in vitro method according to claim 1, wherein melanoma is selected from the group consisting of:
lentigo maligna (lentiginous melanoma), lentigo maligna melanoma (a melanoma that has evolved from a Lentigo maligna), superficial spreading melanoma (superficially spreading melanoma), acral lentiginous melanoma, mucosal melanoma, nodular melanoma, polypoid melanoma (a virulent variant of nodular melanoma), desmoplastic melanoma (neurotropic melanoma, or spindled melanoma), amelanotic melanoma, soft-tissue melanoma (clear-cell sarcoma), small-cell melanoma (melanoma with small nevus-like cells), Spitzoid melanoma (melanoma with features of a Spitz nevus) and uveal melanoma.

9. An in vitro method for stratifying a melanoma disease according to claim 1, wherein the inhibitor is a compound of general formula (I) 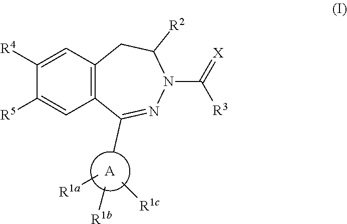
in which
X represents an oxygen or sulphur atom
A represents a monocyclic heteroaryl ring which has 5 or 6 ring atoms or represents a phenyl ring, and
R^{1a} represents hydrogen, halogen, cyano, carboxyl, amino or aminosulphonyl,
or
represents a C₁-C₆-alkoxy, C₁-C₃-alkoxy-C₁-C₃-alkyl, C₁-C₃-alkoxy-C₂-C₃-alkoxy, C₁-C₆-alkylamino, C₁-C₆-alkylcarbonylamino, C₁-C₆-alkylamino-C₁-C₆-alkyl, N-(heterocyclyl)-C₁-C₆-alkyl, N-(heterocyclyl)-C₁-C₆-alkoxy, hydroxy-C₁-C₆-alkyl, hydroxy-C₁-C₆-alkoxy, halo-C₁-C₆-alkyl, halo-C₁-C₆-alkoxy, C₁-C₆-alkylcarbonyl or C₁-C₆-alkoxycarbonyl radical,
or
represents a monocyclic heterocyclyl radical having 3 to 8 ring atoms, which may optionally be mono- or polysubstituted by identical or different substituents from the group consisting of halogen, cyano, nitro, hydroxy, amino, oxo, carboxyl,
C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, pyridinyl, —NR⁶C(═O)—R⁹, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, and/or a monocyclic heterocyclyl radical having 3 to 8 ring atoms,
or
represents a monocyclic heteroaryl radical having 5 or 6 ring atoms, which may optionally be mono- or polysubstituted by identical or different substituents from the group consisting of halogen, hydroxy, amino, cyano, nitro, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, and/or by a monocyclic heterocyclyl radical having 3 to 8 ring atoms, and/or by a monocyclic heteroaryl radical having 5 or 6 ring atoms, and/or by a phenyl radical which for its part may optionally be mono- or polysubstituted by halogen, C₁-C₃-alkyl and/or C₁-C₃-alkoxy,
or
represents a phenyl radical, which may optionally be mono- or polysubstituted by identical or different substituents from the group consisting of halogen, amino, hydroxy, cyano, nitro, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, —C(═O)NR⁶R⁷, —C(═O)R⁸, C₁-C₃-alkylsulphinyl, C₁-C₃-alkylsulphonyl, —S(═O)₂NH₂, C₁-C₃-alkylsulphonylamino, C₁-C₃-alkylaminosulphonyl, C₃-C₆-cycloalkylaminosulphonyl, halo-C₁-C₆-alkyl, halo-C₁-C₆-alkoxy, hydroxy-C₁-C₆-alkyl, C₃-C₁₀-cycloalkyl, and/or a monocyclic heterocyclyl radical having 3 to 8 ring atoms, and/or a monocyclic heteroaryl radical having 5 or 6 ring atoms and which for its part may optionally be mono- or polysubstituted by identical or different substituents from the group consisting of halogen, C₁-C₃-alkyl and C₁-C₃-alkoxy, and
R^{1b} and R^{1c} independently of one another being able to represent hydrogen, halogen, hydroxy, cyano, nitro and/or a C₁-C₆-alkyl, C₁-C₆-alkoxy, C₁-C₆-alkoxy-C₁-C₆-alkyl, halo-C₁-C₆-alkyl, halo-C₁-C₆-alkoxy, C₃-C₁₀-cycloalkyl radical and/or a monocyclic heterocyclyl radical having 3 to 8 ring atoms, and
R² represents a C₁-C₃-alkyl or trifluoromethyl or a C₃- or C₄-cycloalkyl radical,
and
R³ represents cyclopropyl, C₁-C₃-alkyl, C₁-C₃-alkoxy, amino, cyclopropylamino or C₁-C₃-alkylamino, and
R⁴ and R⁵ independently of one another represent hydrogen, hydroxy, cyano, nitro, amino, aminocarbonyl, fluorine, chlorine, bromine, C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl,
or
represent C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl which may optionally be mono- or polysubstituted by identical or different substituents from the group consisting of halogen, amino, hydroxy, carboxyl, hydroxy-C₁-C₆-alkyl, C₁-C₆-alkoxy, C₁-C₆-alkoxy-C₁-C₆-alkyl, C₁-C₆-alkylamino, amino-C₁-C₆-alkyl, monocyclic heterocyclyl having 3 to 8 ring atoms and/or monocyclic heteroaryl having 5 or 6 ring atoms, where the monocyclic heterocyclyl and heteroaryl radicals mentioned for their part may optionally be monosubstituted by C₁-C₃-alkyl,
or
represent C₃-C₁₀-cycloalkyl which may optionally be mono- or polysubstituted by identical or different substituents from the group consisting of halogen, amino, hydroxy, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, and/or a monocyclic heterocyclyl radical having 3 to 8 ring atoms,
or
represent monocyclic heteroaryl which has 5 or 6 ring atoms and which may optionally be mono- or polysubstituted by identical or different substituents from the group consisting of halogen, amino, hydroxy, cyano, nitro, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, —C(═O)R⁸, —S(═O)₂R⁹, —NR⁶R⁷, and/or a monocyclic heterocyclyl radical having 3 to 8 ring atoms,
or
represent monocyclic heterocyclyl having 3 to 8 ring atoms and which may optionally be mono- or polysubstituted by identical or different substituents from the group consisting of halogen, amino, hydroxy, cyano, oxo, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, hydroxy-C₁-C₆-alkyl, halo-C₁-C₆-alkyl, halo-C₁-C₆-alkoxy, C₃-C₁₀-cycloalkyl, —C(═O)R⁸, —S(═O)₂R⁹, —NR⁶R⁷, and/or a monocyclic heterocyclyl radical having 3 to 8 ring atoms,
or
represent phenyl which may optionally be mono- or polysubstituted by identical or different substituents from the group consisting of halogen, amino, hydroxy, cyano, nitro, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylaminocarbonyl, C₁-C₆-alkylaminosulphonyl, C₁-C₆-alkylamino-C₁-C₆-alkyl, hydroxy-C₁-C₆-alkyl, halo-C₁-C₆-alkyl, halo-C₁-C₆-alkoxy, C₃-C₁₀-cycloalkyl and/or a monocyclic heterocyclyl radical having 3 to 8 ring atoms, and
R⁶ and R⁷ independently of one another represent hydrogen, C₁-C₃-alkyl, cyclopropyl or di-C₁-C₃-alkyl-amino-C₁-C₃-alkyl or fluoropyridyl, and
R⁸ represents hydroxy, C₁-C₆-alkyl, halo-C₁-C₃-alkyl, hydroxy-C₁-C₃-alkyl, C₁-C₃-alkoxy-C₁-C₃-alkyl, C₃-C₈-cycloalkyl, phenyl, monocyclic heterocyclyl having 3 to 8 ring atoms or monocyclic heteroaryl having 5 or 6 ring atoms, where phenyl, heteroaryl and heterocyclyl may optionally be mono- or disubstituted by halogen, C₁-C₃-alkoxy or C₁-C₃-alkyl, and
R⁹ represents hydrogen, C₁-C₆-alkyl or C₁-C₄-alkoxy, and their polymorphs, enantiomers, diastereomers, racemates, tautomers, solvates, physiologically acceptable salts and solvates of these salts.

10. An in vitro method for stratifying a melanoma disease according to claim 1, wherein the inhibitor is selected from the group consisting of: (a) a compound of general formula (I) wherein
X represents an oxygen atom, and
A represents a phenyl or pyridyl ring, and
R^{1a} represents hydrogen, halogen, cyano, carboxyl, amino or amino sulphonyl, or represents a C₁-C₆-alkoxy, C₁-C₃-alkoxy-C₁-C₃-alkyl, C₁-C₃-alkoxy-C₂-C₃-alkoxy, C₁-C₆-alkylamino, C₁-C₆-alkylcarbonylamino, C₁-C₆-alkylamino-C₁-C₆-alkyl, N-(heterocyclyl)-C₁-C₆-alkyl, N-(heterocyclyl)-C₁-C₆-alkoxy, hydroxy-C₁-C₆-alkyl, hydroxy-C₁-C₆-alkoxy, halo-C₁-C₆-alkyl, halo-C₁-C₆-alkoxy, C₁-C₆-alkylcarbonyl or C₁-C₆-alkoxycarbonyl radical,
or
represents a monocyclic heterocyclyl radical having 4 to 7 ring atoms, which may optionally be mono- or polysubstituted by identical or different substituents from the group consisting of halogen, cyano, nitro, hydroxy, amino, oxo, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, phenyl, halophenyl, phenyl-C₁-C₆-alkyl, pyridinyl, —NR⁶C(═O)R⁹, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, and/or by a monocyclic heterocyclyl radical having 4 to 7 ring atoms
or
represents a monocyclic heteroaryl radical having 5 or 6 ring atoms, which may optionally be mono- or polysubstituted by identical or different substituents from the group consisting of halogen, hydroxy, amino, cyano, nitro, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, and/or by a monocyclic heterocyclyl radical having 4 to 7 ring atoms and/or by a monocyclic heteroaryl radical having 5 or 6 ring atoms and/or by a phenyl radical which for its part may optionally be mono- or polysubstituted by identical or different substituents from the group consisting of halogen, C₁-C₃-alkyl and/or C₁-C₃-alkoxy,
or
represents a phenyl radical, which may optionally be mono- or polysubstituted by identical or different substituents from the group consisting of halogen, amino, hydroxy, cyano, nitro, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, —C(═O)NR⁶R⁷, C(═O)R⁸, C₁-C₃-alkylsulphinyl, C₁-C₃-alkylsulphonyl, —S(═O)₂NH₂, C₁-C₃-alkylsulphonylamino, C₁-C₃-alkylaminosulphonyl, C₃-C₆-cycloalkylaminosulphonyl, halo-C₁-C₆-alkyl, halo-C₁-C₆-alkoxy, hydroxy-C₁-C₆-alkyl, C₃-C₁₀-cycloalkyl, and/or a monocyclic heterocyclyl radical having 4 to 7 ring atoms and/or a monocyclic heteroaryl radical having 5 or 6 ring atoms and which for its part may optionally be mono- or polysubstituted by identical or different substituents from the group consisting of halogen, C₁-C₃-alkyl and C₁-C₃-alkoxy, and
R^{1b} and R^{1c} independently of one another represent hydrogen, halogen, hydroxy, cyano, nitro or a C₁-C₆-alkyl, C₁-C₆-alkoxy, C₁-C₆-alkoxy-C₁-C₆-alkyl, halo-C₁-C₆-alkyl, halo-C₁-C₆-alkoxy, C₃-C₁₀-cycloalkyl radical and/or a monocyclic heterocyclyl radical having 4 to 7 ring atoms, and
R² represents methyl, ethyl or isopropyl, and
R³ represents cyclopropyl, C₁-C₃-alkyl, C₁-C₃-alkoxy, amino, cyclopropylamino or C₁-C₃-alkylamino, and
R⁴ and R⁵ independently of one another represent hydrogen, hydroxy, cyano, nitro, amino, aminocarbonyl, fluorine, chlorine, bromine, C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl,
or
represent C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl which may optionally be mono- or polysubstituted by identical or different substituents from the group consisting of halogen, amino, hydroxy, carboxyl, hydroxy-C₁-C₆-alkyl, C₁-C₆-alkoxy, C₁-C₆-alkoxy-C₁-C₆-alkyl, C₁-C₆-alkylamino, amino-C₁-C₆-alkyl, a monocyclic heterocyclyl having 4 to 7 ring atoms and/or a monocyclic heteroaryl having 5 or 6 ring atoms, where the monocyclic heterocyclyl and heteroaryl radicals mentioned for their part may optionally be monosubstituted by C₁-C₃-alkyl,
or
represent a C₃-C₁₀-cycloalkyl radical, which may optionally be mono- or polysubstituted by identical or different substituents from the group consisting of halogen, amino, hydroxy, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, and/or a monocyclic heterocyclyl radical having 4 to 7 ring atoms,
or
represent monocyclic heteroaryl having 5 or 6 ring atoms and which may optionally be mono- or polysubstituted by identical or different substituents from the group consisting of halogen, amino, hydroxy, cyano, nitro, carboxyl, C₁-C₆-alkyl, C₁-C₆-alkoxy, C₁-C₆-alkoxy-C₁-C₆-alkyl, hydroxy-C₁-C₆-alkyl, C₁-C₆-alkylamino, amino-C₁-C₆-alkyl, C₁-C₆-alkylamino-C₁-C₆-alkyl, halo-C₁-C₆-alkyl, halo-C₁-C₆-alkoxy, C₃-C₁₀-cycloalkyl, —C(═O)R⁸, —S(═O)₂R⁹, —NR⁶R⁷, and/or a monocyclic heterocyclyl radical having 4 to 7 ring atoms,
or
represent monocyclic heterocyclyl having 4 to 7 ring atoms and which may optionally be mono- or polysubstituted by identical or different substituents from the group consisting of halogen, amino, hydroxy, cyano, oxo, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylamino-C₁-C₆-alkyl, hydroxy-C₁-C₆-alkyl, halo-C₁-C₆-alkyl, halo-C₁-C₆-alkoxy, C₃-C₁₀-cycloalkyl, —C(═O)R⁸, —S(═O)₂R⁹, —NR⁶R⁷, and/or a monocyclic heterocyclyl radical having 4 to 7 ring atoms,
or
represent a phenyl radical, which may optionally be mono- or polysubstituted by identical or different substituents from the group consisting of halogen, amino, hydroxy, cyano, nitro, carboxyl, C₁-C₆-alkyl, C₁-C₆-alkoxy, C₁-C₆-alkoxy-C₁-C₆-alkyl, C₁-C₆-alkylamino, amino-C₁-C₆-alkyl, C₁-C₆-alkylaminocarbonyl, C₁-C₆-alkylaminosulphonyl, C₁-C₆-alkylamino-C₁-C₆-alkyl, hydroxy-C₁-C₆-alkyl, halo-C₁-C₆-alkyl, halo-C₁-C₆-alkoxy, C₃-C₁₀-cycloalkyl, and/or a monocyclic heterocyclyl radical having 4 to 7 ring atoms,
and
R⁶ and R⁷ independently of one another represent hydrogen, C₁-C₃-alkyl, cyclopropyl, di-C₁-C₃-alkyl-amino-C₁-C₃-alkyl or fluoropyridyl, and
R⁸ represents hydroxy, C₁-C₆-alkyl, halo-C₁-C₃-alkyl, hydroxy-C₁-C₃-alkyl, C₁-C₃-alkoxy-C₁-C₃-alkyl, C₃-C₈-cycloalkyl, phenyl, monocyclic heterocyclyl having 5 or 6 ring atoms, and
R⁹ represents hydrogen, C₁-C₆-alkyl or C₁-C₄-alkoxy, and their polymorphs, enantiomers, diastereomers, racemates, tautomers, solvates, physiologically acceptable salts and solvates of these salts:,
(b) a compound of general formula (I) wherein
X represents an oxygen atom, and
A represents a phenyl or pyridyl ring, and
R^{1a} represents hydrogen, halogen, cyano, carboxyl, amino or aminosulphonyl, or represents a C₁-C₆-alkoxy, C₁-C₆-alkoxy-C₁-C₃-alkyl, C₁-C₃-alkoxy-C₂-C₃-alkoxy, C₁-C₃-alkylamino, C₁-C₃-alkylcarbonylamino, C₁-C₃-alkylamino-C₁-C₃-alkyl, hydroxy-C₁-C₃-alkyl, fluoro-C₁-C₃-alkyl, fluoro-C₁-C₃-alkoxy, C₁-C₃-alkylcarbonyl or C₁-C₄-alkoxycarbonyl radical,
or
represents a monocyclic heterocyclyl radical having 4 to 7 ring atoms, which may optionally be mono- or polysubstituted by identical or different substituents from the group consisting of halogen, cyano, nitro, hydroxy, amino, oxo, carboxyl, C₁-C₃-alkyl, C₁-C₃-alkoxy, C₁-C₂-alkoxy-C₁-C₂-alkyl, hydroxy-C₁-C₃-alkyl, C₁-C₃-alkylamino, amino-C₁-C₃-alkyl, fluoro-C₁-C₃-alkyl, fluoro-C₁-C₃-alkoxy, C₃-C₆-cycloalkyl, phenyl, halophenyl, phenyl-C₁-C₃-alkyl, pyridinyl, —NR⁶C(═O)—R⁹, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, and/or by a monocyclic heterocyclyl radical having 4 to 7 ring atoms,
or
represents a monocyclic heteroaryl radical having 5 or 6 ring atoms, which may optionally be mono- or polysubstituted by identical or different substituents from the group consisting of halogen, hydroxy, amino, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, fluoro-C₁-C₃-alkyl, fluoro-C₁-C₃-alkoxy, C₃-C₆-cycloalkyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, and/or by a monocyclic heterocyclyl radical having 4 to 7 ring atoms and/or by a monocyclic heteroaryl radical having 5 or 6 ring atoms and/or by a phenyl radical which for its part may optionally be mono- or polysubstituted by identical or different substituents from the group consisting of fluorine, chlorine, bromine, methyl and/or methoxy,
or
represents a phenyl radical, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, —C(═O)NR⁶R⁷, —C(═O)R⁸, C₁-C₃-alkylsulphinyl, C₁-C₃-alkylsulphonyl, -8(═O)₂NH₂, C₁-C₃-alkylsulphonylamino, C₁-C₃-alkylaminosulphonyl, C₃-C₆-cycloalkylaminosulphonyl, fluoro-C₁-C₃-alkyl, fluoro-C₁-C₃-alkoxy, hydroxy-C₁-C₃-alkyl, C₃-C₆-cycloalkyl and/or a monocyclic heterocyclyl radical having 4 to 7 ring atoms and/or a monocyclic heteroaryl radical having 5 or 6 ring atoms and which for its part may optionally be mono- or polysubstituted by identical or different substituents from the group consisting of fluorine, chlorine, bromine, methyl and/or methoxy, and
R^{1b} represents hydrogen, halogen, hydroxy, cyano, nitro or represents a C₁-C₃-alkyl, C₁-C₃-alkoxy, fluoro-C₁-C₃-alkyl or fluoro-C₁-C₃-alkoxy radical, and
R^{1c} represents hydrogen, fluorine, chlorine, bromine or cyano, and
R² represents methyl, ethyl or isopropyl, and
R³ represents cyclopropyl, C₁-C₃-alkyl, C₁-C₃-alkoxy, cyclopropylamino or C₁-C₃-alkylamino, and
R⁴ and R⁵ independently of one another represent hydrogen, hydroxy, cyano, nitro, amino, aminocarbonyl, fluorine, chlorine, bromine, C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl,
or
represent C₁-C₃-alkyl, C₁-C₃-alkoxy, C₁-C₃-alkylamino, which may be mono- or polysubstituted by identical or different substituents from the group consisting of halogen, amino, hydroxy, carboxyl, hydroxy-C₁-C₃-alkyl, C₁-C₃-alkoxy, C₁-C₃-alkylamino, amino-C₁-C₃-alkyl, monocyclic heterocyclyl having 4 to 7 ring atoms, and/or monocyclic heteroaryl having 5 or 6 ring atoms, where the monocyclic heterocyclyl and heteroaryl radicals mentioned for their part may optionally be monosubstituted by C₁-C₃-alkyl,
or
represent a C₃-C₇-cycloalkyl radical, which may optionally be mono- or polysubstituted by identical or different substituents from the group consisting of halogen, amino, hydroxy, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, fluoro-C₁-C₃-alkyl, fluoro-C₁-C₃-alkoxy, and/or a monocyclic heterocyclyl radical having 4 to 7 ring atoms,
or
represent monocyclic heterocyclyl having 5 or 6 ring atoms and which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, hydroxy-C₁-C₃-alkyl, C₁-C₃-alkylamino, amino-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷, and/or a monocyclic heterocyclyl radical having 4 to 7 ring atoms,
or
represent monocyclic heterocyclyl having 4 to 7 ring atoms and which may optionally be mono- or polysubstituted by identical or different substituents from the group consisting of halogen, amino, hydroxy, cyano, oxo, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, hydroxy-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷ and/or a monocyclic heterocyclyl radical having 4 to 7 ring atoms,
or
represent a phenyl radical, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, C₁-C₃-alkylaminocarbonyl, C₁-C₃-alkylaminosulphonyl, hydroxy-C₁-C₃-alkyl, fluoro-C₁-C₃-alkyl, fluoro-C₁-C₃-alkoxy, C₃-C₆-cycloalkyl and/or a monocyclic heterocyclyl radical having 4 to 7 ring atoms, and
R⁶ and R⁷ independently of one another represent hydrogen, C₁-C₃-alkyl, cyclopropyl, di-C₁-C₃-alkylamino-C₁-C₃-alkyl or fluoropyridyl, and
R⁸ represents hydroxy, C₁-C₆-alkyl, hydroxy-C₁-C₃-alkyl, fluoro-C₁-C₃-alkyl, C₁-C₃-alkoxy-C₁-C₃-alkyl, C₃-C₈-cycloalkyl or monocyclic heterocyclyl which has 5 or 6 ring atoms, and
R⁹ represents hydrogen, C₁-C₆-alkyl or C₁-C₄-alkoxy, and their polymorphs, enantiomers, diastereomers, racemates, tautomers, solvates, physiologically acceptable salts and solvates of these salts;
(c) a compound of general formula (I) wherein
X represents an oxygen atom, and
A represents a phenyl or pyridyl ring, and
R^{1a} represents a monocyclic heterocycyl radical having 4 to 7 ring atoms, which may optionally be mono- or polysubstituted by identical or different substituents from the group consisting of halogen, cyano, nitro, hydroxy, amino, oxo, carboxyl, C₁-C₃-alkyl, C₁-C₃-alkoxy, C₁-C₂-alkoxy-C₁-C₂-alkyl, hydroxy-C₁-C₃-alkyl, C₁-C₃-alkylamino, amino-C₁-C₃-alkyl, fluoro-C₁-C₃-alkyl, fluoro-C₁-C₃-alkoxy, C₃-C₆-cycloalkyl, phenyl, halophenyl, phenyl-C₁-C₃-alkyl, pyridinyl, —NR⁶C(═O)—R⁹, —C(═O)—NR⁶R⁷, —C(═O)—R⁸, —S(═O)₂—NR⁶R⁷ —S(═O)—R⁹, —S(═O)₂—R⁹, —NH—S(═O)₂—R⁹, and/or by a monocyclic heterocyclyl radical having 4 to 7 ring atoms,
or
represents a monocyclic heteroaryl radical having 5 or 6 ring atoms, which may optionally be mono- or polysubstituted by identical or different substituents from the group consisting of halogen, hydroxy, amino, cyano, nitro, carboxyl, C₁-C₃-alkyl, C₁-C₃-alkoxy, Cᵢ-C₂-alkoxy-Cᵢ-C₂-alkyl, C₁-C₃-alkylamino, amino-C₁-C₃-alkyl, fluoro-C₁-C₃-alkyl, fluoro-C₁-C₃-alkoxy, C₃-C₆-c yclo alkyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, and/or by a monocyclic heterocyclyl radical having 4 to 7 ring atoms, and/or by a monocyclic heteroaryl radical having 5 or 6 ring atoms, and/or by a phenyl radical which for its part may be mono- or polysubstituted by identical or different substituents from the group consisting of fluorine, chlorine, bromine, methyl or methoxy,
or
represents a phenyl radical, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, —C(═O)NR⁶R⁷, —C(═O)R⁸, C₁-C₃-alkylsulphinyl, C₁-C₃-alkylsulphonyl, —S(═O)₂NH₂, C₁-C₃-alkylsulphonylamino, C₁-C₃-alkylaminosulphonyl, C₃-C₆-cycloalkylaminosulphonyl, fluoro-C₁-C₃-alkyl, fluoro-C₁-C₃-alkoxy, hydroxy-C₁-C₃-alkyl, C₃-C₆-cycloalkyl and/or a monocyclic heterocyclyl radical having 4 to 7 ring atoms, and/or a monocyclic heteroaryl radical having 5 or 6 ring atoms which for its part may optionally be mono- or polysubstituted by identical or different substituents from the group consisting of fluorine, chlorine, bromine, methyl or methoxy, and
R^{1b} represents hydrogen, halogen, hydroxy, cyano, nitro or represents a C₁-C₃-alkyl, C₁-C₃-alkoxy, fluoro-C₁-C₃-alkyl or fluoro-C₁-C₃-alkoxy radical, and
R^{1c} represents hydrogen, fluorine, chlorine, bromine or cyano, and
R² represents methyl, ethyl or isopropyl, and
R³ represents cyclopropyl, C₁-C₃-alkyl, C₁-C₃-alkoxy, cyclopropylamino or C₁-C₃-alkylamino, and
R⁴ and R⁵ independently of one another represent hydrogen, hydroxy, cyano, nitro, amino, aminocarbonyl, fluorine, chlorine, bromine, C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl,
or
represent C₁-C₃-alkyl, C₁-C₃-alkoxy, C₁-C₃-alkylamino, which may be mono- or polysubstituted by identical or different substituents from the group consisting of halogen, amino, hydroxy, carboxyl, hydroxy-C₁-C₃-alkyl, C₁-C₃-alkoxy, C₁-C₃-alkylamino, amino-C₁-C₃-alkyl, monocyclic heterocyclyl having 4 to 7 ring atoms, and/or monocyclic heteroaryl having 5 or 6 ring atoms, where the monocyclic heterocyclyl and heteroaryl radicals mentioned for their part may optionally be monosubstituted by C₁-C₃-alkyl,
or
represent a C₃-C₇-cycloalkyl radical, which may optionally be mono- or polysubstituted by identical or different substituents from the group consisting of halogen, amino, hydroxy, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, fluoro-C₁-C₃-alkyl, fluoro-C₁-C₃-alkoxy, and/or a monocyclic heterocyclyl radical having 4 to 7 ring atoms,
or
represent monocyclic heteroaryl having 5 or 6 ring atoms, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, hydroxy-C₁-C₃-alkyl, C₁-C₃-alkylamino, amino-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷, and/or a monocyclic heterocyclyl radical having 4 to 7 ring atoms,
or
represent monocyclic heterocyclyl having 4 to 7 ring atoms, which may optionally be mono- or polysubstituted by identical or different substituents from the group consisting of halogen, amino, hydroxy, cyano, oxo, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, hydroxy-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷, and/or a monocyclic heterocyclyl radical having 4 to 7 ring atoms,
or
represent a phenyl radical, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, C₁-C₃-alkylaminocarbonyl, C₁-C₃-alkylaminosulphonyl, hydroxy-C₁-C₃-alkyl, fluoro-C₁-C₃-alkyl, fluoro-C₁-C₃-alkoxy, C₃-C₆-cycloalkyl, and/or a monocyclic heterocyclyl radical having 4 to 7 ring atoms, and
R⁶ and R⁷ independently of one another represent hydrogen, C₁-C₃-alkyl, cyclopropyl, di-C₁-C₃-alkylamino-C₁-C₃-alkyl or fluoropyridyl, and
R⁸ represents hydroxy, C₁-C₆-alkyl, hydroxy-C₁-C₃-alkyl, fluoro-C₁-C₃-alkyl, C₁-C₃-alkoxy-C₁-C₃-alkyl, C₃-C₈-cycloalkyl or monocyclic heterocyclyl which has 5 or 6 ring atoms, and
R⁹ represents hydrogen, C₁-C₆-alkyl or C₁-C₄-alkoxy, and their polymorphs, enantiomers, diastereomers, racemates, tautomers, solvates, physiologically acceptable salts and solvates of these salts;
(d) a compound of general formula (I) wherein
X represents an oxygen atom, and
A represents a phenyl or pyridyl ring, and
R^{1a} represents hydrogen, halogen, cyano, carboxyl, amino or aminosulphonyl, or represents a C₁-C₆-alkoxy, C₁-C₃-alkoxy-C₁-C₃-alkyl, C₁-C₃-alkoxy-C₂-C₃-alkoxy, C₁-C₃-alkylamino, C₁-C₃-alkylcarbonylamino, C₁-C₃-alkylamino-C₁-C₃-alkyl, hydroxy-C₁-C₃-alkyl, fluoro-C₁-C₃-alkyl, fluoro-C₁-C₃-alkoxy, C₁-C₃-alkylcarbonyl or C₁-C₄-alkoxycarbonyl radical,
or
represents a monocyclic heterocyclyl radical having 4 to 7 ring atoms, which may optionally be mono- or polysubstituted by identical or different substituents from the group consisting of halogen, cyano, nitro, hydroxy, amino, oxo, carboxyl, C₁-C₃-alkyl, C₁-C₃-alkoxy, C₁-C₂-alkoxy-C₁-C₂-alkyl, hydroxy-C₁-C₃-alkyl, C₁-C₃-alkylamino, amino-C₁-C₃-alkyl, fluoro-C₁-C₃-alkyl, fluoro-C₁-C₃-alkoxy, C₃-C₆-cycloalkyl, phenyl, halophenyl, phenyl-C₁-C₃-alkyl, pyridinyl, —NR⁶C(═O)—R⁹, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, and/or by a monocyclic heterocyclyl radical having 4 to 7 ring atoms,
or
represents a monocyclic heteroaryl radical having 5 or 6 ring atoms, which may optionally be mono- or polysubstituted by identical or different substituents from the group consisting of halogen, hydroxy, amino, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, fluoro-C₁-C₃-alkyl, fluoro-C₁-C₃-alkoxy, C₃-C₆-cycloalkyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, and/or by a monocyclic heterocyclyl radical having 4 to 7 ring atoms, and/or by a monocyclic heteroaryl radical having 5 or 6 ring atoms, and/or by a phenyl radical which for its part may be mono- or polysubstituted by identical or different substituents from the group consisting of fluorine, chlorine, bromine, methyl and/or methoxy,
or
represents a phenyl radical, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, —C(═O)NR⁶R⁷, C(═O)R⁸, C₁-C₃-alkylsulphinyl, C₁-C₃-alkylsulphonyl, —S(═O)₂NH₂, C₁-C₃-alkylsulphonylamino, C₁-C₃-alkylaminosulphonyl, C₃-C₆-cycloalkylaminosulphonyl, fluoro-C₁-C₃-alkyl, fluoro-C₁-C₃-alkoxy, hydroxy-C₁-C₃-alkyl, C₃-C₆-cycloalkyl and/or a monocyclic heterocyclyl radical having 4 to 7 ring atoms, and/or a monocyclic heteroaryl radical having 5 or 6 ring atoms which for its part may optionally be mono- or polysubstituted by identical or different substituents from the group consisting of fluorine, chlorine, bromine, methyl and/or methoxy, and
R^{1b} represents hydrogen, halogen, hydroxy, cyano, nitro or represents a C₁-C₃-alkyl, C₁-C₃-alkoxy, fluoro-C₁-C₃-alkyl or fluoro-C₁-C₃-alkoxy radical, and
R^{1c} represents hydrogen, fluorine, chlorine, bromine or cyano, and
R² represents methyl, ethyl or isopropyl, and
R³ represents cyclopropyl, C₁-C₃-alkyl, C₁-C₃-alkoxy, cyclopropylamino or C₁-C₃-alkylamino, and
R⁴ represents a C₃-C₇-cycloalkyl radical, which may optionally be mono- or polysubstituted by identical or different substituents from the group consisting of halogen, amino, hydroxy, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, fluoro-C₁-C₃-alkyl, fluoro-C₁-C₃-alkoxy, and/ or a monocyclic heterocyclyl radical having 4 to 7 ring atoms,
or
represents monocyclic heteroaryl having 5 or 6 ring atoms, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, hydroxy-C₁-C₃-alkyl, C₁-C₃-alkylamino, amino-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷, and/or a monocyclic heterocyclyl radical having 4 to 7 ring atoms,
or
represents monocyclic heterocyclyl having 4 to 7 ring atoms, which may optionally be mono- or polysubstituted by identical or different substituents from the group consisting of halogen, amino, hydroxy, cyano, oxo, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, hydroxy-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷, and/or a monocyclic heterocyclyl radical having 4 to 7 ring atoms,
or
represents a phenyl radical, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, C₁-C₃-alkylaminocarbonyl, C₁-C₃-alkylaminosulphonyl, hydroxy-C₁-C₃-alkyl, fluoro-C₁-C₃-alkyl, fluoro-C₁-C₃-alkoxy, C₃-C₆-cycloalkyl, and/or a monocyclic heterocyclyl radical having 4 to 7 ring atoms, and
R⁵ represents hydrogen, hydroxy, cyano, nitro, amino, aminocarbonyl, fluorine, chlorine, bromine, C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl,
and
R⁶ and R⁷ independently of one another represent hydrogen, C₁-C₃-alkyl, cyclopropyl, di-C₁-C₃-alkylamino-C₁-C₃-alkyl or fluoropyridyl, and
R⁸ represents hydroxy, C₁-C₆-alkyl, hydroxy-C₁-C₃-alkyl, fluoro-C₁-C₃-alkyl, C₁-C₃-alkoxy-C₁-C₃-alkyl, C₃-C₈-cycloalkyl or monocyclic heterocyclyl which has 5 or 6 ring atoms, and
R⁹ represents hydrogen, C₁-C₆-alkyl or C₁-C₄-alkoxy, and their polymorphs, enantiomers, diastereomers, racemates, tautomers, solvates, physiologically acceptable salts and solvates of these salts;
(e) a compound of general formula (I) wherein
X represents an oxygen atom, and
A represents a phenyl or pyridyl ring, and
R^{1a} represents hydrogen, halogen, cyano, carboxyl, amino or aminosulphonyl, or represents a C₁-C₆-alkoxy, C₁-C₃-alkoxy-C₁-C₃-alkyl, C₁-C₃-alkoxy-C₂-C₃-alkoxy, C₁-C₃-alkylamino, C₁-C₃-alkylcarbonylamino, C₁-C₃-alkylamino-C₁-C₃-alkyl, hydroxy-C₁-C₃-alkyl, fluoro-C₁-C₃-alkyl, fluoro-C₁-C₃-alkoxy, C₁-C₃-alkylcarbonyl or C₁-C₄-alkoxycarbonyl radical,
or
represents a monocyclic heterocyclyl radical having 4 to 7 ring atoms, which may optionally be mono- or polysubstituted by identical or different substituents from the group consisting of halogen, cyano, nitro, hydroxy, amino, oxo, carboxyl, C₁-C₃-alkyl, C₁-C₃-alkoxy, C₁-C₂-alkoxy-C₁-C₂-alkyl, hydroxy-C₁-C₃-alkyl, C₁-C₃-alkylamino, amino-C₁-C₃-alkyl, fluoro-C₁-C₃-alkyl, fluoro-C₁-C₃-alkoxy, C₃-C₆-cycloalkyl, phenyl, halophenyl, phenyl-C₁-C₃-alkyl, pyridinyl, —NR⁶C(═O)—R⁹, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, and/or by a monocyclic heterocyclyl radical having 4 to 7 ring atoms,
or
represents a monocyclic heteroaryl radical having 5 or 6 ring atoms, which may optionally be mono- or polysubstituted by identical or different substituents from the group consisting of halogen, hydroxy, amino, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, fluoro-C₁-C₃-alkyl, fluoro-C₁-C₃-alkoxy, C₃-C₆-cycloalkyl, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹, and/or by a monocyclic heterocyclyl radical having 4 to 7 ring atoms, and/or by a monocyclic heteroaryl radical having 5 or 6 ring atoms, and/or by a phenyl radical which for its part may be mono- or polysubstituted by identical or different substituents from the group consisting of fluorine, chlorine, bromine, methyl and/or methoxy,
or
represents a phenyl radical, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, —C(═O)NR⁶R⁷, —C(═O)R⁸, C₁-C₃-alkylsulphinyl, C₁-C₃-alkylsulphonyl, -8(═O)₂NH₂, C₁-C₃-alkylsulphonylamino, C₁-C₃-alkylaminosulphonyl, C₃-C₆-cycloalkylaminosulphonyl, fluoro-C₁-C₃-alkyl, fluoro-C₁-C₃-alkoxy, hydroxy-C₁-C₃-alkyl, C₃-C₆-cycloalkyl and/or a monocyclic heterocyclyl radical having 4 to 7 ring atoms, and/or a monocyclic heteroaryl radical having 5 or 6 ring atoms which for its part may optionally be mono- or polysubstituted by identical or different substituents from the group consisting of fluorine, chlorine, bromine, methyl and/or methoxy, and
R^{1b} represents hydrogen, halogen, hydroxy, cyano, nitro or represents a C₁-C₃-alkyl, C₁-C₃-alkoxy, fluoro-C₁-C₃-alkyl or fluoro-C₁-C₃-alkoxy radical, and
R^{1c} represents hydrogen, fluorine, chlorine, bromine or cyano, and
R² represents methyl, ethyl or isopropyl, and
R³ represents cyclopropyl, C₁-C₃-alkyl, C₁-C₃-alkoxy, cyclopropylamino or C₁-C₃-alkylamino, and
R⁴ represents hydrogen, hydroxy, cyano, nitro, amino, aminocarbonyl, fluorine, chlorine, bromine, C₁-C₆-alkyl, C₁-C₆-alkoxy, C₁-C₆-alkylamino, C₁-C₆-alkylcarbonylamino, C₁-C₆-alkylaminocarbonyl or C₁-C₆-alkylaminosulphonyl,
and
R⁵ represents a C₃-C₇-cycloalkyl radical, which may optionally be mono- or polysubstituted by identical or different substituents from the group consisting of halogen, amino, hydroxy, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, fluoro-C₁-C₃-alkyl, fluoro-C₁-C₃-alkoxy, and/or a monocyclic heterocyclyl radical having 4 to 7 ring atoms,
or
represents monocyclic heteroaryl having 5 or 6 ring atoms, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, hydroxy-C₁-C₃-alkyl, C₁-C₃-alkylamino, amino-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷, and/or a monocyclic heterocyclyl radical having 4 to 7 ring atoms,
or
represents monocyclic heterocyclyl having 4 to 7 ring atoms, which may optionally be mono- or polysubstituted by identical or different substituents from the group consisting of halogen, amino, hydroxy, cyano, oxo, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, hydroxy-C₁-C₃-alkyl, fluoro-C₁-C₃-alkyl, fluoro-C₁-C₃-alkoxy, C₃-C₆-cycloalkyl, —C(═O)R⁸, —S(═O)₂R⁹, —NR⁶R⁷, and/or a monocyclic heterocyclyl radical having 4 to 7 ring atoms,
or
represents a phenyl radical, which may optionally be mono- or polysubstituted by identical or different substituents from the group consisting of halogen, amino, hydroxy, cyano, nitro, carboxyl, C₁-C₃-alkyl, C₁-C₃-alkoxy, C₁-C₂-alkoxy-C₁-C₂-alkyl, C₁-C₃-alkylamino, amino-C₁-C₃-alkyl, C₁-C₃-alkylaminocarbonyl, C₁-C₃-alkylaminosulphonyl, hydroxy-C₁-C₃-alkyl, fluoro-C₁-C₃-alkyl, fluoro-C₁-C₃-alkoxy, C₃-C₆-cycloalkyl, and/or a monocyclic heterocyclyl radical having 4 to 7 ring atoms, and
R⁶ and R⁷ independently of one another represent hydrogen, C₁-C₃-alkyl, cyclopropyl, di-C₁-C₃-alkylamino-C₁-C₃-alkyl or fluoropyridyl, and
R⁸ represents hydroxy, C₁-C₆-alkyl, hydroxy-C₁-C₃-alkyl, fluoro-C₁-C₃-alkyl, C₁-C₃-alkoxy-C₁-C₃-alkyl, C₃-C₈-cycloalkyl or monocyclic heterocyclyl which has 5 or 6 ring atoms, and
R⁹ represents hydrogen, C₁-C₆-alkyl or C₁-C₄-alkoxy, and their polymorphs, enantiomers, diastereomers, racemates, tautomers, solvates, physiologically acceptable salts and solvates of these salts;
(f) a compound of general formula (I) wherein
X represents an oxygen atom, and
A represents a phenyl or 3-pyridyl ring, and
R^{1a} represents hydrogen or chlorine,
or
represents piperazinyl, pyrrolidinyl, piperidinyl, diazepanyl, oxazinanyl, oxazolidinyl, morpholinyl, thiomorpholinyl, thiazolidinyl or azetidinyl, which may optionally be mono- or polysubstituted by identical or different substituents from the group consisting of fluorine, chlorine, cyano, nitro, hydroxy, oxo, C₁-C₃-alkyl, C₁-C₃-alkoxy, C₁-C₂-alkoxy-C₁-C₂-alkyl, hydroxy-C₁-C₃-alkyl, dimethylamino, trifluoromethyl, difluoroethyl, trifluoroethyl, trifluoromethoxy, cyclopropyl, phenyl, fluorophenyl, phenyl, C₁-C₃-alkyl, pyridinyl, —NR⁶C(═O)—R⁹, —C(═O)—NR⁶R⁷, —C(═O)—R⁸,
—S(═O)₂—NR⁶R⁷, —S(═O)—R⁹, —S(═O)₂—R⁹, and/or —NH—S(═O)₂—R⁹,
or
represents tetrazolyl,
or
represents isoxazolyl, pyrazolyl, thienyl, thiazolyl, imidazolyl, triazolyl, pyrrolyl, oxadiazolyl, pyridinyl or pyrimidinyl, which may optionally be mono- or polysubstituted by identical or different substituents from the group consisting of fluorine, chlorine, hydroxy, amino, cyano, nitro, C₁-C₃-alkyl, C₁-C₃-alkoxy, C₁-C₂-alkoxy-C₁-C₂-alkyl, dimethylamino, trifluoromethyl, difluoroethyl, trifluoroethyl, trifluoromethoxy, cyclopropyl, pyridinyl, phenyl, fluorophenyl, —C(═O)—NR⁶R⁷, —C(═O)—R⁸, —S(═O)₂—NR⁶R⁷, —S(═O)—R⁹, —S(═O)₂—R⁹, and/or —NH—S(═O)₂—R⁹,
or
represents a phenyl radical, which may optionally be mono- or polysubstituted by identical or different substituents from the group consisting of fluorine, chlorine, amino, hydroxy, cyano, nitro, carboxyl, C₁-C₃-alkyl, C₁-C₃-alkoxy, C₁-C₂-alkoxy-C₁-C₂-alkyl, dimethylamino, —C(═O)NR⁶R⁷, —C(═O)R⁸, C₁-C₃-alkylsulphinyl, C₁-C₃-alkylsulphonyl, -s(═O)₂NH₂, C₁-C₃-alkylsulphonylamino, C₁-C₃-alkylaminosulphonyl, C₃-C₆-cycloalkylaminosulphonyl, trifluoromethyl, difluoroethyl, trifluoroethyl, trifluoromethoxy, hydroxy-C₁-C₃-alkyl, cyclopropyl, chlorothienyl, morpholino and/or pyridinyl, and
R^{1b} represents hydrogen, fluorine, bromine or cyano,
R^{1c} represents hydrogen or bromine, and
R² represents methyl, ethyl or isopropyl, and
R³ represents cyclopropyl, methyl, ethyl, methoxy, ethoxy, cyclopropylamino, methylamino or ethylamino, and
R⁴ and R⁵ independently of one another represent hydrogen, hydroxy, cyano, amino, chlorine C₁-C₆-alkyl, methoxy, ethoxy or C₁-C₃-alkylcarbonylamino,
or
represent difluoromethoxy or trifluoromethoxy,
or represent C₁-C₃-alkoxy, which may be substituted by pyridinyl, morpholinyl, pyrrolidinyl or piperazinyl,
in which pyridinyl and piperazinyl in turn may be optionally substituted by C₁-C₃-alkyl,
or
represent cyclopropyl,
or
represent pyridinyl, pyrazolyl, triazolyl or isoxazolyl, which may be optionally mono- or polysubstituted by identical or different substituents from the group consisting of hydroxy and/or methyl,
or
represent pyrrolidinyl, morpholinyl, piperidinyl, piperazinyl, oxazolidinyl, thiomorpholinyl, which may optionally be mono- or polysubstituted by oxo, methyl and/or —S(═O)₂R⁹,
or
represent phenyl optionally substituted by C₁-C₃-alkylaminosulphonyl or fluorine, and
R⁶ and R⁷ independently of one another represent hydrogen, C₁-C₃-alkyl, cyclopropyl, di-C₁-C₃-alkylamino-C₁-C₃-alkyl or fluoropyridyl, and
R⁸ represents hydroxy, C₁-C₃-alkyl, hydroxy-C₁-C₃-alkyl, trifluoromethyl, pyrrolidinyl, morpholinyl or piperidinyl, and
R⁹ represents C₁-C₄-alkyl or C₁-C₄-alkoxy, and their polymorphs, enantiomers, diastereomers, racemates, tautomers, solvates, physiologically acceptable salts and solvates of these salts;
(g) a compound of general formula (I) wherein
X represents an oxygen atom, and
A represents a phenyl or 3-pyridyl ring, and
R^{1a} represents hydrogen or chlorine,
or
represents piperazinyl, pyrrolidinyl, piperidinyl, diazepanyl, oxazinanyl, oxazolidinyl, morpholinyl, thiomorpholinyl, thiazolidinyl or azetidinyl, which may optionally be mono- or polysubstituted by identical or different substituents from the group consisting of fluorine, hydroxy, oxo, C₁-C₃-alkyl, methoxy, hydroxy-C₁-C₃-alkyl, dimethylamino, difluoroethyl, trifluoroethyl, benzyl, —NR⁶C(═O)—R⁹, —C(═O)—NR⁶R⁷, —C(═O)—R⁸, and/or
—S(═O)₂—R⁹,
or
represents tetrazolyl,
or
represents isoxazolyl, pyrazolyl, thienyl, thiazolyl, imidazolyl, triazolyl, pyrrolyl, oxadiazolyl, pyridinyl or pyrimidinyl, which may optionally be mono- or polysubstituted by identical or different substituents from the group consisting of fluorine, chlorine, hydroxy, cyano, C₁-C₂-alkyl, methoxy, methoxymethyl, trifluoromethyl, cyclopropyl, pyridinyl, phenyl, fluorophenyl and/or —C(═O)—R⁸,
or
represents a phenyl radical, which may optionally be mono- or polysubstituted by identical or different substituents from the group consisting of fluorine, chlorine, hydroxy, cyano, nitro, carboxyl, C₁-C₃-alkyl, methoxy, —C(═O)NR⁶R⁷, —C(═O)R⁸,
C₁-C₃-alkylsulphinyl, C₁-C₃-alkylsulphonyl, —S(═O)₂NH₂ C₁-C₃-alkylsulphonylamino, C₁-C₃-alkylaminosulphonyl, C₃-C₆-cyclo-alkylaminosulphonyl, trifluoromethyl, trifluoromethoxy, hydroxy-C₁-C₃-alkyl, cyclopropyl, chlorothienyl and/or morpholino, and
R^{1b} represents hydrogen, fluorine, bromine or cyano, and
R^{1c} represents hydrogen or bromine, and
R² represents methyl, ethyl or isopropyl, and
R³ represents cyclopropyl, methyl, ethyl, methoxy, ethoxy, cyclopropylamino, methylamino or ethylamino, and
R⁴ and R⁵ independently of one another represent hydrogen, hydroxy, cyano, amino, chlorine, C₁-C₆-alkyl, methoxy, ethoxy or C₁-C₃-alkylcarbonylamino,
or
represent difluoromethoxy or trifluoromethoxy,
or represent C₁-C₃-alkoxy, which may be substituted by pyridinyl, morpholinyl, pyrrolidinyl or piperazinyl,
in which pyridinyl and piperazinyl in turn may be optionally substituted by C₁-C₃-alkyl,
or
represent cyclopropyl,
or
represent pyridinyl, pyrazolyl, triazolyl or isoxazolyl, optionally mono- or polysubstituted by identical or different substituents from the group consisting of hydroxy and/or methyl,
or
represent pyrrolidinyl, morpholinyl, piperidinyl, piperazinyl, oxazolidinyl, thiomorpholinyl, which may optionally be mono- or polysubstituted by methyl, oxo and/or —S(═O)₂R⁹
or
represent phenyl optionally substituted by C₁-C₃-alkylaminosulphonyl or fluorine, and
R⁶ and R⁷ independently of one another represent hydrogen, C₁-C₃-alkyl, cyclopropyl, di-C₁-C₃-alkylamino-C₁-C₃-alkyl or fluoropyridyl, and
R⁸ represents hydroxy, C₁-C₃-alkyl, hydroxy-C₁-C₃-alkyl, trifluoromethyl, pyrrolidinyl, morpholinyl or piperidinyl, and
R⁹ represents C₁-C₄-alkyl or C₁-C₄-alkoxy, and their polymorphs, enantiomers, diastereomers, racemates, tautomers, solvates, physiologically acceptable salts and solvates of these salts;
(h) a compound of general formula (I) wherein
X represents an oxygen atom, and
A represents a phenyl ring, and
R^{1a} represents piperazinyl, pyrrolidinyl, piperidinyl, diazepanyl, oxazinanyl, oxazolidinyl, morpholinyl, thiomorpholinyl, thiazolidinyl or azetidinyl, which may optionally be mono- or polysubstituted by identical or different substituents from the group consisting of fluorine, hydroxy, oxo, C₁-C₃-alkyl, methoxy, hydroxy-C₁-C₃-alkyl, dimethylamino, difluoroethyl, trifluoroethyl, benzyl, —NR⁶C(═O)—R⁹, —C(═O)—NR⁶R⁷, —C(═O)—R⁸, and/or —S(═O)₂—R⁹,
or
represents tetrazolyl,
or
represents isoxazolyl, pyrazolyl, thienyl, thiazolyl, imidazolyl, triazolyl, pyrrolyl, oxadiazolyl, pyridinyl or pyrimidinyl, which may optionally be mono- or polysubstituted by identical or different substituents from the group consisting of fluorine, chlorine, hydroxy, cyano, C₁-C₂-alkyl, methoxy, methoxymethyl, trifluoromethyl, cyclopropyl, pyridinyl, phenyl, fluorophenyl and/or —C(═O)—R⁸,
or
represents a phenyl radical, which may optionally be mono- or polysubstituted by identical or different substituents from the group consisting of fluorine, chlorine, hydroxy, cyano, nitro, carboxyl, C₁-C₃-alkyl, methoxy, —C(═O)NR⁶R⁷, C(═O)R⁸, C₁-C₃-alkylsulphinyl, C₁-C₃-alkylsulphonyl, —S(═O)₂NH₂, C₁-C₃-alkylsulphonylamino, C₁-C₃-alkylaminosulphonyl, C₃-C₆-cycloalkylaminosulphonyl, trifluoromethyl, trifluoromethoxy, hydroxy-C₁-C₃-alkyl, cyclopropyl, chlorothienyl and/or morpholino,
R^{1b} represents hydrogen, fluorine, bromine or cyano, and
R^{1c} represents hydrogen, and
R² represents methyl or ethyl, and
R³ represents methylamino, and
R⁴ and R⁵ independently of one another represent hydrogen, hydroxy, cyano, chlorine, C₁-C₆-alkyl, methoxy, ethoxy or C₁-C₃-alkylcarbonylamino,
or
represent difluoromethoxy or trifluoromethoxy, and
R⁶ and R⁷ independently of one another represent hydrogen, C₁-C₃-alkyl, cyclopropyl or di-C₁-C₃-alkylamino-C₁-C₃-alkyl, and
R⁸ represents hydroxy, C₁-C₃-alkyl, hydroxy-C₁-C₃-alkyl, trifluoromethyl, pyrrolidinyl, morpholinyl or piperidinyl, and
R⁹ represents C₁-C₄-alkyl or C₁-C₄-alkoxy, and their polymorphs, tautomers, solvates, physiologically acceptable salts and solvates of these salts, and
where the stereocentre, which is represented by the carbon atom of the benzodiazepine skeleton which is bound to R², is present either in racemic form or predominantly or completely in the (S) configuration;
(i) a compound of general formula (I) wherein
X represents an oxygen atom, and
A represents a phenyl ring, and
R^{1a} represents hydrogen or chlorine, and
R^{1b} represents hydrogen, fluorine, bromine or cyano, and
R^{1c} represents hydrogen, and
R² represents methyl or ethyl, and
R³ represents methylamino, and
R⁴ represents cyclopropyl,
or
represents pyridinyl, pyrazolyl, triazolyl or isoxazolyl, which may optionally be mono- or polysubstituted by identical or different substituents from the group consisting of hydroxy and/or methyl,
or
represents pyrrolidinyl, morpholinyl, piperidinyl, piperazinyl, oxazolidinyl or thiomorpholinyl, which may optionally be mono- or polysubstituted by methyl, oxo and/or —S(═O)₂R⁹,
or
represents phenyl optionally substituted by C₁-C₃-alkylaminosulphonyl or fluorine, and
R⁵ represents hydrogen, hydroxy, cyano, chlorine, C₁-C₆-alkyl, methoxy, ethoxy or C₁-C₃-alkylcarbonylamino,
or
represents difluoromethoxy or trifluoromethoxy, and
R⁶ and R⁷ independently of one another represent hydrogen, C₁-C₃-alkyl, cyclopropyl or di-C₁-C₃-alkylamino-C₁-C₃-alkyl, and
R⁸ represents hydroxy, C₁-C₃-alkyl, hydroxy-C₁-C₃-alkyl, trifluoromethyl, pyrrolidinyl, morpholinyl or piperidinyl, and
R⁹ represents C₁-C₄-alkyl or C₁-C₄-alkoxy, and their polymorphs, tautomers, solvates, physiologically acceptable salts and solvates of these salts, and
where the stereocentre, which is represented by the carbon atom of the benzodiazepine skeleton which is bound to R², is present either in racemic form or predominantly or completely in the (S) configuration;
(j) a compound of general formula (I) wherein
X represents an oxygen atom, and
A represents a phenyl ring, and
R^{1a} represents hydrogen or chlorine, and
R^{1b} represents hydrogen, fluorine, bromine or cyano, and
R^{1c} represents hydrogen, and
R² represents methyl or ethyl, and
R³ represents methylamino, and
R⁴ represents hydrogen, chlorine, methoxy or ethoxy,
or
represents difluoromethoxy or trifluoromethoxy, and
R⁵ represents cyclopropyl,
or
represents pyridinyl or pyrazolyl, which may optionally be substituted one or more times by methyl,
or
represents morpholinyl, piperidinyl, piperazinyl or thiomorpholinyl, which may optionally be mono- or polysubstituted by methyl, oxo and/or —S(═O)₂R⁹,
or
represents phenyl which is substituted by C₁-C₃-alkylaminosulphonyl, and
R⁶ and R⁷ independently of one another represent hydrogen, C₁-C₃-alkyl, cyclopropyl or di-C₁-C₃-alkylamino-C₁-C₃-alkyl, and
R⁸ represents hydroxy, C₁-C₃-alkyl, hydroxy-C₁-C₃-alkyl, trifluoromethyl, pyrrolidinyl, morpholinyl or piperidinyl, and
R⁹ represents C₁-C₄-alkyl or C₁-C₄-alkoxy, and their polymorphs, tautomers, solvates, physiologically acceptable salts and solvates of these salts, and
where the stereocentre, which is represented by the carbon atom of the benzodiazepine skeleton which is bound to R², is present either in racemic form or predominantly or completely in the (S) configuration;
(k) a compound of general formula (I) wherein
X represents an oxygen atom, and
A represents a phenyl ring, and
R^{1a} represents piperazinyl, pyrrolidinyl, piperidinyl, diazepanyl, oxazinanyl, oxazolidinyl, morpholinyl, thiomorpholinyl, thiazolidinyl or azetidinyl, which may optionally be mono- or polysubstituted by identical or different substituents from the group consisting of fluorine, hydroxy, oxo, C₁-C₃-alkyl, methoxy, hydroxy-C₁-C₃-alkyl, dimethylamino, difluoroethyl, trifluoroethyl, benzyl, —NR⁶C(═O)—R⁹, —C(═O)—NR⁶R⁷, —C(═O)—R⁸ and/or
—S(═O)₂—R⁹,
or
represents isoxazolyl or pyrazolyl, which may optionally be substituted one or more times by identical or different C₁-C₂-alkyls, and
R^{1b} represents hydrogen, fluorine, bromine or cyano, and
R^{1c} represents hydrogen, and
R² represents methyl, and
R³ represents methylamino, and
R⁴ and R⁵ independently of one another represent hydrogen, hydroxy, cyano, chlorine, C₁-C₆-alkyl, methoxy, ethoxy or C₁-C₃-alkylcarbonylamino,
or
represent difluoromethoxy or trifluoromethoxy, and
R⁶ and R⁷ independently of one another represent hydrogen, C₁-C₃-alkyl, cyclopropyl or di-C₁-C₃-alkylamino-C₁-C₃-alkyl, and
R⁸ represents hydroxy, C₁-C₃-alkyl, hydroxy-C₁-C₃-alkyl, trifluoromethyl, pyrrolidinyl, morpholinyl or piperidinyl, and
R⁹ represents C₁-C₄-alkyl or C₁-C₄-alkoxy, and their polymorphs, tautomers, solvates, physiologically acceptable salts and solvates of these salts, and
where the stereocentre, which is represented by the carbon atom of the benzodiazepine skeleton which is bound to R², is present either in racemic form or predominantly or completely in the (S) configuration; and
(1) a compound of general formula (I) wherein
X represents an oxygen atom, and
A represents a phenyl ring, and
R^{1a} represents piperazinyl, pyrrolidinyl, piperidinyl, morpholinyl, thiomorpholinyl or azetidinyl, which may optionally be mono- or polysubstituted by identical or different substituents from the group consisting of hydroxy, oxo, C₁-C₃-alkyl, methoxy, dimethylamino, difluoroethyl, trifluoroethyl, —NR⁶C(═O)—R⁹, —C(═O)—NR⁶R⁷ and/or
—C(═O)—R⁸, and
R^{1b} represents hydrogen, fluorine, bromine or cyano, and
R^{1c} represents hydrogen, and
R² represents methyl, and
R³ represents methylamino, and
R⁴ and R⁵ independently of one another represent hydrogen, chlorine, methoxy or ethoxy,
or
represent difluoromethoxy or trifluoromethoxy, and
R⁶ and R⁷ independently of one another represent hydrogen or C₁-C₃-alkyl, and
R⁸ represents methyl, and
R⁹ represents methyl, and their polymorphs, tautomers, solvates, physiologically acceptable salts and solvates of these salts, and
where the stereocentre, which is represented by the carbon atom of the benzodiazepine skeleton which is bound to R², is present either in racemic form or predominantly or completely in the (S) configuration.

11-21 (canceled)

22. An in vitro method for stratifying a melanoma disease according to claim 1, wherein the inhibitor is one of the following a compounds:
(±)-1-(4-chlorophenyl)-N,4-dimethyl-8-(trifluoromethoxy)-4,5-dihydro-3H-2,3-benzodiazepine-3-carboxamide;
(4S)-1-(4-chlorophenyl)-N,4-dimethyl-8-(trifluoromethoxy)-4,5-dihydro-3H-2,3-benzodiazepine-3-carboxamide;
(4R)-1-(4-chlorophenyl)-N,4-dimethyl-8-(trifluoromethoxy)-4,5-dihydro-3H-2,3-benzodiazepine-3-carboxamide;
(±)-1-[4-(3,5-dimethyl-4-isoxazolyl)phenyl]-7,8-dimethoxy-N,4-dimethyl-4,5-dihydro-3H-2,3-benzodiazepine-3-carboxamide;
(4R)-1-[4-(3,5-dimethyl-4-isoxazolyl)phenyl]-7,8-dimethoxy-N,4-dimethyl-4,5-dihydro-3H-2,3-benzodiazepine-3-carboxamide;
(4S)-1-[4-(3,5-dimethyl-4-isoxazolyl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[4-(1H-pyrazol-3-yl)phenyl]-4,5-dihydro-3H-2,3-benzodiazepine-3-carboxamide;
(±)-1-[4-(2-chloropyridin-3-yl)phenyl]-7,8-dimethoxy-N,4-dimethyl-4,5-dihydro-3H-2,3-benzodiazepine-3-carboxamide;
(±)-5-(4-{7,8-dimethoxy-4-methyl-3-[(methylamino)carbonyl]-4,5-dihydro-3H-2,3-benzodiazepin-1-yl}phenyl)thiophene-2-carboxylic acid;
(±)-4 ′-{7,8-dimethoxy-4-methyl-3-Rmethylamino)carbonyll -4,5-dihydro-3H-2,3-benzodiazepin-1-yl}biphenyl-2-carboxylic acid;
(±)-7,8-dimethoxy-N,4-dimethyl-1-[4-(morpholin-4-yl)phenyl]-4,5-dihydro-3H-2,3-benzodiazepine-3-carboxamide;
(±)-1-(4-chlorophenyl)-N,4-dimethyl-8-(pyridin-4-yl)-4,5-dihydro-3H-2,3-benzodiazepine-3-carboxamide;
(±)-1-(4-chlorophenyl)-8-cyclopropyl-N,4-dimethyl-4,5-dihydro-3H-2,3-benzodiazepine-3-carboxamide;
(±)-1-(4-chlorophenyl)-N,4-dimethyl-8-{4-[(methylamino)sulphonyl]phenyl}-4,5-dihydro-3H-2,3-benzodiazepine-3-carboxamide;
(±)-1-(4-chlorophenyl)-N,4-dimethyl-8-(morpholin-4-yl)-4,5-dihydro-3H-2,3-benzodiazepine-3-carboxamide;
(4S)-1-(4-chlorophenyl)-N,4-dimethyl-8-(morpholin-4-yl)-4,5-dihydro-3H-2,3-benzodiazepine-3-carboxamide;
(4R)-1-(4-chlorophenyl)-N,4-dimethyl-8-(morpholin-4-yl)-4,5-dihydro-3H-2,3-benzodiazepine-3-carboxamide;
(±)-1-(4-chlorophenyl)-N,4-dimethyl-8-(4-methylpiperazin-1-yl)-4,5-dihydro-3H-2,3-benzodiazepine-3-carboxamide;
(±)-1-(4-chlorophenyl)-N,4-dimethyl-8-(piperidin-1-yl)-4,5-dihydro-3H-2,3-benzodiazepine-3-carboxamide;
(±)-8-methoxy-N,4-dimethyl-1-(pyridin-3-yl)-4,5-dihydro-3H-2,3-benzodiazepine-3-carboxamide;
(±)-7-chloro-1-(4-chlorophenyl)-N,4-dimethyl-8-(trifluoromethoxy)-4,5-dihydro-3H-2,3-benzodiazepine-3-carboxamide;
7-chloro-1-(4-chlorophenyl)-N,4-dimethyl-8-(trifluoromethoxy)-4,5-dihydro-3H-2,3-benzodiazepine-3-carboxamide, enantiomer 1;
(4S)-1-[4-(3,5-dimethyl-1H-pyrazol-1-yl)phenyl]-8-methoxy-N,4-dimethyl-4,5-dihydro-3H-2,3-benzodiazepine-3-carboxamide;
(4S)-8-methoxy-N,4-dimethyl-1-[4-(morpholin-4-yl)phenyl]-4,5-dihydro-3H-2,3-benzodiazepine-3-carboxamide;
(4S)-1-[4-(4-isoxazolyl)phenyl]-8-methoxy-N,4-dimethyl-4,5-dihydro-3H-2,3-benzodiazepine-3-carboxamide;
(4S)-8-methoxy-N,4-dimethyl-1-[4-(1-methyl-1H-pyrazol-5-yl)phenyl]-4,5-dihydro-3H-2,3-benzodiazepine-3-carboxamide;
(4S)-1-[4-(3,5-dimethyl-4-isoxazolyl)phenyl]-8-methoxy-N,4-dimethyl-4,5-dihydro-3H-2,3-benzodiazepine-3-carboxamide;
(4S)-8-methoxy-N,4-dimethyl-1-[4-(1-methyl-1H-pyrazol-4-yl)phenyl]-4,5-dihydro-3H-2,3-benzodiazepine-3-carboxamide;
(4S)-8-methoxy-N,4-dimethyl-1-[4-(1,3,5-trimethyl-1H-pyrazol-4-yl)phenyl]-4,5-dihydro-3H-2,3-benzodiazepine-3-carboxamide;
(4S)-8-methoxy-N,4-dimethyl-1-[4-(1H-pyrazol-5-yl)phenyl]-4,5-dihydro-3H-2,3-benzodiazepine-3-carboxamide;
(4S)-1-[4-(3-cyclopropyl-5-ethyl-1H-pyrazol-1-yl)phenyl]-8-methoxy-N,4-dimethyl-4,5-dihydro-3H-2,3-benzodiazepine-3-carboxamide;
(4S)-1-[4-(5-cyclopropyl-3-ethyl-1H-pyrazol-1-yl)phenyl]-8-methoxy-N,4-dimethyl-4,5-dihydro-3H-2,3-benzodiazepine-3-carboxamide;
(4S)-8-methoxy-1-{4-[3-(methoxymethyl)-5-methyl-1H-pyrazol-1-yl]phenyl}-N,4-dimethyl-4,5-dihydro-3H-2,3-benzodiazepine-3-carboxamide;
(4S)-8-methoxy-1-{4-[5-(methoxymethyl)-3-methyl-1H-pyrazol-1-yl]phenyl}-N,4-dimethyl-4,5-dihydro-3H-2,3-benzodiazepine-3-carboxamide;
(4S)-1-{4-[5-cyclopropyl-3-(pyridin-2-yl)-1H-pyrazol-1-yl]phenyl}-8-methoxy-N,4-dimethyl-4,5-dihydro-3H-2,3-benzodiazepine-3-carboxamide;
(4S)-1-{4-[3-cyclopropyl-5-(pyridin-2-yl)-1H-pyrazol-1-yl]phenyl}-8-methoxy-N,4-dimethyl-4,5-dihydro-3H-2,3-benzodiazepine-3-carboxamide;
(4S)-8-methoxy-N,4-dimethyl-1-[4-(1H-tetrazol-1-yl)phenyl]-4,5-dihydro-3H-2,3-benzodiazepine-3-carboxamide;
(±)-1-[3-(3,5-dimethylisoxazol-4-yl)phenyl]-7,8-dimethoxy-N,4-dimethyl-4,5-dihydro-3H-2,3-benzodiazepine-3-carboxamide;
(±)-1-(4-chlorophenyl)-N,4-dimethyl-8-[2-(morpholin-4-yl)ethoxy]-4,5-dihydro-3H-2,3-benzodiazepine-3-carboxamide;
(±)-1-(4-chlorophenyl)-N,4-dimethyl-8-[2-(pyrrolidin-1-yl)ethoxy]-4,5-dihydro-3H-2,3-benzodiazepine-3-carboxamide;
(±)-7,8-dimethoxy-N,4-dimethyl-1-[4-(2-oxooxazolidin-3-yl)phenyl]-4,5-dihydro-3H-2,3-benzodiazepine-3-carboxamide;
(4S)-7,8-dimethoxy-N,4-dimethyl-1-[4-(2-oxooxazolidin-3-yl)phenyl]-4,5-dihydro-3H-2,3-benzodiazepine-3-carboxamide;
(±)-7,8-dimethoxy-N,4-dimethyl-1-[4-(2-oxopiperidin-1-yl)phenyl]-4,5-dihydro-3H-2,3-benzodiazepine-3-carboxamide;
(±)-1-[4-(4-benzyl-2-oxopiperazin-1-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[4-(4-methyl-2-oxo-1,4-diazepan-1-yl)phenyl]-4,5-dihydro-3H-2,3-benzodiazepine-3-carboxamide;
(±)-7,8-dimethoxy-N,4-dimethyl-1-[4-(2-oxo-1,3-oxazinan-3-yl)phenyl]-4,5-dihydro-3H-2,3-benzodiazepine-3-carboxamide;
(±)-7,8-dimethoxy-N,4-dimethyl-1-[4-(2-oxopyrrolidin-1-yl)phenyl]-4,5-dihydro-3H-2,3-benzodiazepine-3-carboxamide;
(±)-7,8-dimethoxy-N,4-dimethyl-1-[4-(3-oxomorpholin-4-yl)phenyl]-4,5-dihydro-3H-2,3-benzodiazepine-3-carboxamide;
(4S)-7,8-dimethoxy-N,4-dimethyl-1-[4-(3-oxomorpholin-4-yl)phenyl]-4,5-dihydro-3H-2,3-benzodiazepine-3-carboxamide;
(±)-7,8-dimethoxy-N,4-dimethyl-1-[4-(2-methyl-5-oxomorpholin-4-yl)phenyl]-4,5-dihydro-3H-2,3-benzodiazepine-3-carboxamide (stereoisomer mixture);
(±)-7,8-dimethoxy-N,4-dimethyl-1-[4-(2-methyl-3-oxomorpholin-4-yl)phenyl]-4,5-dihydro-3H-2,3-benzodiazepine-3-carboxamide (stereoisomer mixture);
(±)-7,8-dimethoxy-N,4-dimethyl-1-[4-(4-methyl-2-oxopiperazin-1-yl)phenyl]-4,5-dihydro-3H-2,3-benzodiazepine-3-carboxamide;
(4S)-7,8-dimethoxy-N,4-dimethyl-1-[4-(4-methyl-2-oxopiperazin-1-yl)phenyl]-4,5-dihydro-3H-2,3-benzodiazepine-3-carboxamide;
(4R)-7,8-dimethoxy-N,4-dimethyl-1-[4-(4-methyl-2-oxopiperazin-1-yl)phenyl]-4,5-dihydro-3H-2,3-benzodiazepine-3-carboxamide;
(±)-7,8-dimethoxy-N,4-dimethyl-1-[4-(2-oxopiperazin-1-yl)phenyl]-4,5-dihydro-3H-2,3-benzodiazepine-3-carboxamide;
(±)-7,8-dimethoxy-N,4-dimethyl-1-[4-(1-methyl-1H-1,2,3-triazol-4-yl)phenyl]-4,5-dihydro-3H-2,3-benzodiazepine-3-carboxamide;
(4S)-7,8-dimethoxy-N,4-dimethyl-1-[4-(1-methyl-1H-1,2,3-triazol-4-yl)phenyl]-4,5-dihydro-3H-2,3-benzodiazepine-3-carboxamide;
(±)-1-[4-(2,4-dimethylthiazol-5-yl)phenyl]-7,8-dimethoxy-N,4-dimethyl-4,5-dihydro-3H-2,3-benzodiazepine-3-carboxamide;
(4S)-1-[4-(2,4-dimethylthiazol-5-yl)phenyl]-7,8-dimethoxy-N,4-dimethyl-4,5-dihydro-3H-2,3-benzodiazepine-3-carboxamide;
(4R)-1-[4-(2,4-dimethylthiazol-5-yl)phenyl]-7,8-dimethoxy-N,4-dimethyl-4,5-dihydro-3H-2,3-benzodiazepine-3-carboxamide;
(±)-1-[4-(1,2-dimethyl-1H-imidazol-5-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-{4-[2-(trifluoromethyl)pyridin-3-yl]phenyl}-4,5-dihydro-3H-2,3-benzodiazepine-3-carboxamide;
(±)-1-[4-(6-hydroxypyridin-3-yl)phenyl]-7,8-dimethoxy-N,4-dimethyl-4,5-dihydro-3H-2,3-benzodiazepine-3-carboxamide;
(4S)-1-[4-(6-hydroxypyridin-3-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-{4-[6-(trifluoromethyl)pyridin-3-yl]phenyl}-4,5-dihydro-3H-2,3-benzodiazepine-3-carboxamide;
(±)-7,8-dimethoxy-N,4-dimethyl-1-[4-(1,3,5-trimethyl-1H-pyrazol-4-yl)phenyl]-4,5-dihydro-3H-2,3-benzodiazepine-3-carboxamide;
(4R)-7,8-dimethoxy-N,4-dimethyl-1-[4-(1,3,5-trimethyl-1H-pyrazol-4-yl)phenyl]-4,5-dihydro-3H-2,3-benzodiazepine-3-carboxamide;
(4S)-7,8-dimethoxy-N,4-dimethyl-1-[4-(1,3,5-trimethyl-1H-pyrazol-4-yl)phenyl]-4,5-dihydro-3H-2,3-benzodiazepine-3-carboxamide;
(±)-1-[4-(isoxazol-4-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[3-(1,3,5-trimethyl-1H-pyrazol-4-yl)phenyl]-4,5-dihydro-3H-2,3-benzodiazepine-3-carboxamide;
(±)-7,8-dimethoxy-N,4-dimethyl-1-[3-(1-methyl-1H-pyrazol-4-yl)phenyl]-4,5-dihydro-3H-2,3-benzodiazepine-3-carboxamide;
(±)-7,8-dimethoxy-N,4-dimethyl-1-[3-(1-methyl-1H-pyrazol-5-yl)phenyl]-4,5-dihydro-3H-2,3-benzodiazepine-3-carboxamide;
(±)-1-[4-fluoro-3-(1,3,5-trimethyl-1H-pyrazol-4-yl)phenyl]-7,8-dimethoxy-N,4-dimethyl-4,5-dihydro-3H-2,3-benzodiazepine-3-carboxamide;
(±)-1-[4-fluoro-3-(1-methyl-1H-pyrazol-4-yl)phenyl]-7,8-dimethoxy-N,4-dimethyl-4,5-dihydro-3H-2,3-benzodiazepine-3-carboxamide;
(±)-1-[3-(3,5-dimethylisoxazo1-4-yl)-4-fluoroophenyl]-7,8-dimethoxy-N,4-dimethyl-4,5-dihydro-3H-2,3-benzodiazepine-3-carboxamide;
(±)-7,8-dimethoxy-N,4-dimethyl-1-(3′-nitrobiphenyl-4-yl)-4,5-dihydro-3H-2,3-benzodiazepine-3-carboxamide;
(±)-1-(biphenyl-4-yl)-7,8-dimethoxy-N,4-dimethyl-4,5-dihydro-3H-2,3-benzodiazepine-3-carboxamide;
(±)-1-(2′,4′-dichlorobiphenyl-4-yl)-7,8-dimethoxy-N,4-dimethyl-4,5-dihydro-3H-2,3-benzodiazepine-3-carboxamide;
(±)-1-(4′-fluorobiphenyl-4-yl)-7,8-dimethoxy-N,4-dimethyl-4,5-dihydro-3H-2,3-benzodiazepine-3-carboxamide;
(±)-1-(4′-chlorobiphenyl-4-yl)-7,8-dimethoxy-N,4-dimethyl-4,5-dihydro-3H-2,3-benzodiazepine-3-carboxamide;
(±)-7,8-dimethoxy-N,4-dimethyl-1-(4′-methylbiphenyl-4-yl)-4,5-dihydro-3H-2,3-benzodiazepine-3-carboxamide;
(±)-7,8-dimethoxy-1-(4′-methoxybiphenyl-4-yl)-N,4-dimethyl-4,5-dihydro-3H-2,3-benzodiazepine-3-carboxamide;
(±)-7,8-dimethoxy-1-[4-(6-methoxypyridin-3-yl)phenyl]-N,4-dimethyl-4,5-dihydro-3H-2,3-benzodiazepine-3-carboxamide;
(±)-7,8-dimethoxy-N,4-dimethyl-1-[4′-(methylsulphinyl)biphenyl-4-yl]-4,5-dihydro-3H-2,3-benzodiazepine-3-carboxamide;
(±)-7,8-dimethoxy-N,4-dimethyl-1-{2′-[(methylsulphonyl)amino]biphenyl-4-yl}-4,5-dihydro-3H-2,3-benzodiazepine-3-carboxamide;
(±)-7,8-dimethoxy-N,4-dimethyl-1-[2′-(methylsulphonyl)biphenyl-4-yl]-4,5-dihydro-3H-2,3-benzodiazepine-3-carboxamide;
(±)-7,8-dimethoxy-N,4-dimethyl-1-{4′-[(methylsulphonyl)amino]biphenyl-4-yl}-4,5-dihydro-3H-2,3-benzodiazepine-3-carboxamide;
(±)-7,8-dimethoxy-N,4-dimethyl-1-{3′-[(methylsulphonyl)amino]biphenyl-4-yl}-4,5-dihydro-3H-2,3-benzodiazepine-3-carboxamide;
(±)-7,8-dimethoxy-N,4-dimethyl-1-(2′-methylbiphenyl-4-yl)-4,5-dihydro-3H-2,3-benzodiazepine-3-carboxamide;
(±)-7,8-dimethoxy-N,4-dimethyl-1-[3′-(methylsulphonyl)biphenyl-4-yl]-4,5-dihydro-3H-2,3-benzodiazepine-3-carboxamide;
(±)-7,8-dimethoxy-1-[4-(2-methoxypyrimidin-5-yl)phenyl]-N,4-dimethyl-4,5-dihydro-3H-2,3-benzodiazepine-3-carboxamide;
(±)-1-(3′-cyano-4′-fluorobiphenyl-4-yl)-7,8-dimethoxy-N,4-dimethyl-4,5-dihydro-3H-2,3-benzodiazepine-3-carboxamide;
(±)-7,8-dimethoxy-1-[4-(2-methoxypyridin-3-yl)phenyl]-N,4-dimethyl-4,5-dihydro-3H-2,3-benzodiazepine-3-carboxamide;
(±)-1-(3′-carbamoylbiphenyl-4-yl)-7,8-dimethoxy-N,4-dimethyl-4,5-dihydro-3H-2,3-benzodiazepine-3-carboxamide;
(±)-7,8-dimethoxy-N,4-dimethyl-1-[4′-(pyrrolidin-1-ylcarbonyl)biphenyl-4-yl]-4,5-dihydro-3H-2,3-benzodiazepine-3-carboxamide;
(±)-7,8-dimethoxy-N,4-dimethyl-1-[4′-(morpholin-4-ylcarbonyl)biphenyl-4-yl]-4,5-dihydro-3H-2,3-benzodiazepine-3-carboxamide;
(±)-7,8-dimethoxy-1-[4-(5-methoxypyridin-3-yl)phenyl]-N,4-dimethyl-4,5-dihydro-3H-2,3-benzodiazepine-3-carboxamide;
(±)-7,8-dimethoxy-N,4-dimethyl-1-[4-(5-methylpyridin-3-yl)phenyl]-4,5-dihydro-3H-2,3-benzodiazepine-3-carboxamide;
(±)-7,8-dimethoxy-N,4-dimethyl-1-[4-(4-methylpyridin-3-yl)phenyl]-4,5-dihydro-3H-2,3-benzodiazepine-3-carboxamide;
(±)-1-[4′-(cyclopropylcarbamoyl)biphenyl-4-yl]-7,8-dimethoxy-N,4-dimethyl-4,5-dihydro-3H-2,3-benzodiazepine-3-carboxamide;
(±)-1-[4-(3-fluoropyridin-4-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[3′-(trifluoromethyl)biphenyl-4-yl]-4,5-dihydro-3H-2,3-benzodiazepine-3-carboxamide;
(±)-7,8-dimethoxy-N,4-dimethyl-1-[4′-(trifluoromethyl)biphenyl-4-yl]-4,5-dihydro-3H-2,3-benzodiazepine-3-carboxamide;
(±)-7,8-dimethoxy-1-(3′-methoxybiphenyl-4-yl)-N,4-dimethyl-4,5-dihydro-3H-2,3-benzodiazepine-3-carboxamide;
(±)-1-[4′-(5-chlorothien-2-yl)biphenyl-4-yl]-7,8-dimethoxy-N,4-dimethyl-4,5-dihydro-3H-2,3-benzodiazepine-3-carboxamide;
(±)-1-(3′-fluorobiphenyl-4-yl)-7,8-dimethoxy-N,4-dimethyl-4,5-dihydro-3H-2,3-benzodiazepine-3-carboxamide;
(±)-7,8-dimethoxy-1-(2′-methoxybiphenyl-4-yl)-N,4-40dimethyl-4,5-dihydro-3H-2,3-benzodiazepine-3-carboxamide;
(±)-7,8-dimethoxy-N,4-dimethyl-1-[2′-(trifluoromethyl)biphenyl-4-yl]-4,5-dihydro-3H-2,3-benzodiazepine-3-carboxamide;
(±)-1-(2′-chlorobiphenyl-4-yl)-7,8-dimethoxy-N,4-dimethyl-4,5-dihydro-3H-2,3-benzodiazepine-3-carboxamide;
(±)-1-(2′-fluorobiphenyl-4-yl)-7,8-dimethoxy-N,4-dimethyl-4,5-dihydro-3H-2,3-benzodiazepine-3-carboxamide;
(±)-1-[4′-(hydroxymethyl)biphenyl-4-yl]-7,8-dimethoxy-N,4-dimethyl-4,5-dihydro-3H-2,3-benzodiazepine-3-carboxamide;
(±)-7,8-dimethoxy-N,4-dimethyl-1-[4′-(trifluoromethoxy)biphenyl-4-yl]-4,5-dihydro-3H-2,3-benzodiazepine-3-carboxamide;
(±)-7,8-dimethoxy-N,4-dimethyl-1-[3′-(pyrrolidin-1-ylcarbonyl)biphenyl-4-yl]-4,5-dihydro-3H-2,3-benzodiazepine-3-carboxamide;
(±)-7,8-dimethoxy-N,4-dimethyl-1-[3′-(piperidin-1-ylcarbonyl)biphenyl-4-yl]-4,5-dihydro-3H-2,3-benzodiazepine-3-carboxamide;
(±)-7,8-dimethoxy-N,4-dimethyl-1-[3′-(morpholin-4-ylcarbonyl)biphenyl-4-yl]-4,5-dihydro-3H-2,3-benzodiazepine-3-carboxamide;
(±)-1-[3′-(cyclopropylcarbamoyl)biphenyl-4-yl]-7,8-dimethoxy-N,4-dimethyl-4,5-dihydro-3H-2,3-benzodiazepine-3-carboxamide;
(±)-1-(2′,4′-difluorobiphenyl-4-yl)-7,8-dimethoxy-N,4-dimethyl-4,5-dihydro-3H-2,3-benzodiazepine-3-carboxamide;
(±)-7,8-dimethoxy-N,4-dimethyl-1-[4-(1-methyl-1H-pyrazol-5-yl)phenyl]-4,5-dihydro-3H-2,3-benzodiazepine-3-carboxamide;
(±)-7,8-dimethoxy-N,4-dimethyl-1-(4′-nitrobiphenyl-4-yl)-4,5-dihydro-3H-2,3-benzodiazepine-3-carboxamide;
(±)-7,8-dimethoxy-N,4-dimethyl-1-[4-(pyridin-3-yl)phenyl]-4,5-dihydro-3H-2,3-benzodiazepine-3-carboxamide;
(±)-7,8-dimethoxy-1-[4-(4-methoxypyridin-3-yl)phenyl]-N,4-dimethyl-4,5-dihydro-3H-2,3-benzodiazepine-3-carboxamide;
(±)-1-(3′-cyanobiphenyl-4-yl)-7,8-dimethoxy-N,4-dimethyl-4,5-dihydro-3H-2,3-benzodiazepine-3-carboxamide;
(±)-1-(4′-cyanobiphenyl-4-yl)-7,8-dimethoxy-N,4-dimethyl-4,5-dihydro-3H-2,3-benzodiazepine-3-carboxamide;
(±)-7,8-dimethoxy-N,4-dimethyl-1-[2′-(trifluoromethoxy)biphenyl-4-yl]-4,5-dihydro-3H-2,3-benzodiazepine-3-carboxamide;
(±)-7,8-dimethoxy-N,4-dimethyl-1-[4′-(methylsulphonyl)biphenyl-4-yl]-4,5-dihydro-3H-2,3-benzodiazepine-3-carboxamide;
(±)-1-(2′-cyanobiphenyl-4-yl)-7,8-dimethoxy-N,4-dimethyl-4,5-dihydro-3H-2,3-benzodiazepine-3-carboxamide;
(±)-7,8-dimethoxy-N,4-dimethyl-1-[4′-(morpholin-4-yl)biphenyl-4-yl]-4,5-dihydro-3H-2,3-benzodiazepine-3-carboxamide;
(±)-7,8-dimethoxy-N,4-dimethyl-1-[4-(pyrimidin-5-yl)phenyl]-4,5-dihydro-3H-2,3-benzodiazepine-3-carboxamide;
(±)-1-[2′-(hydroxymethyl)biphenyl-4-yl]-7,8-dimethoxy-N,4-dimethyl-4,5-dihydro-3H-2,3-benzodiazepine-3-carboxamide;
(±)-1-(3′-{[2-(dimethylamino)ethyl]carbamoyl }biphenyl-4-yl)-7,8-dimethoxy-N,4-dimethyl-4,5-dihydro-3H-2,3-benzodiazepine-3-carboxamide;
(±)-7,8-dimethoxy-N,4-dimethyl-1-(3′-sulphamoylbiphenyl-4-yl)-4,5-dihydro-3H-2,3-benzodiazepine-3-carboxamide;
(±)-7,8-dimethoxy-N,4-dimethyl-1-[4′-(methylsulphamoyl)biphenyl-4-yl]-4,5-dihydro-3H-2,3-benzodiazepine-3-carboxamide;
(±)-7,8-dimethoxy-N,4-dimethyl-1-[4-(1-methyl-1H-pyrrol-2-yl)phenyl]-4,5-dihydro-3H-2,3-benzodiazepine-3-carboxamide;
(±)-7,8-dimethoxy-N,4-dimethyl-1-[4-(6-methylpyridin-3-yl)phenyl]-4,5-dihydro-3H-2,3-benzodiazepine-3-carboxamide;
(±)-1-[4′-(cyclopropylsulphamoyl)biphenyl-4-yl]-7,8-dimethoxy-N,4-dimethyl-4,5-dihydro-3H-2,3-benzodiazepine-3-carboxamide;
(±)-1-(3′-fluoro-5′-hydroxybiphenyl-4-yl)-7,8-dimethoxy-N,4-dimethyl-4,5-dihydro-3H-2,3-benzodiazepine-3-carboxamide;
(±)-1-(3′-fluoro-5′-methylbiphenyl-4-yl)-7,8-dimethoxy-N,4-dimethyl-4,5-dihydro-3H-2,3-benzodiazepine-3-carboxamide;
(±)-7,8-dimethoxy-N,4-dimethyl-1-[3′-(methylsulphamoyl)biphenyl-4-yl]-4,5-dihydro-3H-2,3-benzodiazepine-3-carboxamide;
(±)-1-[4-(5-fluoropyridin-3-yl)phenyl]-7,8-dimethoxy-N,4-dimethyl-4,5-dihydro-3H-2,3-benzodiazepine-3-carboxamide;
(±)-1-[4-(4-fluoropyridin-3-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[4-(2-methylpyridin-3-yl)phenyl]-4,5-dihydro-3H-2,3-benzodiazepine-3-carboxamide;
(±)-7,8-dimethoxy-1-[4-(2-methoxypyridin-4-yl)phenyl]-N,4-dimethyl-4,5-dihydro-3H-2,3-benzodiazepine-3-carboxamide;
(±)-1-[4-(5-cyanopyridin-3-yl)phenyl]-7,8-dimethoxy-N,4-dimethyl-4,5-dihydro-3H-2,3-benzodiazepine-3-carboxamide;
(4R)-7,8-dimethoxy-N,4-dimethyl-1-[4-(morpholin-4-yl)phenyl]-4,5-dihydro-3H-2,3-benzodiazepine-3-carboxamide;
(4S)-7,8-dimethoxy-N,4-dimethyl-1-[4-(morpholin-4-yl)phenyl]-4,5-dihydro-3H-2,3-benzodiazepine-3-carboxamide;
(4S)-7,8-dimethoxy-N,4-dimethyl-1-[4-(4-methylpiperazin-1-yl)phenyl]-4,5-dihydro-3H-2,3-benzodiazepine-3-carboxamide;
(±)-1-[4-(azetidin-1-yl)phenyl]-7,8-dimethoxy-N,4-dimethyl-4,5-dihydro-3H-2,3-benzodiazepine-3-carboxamide;
(4R)-1-[4-(azetidin-1-yl)phenyl]-7,8-dimethoxy-N,4-dimethyl-4,5-dihydro-3H-2,3-benzodiazepine-3-carboxamide;
(4S)-1-[4-(azetidin-1-yl)phenyl]-7,8-dimethoxy-N,4-dimethyl-4,5-dihydro-3H-2,3-benzodiazepine-3-carboxamide;
(±)-1-[4-(3-fluoroazetidin-1-yl)phenyl]-7,8-dimethoxy-N,4-dimethyl-4,5-dihydro-3H-2,3-benzodiazepine-3-carboxamide;
(4R)-1-[4-(3-fluoroazetidin-1-yl)phenyl]-7,8-dimethoxy-N,4-dimethyl-4,5-dihydro-3H-2,3-benzodiazepine-3-carboxamide;
(4S)-1-[4-(3-fluoroazetidin-1-yl)phenyl]-7,8-dimethoxy-N,4-dimethyl-4,5-dihydro-3H-2,3-benzodiazepine-3-carboxamide;
(±)-1-[4-(4-hydroxypiperidin-1-yl)phenyl]-7,8-dimethoxy-N,4-dimethyl-4,5-dihydro-3H-2,3-benzodiazepine-3-carboxamide;
(4S)-1-[4-(4-hydroxypiperidin-1-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[4-(piperazin-1-yl)phenyl]-4,5-dihydro-3H-2,3-benzodiazepine-3-carboxamide;
(4S)-7,8-dimethoxy-N,4-dimethyl-1-[4-(piperazin-1-yl)phenyl]-4,5-dihydro-3H-2,3-benzodiazepine-3-carboxamide;
(±)-7,8-dimethoxy-N,4-dimethyl-1-[4-(4-methylpiperazin-1-yl)phenyl]-4,5-dihydro-3H-2,3-benzodiazepine-3-carboxamide;
(±)-1-[4-(4-acetylpiperazin-1-yl)phenyl]-7,8-dimethoxy-N,4-dimethyl-4,5-dihydro-3H-2,3-benzodiazepine-3-carboxamide;
(4R)-1-[4-(4-acetylpiperazin-1-yl)phenyl]-7,8-dimethoxy-N,4-dimethyl-4,5-dihydro-3H-2,3-benzodiazepine-3-carboxamide;
(4S)-1-[4-(4-acetylpiperazin-1-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-{4-[4-(trifluoroacetyl)piperazin-1-yl]phenyl}-4,5-dihydro-3H-2,3-benzodiazepine-3-carboxamide;
(±)-1-{4-[4-(2-hydroxy-2-methylpropanoyl)piperazin-1-yl]phenyl}-7,8-dimethoxy-N,4-dimethyl-4,5-dihydro-3H-2,3-benzodiazepine-3-carboxamide;
(±)-7,8-dimethoxy-N,4-dimethyl-1-{4-[4-(methylsulphonyl)piperazin-1-yl]phenyl}-4,5-dihydro-3H-2,3-benzodiazepine-3-carboxamide;
(4S)-1-[4-(1,1-dioxidothiomorpholin-4-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[4-(3-oxopiperazin-1-yl)phenyl]-4,5-dihydro-3H-2,3-benzodiazepine-3-carboxamide;
(±)-7,8-dimethoxy-N,4-dimethyl-1-[4-(4-methyl-3-oxopiperazin-1-yl)phenyl]-4,5-dihydro-3H-2,3-benzodiazepine-3-carboxamide;
(±)-7,8-dimethoxy-N,4-dimethyl-1-[4-(piperidin-1-yl)phenyl]-4,5-dihydro-3H-2,3-benzodiazepine-3-carboxamide;
(±)-7,8-dimethoxy-N,4-dimethyl-1-[3-(morpholin-4-yl)phenyl]-4,5-dihydro-3H-2,3-benzodiazepine-3-carboxamide;
(±)-1-[3-(3,3-difluoroazetidin-1-yl)phenyl]-7,8-dimethoxy-N,4-dimethyl-4,5-dihydro-3H-2,3-benzodiazepine-3-carboxamide;
(±)-1-[3-(azetidin-1-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[3-(4-methylpiperazin-1-yl)phenyl]-4,5-dihydro-3H-2,3-benzodiazepine-3-carboxamide;
(±)-1-[4-fluoro-3-(morpholin-4-yl)phenyl]-7,8-dimethoxy-N,4-dimethyl-4,5-dihydro-3H-2,3-benzodiazepine-3-carboxamide;
(±)-1-[3-(3,3-difluoroazetidin-1-yl)-4-fluorophenyl]-7,8-dimethoxy-N,4-dimethyl-4,5-dihydro-3H-2,3-benzodiazepine-3-carboxamide;
(±)-1-[4-fluoro-3-(4-hydroxypiperidin-1-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-[4-(5-methyl-3-phenyl-1H-pyrazol-1-yl)phenyl]-4,5-dihydro-3H-2,3-benzodiazepine-3-carboxamide;
(4S)-7,8-dimethoxy-N,4-dimethyl-1-[4-(5-methyl-3-phenyl-1H-pyrazol-1-yl)phenyl]-4,5-dihydro-3H-2,3-benzodiazepine-3-carboxamide;
(±)-1-[4-(5-cyclopropyl-3-phenyl-1H-pyrazol-1-yl)phenyl]-7,8-dimethoxy-N,4-dimethyl-4,5-dihydro-3H-2,3-benzodiazepine-3-carboxamide;
(4S)-1-[4-(5-cyclopropyl-3-phenyl-1H-pyrazol-1-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-{4-[3-phenyl-5-(trifluoromethyl)-1H-pyrazol-1-yl]phenyl}-4,5-dihydro-3H-2,3-benzodiazepine-3-carboxamide;
(±)-1-{4-[3-(4-fluorophenyl)-1H-pyrazol-1-yl]phenyl}-7,8-dimethoxy-N,4-dimethyl-4,5-dihydro-3H-2,3-benzodiazepine-3-carboxamide;
(4S)-1-{4-[3-(4-fluorophenyl)-1H-pyrazol-1-yl]phenyl}-7,8-dimethoxy-N,4-dimethyl-4,5-dihydro-3H-2,3-benzodiazepine-3-carboxamide;
(4R)-1-{4-[3-(4-fluorophenyl)-1H-pyrazol-1-yl]phenyl}-7,8-dimethoxy-N,4-dimethyl-4,5-dihydro-3H-2,3-benzodiazepine-3-carboxamide;
(±)-7,8-dimethoxy-N,4-dimethyl-1-{4-[5-methyl-3-(trifluoromethyl)-1H-pyrazol-1-yl]phenyl}-4,5-dihydro-3H-2,3-benzodiazepine-3-carboxamide;
(±)-7,8-dimethoxy-N,4-dimethyl-1-[4-(1H-1,2,4-triazol-1-yl)phenyl]-4,5-dihydro-3H-2,3-benzodiazepine-3-carboxamide;
(±)-7,8-dimethoxy-N,4-dimethyl-1-[4-(5-methyl-1H-1,2,4-triazol-1-yl)phenyl]-4,5-dihydro-3H-2,3-benzodiazepine-3-carboxamide;
(±)-1-[4-(3,5-dimethyl-1H-1,2,4-triazol-1-yl)phenyl]-7,8-dimethoxy-N,4-dimethyl-4,5-dihydro-3H-2,3-benzodiazepine-3-carboxamide;
(±)-8-tert-butyl-1-[4-(3,5-dimethylisoxazol-4-yl)phenyl]-N,4-dimethyl-4,5-dihydro-3H-2,3-benzodiazepine-3-carboxamide;
(±)-7-chloro-1-[4-(3,5-dimethylisoxazol-4-yl)phenyl]-N,4-dimethyl-8-(trifluoromethoxy)-4,5-dihydro-3H-2,3-benzodiazepine-3-carboxamide;
(4S)-7-chloro-1-[4-(3,5-dimethylisoxazol-4-yl)phenyl]-N,4-dimethyl-8-(trifluoromethoxy)-4,5-dihydro-3H-2,3-benzodiazepine-3-carboxamide;
(4R)-7-chloro-1-[4-(3,5-dimethylisoxazol-4-yl)phenyl]-N,4-dimethyl-8-(trifluoromethoxy)-4,5-dihydro-3H-2,3-benzodiazepine-3-carboxamide;
(±)-8-chloro-1-[4-(3,5-dimethylisoxazol-4-yl)phenyl]-7-methoxy-N,4-dimethyl-4,5-dihydro-3H-2,3-benzodiazepine-3-carboxamide;
(4S)-8-chloro-1-[4-(3,5-dimethylisoxazol-4-yl)phenyl]-7-methoxy-N,4-dimethyl-4,5-dihydro-3H-2,3-benzodiazepine-3-carboxamide;
(4R)-8-chloro-1-[4-(3,5-dimethylisoxazol-4-yl)phenyl]-7-methoxy-N,4-dimethyl-4,5-dihydro-3H-2,3-benzodiazepine-3-carboxamide;
(±)-1-[4-(3,5-dimethylisoxazol-4-yl)phenyl]-N,4,8-trimethyl-4,5-dihydro-3H-2,3-benzodiazepine-3-carboxamide;
(±)-7,8-bis(difluoromethoxy)-1-[4-(3,5-dimethylisoxazol-4-yl)phenyl]-N,4-dimethyl-4,5-dihydro-3H-2,3-benzodiazepine-3-carboxamide;
(±)-1-[4-(3,5-dimethylisoxazol-4-yl)phenyl]-7,8-diethoxy-N,4-dimethyl-4,5-dihydro-3H-2,3-benzodiazepine-3-carboxamide;
(±)-7-(difluoromethoxy)-1-[4-(3,5-dimethylisoxazol-4-yl)phenyl]-8-methoxy-N,4-dimethyl-4,5-dihydro-3H-2,3-benzodiazepine-3-carboxamide;
(4S)-7-(difluoromethoxy)-1-[4-(3,5-dimethylisoxazol-4-yl)phenyl]-8-methoxy-N,4-dimethyl-4,5-dihydro-3H-2,3-benzodiazepine-3-carboxamide;
(4R)-7-(difluoromethoxy)-1-[4-(3,5-dimethylisoxazol-4-yl)phenyl]-8-methoxy-N,4-dimethyl-4,5-dihydro-3H-2,3-benzodiazepine-3-carboxamide;
(±)-7-(difluoromethoxy)-8-methoxy-N,4-dimethyl-1-[4-(4-methylpiperazin-1-yl)phenyl]-4,5-dihydro-3H-2,3-benzodiazepine-3-carboxamide;
(4R)-7-(difluoromethoxy)-8-methoxy-N,4-dimethyl-1-[4-(4-methylpiperazin-1-yl)phenyl]-4,5-dihydro-3H-2,3-benzodiazepine-3-carboxamide;
(4S)-7-(difluoromethoxy)-8-methoxy-N,4-dimethyl-1-[4-(4-methylpiperazin-1-yl)phenyl]-4,5-dihydro-3H-2,3-benzodiazepine-3-carboxamide;
(±)-1-(4-chlorophenyl)-N,4-dimethyl-8-[3-(4-methylpiperazin-1-yl)propoxy]-4,5-dihydro-3H-2,3-benzodiazepine-3-carboxamide;
(4S)-1-(4-chlorophenyl)-N,4-dimethyl-8-[3-(4-methylpiperazin-1-yl)propoxy]-4,5-dihydro-3H-2,3-benzodiazepine-3-carboxamide;
(±)-1-(4-chlorophenyl)-N,4-dimethyl-8-[3-(morpholin-4-yl)propoxy]-4,5-dihydro-3H-2,3-benzodiazepine-3-carboxamide;
(±)-1-(4-chlorophenyl)-N,4-dimethyl-8-[2-(4-methylpiperazin-1-yl)ethoxy]-4,5-dihydro-3H-2,3-benzodiazepine-3-carboxamide;
(±)-1-(4-chlorophenyl)-N,4-dimethyl-8-[(6-methylpyridin-2-yl)methoxy]-4,5-dihydro-3H-2,3-benzodiazepine-3-carboxamide;
(±)-1-[4-(3,5-dimethylisoxazol-4-yl)phenyl]-8-hydroxy-N,4-dimethyl-4,5-dihydro-3H-2,3-benzodiazepine-3-carboxamide;
(±)-1-[4-(3,5-dimethylisoxazol-4-yl)phenyl]-N,4-dimethyl-8-[3-(morpholin-4-yl)propoxy]-4,5-dihydro-3H-2,3-benzodiazepine-3-carboxamide;
(±)-7-cyano-1-[4-(3,5-dimethylisoxazol-4-yl)phenyl]-8-methoxy-N,4-dimethyl-4,5-dihydro-3H-2,3-benzodiazepine-3-carboxamide;
(±)-8-acetamido-N,4-dimethyl-1-[4-(morpholin-4-yl)phenyl]-4,5-dihydro-3H-2,3-benzodiazepine-3-carboxamide;
(±)-8-acetamido-N,4-dimethyl-1-[4-(4-methylpiperazin-1-yl)phenyl]-4,5-dihydro-3H-2,3-benzodiazepine-3-carboxamide;
(±)-8-acetamido-1-[4-(3,5-dimethylisoxazol-4-yl)phenyl]-N,4-dimethyl-4,5-dihydro-3H-2,3-benzodiazepine-3-carboxamide;
(±)-1-(4-chlorophenyl)-8-(3,5-dimethyl-1H-pyrazol-1-yl)-N,4-dimethyl-4,5-dihydro-3H-2,3-benzodiazepine-3-carboxamide;
(±)-1-[4-(3,5-dimethylisoxazol-4-yl)phenyl]-8-(3,5-dimethyl-1H-p yrazol-1-yl)-N,4-dimethyl-4,5-dihydro-3H-2,3-benzodiazepine-3-carboxamide;
(±)-1-[4-(3,5-dimethylisoxazol-4-yl)phenyl]-N,4-dimethyl-8-(morpholin-4-yl)-4,5-dihydro-3H-2,3-benzodiazepine-3-c arbox amide ;
(4R)-1-[4-(3,5-dimethyli sox azol-4-yl)phenyl]-N,4-dimethyl-8-(morpholin-4-yl)-4,5-dihydro-3H-2,3-benzodiazepine-3-c arbox amide ;
(4S)-1-[4-(3,5-dimethylisoxazol-4-yl)phenyl]-N,4-dimethyl-8-(morpholin-4-yl)-4,5-dihydro-3H-2,3-benzodiazepine-3-c arbox amide ;
(4S)-8-methoxy-N,4-dimethyl-1-[4-(3-oxomorpholin-4-yl)phenyl]-4,5-dihydro-3H-2,3-benzodiazepine-3-carboxamide;
(±)-1-{4-[4-(2-hydroxyethyl)piperazin-1-yl]phenyl}-7,8-dimethoxy-N,4-dimethyl-4,5-dihydro-3H-2,3-benzodiazepine-3-carboxamide;
(±)-7,8-dimethoxy-1-[4-(4-methoxypiperidin-1-yl)phenyl]-N,4-dimethyl-4,5-dihydro-3H-2,3-benzodiazepine-3-c arbox amide ;
(4R)-7,8-dimethoxy-1-[4-(4-methoxypiperidin-1-yl)phenyl]-N,4-dimethyl-4,5-dihydro-3H-2,3-benzodiazepine-3-c arbox amide ;
(4S)-7,8-dimethoxy-1-[4-(4-methoxypiperidin-1-yl)phenyl]-N,4-dimethyl-4,5-dihydro-3H-2,3-benzodiazepine-3-c arbox amide ;
(±)-1-{4-[4-(dimethylamino)piperidin-1-yl]phenyl}-7,8-dimethoxy-N,4-dimethyl-4,5-dihydro-3H-2,3-benzodiazepine-3-carboxamide;
(4R)-1-{4-[4-(dimethylamino)piperidin-1-yl]phenyl}-7,8-dimethoxy-N,4-dimethyl-4,5-dihydro-3H-2,3-benzodiazepine-3-carboxamide;
(4S)-1-{4-[4-(dimethylamino)piperidin-1-yl]phenyl}-7,8-dimethoxy-N,4-dimethyl-4,5-dihydro-3H-2,3-benzodiazepine-3-carboxamide;
(±)-1-[4-(3,3-difluoroazetidin-1-yl)phenyl]-7,8-dimethoxy-N,4-dimethyl-4,5-dihydro-3H-2,3-benzodiazepine-3-carboxamide;
(±)-1-[4-(4-acetamidopiperidin-1-yl)phenyl]-7,8-dimethoxy-N,4-dimethyl-4,5-dihydro-3H-2,3-benzodiazepine-3-carboxamide;
(±)-1-{4-[4-(2-hydroxyethyl)piperidin-1-yl]phenyl}-7,8-dimethoxy-N,4-dimethyl-4,5-dihydro-3H-2,3-benzodiazepine-3-carboxamide;
(±)-1-[4-(3-hydroxyazetidin-1-yl)phenyl]-7,8-dimethoxy-N,4-dimethyl-4,5-dihydro-3H-2,3-benzodiazepine-3-carboxamide;
(±)-1-[4-(3-hydroxy-3-methylazetidin-1-yl)phenyl]-7,8-dimethoxy-N,4-dimethyl-4,5-dihydro-3H-2,3-benzodiazepine-3-carboxamide;
(4R)-1-[4-(3-hydroxy-3-methylazetidin-1-yl)phenyl]-7,8-dimethoxy-N,4-dimethyl-4,5-dihydro-3H-2,3-benzodiazepine-3-carboxamide;
(4S)-1-[4-(3-hydroxy-3-methylazetidin-1-yl)phenyl]-7,8-dimethoxy-N,4-dimethyl-4,5-dihydro-3H-2,3-benzodiazepine-3-carboxamide;
(±)-1-[4-(4-Isopropylpiperazin-1-yl)phenyl]-7,8-dimethoxy-N,4-dimethyl-4,5-dihydro-3H-2,3-benzodiazepine-3-carboxamide;
(±)-7,8-dimethoxy-1-[4-(3-methoxyazetidin-1-yl)phenyl]-N,4-dimethyl-4,5-dihydro-3H-2,3-benzodiazepine-3-carboxamide;
(±)-1-[4-(4-hydroxy-4-methylpiperidin-1-yl)phenyl]-7,8-dimethoxy-N,4-dimethyl-4,5-dihydro-3H-2,3-benzodiazepine-3-carboxamide;
(4R)-1-[4-(4-hydroxy-4-methylpiperidin-1-yl)phenyl]-7,8-dimethoxy-N,4-dimethyl-4,5-dihydro-3H-2,3-benzodiazepine-3-carboxamide;
(4S)-1-[4-(4-hydroxy-4-methylpiperidin-1-yl)phenyl]-7,8-dimethoxy-N,4-dimethyl-4,5-dihydro-3H-2,3-benzodiazepine-3-carboxamide;
(±)-1-[4-(4-isopropylpiperazin-1-yl)phenyl]-7,8-dimethoxy-N,4-dimethyl-4,5-dihydro-3H-2,3-benzodiazepine-3-carboxamide;
(±)-7,8-dimethoxy-1-[4-(3-methoxyazetidin-1-yl)phenyl]-N,4-dimethyl-4,5-dihydro-3H-2,3-benzodiazepine-3-carboxamide;
(±)-1-[4-(4-hydroxy-4-methylpiperidin-1-yl)phenyl]-7,8-dimethoxy-N,4-dimethyl-4,5-dihydro-3H-2,3-benzodiazepine-3-carboxamide;
(4R)-1-[4-(4-hydroxy-4-methylpiperidin-1-yl)phenyl]-7,8-dimethoxy-N,4-dimethyl-4,5-dihydro-3H-2,3-benzodiazepine-3-carboxamide;
(4S)-1-[4-(4-hydroxy-4-methylpiperidin-1-yl)phenyl]-7,8-dimethoxy-N,4-dimethyl-4,5-dihydro-3H-2,3-benzodiazepine-3-carboxamide;
(±)-7,8-dimethoxy-N,4-dimethyl-1-{4-[4-(methylcarbamoyl)piperidin-1-yl]phenyl}-4,5-dihydro-3H-2,3-benzodiazepine-3-carboxamide;
(±)-7,8-dimethoxy-N,4-dimethyl-1-{4-[4-(methylcarbamoyl)piperidin-1-yl]phenyl}-4,5-dihydro-3H-2,3-benzodiazepine-3-carboxamide;
(4R)-7,8-dimethoxy-N ,4-dimethyl-1-{4-[4-(methylcarbamoyl)piperidin-1-yl]phenyl}-4,5-dihydro-3H-2,3-benzodiazepine-3-carboxamide;
(±)-1-{4-[(3S)-3-hydroxypyrrolidin-1-yl]phenyl}-7,8-dimethoxy-N,4-dimethyl-4,5-dihydro-3H-2,3-benzodiazepine-3-carboxamide;
(4S)-1-{4-[(3S)-3-hydroxypyrrolidin-1-yl]phenyl}-7,8-dimethoxy-N,4-dimethyl-4,5-dihydro-3H-2,3-benzodiazepine-3-carboxamide;
(±)-tert-butyl (1-{4-[7,8-dimethoxy-4-methyl-3-(methylcarbamoyl)-4,5-dihydro-3H-2,3-benzodiazepin-1-yl]phenyl}-4-methylpiperidin-4-yl)carbamate;
(±)-1-{4-[(2S,5R)-2,5-dimethylpiperazin-1-yl]phenyl}-7,8-dimethoxy-N,4-dimethyl-4,5-dihydro-3H-2,3-benzodiazepine-3-carboxamide;
(±)-1-{4-[4-(2,2-difluoroethyl)piperazin-1-yl]phenyl}-7,8-dimethoxy-N,4-dimethyl-4,5-dihydro-3H-2,3-benzodiazepine-3-carboxamide;
(4S)-7,8-dimethoxy-N,4-dimethyl-1-[4-(3-oxopiperazin-1-yl)phenyl]-4,5-dihydro-3H-2,3-benzodiazepine-3-carboxamide;
(4S)-1-{4-[(2R,6S)-2,6-dimethylmorpholin-4-yl]phenyl}-7,8-dimethoxy-N,4-dimethyl-4,5-dihydro-3H-2,3-benzodiazepine-3-carboxamide;
(±)-7,8-dimethoxy-N,4-dimethyl-1-[4-(4-oxopiperidin-1-yl)phenyl]-4,5-dihydro-3H-2,3-benzodiazepine-3-carboxamide;
(±)-7,8-dimethoxy-N,4-dimethyl-1-{4-[4-(2,2,2-trifluoroethyl)piperazin-1-yl]phenyl}-4,5-dihydro-3H-2,3-benzodiazepine-3-carboxamide;
(4S)-1-{4-[(3R,5S)-3,5-dimethylpiperazin-1-yl]phenyl}-7,8-dimethoxy-N,4-dimethyl-4,5-dihydro-3H-2,3-benzodiazepine-3-carboxamide;
(±)-7,8-dihydroxy-N,4-dimethyl-1-[4-(4-methylpiperazin-1-yl)phenyl]-4,5-dihydro-3H-2,3-benzodiazepine-3-carboxamide;
(±)-7,8-diethoxy-N,4-dimethyl-1-[4-(4-methylpiperazin-1-yl)phenyl]-4,5-dihydro-3H-2,3-benzodiazepine-3-carboxamide;
(±)-4-ethyl-7,8-dimethoxy-N-methyl-1-[4-(4-methylpiperazin-1-yl)phenyl]-4,5-dihydro-3H-2,3-benzodiazepine-3-carboxamide;
(4S)-4-ethyl-7,8-dimethoxy-N-methyl-1-[4-(4-methylpiperazin-1-yl)phenyl]-4,5-dihydro-3H-2,3-benzodiazepine-3-carboxamide;
(±)-4-ethyl-7,8-dimethoxy-N-methyl-1-[4-(piperazin-1-yl)phenyl]-4,5-dihydro-3H-2,3-benzodiazepine-3-carboxamide;
(4S)-4-ethyl-7,8-dimethoxy-N-methyl-1-[4-(piperazin-1-yl)phenyl]-4,5-dihydro-3H-2,3-benzodiazepine-3-carboxamide;
(4R)-4-ethyl-7,8-dimethoxy-N-methyl-1-[4-(piperazin-1-yl)phenyl]-4,5-dihydro-3H-2,3-benzodiazepine-3-carboxamide;
(±)-4-ethyl-7,8-dimethoxy-N-methyl-1-{4-[4-(methylsulphonyl)piperazin-1-yl]phenyl}-4,5-dihydro-3H-2,3-benzodiazepine-3-carboxamide;
(4S)-4-ethyl-7,8-dimethoxy-N-methyl-1-{4-[4-(methylsulphonyl)piperazin-1-yl]phenyl}-4,5-dihydro-3H-2,3-benzodiazepine-3-carboxamide;
(4R)-4-ethyl-7,8-dimethoxy-N-methyl-1-{4-[4-(methylsulphonyl)piperazin-1-yl]phenyl}-4,5-dihydro-3H-2,3-benzodiazepine-3-carboxamide;
(±)-4-ethyl-1-[4-(3-fluorazetidin-1-yl)phenyl]-7,8-dimethoxy-N-methyl-4,5-dihydro-3H-2,3-benzodiazepine-3-carboxamide;
(±)-1-[4-(4-acetylpiperazin-1-yl)phenyl]-4-ethyl-7,8-dimethoxy-N-methyl-4,5-dihydro-3H-2,3-benzodiazepine-3-carboxamide;
(±)-1-[4-(1,1-dioxidothiomorpholin-4-yl)phenyl]-4-ethyl-7,8-dimethoxy-N-methyl-4,5-dihydro-3H-2,3-benzodiazepine-3-carboxamide;
(±)-4-ethyl-1-[4-(4-hydroxypiperidin-1-yl)phenyl]-7,8-dimethoxy-N-methyl-4,5-dihydro-3H-2,3-benzodiazepine-3-carboxamide;
(±)-4-ethyl-7,8-dimethoxy-N-methyl-1-[4-(morpholin-4-yl)phenyl]-4,5-dihydro-3H-2,3-benzodiazepine-3-carboxamide;
(4S)-4-ethyl-7,8-dimethoxy-N-methyl-1-[4-(morpholin-4-yl)phenyl]-4,5-dihydro-3H-2,3-benzodiazepine-3-carboxamide;
(±)-8-chloro-1-[4-(1,1-dioxidothiomorpholin-4-yl)phenyl]-N,4-dimethyl-7-(trifluoromethoxy)-4,5-dihydro-3H-2,3-benzodiazepine-3-carboxamide;
(±)-8-chloro-N,4-dimethyl-1-[4-(piperazin-1-yl)phenyl]-7-(trifluoromethoxy)-4,5-dihydro-3H-2,3-benzodiazepine-3-carboxamide;
(±)-8-chloro-1-[4-(4-hydroxypiperidin-1-yl)phenyl]-N,4-dimethyl-7-(trifluoromethoxy)-4,5-dihydro-3H-2,3-benzodiazepine-3-carboxamide;
(±)-8-chloro-N,4-dimethyl-1-[4-(4-methylpiperazin-1-yl)phenyl]-7-(trifluoromethoxy)-4,5-dihydro-3H-2,3-benzodiazepine-3-carboxamide;
(±)-8-chloro-N,4-dimethyl-1-{4-[4-(methylsulphonyl)piperazin-1-yl]phenyl}-7-(trifluoromethoxy)-4,5-dihydro-3H-2,3-benzodiazepine-3-carboxamide;
(±)-8-(1,1-dioxidothiomorpholin-4-yl)-1-[4-(1,1-dioxidothiomorpholin-4-yl)phenyl]-N,4-dimethyl-7-(trifluoromethoxy)-4,5-dihydro-3H-2,3-benzodiazepine-3-carboxamide;
(±)-1-(4-chlorophenyl)-8-(1,1-dioxidothiomorpholin-4-yl)-N,4-dimethyl-7-(trifluoromethoxy)-4,5-dihydro-3H-2,3-benzodiazepine-3-carboxamide;
(±)-1-[4-(4-acetylpiperazin-1-yl)phenyl]-N,4-dimethyl-7-(trifluoromethoxy)-4,5-dihydro-3H-2,3-benzodiazepine-3-carboxamide;
(±)-1-{4-[4-(2-hydroxyethyl)piperazin-1-yl]phenyl}-N,4-dimethyl-7-(trifluoromethoxy)-4,5-dihydro-3H-2,3-benzodiazepine-3-carboxamide;
(±)-8-methoxy-N,4-dimethyl-1-[4-(4-methylpiperazin-1-yl)phenyl]-7-(trifluoromethoxy)-4,5-dihydro-3H-2,3-benzodiazepine-3-carboxamide;
(±)-4-ethyl-7,8-dimethoxy-N-methyl-1-[4-(1,3,5-trimethyl-1H-pyrazol-4-yl)phenyl]-4,5-dihydro-3H-2,3-benzodiazepine-3-carboxamide;
(±)-4-ethyl-1-(4′-fluorobiphenyl-4-yl)-7,8-dimethoxy-N-methyl-4,5-dihydro-3H-2,3-benzodiazepine-3-carboxamide;
(±)-4-ethyl-7,8-dimethoxy-N-methyl-1-[4-(1-methyl-1H-1,2,3-triazol-4-yl)phenyl]-4,5-dihydro-3H-2,3-benzodiazepine-3-carboxamide;
(±)-1-[4-(3,5-dimethyl-1,2-oxazol-4-yl)phenyl]-4-ethyl-7,8-dimethoxy-N-methyl-4,5-dihydro-3H-2,3-benzodiazepine-3-carboxamide;
(±)-4-isopropyl-7,8-dimethoxy-N-methyl-1-[4-(1,3,5-trimethyl-1H-pyrazol-4-yl)phenyl]-4,5-dihydro-3H-2,3-benzodiazepine-3-carboxamide;
(±)-4-ethyl-7,8-dimethoxy-N-methyl-1-[4-(2-oxopyrrolidin-1-yl)phenyl]-4,5-dihydro-3H-2,3-benzodiazepine-3-carboxamide;
(±)-4-ethyl-7,8-dimethoxy-N-methyl-1-[4-(3-oxomorpholin-4-yl)phenyl]-4,5-dihydro-3H-2,3-benzodiazepine-3-carboxamide;
(±)-4-ethyl-7,8-dimethoxy-N-methyl-1-[4-(2-oxopiperidin-1-yl)phenyl]-4,5-dihydro-3H-2,3-benzodiazepine-3-carboxamide;
(±)-4-ethyl-7,8-dimethoxy-N-methyl-1-[4-(2-oxo-1,3-oxazolidin-3-yl)phenyl]-4,5-dihydro-3H-2,3-benzodiazepine-3-carboxamide;
(4S)-7,8-dimethoxy-N,4-dimethyl-1-[4-(4-methyl-2-oxo-1,4-diazepan-1-yl)phenyl]-4,5-dihydro-3H-2,3-benzodiazepine-3-carboxamide;
(±)-1-(4-chlorophenyl)-N,4-dimethyl-7-(2-oxo-1,3-oxazolidin-3-yl)-4,5-dihydro-3H-2,3-benzodiazepine-3-carboxamide;
(4S)-1-(4-chlorophenyl)-N,4-dimethyl-7-(2-oxo-1,3-oxazolidin-3-yl)-4,5-dihydro-3H-2,3-benzodiazepine-3-carboxamide;
(±)-1-(4-chlorophenyl)-N,4-dimethyl-7-(2-oxopiperidin-1-yl)-4,5-dihydro-3H-2,3-benzodiazepine-3-carboxamide;
(±)-1-(4-chlorophenyl)-N,4-dimethyl-7-(3-oxomorpholin-4-yl)-4,5-dihydro-3H-2,3-benzodiazepine-3-carboxamide;
(±)-1-(4-chlorophenyl)-N,4-dimethyl-7-(morpholin-4-yl)-4,5-dihydro-3H-2,3-benzodiazepine-3-carboxamide;
(±)-1-(4-chlorophenyl)-N,4-dimethyl-7-(pyrrolidin-1-yl)-4,5-dihydro-3H-2,3-benzodiazepine-3-carboxamide;
(±)-1-(4-chlorophenyl)-7-(1,1-dioxidothiomorpholin-4-yl)-N,4-dimethyl-4,5-dihydro-3H-2,3-benzodiazepine-3-carboxamide;
(±)-1-(4-chlorophenyl)-N,4-dimethyl-7-(4-methylpiperazin-1-yl)-4,5-dihydro-3H-2,3-benzodiazepine-3-carboxamide;
(±)-N,4-dimethyl-7-(4-methylpiperazin-1-yl)-1-[4-(4-methylpiperazin-1-yl)phenyl]-4,5-dihydro-3H-2,3-benzodiazepine-3-carboxamide;
(±)-N,4-dimethyl-7-(4-methyl-3-oxopiperazin-1-yl)-1-[4-(4-methyl-3-oxopiperazin-1-yl)phenyl]-4,5-dihydro-3H-2,3-benzodiazepine-3-carboxamide;
(±)-1-(4-chlorophenyl)-7-(4-fluorophenyl)-N,4-dimethyl-4,5-dihydro-3H-2,3-benzodiazepine-3-carboxamide;
(±)-1-(4-chlorophenyl)-N,4-dimethyl-7-(pyridin-4-yl)-4,5-dihydro-3H-2,3-benzodiazepine-3-carboxamide;
(±)-1-(4-chlorophenyl)-7-(6-hydroxypyridin-3-yl)-N,4-dimethyl-4,5-dihydro-3H-2,3-benzodiazepine-3-carboxamide;
(±)-1-(4-chlorophenyl)-7-(3,5-dimethyl-1,2-oxazol-4-yl)-N,4-dimethyl-4,5-dihydro-3H-2,3-benzodiazepine-3-carboxamide;
(±)-1-(4-chlorophenyl)-N,4-dimethyl-7-(1-methyl-1H-1,2,3-triazol-4-yl)-4,5-dihydro-3H-2,3-benzodiazepine-3-carboxamide;
(4R)-1-[4-(4-hydroxypiperidin-1-yl)phenyl]-7,8-dimethoxy-N,4-dimethyl-4,5-dihydro-3H-2,3-benzodiazepine-3-carboxamide;
(4R)-7,8-dimethoxy-N,4-dimethyl-1-[4-(4-methylpiperazin-1-yl)phenyl]-4,5-dihydro-3H-2,3-benzodiazepine-3-carboxamide;
(±)-1-[4-(1,1-dioxido-1,2-thiazolidin-2-yl)phenyl]-7,8-dimethoxy-N,4-dimethyl-4,5-dihydro-3H-2,3-benzodiazepine-3-carboxamide;
(±)-1-{7,8-dimethoxy-4-methyl-1-[4-(4-methylpiperazin-1-yl)phenyl]-4,5-dihydro-3H-2,3-benzodiazepin-3-yl }ethanone;
1-{(4S)-7,8-dimethoxy-4-methyl-1-[4-(4-methylpiperazin-1-yl)phenyl]-4,5-dihydro-3H-2,3-benzodiazepin-3-yl }ethanone;
(±)-1-{1-[4-(3,5-dimethyl-1,2-oxazol-4-yl)phenyl]-7,8-dimethoxy-4-methyl-4,5-dihydro-3H-2,3-benzodiazepin-3-yl}ethanone;
(±)-7,8-dimethoxy-N,4-dimethyl-1-[4-(5-methyl-1,3 ,4-oxadiazol-2-yl)phenyl]-4,5-dihydro-3H-2,3-benzodiazepine-3-carboxamide;
(±)-7,8-dimethoxy-N,4-dimethyl-1-[4-(3-methyl-1,2,4-oxadiazol-5-yl)phenyl]-4,5-dihydro-3H-2,3-benzodiazepine-3-carboxamide;
(±)-N,4-dimethyl-8-(trifluoromethoxy)-1-[441,3,5-trimethyl-1H-p yrazol-4-yl)phenyl]-4,5-dihydro-3H-2,3-benzodiazepine-3-carboxamide;
(4S)-N,4-dimethyl-8-(trifluoromethoxy)-1-[441,3,5-trimethyl-1H-p yrazol-4-yl)phenyl]-4,5-dihydro-3H-2,3-benzodiazepine-3-carboxamide;
(4R)-N,4-dimethyl-8-(trifluoromethoxy)-1-[441,3,5-trimethyl-1H-pyrazol-4-yl)phenyl]-4,5-dihydro-3H-2,3-benzodiazepine-3-carboxamide;
(±)-N,4-dimethyl-1-[4-(4-methyl-1-piperazinyl)phenyl]-8-(trifluoromethoxy)-4,5-dihydro-3H-2,3-benzodiazepine-3-carboxamide;
(4R)-N,4-dimethyl-1-[4-(4-methyl-1-piperazinyl)phenyl]-8-(trifluoromethoxy)-4,5-dihydro-3H-2,3-benzodiazepine-3-carboxamide;
(4S)-N,4-dimethyl-1-[4-(4-methyl-1-piperazinyl)phenyl]-8-(trifluoromethoxy)-4,5-dihydro-3H-2,3-benzodiazepine-3-carboxamide;
(±)-1-[4-(4-hydroxy-1-piperidinyl)phenyl]-N,4-dimethyl-8-(trifluoromethoxy)-4,5-dihydro-3H-2,3-benzodiazepine-3-carboxamide;
(±)-1-[2,4-dibromo-5-(4-methylpiperazin-1-yl)phenyl]-7,8-dimethoxy-N,4-dimethyl-4,5-dihydro-3H-2,3-benzodiazepine-3-carboxamide;
(4S)-1-[3-bromo-4-(4-methylpiperazin-1-yl)phenyl]-7,8-dimethoxy-N,4-dimethyl-4,5-dihydro-3H-2,3-benzodiazepine-3-carboxamide;
(4S)-1-[3-cyano-4-(4-methylpiperazin-1-yl)phenyl]-7,8-dimethoxy-N,4-dimethyl-4,5-dihydro-3H-2,3-benzodiazepine-3-carboxamide;
(±)-7,8-dimethoxy-4-methyl-1-[4-(4-methylpiperazin-1-yl)phenyl]-3-(1-oxopropyl)-4,5-dihydro-3H-2,3-benzodiazepine;
(±)-3-(cyclopropylcarbonyl)-7,8-dimethoxy-4-methyl-1-[4-(4-methylpiperazin-1-yl)phenyl]-4,5-dihydro-3H-2,3-benzodiazepine;
(±)-N-cyclopropyl-7,8-dimethoxy-4-methyl-1-[4-(4-methylpiperazin-1-yl)phenyl]-4,5-dihydro-3H-2,3-benzodiazepine;
(±)-7,8-dimethoxy-N,4-dimethyl-1-[4-(4-methylpiperazin-1-yl)phenyl]-4,5-dihydro-3H-2,3-benzodiazepin-3-carbothioamide;
methyl (±)-7,8-dimethoxy-4-methyl-1-[4-(4-methylpiperazin-1-yl)phenyl]-4,5-dihydro-3H-2,3-benzodiazepine-3-carboxylate;
ethyl (±)-7,8-dimethoxy-4-methyl-1-[4-(4-methylpiperazin-1-yl)phenyl]-4,5-dihydro-3H-2,3-benzodiazepine-3-carboxylate;
(±)-N-ethyl-7,8-dimethoxy-4-methyl-1-[4-(4-methylpiperazin-1-yl)phenyl]-4,5-dihydro-3H-2,3-benzodiazepine-3-carboxamide
and
(4S)-N-ethyl-7,8-dimethoxy-4-methyl-1-[4-(4-methylpiperazin-1-yl)phenyl]-4,5-dihydro-3H-2,3-benzodiazepine-3-carboxamide.

23. An in vitro method for stratifying melanoma according to claim 1, wherein the inhibitor is of formula 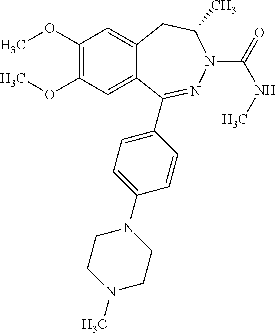 [SMILES: CNC(=O)N1N=C(c2ccc(N3CCN(C)CC3)cc2)c2cc(OC)c(OC)cc2C[C@@H]1C]
((4S)-7,8-dimethoxy-N,4-dimethyl-1-[4-(4-methylpiperazin-1-yl)phenyl]-4,5-dihydro-3H-2,3-benzodiazepine-3-carboxamide).

24. An in vitro method for stratifying melanoma according to claim 1, wherein the inhibitor is [(R,S)-4-(4-Chlorophenyl)-2,3,9-trimethyl-6H-1-thia-5,7,8,9a-tetraaza-cyclopenta[e]azulen-6-yl]-acetic acid tert-butyl ester of formula

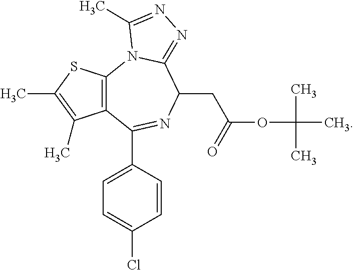 [SMILES: Cc1sc2c(c1C)C(c1ccc(Cl)cc1)=NC(CC(=O)OC(C)(C)C)c1nnc(C)n1-2]

25. An in vitro method according to claim 1, wherein the respective mRNA, or derived cDNA, or protein expression levels indicative of BET responders show at least a 1.5 fold difference relative to BET non-responders

26-28 (canceled)

29. A method for treating melanoma comprising administering a BET inhibitor of claim 9 to a patient in need thereof, wherein a sample of body fluid or tumor tissue of said patient has been stratified in vitro and it has been determined whether said patient suffering from melanoma will respond to treatment with a BET inhibitor, by
i) determining the expression level of the stratification markers PPARGC 1A or MITF by measurement of the respective mRNA or derived cDNA expression levels in a sample of body fluid or tumor tissue of said patient, and comparing the expression level with that of normal human melanocytes,
and/or
ii) determining the protein level of the stratification markers PPARGC 1A or MITF in a melanoma patient in a sample of body fluid or tumor tissue of said patient, and comparing it with that of normal human melanocytes,
and/or
iii) determining the basal OCR in tumor tissue or circulating tumor cells of a patient before and after treatment with a BET inhibitor, and comparing them with untreated and treated normal human melanocytes,
wherein the presence in said in vitro sample of an elevated mRNA, or derived cDNA, and/or protein expression level of PPARGC1A, PPARGC1B and/or MITF, and/or a lowered OCR following treatment with a BET inhibitor is detected, a therapeutically effective amount of a BET inhibitor is administered to the melanoma patient.

30. A method for treating melanoma, comprising administering a BET inhibitor of claim 9 to a patient in need thereof, wherein a sample of body fluid or tumor tissue of said patient has been stratified in vitro and it has been determined whether said patient suffering from melanoma will respond to treatment with a BET inhibitor, by
i) determining the expression level of the stratification markers PPARGC1A measurement of the respective mRNA or derived cDNA expression levels in a sample of body fluid or tumor tissue of said patient, and comparing the expression level with that of normal human melanocytes,
or
ii) determining the protein level of the stratification markers PPARGC1A in a melanoma patient in a sample of body fluid or tumor tissue of said patient, and comparing it with that of normal human melanocytes,
and/or
iii) determining the basal OCR in tumor tissue or circulating tumor cells of a patient before and after treatment with a BET inhibitor, and comparing them with untreated and treated normal human melanocytes,
wherein the presence in said in vitro sample of an elevated mRNA, or derived cDNA, or protein expression level of PPARGC1A and/or a lowered OCR following treatment with a BET inhibitor is detected, a therapeutically effective amount of a BET inhibitor is administered to the melanoma patient.

31. A method for treating melanoma, comprising administering a BET inhibitor of claim 9 to a patient in need thereof, wherein a sample of body fluid or tumor tissue of said patient has been stratified in vitro and it has been determined whether said patient suffering from melanoma will respond to treatment with a BET inhibitor, by
i) determining the expression level of the stratification markers PPARGC1A by measurement of the respective mRNA or derived cDNA expression levels in a sample of body fluid or tumor tissue of said patient, and comparing the expression level with that of normal human melanocytes,
or
ii) determining the protein level of the stratification markers PPARGC1A in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,
wherein the presence in said in vitro sample of an elevated mRNA, or derived cDNA, or protein expression level of PPARGC1A, and a lowered OCR following treatment with a BET inhibitor is detected, a therapeutically effective amount of a BET inhibitor is administered to the melanoma patient.

32-35. (canceled)

36. A kit for in vitro stratification for determining whether a patient suffering from melanoma will respond to treatment with a BET inhibitor of claim 9, comprising the steps of:
i) determining the expression level of the stratification markers PPARGC1A or MITF by measurement of the respective mRNA or derived cDNA expression levels in a sample of body fluid or tumor tissue of said patient, and comparing the expression level with that of normal human melanocytes,
and/or
ii) determining the protein level of the stratification markers PPARGC1A or MITF in a melanoma patient in a sample of body fluid or tumor tissue of said patient, and comparing it with that of normal human melanocytes,
and/or
iii) determining the basal OCR in tumor tissue or circulating tumor cells of a patient before and after treatment with a BET inhibitor, and comparing them with untreated and treated normal human melanocytes,
wherein the presence in said in vitro sample of an elevated mRNA or derived cDNA expression level of PPARGC1A or MITF and/or protein expression level of PPARGC1A or MITF and/or a lowered OCR following treatment with a BET inhibitor in comparison with the untreated sample is suggestive of a better response to the treatment of melanoma in said patient.

37. A kit for in vitro stratification for determining whether a patient suffering from melanoma will respond to treatment with a BET inhibitor of claim 9, comprising the steps of:
i) determining the expression level of the stratification markers PPARGC1A measurement of the respective mRNA or derived cDNA expression levels in a sample of body fluid or tumor tissue of said patient, and comparing the expression level with that of normal human melanocytes,
or
ii) determining the protein level of the stratification markers PPARGC1A in a melanoma patient in a sample of body fluid or tumor tissue of said patient, and comparing it with that of normal human melanocytes,
and/or
iii) determining the basal OCR in tumor tissue or circulating tumor cells of a patient before and after treatment with a BET inhibitor, and comparing them with untreated and treated normal human melanocytes,
wherein the presence in said in vitro sample of an elevated mRNA or derived cDNA expression level of PPARGC1A and/or a lowered OCR following treatment with a BET inhibitor in comparison with the untreated sample is suggestive of a better response to the treatment of melanoma in said patient.

38. A kit for in vitro stratification for determining whether a patient suffering from melanoma will respond to treatment with a BET inhibitor of the claim 9, comprising the steps of:
i) determining the expression level of the stratification markers PPARGC1A by measurement of the respective mRNA or derived cDNA expression levels in a sample of body fluid or tumor tissue of said patient, and comparing the expression level with that of normal human melanocytes,
or
ii) determining the protein level of the stratification markers PPARGC1A in a melanoma patient in a sample of body fluid or tumor tissue of said patient, and comparing it with that of normal human melanocytes,
and
iii) determining the basal OCR in tumor tissue or circulating tumor cells of a patient before and after treatment with a BET inhibitor, and comparing them with untreated and treated normal human melanocytes,
wherein the presence in said in vitro sample of an elevated mRNA or derived cDNA expression level of PPARGC1A or a protein expression level of PPARGC1A and a lowered OCR following treatment with a BET inhibitor in comparison with the untreated sample is suggestive of a better response to the treatment of melanoma in said patient.

39-44. (canceled)
